(19) United States
(12) Patent Application Publication
(10) Pub. No.: US 2026/0131016 A1
(43) Pub. Date: May 14, 2026

(54) NOVEL AURISTATIN ANALOGS AND IMMUNOCONJUGATES THEREOF

(71) Applicant: ADCENTRX THERAPEUTICS INC., San Diego, CA (US)

(72) Inventors: Richard Hui Li, San Diego, CA (US); Dong Jun Lee, San Diego, CA (US)

(21) Appl. No.: 18/704,394

(22) PCT Filed: Nov. 2, 2022
(86) PCT No.: PCT/US2022/048735

§ 371 Date: Apr. 24, 2024

Related U.S. Application Data
(60) Provisional application No. 63/275,177, filed on Nov. 3, 2021.
(60) Provisional application No. 63/295,476, filed on Dec. 30, 2021.

(51) Int. Cl. A61K 47/68; C07K 5/02

(52) U.S. Cl. CPC A61K 47/68031; A61K 47/6851; C07K 5/0205

(57) ABSTRACT

The invention provides novel auristatin analogs and immunoconjugates thereof, as well as pharmaceutical compositions and methods of preparation and use for treating various diseases and disorders (e.g., cancer).

### PRIORITY CLAIMS AND RELATED PATENT APPLICATIONS

This application claims the benefit of priority to U.S. Provisional Application Ser. No. 63/275,177, filed Nov. 3, 2021, and 63/295,476, filed Dec. 30, 2021, the entire content of each of which is incorporated herein by reference.

### TECHNICAL FIELD OF THE INVENTION

The invention generally relates to novel compounds and therapeutic uses thereof. More particularly, the invention provides novel auristatin analogs and immunoconjugates thereof, as well as pharmaceutical compositions and methods of preparation and use for treating various diseases and disorders (e.g., cancer).

### BACKGROUND OF THE INVENTION

Cytotoxic agents, which are commonly employed chemotherapy agents due to their high cytotoxicity, often suffer from rapid plasma clearance and low selectively towards cancer cells. Monoclonal antibody therapies are characterized by high selectivity and long plasma half-lives but often with limited cytotoxicity. Antibody-drug conjugates (ADCs), a class of therapies with high cytotoxicity and long plasma half-lives, represent a promising therapeutic modality in cancer treatment. Eleven ADCs have been approved by the FDA to date, including gemtuzumab ozogamicin (Mylotarg™), the first ADC approved by the FDA in 2000. (See, e.g., Drago et al. 2021 Nature Reviews 18, 327-344; Mckertish et al. 2021 Biomedicines 9, 872; Khongorzui et al. 2020 Molecular Cancer Res. 18:3-19; Bross et al. 2001 Clin. Cancer Res. 7, 1490-1496; Hamann et al. 2002 Bioconjug. Chem. 13, 47-58; Lamb, 2017 Drugs 77, 1603-1610.)

Auristatins are a family of complex analogues to the native antineoplastic product dolastatin 10. These cytotoxic agents are 100 to 1,000 times more toxic than Doxorubicin, a conventional cancer chemotherapy medication.

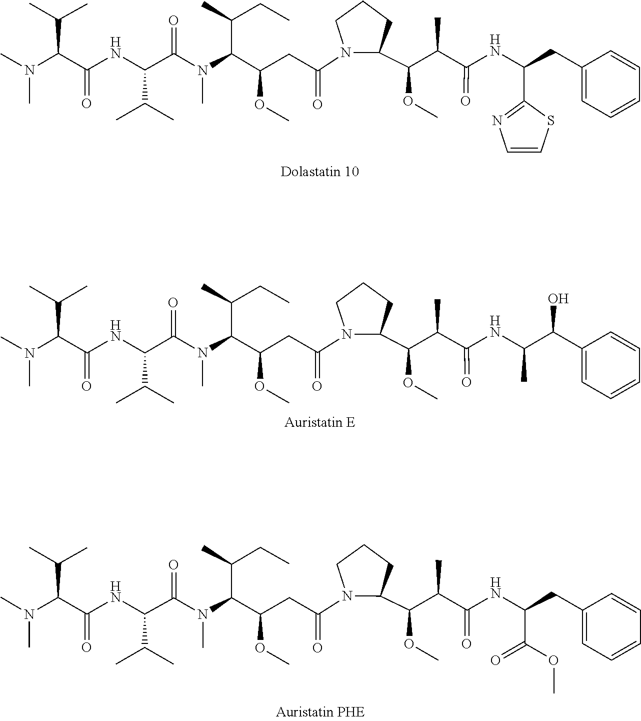 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C)C(C)C ; CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)C)C(C)C]

It is believed that auristatins lead to the arrest of cancer cells in the mitosis stage and eventually apoptosis. Auristatins-based ADCs have been subjects of clinical studies in recent years, some of which have been approved by the FDA, for example, brentuximab vedotin (Adcetris™) first approved in 2011. (See, e.g., McGinn et al. 2012 Clin. Cancer Res. 18, 5845-5849; Deng et al. 2013 Clin. Cancer Res. 19, 22-27; U.S. Pat. No. 6,884,869 B2; U.S. Pat. No. 7,498,298 B2; WO 2015/095301 A2; WO 2015/151079 A2; WO 2015/151081 A2; WO 2016/123412 A1; WO 2011/097627 A1; WO 2001/018032 A2.)

Despite significant progress in clinical development of ADCs in recent years, their design and development involve many challenges including lack of stability, high aggregation propensity and limited bioavailability as well as limited numbers of potent cytotoxic agents that suitable for development.

Novel auristatin analogs that are potent and suitable for development and immunoconjugates based on novel auristatins are highly desired.

### SUMMARY OF THE INVENTION

The invention provides novel auristatin analogs that possess high cytotoxicity and favorable stability and other characteristics making them suitable for use in immunoconjugates. The auristatin analogs disclosed herein are characterized by unique N-substitution at position 5 (P5) position in synergistic combination with a nearby anilino group for conjugation to a linker, with further variations at position 1 (P1) for fine-tuning of the payload to suit different ADC constructs and applications. The high potency, high stability, low immunogenicity, as well as increased permeability and satisfactory solubility render these compounds ideally suited as cytotoxic agents for development of immunoconjugates as novel therapeutics for cancer.

In one aspect, the invention generally relates to a compound having the structural formula (I):

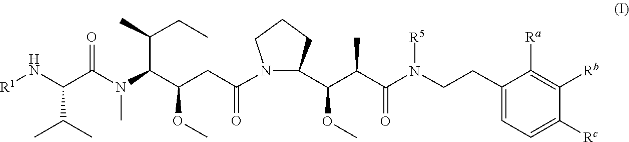

or a pharmaceutically acceptable salt thereof,
wherein
R¹ is

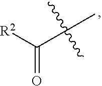

wherein R² is a unsubstituted or substituted C₁-C₆ alkyl, heteroalkyl, cycloalkyl or cycloheteroalkyl;
each of R^{a}, R^{b} and R^{c} is selected from H and NR^{x}R^{y}, provided that only one of R^{a}, R^{b} and R^{c} is NR^{x}R^{y} and each of the others is H;
each of R^{x} and R^{y} is independently selected from R, R^{r} and L-R^{z}, provided that when one of R^{x} and R^{y} is L-R^{z} or R^{r}, the other is R;
R⁵ is CR′₃, wherein each R′ is independently H or F;
L is a linker;
R^{r} is (C≡O)—O—(CH₂)ₚ—R^{v} or (C═O)—(CH₂)_{q}—R^{v};
R^{v} is R, OR, NHR, NR₂, an aryl group or an amino acid;
p is 0, 1, 2, 3, 4, 5 or 6;
q is 0, 1, 2, 3, 4, 5 or 6;
R^{z} comprises a functional or reactive group; and
R is H or a C₁-C₃ alkyl.

In another aspect, the invention generally relates to a composition comprising a compound disclosed herein, such as according to any one of formulae (I)-(V⁶) and in Table 1 herein, or a pharmaceutically acceptable salt thereof, and optionally a pharmaceutically acceptable excipient, carrier or diluent.

In yet another aspect, the invention generally relates to an immunoconjugate having the structural formula (VI):

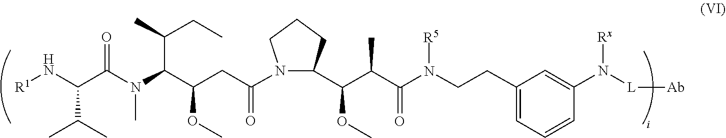

or a pharmaceutically acceptable salt thereof,
wherein
Ab represents an antigen binding moiety;
R¹ is

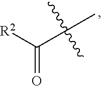

wherein R² is a unsubstituted or substituted C₁-C₆ alkyl, heteroalkyl, cycloalkyl or cycloheteroalkyl;
each of R^{x} and R^{y} is independently selected from R and L-R^{z}, provided that when one of R^{x} and R^{y} is NR^{z}, the other is R;
R⁵ is CR′₃, wherein each R′ is independently H or F;
L is a linker; and
R is H or a C₁-C₃ alkyl; and
i is an integer in the range of 1 to about 20.

In yet another aspect, the invention generally relates to an immunoconjugate having the structural formula (VII):

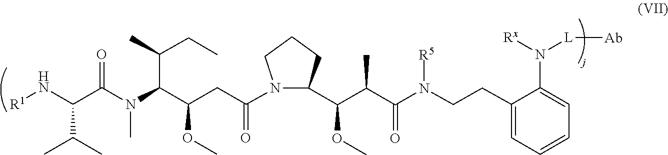

or a pharmaceutically acceptable salt thereof,
wherein
Ab represents an antigen binding moiety;
R¹ is

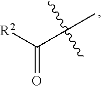

wherein R² is a unsubstituted or substituted C₁-C₆ alkyl, heteroalkyl, cycloalkyl or cycloheteroalkyl;
each of R^{x} and R^{y} is independently selected from R and L-R^{z}, provided that when one of R^{x} and R^{y} is NR^{z}, the other is R;
R⁵ is CR′₃, wherein each R′ is independently H or F;
L is a linker; and
R is H or a C₁-C₃ alkyl; and
j is an integer in the range of 1 to about 20.

In yet another aspect, the invention generally relates to an immunoconjugate having the structural formula (VIII):

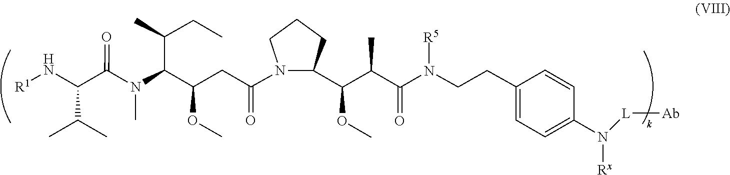

or a pharmaceutically acceptable salt thereof,
wherein
Ab represents an antigen binding moiety;
R¹ is

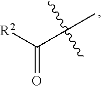

wherein R² is a unsubstituted or substituted C₁-C₆ alkyl, heteroalkyl, cycloalkyl or cycloheteroalkyl;
each of R^{x} and R^{y} is independently selected from R and L-R^{z}, provided that when one of R^{x} and R^{y} is NR^{z}, the other is R;
R⁵ is CR′₃, wherein each R′ is independently H or F;
L is a linker; and
R is H or a C₁-C₃ alkyl; and
k is an integer in the range of 1 to about 20.

In yet another aspect, the invention generally relates to a pharmaceutical composition comprising an immunoconjugate disclosed herein, or a pharmaceutically acceptable salt thereof, and a pharmaceutically acceptable excipient, carrier or diluent.

In yet another aspect, the invention generally relates to a combination comprising a therapeutically effective amount of an immunoconjugate disclosed herein, and one or more therapeutically active co-agent(s) and/or adjuvant(s).

In yet another aspect, the invention generally relates to a method for treating or reducing a disease or condition, comprising administering to a subject in need thereof a therapeutically effective amount of an immunoconjugate disclosed herein.

In yet another aspect, the invention generally relates to use of an immunoconjugate disclosed herein for the manufacture of a medicament.

In yet another aspect, the invention generally relates to use of an immunoconjugate disclosed herein for use in treating a disease or condition (e.g., cancer).

### DETAILED DESCRIPTION OF THE INVENTION

The invention is based in part on the discovery of novel auristatin analogs that possess favorable potency, stability and other profiles as payloads for immunoconjugates. Key structural improvements to existing auristatins include N-methyl substitution at P5 in synergistic combination with a nearby anilino group for conjugation with a linker. These modifications led to an increase in permeability of payloads and enable linker installation through the C-termini. Further fine-tuning of the payload molecule can be achieved through modifications at P1 to suit a wide range of ADC constructs and applications. For example, the anilino group allow ADC constructs that tend to better preserve the potency of the payload. In addition, the highly potent and stabile cytotoxic agents also enjoy satisfactory solubility and low immunogenicity making them suitable for development as immunoconjugates and novel therapeutics for cancer.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

The following terms, unless indicated otherwise according to the context wherein the terms are found, are intended to have the following meanings.

Ranges provided herein are understood to be shorthand for all of the values within the range. For example, a range of 1 to 16 is understood to include any number, combination of numbers, or sub-range from the group consisting 1, 2, 3, 4, 5, 6, 7, 8, 9, 10, 11, 12, 13, 14, 15 or 16.

As used herein, “at least” a specific value is understood to be that value and all values greater than that value.

As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

Any compositions or methods disclosed herein can be combined with one or more of any of the other compositions and methods provided herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Certain compounds of the present invention may exist in particular geometric or stereoisomeric forms. The present invention contemplates all such compounds, including cis- and trans-isomers, atropisomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the R- or S-configuration. For optically active compounds, it is often preferred to use one enantiomer to the substantial exclusion of the other enantiomer.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

A mixture of isomers can be separated on the basis of the physicochemical differences of the constituents, into the pure or substantially pure geometric or optical isomers, diastereomers, racemates, for example, by chromatography and/or fractional crystallization.

Definitions of specific functional groups and chemical terms are described in more detail below. When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Where substituent groups are specified by their conventional chemical formulae, written from left to right, they equally encompass the chemically identical substituents that would result from writing the structure from right to left, e.g., —C(═O)—O— is equivalent to —O—C(═O)—.

Structures of compounds of the invention are limited by principles of chemical bonding known to those skilled in the art. Accordingly, where a group may be substituted by one or more of a number of substituents, such substitutions are selected so as to comply with principles of chemical bonding and to give compounds that are not inherently unstable and/or would be known to one of ordinary skill in the art as likely to be unstable under ambient conditions (e.g., aqueous, neutral, and several known physiological conditions).

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more of substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

As used herein, the term “alkoxy” refers to the group —O-alkyl, including from 1 to 10 carbon atoms (C₁₋₁₀) of a straight, branched, saturated cyclic configuration and combinations thereof, attached to the parent molecular structure through an oxygen. Examples include methoxy, ethoxy, propoxy, isopropoxy, butoxy, t-butoxy, pentoxy, cyclopropyloxy, cyclohexyloxy and the like. “Lower alkoxy” refers to alkoxy groups containing one to six carbons. In some embodiments, C₁₋₃ alkoxy is an alkoxy group that encompasses both straight and branched chain alkyls of from 1 to 3 carbon atoms. Unless stated otherwise in the specification, an alkoxy group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein.

As used herein, the terms “aromatic” or “aryl” refer to a radical with 6 to 14 ring atoms (e.g., C₆₋₁₄ aromatic or C₆₋₁₄ aryl) that has at least one ring having a conjugated pi electron system which is carbocyclic (e.g., phenyl, fluorenyl, and naphthyl). In some embodiments, the aryl is a C₆₋₁₀ aryl group. For example, bivalent radicals formed from substituted benzene derivatives and having the free valences at ring atoms are named as substituted phenylene radicals. In other embodiments, bivalent radicals derived from univalent polycyclic hydrocarbon radicals whose names end in“-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 14 aryl” refers to each integer in the given range; e.g., “6 to 14 ring atoms” means that the aryl group can consist of 6 ring atoms, 7 ring atoms, etc., up to and including 14 ring atoms. The term includes monocyclic or fused-ring polycyclic (i.e., rings which share adjacent pairs of ring atoms) groups. Polycyclic aryl groups include bicycles, tricycles, tetracycles, and the like. In a multi-ring group, only one ring is required to be aromatic, so groups such as indanyl are encompassed by the aryl definition. Non-limiting examples of aryl groups include phenyl, phenalenyl, naphthalenyl, tetrahydronaphthyl, phenanthrenyl, anthracenyl, fluorenyl, indolyl, indanyl, and the like. Unless stated otherwise in the specification, an aryl moiety can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein.

As used herein, the terms “cycloalkyl” and “carbocyclyl” each refers to a monocyclic or polycyclic radical that contains only carbon and hydrogen, and can be saturated or partially unsaturated. Unless stated otherwise in the specification, the term is intended to include both substituted and unsubstituted cycloalkyl groups. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₋₁₃ cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl (C₇), cycloheptadienyl (C₇), cycloheptatrienyl (C₇), cyclooctyl (C₈), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)N(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

As used herein, the term “heterocycloalkyl” refers to a cycloalkyl radical, which have one or more skeletal chain atoms selected from an atom other than carbon, e.g., O, N, S, P or combinations thereof. Unless stated otherwise in the specification, the term is intended to include both substituted and unsubstituted heterocycloalkyl groups. Illustrative examples of heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, 1-cyclopropyl-4-methyl-piperazin-2-yl. 2-pyrrolinyl, 3-pyrrolinyl, dihydro-2H-pyranyl, 1,2,3,4-tetrahydropyridine, 3,4-dihydro-2H-[1,4]oxazine, etc.

As used herein, the term “halogen” refers to fluorine (F), chlorine (Cl), bromine (Br), or iodine (I). As used herein, the term “halide” or “halo”, means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine, such as, but not limited to, trifluoromethyl, difluoromethyl, 2,2,2-trifluoroethyl, 1-fluoromethyl-2-fluoroethyl, and the like. Each of the alkyl, alkenyl, alkynyl and alkoxy groups are as defined herein and can be optionally further substituted as defined herein.

As used herein, the term “heteroatom” refers to oxygen (O), nitrogen (N), sulfur (S), and phosphorus (P).

As used herein, the term “heteroalkyl” refers to an alkyl radical, which have one or more skeletal chain atoms selected from an atom other than carbon, e.g., oxygen, nitrogen, sulfur, phosphorus or combinations thereof. A numerical range can be given, e.g., C₁₋₄ heteroalkyl, which refers to the chain length in total, which in this example is 4 atoms long. For example, a —CH₂OCH₂CH₃ radical is referred to as a “C₄” heteroalkyl, which includes the heteroatom center in the atom chain length description. Connection to the parent molecular structure can be through either a heteroatom or a carbon in the heteroalkyl chain. For example, an N-containing heteroalkyl moiety refers to a group in which at least one of the skeletal atoms is a nitrogen atom. One or more heteroatom(s) in the heteroalkyl radical can be optionally oxidized. One or more nitrogen atoms, if present, can also be optionally quaternized. For example, heteroalkyl also includes skeletal chains substituted with one or more nitrogen oxide (—O—) substituents. Exemplary heteroalkyl groups include, without limitation, ethers such as methoxyethanyl (—CH₂CH₂OCH₃), ethoxymethanyl (—CH₂OCH₂CH₃), (methoxymethoxy)ethanyl (—CH₂CH₂OCH₂OCH₃), (methoxymethoxy) methanyl (—CH₂OCH₂OCH₃) and (methoxyethoxy)methanyl (—CH₂OCH₂CH₂OCH₃) and the like; amines such as (—CH₂CH₂NHCH₃, —CH₂CH₂N(CH₃)₂, —CH₂NHCH₂CH₃, —CH₂N(CH₂CH₃)(CH₃)) and the like.

As used herein, the term “heteroaryl” or, alternatively, “heteroaromatic” refers to a refers to a radical of a 5-18 membered monocyclic or polycyclic (e.g., bicyclic, tricyclic, tetracyclic and the like) aromatic ring system (e.g., having 6, 10 or 14 π electrons shared in a cyclic array) having ring carbon atoms and 1-6 ring heteroatoms provided in the aromatic ring system, wherein each heteroatom is independently selected from nitrogen, oxygen, phosphorous and sulfur (“5-18 membered heteroaryl”). Heteroaryl polycyclic ring systems can include one or more heteroatoms in one or both rings. Whenever it appears herein, a numerical range such as “5 to 18” refers to each integer in the given range; e.g., “5 to 18 ring atoms” means that the heteroaryl group can consist of 5 ring atoms, 6 ring atoms, etc., up to and including 18 ring atoms. In some instances, a heteroaryl can have 5 to 14 ring atoms. In some embodiments, the heteroaryl has, for example, bivalent radicals derived from univalent heteroaryl radicals whose names end in “-yl” by removal of one hydrogen atom from the atom with the free valence are named by adding “-ene” to the name of the corresponding univalent radical, e.g., a pyridyl group with two points of attachment is a pyridylene.

For example, an N-containing “heteroaromatic” or “heteroaryl” moiety refers to an aromatic group in which at least one of the skeletal atoms of the ring is a nitrogen atom. One or more heteroatom(s) in the heteroaryl radical can be optionally oxidized. One or more nitrogen atoms, if present, can also be optionally quaternized. Heteroaryl also includes ring systems substituted with one or more nitrogen oxide (—O—) substituents, such as pyridinyl N-oxides. The heteroaryl is attached to the parent molecular structure through any atom of the ring(s).

“Heteroaryl” also includes ring systems wherein the heteroaryl ring, as defined above, is fused with one or more aryl groups wherein the point of attachment to the parent molecular structure is either on the aryl or on the heteroaryl ring, or wherein the heteroaryl ring, as defined above, is fused with one or more cycloalkyl or heterocyclyl groups wherein the point of attachment to the parent molecular structure is on the heteroaryl ring. For polycyclic heteroaryl groups wherein one ring does not contain a heteroatom (e.g., indolyl, quinolinyl, carbazolyl and the like), the point of attachment to the parent molecular structure can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, phosphorous, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, phosphorous, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, phosphorous, and sulfur (“5-6 membered heteroaryl”). In some embodiments, the 5-6 membered heteroaryl has 1-3 ring heteroatoms selected from nitrogen, oxygen, phosphorous, and sulfur. In some embodiments, the 5-6 membered heteroaryl has 1-2 ring heteroatoms selected from nitrogen, oxygen, phosphorous, and sulfur. In some embodiments, the 5-6 membered heteroaryl has 1 ring heteroatom selected from nitrogen, oxygen, phosphorous, and sulfur.

Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzopyranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno [2,3-d]pyrimidinyl, 5,6-dihydrobenzo[h]quinazolinyl, 5,6-dihydrobenzo[h]cinnolinyl, 6,7-dihydro-5H benzo[6,7]cyclohepta[1,2-c]pyridazinyl, dibenzofuranyl, dibenzothiophenyl, furanyl, furazanyl, furanonyl, furo [3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo [4,5]thieno [2,3-d]pyrimdinyl, 6,7,8,9-tetrahydro-5H-cyclohepta[4,5]thieno [2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno [2,3-c]pridinyl, and thiophenyl (i.e., thienyl). Unless stated otherwise in the specification, a heteroaryl moiety can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein.

As used herein, the terms “administer” and “administering” refer to oral administration, administration as a suppository, topical contact, intravenous, parenteral, intraperitoneal, intramuscular, intralesional, intrathecal, intracranial, inhalation, intraocular, intranasal or subcutaneous administration, or the implantation of a slow-release device, e.g., a mini-osmotic pump, to a subject. Suitable routes of administration for a particular patient will depend on the nature and severity of the disease or condition being treated or the nature of the therapy being used and on the nature of the active compound.

Administration may be by any suitable route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the term “co-administer” refers to the presence of two pharmacological agents in the blood at the same time. The two pharmacological agents can be administered concurrently or sequentially.

As used herein, the term “affinity” refers to the strength of interaction between an antigen binding moiety (e.g., antibody) and antigen at single antigenic sites.

As used herein, the term “agonist” refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor.

As used herein, the term “antagonist” refers to a compound that competes with an agonist or inverse agonist for binding to a receptor, thereby blocking the action of an agonist or inverse agonist on the receptor. However, an antagonist has no effect on constitutive receptor activity.

As used herein, the term “amino acid” refers to a molecule of the general formula NH₂—CHR—COOH, wherein “R” is one of a number of different side chains, or a residue within a peptide bearing the parent amino acid. Amino acids include naturally occurring amino acids with “R” being a substituent found in naturally occurring amino acids. “R” can also be a substituent that is not found in naturally occurring amino acids. The term “amino acid residue” refers to the portion of the amino acid which remains after losing a water molecule when it is joined to another amino acid. The term “modified amino acid” refers to an amino acid bearing an “R” substituent that does not correspond to one of the twenty genetically coded amino acids.

As used herein, the term “antigen” as used herein is meant any substance that causes the immune system to produce antibodies or specific cell-mediated immune responses against it. A disease associated antigen is any substance that is associated with any disease that causes the immune system to produce antibodies or a specific-cell mediated response against it. An antigen is capable of being recognized by the immune system and/or being capable of inducing a humoral immune response and/or cellular immune response leading to the activation of B- and/or T-lymphocytes. An antigen can have one or more epitopes (B- and/or T-cell epitopes). An antigen will preferably react, typically in a highly selective manner, with its corresponding antibody or TCR and not with the multitude of other antibodies or TCRs which may be evoked by other antigens. Antigens as used herein may also be mixtures of several individual antigens.

As used herein, the term “antigen binding moiety” refers to a moiety capable of binding specifically to an antigen, and includes but is not limited to antibodies and antibody fragments, peptides and small molecule ligands.

As used herein, the term “antibody” refers to molecules that are capable of binding an epitope or antigenic determinant. The term is meant to include whole antibodies and antigen-binding fragments thereof. The term encompasses polyclonal, monoclonal, chimeric, Fabs, Fvs, single-chain antibodies and single or multiple immunoglobulin variable chain or CDR domain designs as well as bispecific and multispecific antibodies. Antibodies can be from any animal origin. Preferably, the antibodies are mammalian, e.g., human, murine, rabbit, goat, guinea pig, camel, horse and the like, or other suitable animals. Antibodies may recognize polypeptide or polynucleotide antigens. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid antibody molecules, F(ab)₂ and F(ab) fragments; Fv molecules (for example, noncovalent heterodimers), dimeric and trimeric antibody fragment constructs; minibodies, humanized antibody molecules, and any functional fragments obtained from such molecules, wherein such fragments retain specific binding.

As used herein, the term “antigen binding fragment” refers to one or more portions of an antibody that retain the ability to specifically interact with, e.g., by binding, steric hindrance, stabilizing/destabilizing, spatial distribution, an epitope of an antigen.

Examples of binding fragments include, but are not limited to, single-chain Fvs (scFv), disulfide-linked Fvs (sdFv), Fab fragments, F(ab′) fragments, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al. 1989 Nature 341:544-546,), which consists of a V_{H} domain; and an isolated complementarity determining region (CDR), or other epitope-binding fragments of an antibody.

Additionally, the two domains of the Fv fragment, V_{L} and V_{H} can be joined using recombinant methods by a synthetic linker that enables them to be made as a single protein chain in which the V_{L} and V_{H} regions pair to form monovalent molecules. (known as single chain Fv (“scFv”); see, e.g., Bird et al., 1988 Science 242:423-426; and Huston et al. 1988 Proc. Natl. Acad. Sci. 85:5879-5883.) Such single chain antibodies are also intended to be encompassed within the term “antigen binding fragment.” These antigen binding fragments are obtained using conventional techniques known to those of skill in the art, and the fragments are screened for utility in the same manner as are intact antibodies.

Antigen binding fragments can also be incorporated into single domain antibodies, maxibodies, minibodies, nanobodies, intrabodies, diabodies, triabodies, tetrabodies, v-NAR and bis-scFv. (See, e.g., Hollinger and Hudson, 2005 Nature Biotechnology 23:1 126-1136.) Antigen binding fragments can be grafted into scaffolds based on polypeptides such as fibronectin type III (Fn3). (See, e.g., U.S. Pat. No. 6,703,199, which describes fibronectin polypeptide monobodies.) Antigen binding fragments can be incorporated into single chain molecules comprising a pair of tandem Fv segments (V_{H}-C_{H}1-V_{H}-C_{H}1) which, together with complementary light chain polypeptides, form a pair of antigen binding regions. (Zapata et al., 1995 Protein Eng. 8:1057-1062; U.S. Pat. No. 5,641,870.)

As used herein, the term “bispecific antibody” or “bispecific” refers to an antibody, typically a monoclonal antibody, having binding specificities for at least two different antigenic epitopes. The epitopes can be from the same antigen or from two different antigens. Methods for making bispecific antibodies are known in the art. For example, bispecific antibodies can be produced recombinantly using the co-expression of two immunoglobulin heavy chain/light chain pairs. Alternatively, bispecific antibodies can be prepared using chemical linkage. Bispecific antibodies include bispecific antibody fragments. (See, e.g., Milstein et al. 1983 Nature 305:537-39; Brennan et al. 1985 Science 229:81; Hollinger et al. 1994 Proc. Natl. Acad. Sci. U.S.A. 90:6444-48; Gruber et al. 1994 J. Immunol. 152:5368-74.)

As used herein, the term “chimeric antibody” or “chimeric” refers to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity.

As used herein, the term “human antibody” refers to antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al. 2000 J. Mol. Biol. 296:57-86). Human antibodies may include amino acid residues not encoded by human sequences, e.g., mutations introduced by random or site-specific mutagenesis in vitro or by somatic mutation in vivo, or a substitution to promote stability or manufacturing.

As used herein, the term “humanized antibody” refers to antibodies that contain sequences from non-human (e.g., murine) antibodies as well as human antibodies. Such antibodies are chimeric antibodies which contain minimal sequence derived from non-human immunoglobulin. In general, humanized antibodies comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also comprises at least a portion of an immunoglobulin constant region (Fc), typically that of a human immunoglobulin. (See e.g., Cabilly U.S. Pat. No. 4,816,567; Queen et al. 1989 Proc. Nat′l Acad. Sci. USA 86:10029-10033; ANTIBODY ENGINEERING: A PRACTICAL APPROACH, Oxford University Press 1996.)

As used herein, the term “monoclonal antibody”, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. “Monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by various methods known in the art, including the hybridoma method first described by Kohler et al. 1975 Nature 256: 495, or may be made by recombinant DNA methods (see, e.g., U.S. Pat. No. 4,816,567). “Monoclonal antibodies” may also be isolated from phage antibody libraries using the techniques described in Clackson et al. 1991 Nature 352: 624-628 and Marks et al. 1991 J. Mol. Biol. 222: 581-597, for example. These monoclonal antibodies will usually bind with at least a Kd of about 1 M, more usually at least about 300 nM, typically at least about 30 nM, preferably at least about 10 nM.

As used herein, the term “biologically active” entity, or an entity having “biological activity,” is one having structural, regulatory, or biochemical functions of a naturally occurring molecule or any function related to or associated with a metabolic or physiological process. A biologically active polypeptide or fragment thereof includes one that can participate in a biological process or reaction and/or can produce a desired effect. The biological activity can include an improved desired activity, or a decreased undesirable activity. For example, an entity demonstrates biological activity when it participates in a molecular interaction with another molecule, when it has therapeutic value in alleviating a disease condition, when it has prophylactic value in inducing an immune response, or when it has diagnostic and/or prognostic value in determining the presence of a molecule. A biologically active protein or polypeptide can be naturally-occurring or it can be synthesized from known components, e.g., by recombinant or chemical synthesis and can include heterologous components.

As used herein, the terms “cancer” and “cancerous” refer to or describe the physiological condition in mammals that is typically characterized by unregulated cell growth. Examples of cancer include but are not limited to, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer.

As used herein, the term “cleavable” linker refers to a linker or linker component that connects two moieties by covalent connections, but breaks down to sever the covalent connection between the moieties under physiologically relevant conditions. Typically, a cleavable linker is severed in vivo more rapidly in an intracellular environment than when outside a cell, causing release of a payload to preferentially occur inside the targeted cell. Cleavage may be enzymatic or non-enzymatic. A payload is typically released from an antibody without degrading the antibody. Cleavage may leave some portion of a linker or linker component attached to the payload, or it may release the payload without any residual part or component of the linker (i.e., traceless release).

As used herein, the term “non-cleavable” linker refers to a linker or linker component that is not especially susceptible to breaking down under physiological conditions, i.e., it is at least as stable as the antibody or antigen binding fragment portion of the immunoconjugate. Such linkers are sometimes referred to as “stable,” meaning they are sufficiently resistant to degradation to keep the payload connected to the antigen binding moiety until the antigen binding moiety is itself at least partially degraded. In such a case, the degradation of Ab precedes cleavage of the linker in vivo. Degradation of the antibody portion of an immunoconjugate having a stable or non-cleavable linker may leave some or all of the linker, and one or more amino acid groups from an antibody, attached to the payload or drug moiety that is delivered in vivo.

As used herein, the term “cell” refers to any prokaryotic, eukaryotic, primary cell or immortalized cell line, any group of such cells as in, a tissue or an organ. Preferably the cells are of mammalian (e.g., human) origin and can be infected by one or more pathogens.

The terms “cytotoxic agent” and “payload” are used interchangeably herein and refer to a compound or substance that inhibits or prevents or stops the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof.

As used herein, the terms “disease”, “condition” or “disorder” are used interchangeably herein and refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

As used here, the term “homology” or “homologous” refers to a sequence similarity between two polypeptides or between two polynucleotides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position. A degree of similarity between sequences is a function of the number of matching or homologous positions shared by the sequences. The alignment of two sequences to determine their percent sequence similarity can be done using software programs known in the art, such as, for example, those described in Ausubel et al. 1999 Current Protocols in Molecular Biology, John Wiley and Sons, Baltimore, MD. The term “homologs” of to a given amino acid sequence or a nucleic acid sequence is intended to indicate that the corresponding sequences of the “homologs” having substantial identity or homology to the given amino acid sequence or nucleic acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

An example of algorithm that is suitable for determining percent sequence identity and sequence similarity are the BLAST algorithms, which are described in Altschul et al. 1977 Nuc. Acids Res. 25:3389-3402 and Altschul et al. 1990 J Mol. Biol. 215:403-410, respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, gels, for example, water or water/propylene glycol solutions.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, 1995 J. Biomater Sci. Polym. Ed. 7:623-645; as biodegradable and injectable gel formulations (see, e.g., Gao 1995 Pharm. Res. 12:857-863); or, as microspheres for oral administration (see, e.g., Eyles 1997 J Pharm. Pharmacol. 49:669-674).

As used herein, the term “in need of” a treatment refers to a subject that would benefit biologically, medically or in quality of life from such a treatment.

As used herein, the term “specifically binds” or “selectively binds,” when used in the context of describing the interaction between an antigen (e.g., a protein or a glycan) and an antibody, antibody fragment, or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under certain designated immunoassay conditions, the antibodies or binding agents with a particular binding specificity bind to a particular antigen at least two (2) times the background and do not substantially bind in a significant amount to other antigens present in the sample. In embodiments, under designated immunoassay conditions, the antibody or binding agents with a particular binding specificity bind to a particular antigen at least ten (10) times the background and do not substantially bind in a significant amount to other antigens present in the sample. Specific binding to an antibody or binding agent under such conditions may require the antibody or agent to have been selected for its specificity for a particular protein. As desired or appropriate, this selection may be achieved by subtracting out antibodies that cross-react with molecules from other species (e.g., mouse or rat) or other subtypes. Alternatively, in some embodiments, antibodies or antibody fragments are selected that cross-react with certain desired molecules.

A variety of immunoassay formats may be used to select antibodies specifically immunoreactive with a particular protein. For example, solid-phase ELISA immunoassays are routinely used to select antibodies specifically immunoreactive with a protein. (See, e.g., Harlow & Lane, Using Antibodies, A Laboratory Manual (1998), for a description of immunoassay formats and conditions that can be used to determine specific immunoreactivity.) Typically, a specific or selective binding reaction will produce a signal at least twice over the background signal and more typically at least than 10 to 100 times over the background.

As used herein, the term “therapeutically effective amount” refers to the dose of a therapeutic agent or agents sufficient to achieve the intended therapeutic effect with minimal or no undesirable side effects. A therapeutically effective amount can be readily determined by a skilled physician, e.g., by first administering a low dose of the pharmacological agent(s) and then incrementally increasing the dose until the desired therapeutic effect is achieved with minimal or no undesirable side effects.

The terms “immunoconjugate” and “antibody-drug-conjugate” are used interchangeably herein and refer to a compound with a linkage of an antigen binding moiety (e.g., an antibody or an antigen binding fragment thereof, a peptide or a small molecule ligand) with a cytotoxic agent or payload. The linkage can be covalent bonds or non-covalent interactions and can include chelation. Thus, the terms “immunoconjugate” and “antibody-drug-conjugate” include peptide-drug-conjugates and small molecule-drug-conjugates.” Various linkers and linking strategies are known in the art and can be employed in order to form an immunoconjugate.

As used herein, the terms “inhibition,” “inhibit” and “inhibiting” and the like in reference to a biological target inhibitor interaction refers to negatively affecting (e.g., decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high-performance liquid chromatography. The term “isolated antibody” refers to an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals.

As used herein, the term “modulate” refers to the production, either directly or indirectly, of an increase or a decrease, a stimulation, inhibition, interference, or blockage in a measured activity when compared to a suitable control. A “modulator” of a polypeptide or polynucleotide refers to a substance that affects, for example, increases, decreases, stimulates, inhibits, interferes with, or blocks a measured activity of the polypeptide or polynucleotide, when compared to a suitable control. For example, a “modulator” may bind to and/or activate or inhibit the target with measurable affinity, or directly or indirectly affect the normal regulation of a receptor activity.

As used herein, a “pharmaceutically acceptable form” of a disclosed compound includes, but is not limited to, pharmaceutically acceptable salts, esters, hydrates, solvates, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchlorate acid or with organic acids such as acetic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, p-toluenesulfonate, undecanoate, valerate salts, and the like. In some embodiments, organic acids from which salts can be derived include, for example, acetic acid, propionic acid, glycolic acid, pyruvic acid, lactic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate.” Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism. (See, e.g., Bundgard, H. 1985 Design of Prodrugs, pp. 7-9, 21-24, Elsevier, Amsterdam; Higuchi et al. 1987 “Pro-drugs as Novel Delivery Systems” A.C.S. Symposium Series, Vol. 14, and in Bioreversible Carriers in Drug Design, ed. Edward B. Roche, American Pharmaceutical Association and Pergamon Press, 1987.

Prodrug forms often offer advantages of solubility, tissue compatibility, or delayed release in the mammalian organism. (See, e.g., Bundgard, Design of Prodrugs, pp. 7-9,21-24, Elsevier, Amsterdam 1985 and Silverman, The Organic Chemistry of Drug Design and Drug Action, pp. 352-401, Academic Press, San Diego, Calif., 1992.) Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Other prodrug derivatives may be combined with other features disclosed herein to enhance bioavailability. As such, those of skill in the art will appreciate that certain of the presently disclosed compounds having free amino, amido, hydroxy or carboxylic groups can be converted into prodrugs. Prodrugs include compounds having a carbonate, carbamate, amide or alkyl ester moiety covalently bonded to any of the above substituents disclosed herein.

Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the terms “protein” and “polypeptide” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, a polypeptide may refer to a protein which includes modifications, such as deletions, additions, and substitutions (generally conservative in nature), to the native sequence, as long as the protein maintains the desired activity. These modifications may be deliberate or may be accidental. Amino acids can be referred to herein by either their commonly known three letter symbols or by the one-letter symbols recommended by the IUPAC-IUB Biochemical Nomenclature Commission.

As used herein, the term “receptor” refers to proteins, including glycoproteins or fragments thereof, capable of interacting with another molecule, called the ligand. The ligand is usually an extracellular molecule which, upon binding to the receptor, usually initiates a cellular response, such as initiation of a signal transduction pathway. The receptor need not necessarily be a membrane-bound protein. The ligand may belong to any class of biochemical or chemical compounds.

As used herein, the term “sample” refers to a sample from a human, animal, or to a research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

As used herein, the terms “stimulate” or “stimulating” refer to increase, to amplify, to augment, to boost a physiological activity, e.g., an immune response. Stimulation can be a positive alteration. For example, an increase can be by 5%, 10%, 25%, 50%, 75%, or even 90-100%. Other exemplary increases include 2-fold, 5-fold, 10-fold, 20-fold, 40-fold, or even 100-fold.

As used herein, the term “subject” refers to any animal (e.g., a mammal), including, but not limited to humans, non-human primates, rodents, and the like, which is to be the recipient of a particular treatment. A subject to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), rodents (e.g., rats and/or mice), etc. In certain embodiments, the non-human animal is a mammal. The non-human animal may be a male or female at any stage of development. A non-human animal may be a transgenic animal. Typically, the terms “subject” and “patient” are used interchangeably herein in reference to a human subject.

As used herein, the terms “suppress” or “suppressing” refer to decrease, to attenuate, to diminish, to arrest, or to stabilize a physiological activity, e.g., an immune response. Suppression can be a negative alteration. For example, a decrease can be by 5%, 10%, 25%, 50%, 75%, or even 90-100%. Exemplary decreases include 2-fold, 5-fold, 10-fold, 20-fold, 40-fold, or even 100-fold.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. Treating or treatment thus refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, for example, the results of a physical examination, neuropsychiatric exams, and/or a psychiatric evaluation. As compared with an equivalent untreated control, such reduction or degree of amelioration may be at least 5%, 10%, 20%, 40%, 50%, 60%, 80%, 90%, 95%, or 100% as measured by any standard technique.

Treatment methods include administering to a subject a therapeutically effective amount of a compound described herein. The administering step may be a single administration or may include a series of administrations. The length of the treatment period depends on a variety of factors, such as the severity of the condition, the patient's age, the concentration of the compound, the activity of the compositions used in the treatment, or a combination thereof. It will also be appreciated that the effective dosage of an agent used for the treatment may increase or decrease over the course of a particular treatment regime. Changes in dosage may result and become apparent by standard diagnostic assays known in the art. In some instances, chronic administration may be required. For example, the compositions are administered to the subject in an amount and for a duration sufficient to treat the patient.

### Auristatin Analogs and Cytotoxins

Various novel auristatin analogs and cytotoxic agents are disclosed herein.

In one aspect, the invention generally relates to a compound having the structural formula (I):

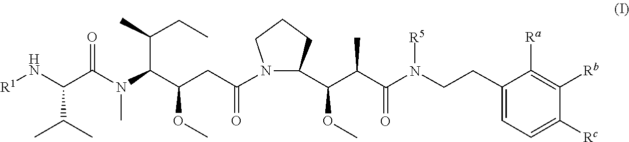

or a pharmaceutically acceptable salt thereof,
wherein
R¹ is

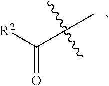

wherein R² is a unsubstituted or substituted C₁-C₆ alkyl, heteroalkyl, cycloalkyl or cycloheteroalkyl;
each of R^{a}, R^{b} and R^{c} is selected from H and NR^{x}R^{y}, provided that only one of R^{a}, R^{b} and R^{c} is NR^{x}R^{y} and each of the others is H;
each of R^{x} and R^{y} is independently selected from R, R^{r} and L-R^{z}, provided that when one of R^{x} and R^{y} is L-R^{z} or R^{r}, the other is R;
R⁵ is CR′₃, wherein each R′ is independently H or F;
L is a linker;
R is (C═O)—O—(CH₂)ₚ—R^{v} or (C═O)—(CH₂)_{q}—R^{v};
R is R, OR, NHR, NR₂, an aryl group or an amino acid;
p is 0, 1, 2, 3, 4, 5 or 6;
q is 0, 1, 2, 3, 4, 5 or 6;
R^{z} comprises a functional or reactive group; and
R is H or a C₁-C₃ alkyl.

In certain embodiments, R⁵ is CH₃. In certain embodiments, R⁵ is CF₃. In certain embodiments, R⁵ is CHF₂. In certain embodiments, R⁵ is CH₂F.

In certain embodiments, R^{a} is NR^{x}R^{y}, R^{b} is H and R^{c} is H. In certain embodiments, R^{a} is H, R^{b} is NR^{x}R^{y} and R^{c} is H. In certain embodiments, R^{a} is H, R^{b} is H and R^{c} is NR^{x}R^{y}.

In certain embodiments, R⁵ is CH₃ while R^{a} is NR^{x}R^{y}, R^{b} is H and R^{c} is H. In certain embodiments, R⁵ is CH₃ while R^{a} is H, R^{b} is H and R^{c} is NR^{x}R^{y}. In certain embodiments, R⁵ is CH₃ while R^{a} is H, R^{b} is H and R^{c} is NR^{x}R^{y}.

In certain embodiments, R⁵ is CF₃ while R^{a} is NR^{x}R^{y}, R^{b} is H and R^{c} is H. In certain embodiments, R⁵ is CF₃ while R^{a} is H, R^{b} is H and R^{c} is NR^{x}R^{y}. In certain embodiments, R⁵ is CF₃ while R^{a} is H, R^{b} is H and R^{c} is NR^{x}R^{y}.

In certain embodiments, R⁵ is CH₃ and R^{c} is H, having the structural formula (II):

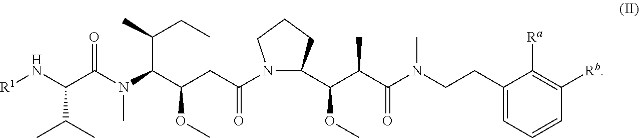

In certain embodiments of (II), R^{a} is H and R^{b} is NR^{x}R^{y}, and the compound has the structural formula (III):

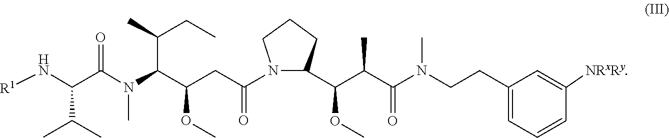

In certain embodiments of (III), R^{x} is H and R^{y} is H, and the compound has the structural formula (III¹):

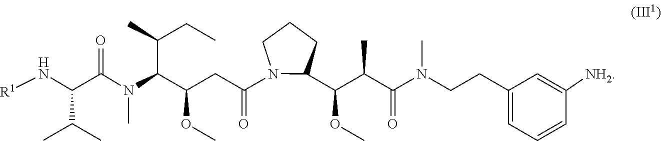

In certain embodiments of (III), R^{x} is H or CH₃ and R^{y} is (C═O)—O—(CH₂)ₚ—R^{v}, wherein R^{v} is R, OR, NHR, NR₂, an aryl group or an amino acid; and p is 0, 1, 2 or 3.

In certain embodiments of (III), R^{x} is H or CH₃ and R^{y} is (C═O)—(CH₂)_{q}—R^{v}, wherein R^{v} is R, OR, NHR, NR₂, an aryl group or an amino acid; and q is 0, 1, 2 or 3.

In certain embodiments of (III), R^{y} is L-R^{z}, and the compound has the structural formula (III²):

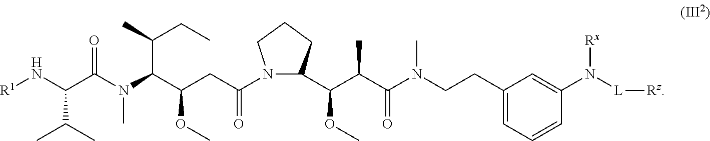

In certain embodiments of (III²), R^{x} is H, and the compound has the structural formula (III³):

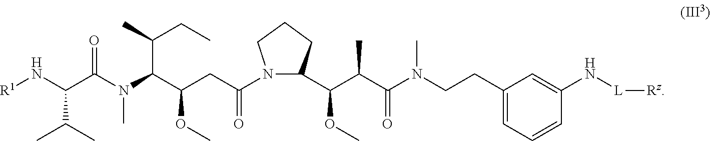

In certain embodiments of (II), R^{a} is NR^{x}R^{y} and R^{b} is H, and the compound has the structural formula (IV):

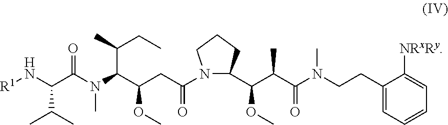

In certain embodiments of (IV), R^{x} is H and R^{y} is H, and the compound has the structural formula (IV¹):

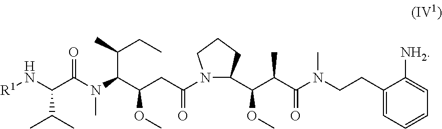

In certain embodiments of (IV), R^{x} is H or CH₃ and R^{y} is (C═O)—O—(CH₂)ₚ—R^{v}, wherein R^{v} is R, OR, NHR, NR₂, an aryl group or an amino acid; and p is 0, 1, 2 or 3.

In certain embodiments of (IV), R^{x} is H or CH₃ and R^{y} is (C═O)—(CH₂)_{q}—R^{v}, wherein R^{v} is R, OR, NHR, NR₂, an aryl group or an amino acid; and q is 0, 1, 2 or 3.

In certain embodiments of (IV), R^{y} is L-R^{z}, and the compound has the structural formula (IV²):

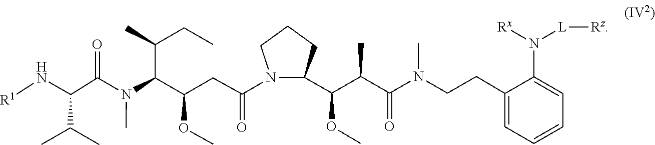

In certain embodiments of (IV²), R^{x} is H, and the compound has the structural formula (IV³):

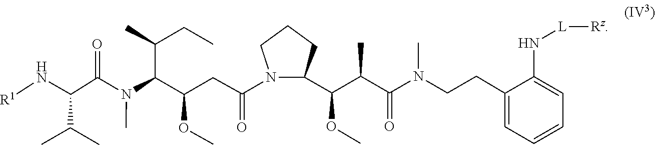

In certain embodiments of (I), R⁵ is CH₃, R^{a} is H, R^{b} is H, and R^{c} is NR^{x}R^{y}, having the structural formula (V):

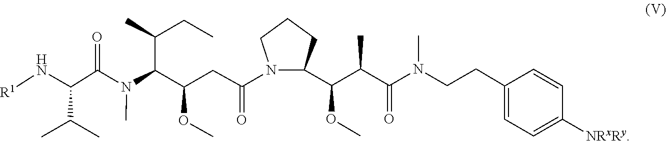

In certain embodiments of (V), R^{x} is H and R^{y} is H, having the structural formula (V¹):

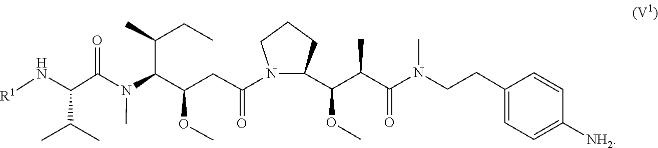

In certain embodiments of (V), R^{x} is H or CH₃ and R^{y} is (C═O)—O—(CH₂)ₚ—R^{v}, wherein R^{v} is R, OR, NHR, NR₂, an aryl group or an amino acid, and p is 0, 1, 2 or 3.

In certain embodiments of (V), R^{x} is H or CH₃ and R^{y} is (C═O)—(CH₂)_{q}—R^{v}, wherein R^{v} is R, OR, NHR, NR₂, an aryl group or an amino acid, and q is 0, 1, 2 or 3.

In certain embodiments of (V), R^{y} is L-R^{z}, having the structural formula (V²):

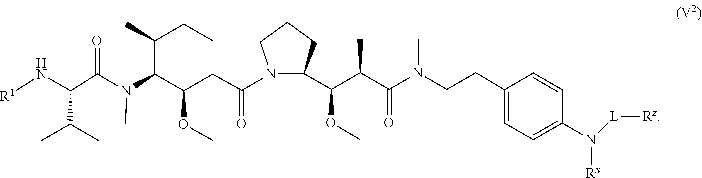

In certain embodiments of (V²), R^{x} is H, having the structural formula (V³):

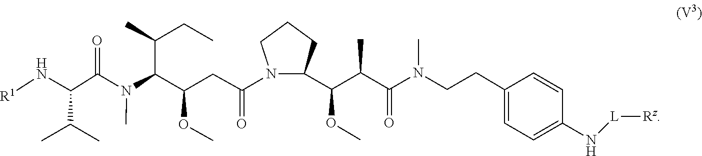

In certain embodiments of (I), R⁵ is CF₃, R^{a} is H, R^{b} is H, and R^{c} is NR^{x}R^{y}, and the compound has the structural formula (V⁴):

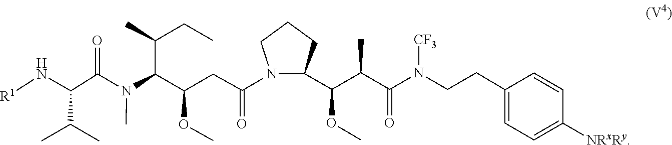

In certain embodiments of (V⁴), R^{x} is H and R^{y} is H, having the structural formula (V⁵):

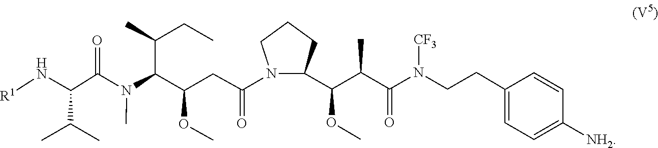

In certain embodiments of (V⁴), R^{x} is H or CH₃ and R^{y} is (C═O)—O—(CH₂)ₚ—R^{v}, wherein R^{v} is R, OR, NHR, NR₂, an aryl group or an amino acid; and p is 0, 1, 2 or 3.

In certain embodiments of (V⁴), R^{x} is H or CH₃ and R^{y} is (C═O)—(CH₂)_{q}—R^{v}, wherein R^{v} is R, OR, NHR, NR₂, an aryl group or an amino acid; and q is 0, 1, 2 or 3.

In certain embodiments of (V⁴), R^{x} is H and R^{y} is L-R^{z}, and the compound has the structural formula (V⁶):

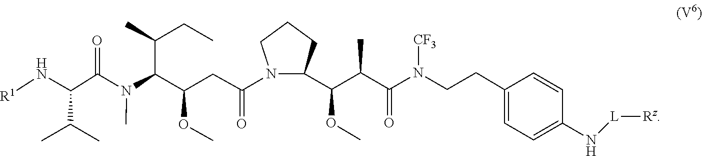

In certain embodiments of any one of formulae (I)-(V⁶) above, R¹ is

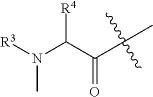

wherein each of R³ and R⁴ is independently H or an unsubstituted or substituted C₁-C₅ alkyl, or together with the N and C atoms they are boned to form a 5- to 7-membered heterocycloalkyl comprising one or more of O, N and S, optionally substituted with one or more of halogen atoms or C₁-C₃ alkyl.

In certain embodiments of any one of formulae (I)-(V⁶) above, R¹ is

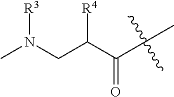

wherein each of R³ and R⁴ is independently H or an unsubstituted or substituted C₁-C₅ alkyl, or together with the N and C atoms they are boned to form a 5- to 7-membered heterocycloalkyl comprising one or more of O, N and S, optionally substituted with one or more of halogen atoms or C₁-C₃ alkyl.

In certain embodiments, R³ is H and R⁴ is H or an unsubstituted or substituted C₁-C₅ alkyl (e.g., methyl, ethyl, propyl, isopropyl, n-butyl, isobutyl, t-butyl).

In certain embodiments, R³ is methyl, optionally substituted with one or more halogen atoms (e.g., F, Cl), and R⁴ is H or an unsubstituted or substituted C₁-C₅ alkyl (e.g., methyl, ethyl, propyl, isopropyl, n-butyl, isobutyl, t-butyl).

In certain embodiments, R³ is ethyl, optionally substituted with one or more halogen atoms (e.g., F, Cl), and R⁴ is H or an unsubstituted or substituted C₁-C₅ alkyl (e.g., e.g., methyl, ethyl, propyl, isopropyl, n-butyl, isobutyl, t-butyl).

In certain embodiments, R³ is propyl or isopropyl, optionally substituted with one or more halogen atoms (e.g., F, Cl), and R⁴ is H or an unsubstituted or substituted C₁-C₅ alkyl (e.g., e.g., methyl, ethyl, propyl, isopropyl, n-butyl, isobutyl, t-butyl).

In certain embodiments, R⁴ is H. In certain embodiments, R⁴ is methyl. In certain embodiments, R⁴ is isopropyl.

In certain embodiments, R³ and R⁴, together with the N and C atoms they are boned to respectively, form a 5-membered heterocycloalkyl, optionally substituted with one or more of F, Cl and Br. In certain embodiments, R³ and R⁴, together with the N and C atoms they are boned to respectively, form a 6-membered heterocycloalkyl, optionally substituted with one or more of F, Cl and Br. In certain embodiments, R³ and R⁴, together with the N and C atoms they are boned to respectively, form a 7-membered heterocycloalkyl, optionally substituted with one or more of F, Cl and Br.

In certain embodiments of any one of formulae (I)-(V⁶), R¹ is selected from:

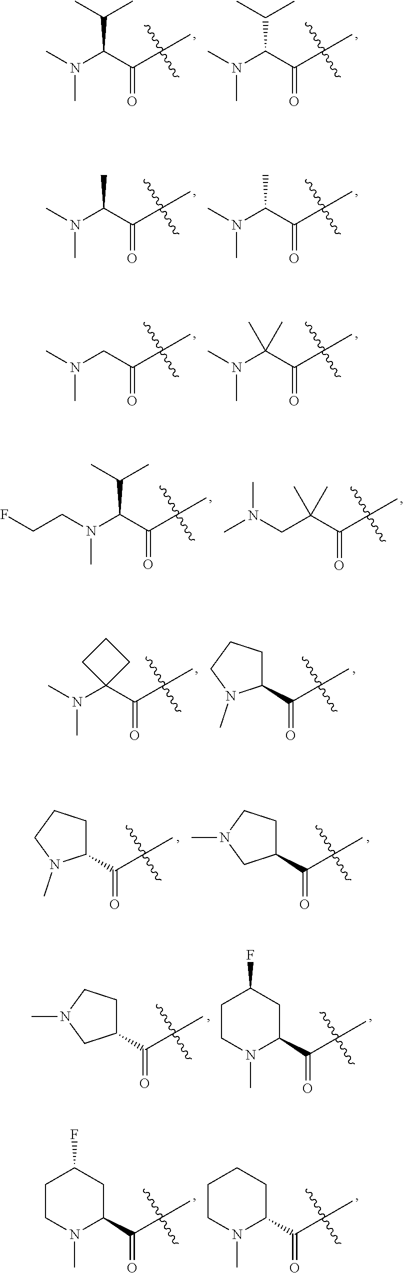

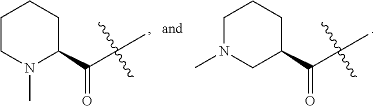

In certain embodiments of any one of formulae (I)-(V⁶), L is a noncleavable linker.

In certain embodiments of any one of formulae (I)-(V⁶), L is a cleavable linker.

In certain embodiments, L is an acid-labile or acid-sensitive linker. In certain embodiments, L is protease-sensitive linker. In certain embodiments, L is lysosomal protease-sensitive linker. In certain embodiments, L is β-glucuronide-sensitive linker. In certain embodiments, L is glutathione-sensitive disulfide linker.

In certain embodiments, L is an unbranched linker, i.e., suitable for conjugation to a single cytotoxic agent or payload per linker.

In certain embodiments, L is a branched linker, e.g., having 2, 3, 4, 5, 6, 7, 8 or more branches, wherein each branch is suitable for conjugation to a cytotoxic agent or payload thereby being suitable for conjugation to more than one cytotoxic agent or payload per linker.

In certain embodiments of any one of formulae (I)-(V⁶), R^{z} if present comprises a functional or reactive group suitable for conjugation to an antigen-binding moiety, for example, a functional or reactive group selected from:
—N₃, —NR^{u}C(═O)CH═CH₂, —SH, —SSR^{t}, —S(═O)₂(CH═CH₂), —(CH₂)₂S(=O)₂(CH═CH₂), —NR^{u}S(=O₂)(CH═CH₂), —NR^{u}C(═O)CH₂R^{w}, —NR^{u}C(═O)CH₂Br, —NR^{u}C(═O)CH₂I, —NHC(═O)CH₂Br, NHC(═O)CH₂I, —ONH₂, —C(═O)NHNH₂, —CO₂H, —NH₂, —NCO, —NCS,

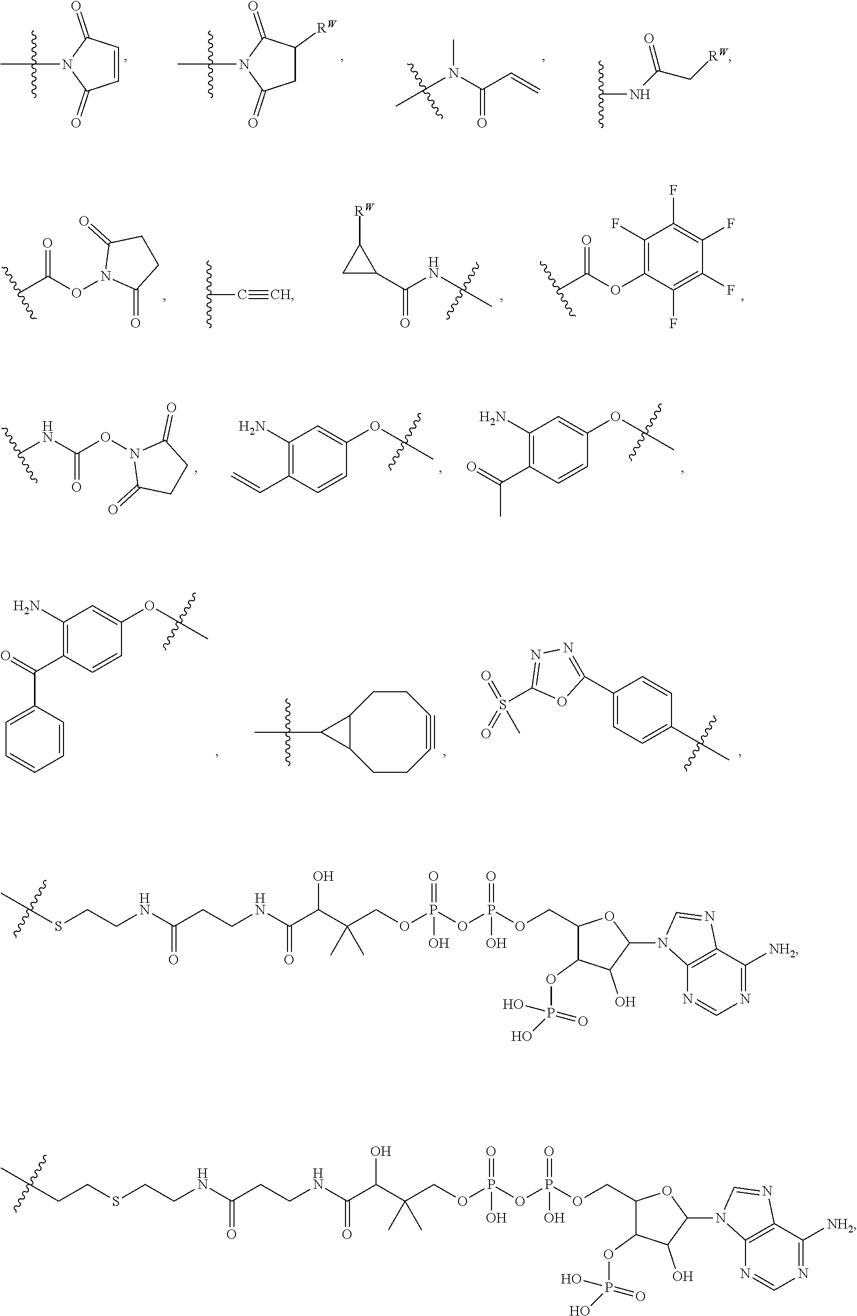

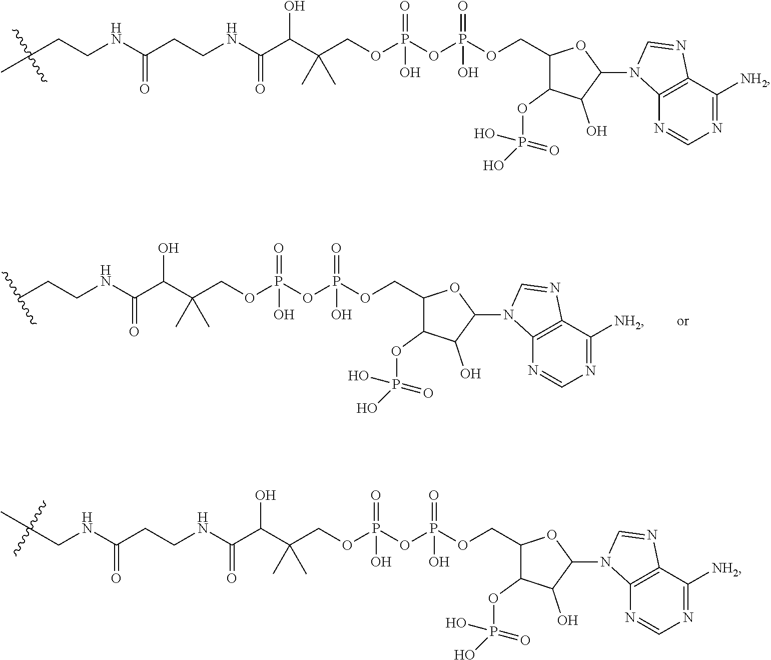

wherein
R^{u} is H or a C₁-C₆ alkyl group,
R^{t} is 2-pyridyl or 4-pyridyl, and
R^{w} is

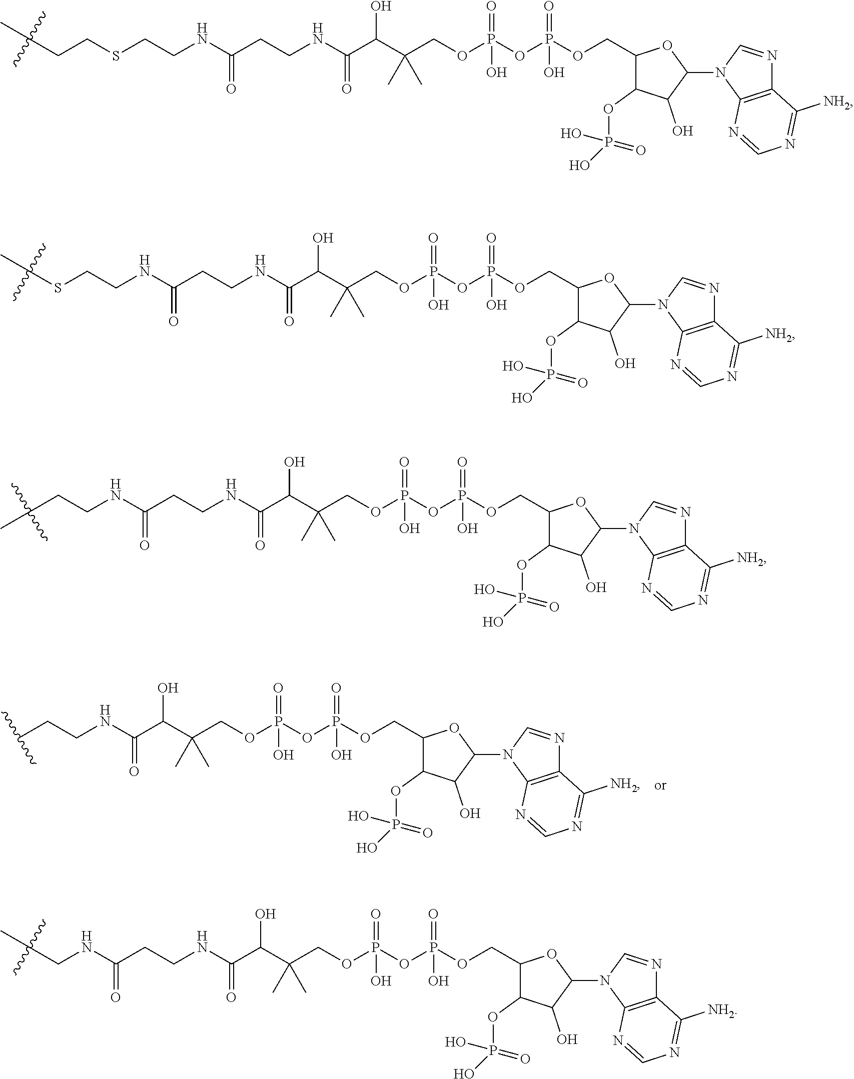 [SMILES: *CCSCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

Additional disclosures on linkers and reactive or functional groups that may be employed in R^{Z} and/or components of L are provided in the sections “Linkers and Linking Technologies” and “Linker-antibody and Linker-payload Attachments” and references cited therein, each of which is incorporated herein by reference.

The invention also includes methods for synthesizing auristatin analogs, including intermediates or precursors thereof.

Non-limiting examples of auristatin analogs of the invention include:

TABLE 1
Examples of Auristatin Analogs

| Compound | |
|---|---|
| No. | Structure |
| 1 | 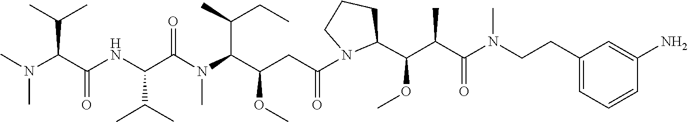 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@H](C(C)C)N(C)C)C(C)C] |
| 2 | 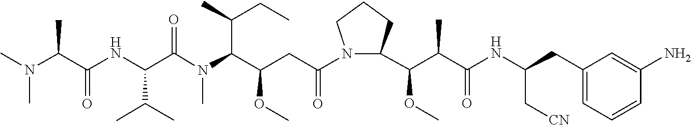 [SMILES: CC[C@H](C)[C@@H]([C@@H](CC(=O)N1CCC[C@H]1[C@H](OC)[C@@H](C)C(=O)N[C@H](CC#N)Cc1cccc(N)c1)OC)N(C)C(=O)[C@@H](NC(=O)[C@H](C)N(C)C)C(C)C] |
| 3 | 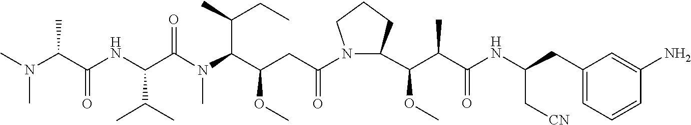 [SMILES: CC[C@H](C)[C@@H]([C@@H](CC(=O)N1CCC[C@H]1[C@H](OC)[C@@H](C)C(=O)N[C@H](CC#N)Cc1cccc(N)c1)OC)N(C)C(=O)[C@@H](NC(=O)[C@@H](C)N(C)C)C(C)C] |
| 4 | 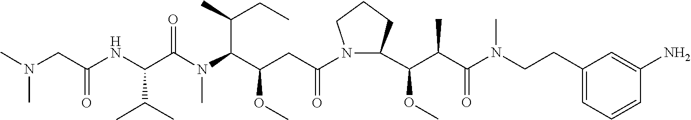 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)CN(C)C)C(C)C] |
| 5 | 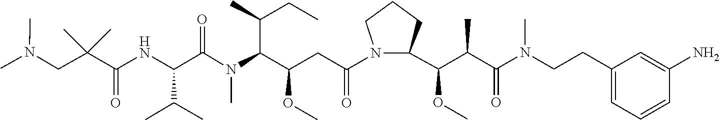 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)C(C)(C)CN(C)C)C(C)C] |
| 6 | 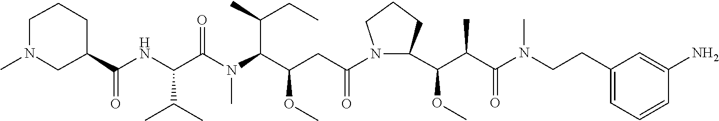 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCCN(C)C1)C(C)C] |
| 7 | 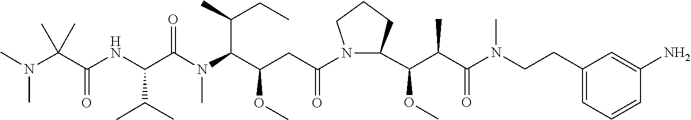 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)C(C)(C)N(C)C)C(C)C] |
| 8 | 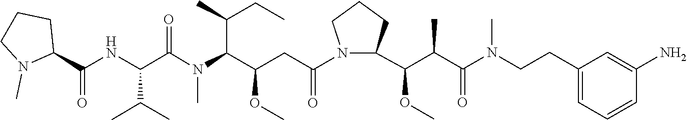 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCCN1C)C(C)C] |
| 9 | 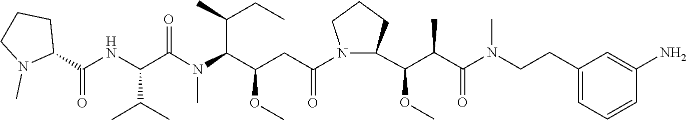 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@H]1CCCN1C)C(C)C] |
| 10 | 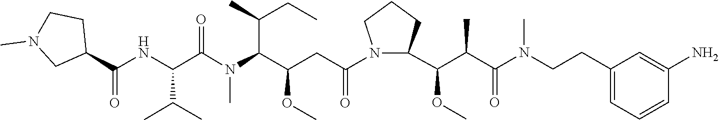 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCN(C)C1)C(C)C] |
| 11 | 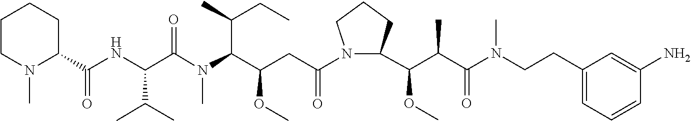 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@H]1CCCCN1C)C(C)C] |
| 12 | 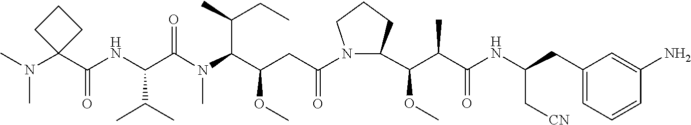 [SMILES: CC[C@H](C)[C@@H]([C@@H](CC(=O)N1CCC[C@H]1[C@H](OC)[C@@H](C)C(=O)N[C@H](CC#N)Cc1cccc(N)c1)OC)N(C)C(=O)[C@@H](NC(=O)C1(N(C)C)CCC1)C(C)C] |
| 13 | 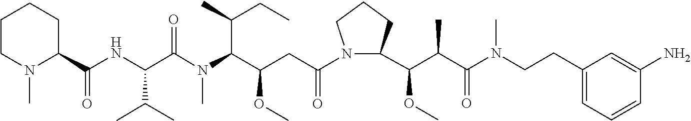 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCCCN1C)C(C)C] |
| 14 | 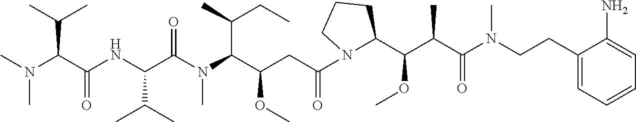 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@H](C(C)C)N(C)C)C(C)C] |
| 15 | 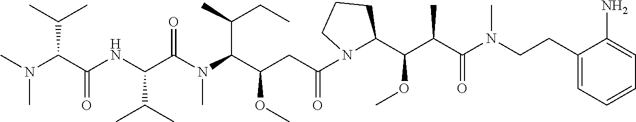 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@@H](C(C)C)N(C)C)C(C)C] |
| 16 | 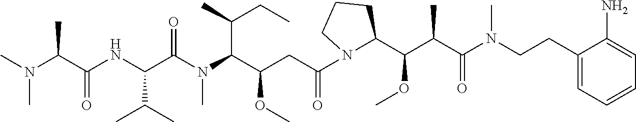 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@H](C)N(C)C)C(C)C] |
| 17 | 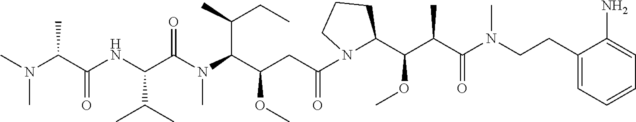 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@@H](C)N(C)C)C(C)C] |
| 18 | 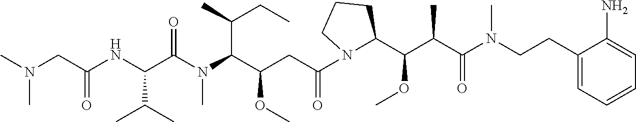 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)CN(C)C)C(C)C] |
| 19 | 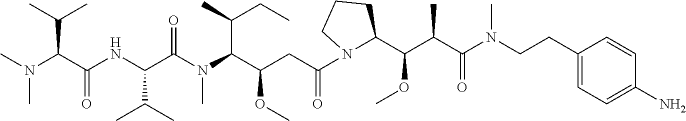 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@H](C(C)C)N(C)C)C(C)C] |
| 23 | 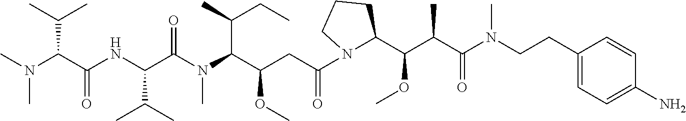 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@@H](C(C)C)N(C)C)C(C)C] |
| 24 | 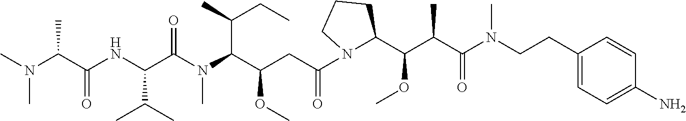 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@@H](C)N(C)C)C(C)C] |
| 25 | 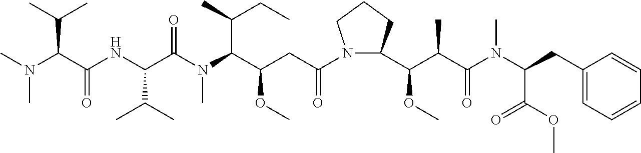 [SMILES: CC[C@H](C)[C@@H]([C@@H](CC(=O)N1CCC[C@H]1[C@H](OC)[C@@H](C)C(=O)N(C)[C@@H](Cc1ccccc1)C(=O)OC)OC)N(C)C(=O)[C@@H](NC(=O)[C@H](C(C)C)N(C)C)C(C)C] |
| 26 | 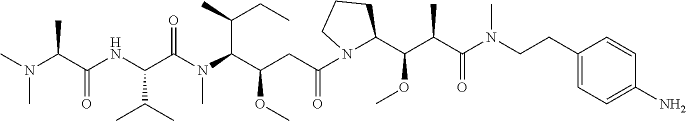 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@H](C)N(C)C)C(C)C] |
| 27 | 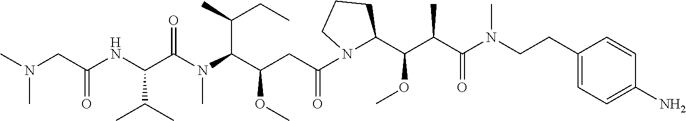 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)CN(C)C)C(C)C] |

In another aspect, the invention generally relates to a drug-linker conjugated formed by conjugation of a compound disclosed herein with a linker.

Non-limiting examples of linker-conjugated auristatin analogs include:

TABLE 2
Examples of Auristatin Analogs

| Compound | |
|---|---|
| No. | Structure |
| 28 | 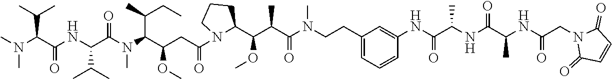 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@H](C(C)C)N(C)C)C(C)C] |
| 29 | 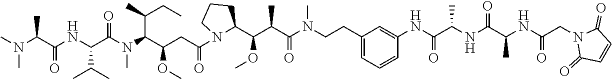 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@H](C)N(C)C)C(C)C] |
| 30 | 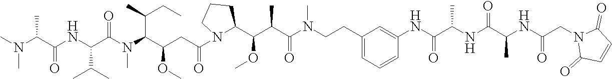 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@@H](C)N(C)C)C(C)C] |
| 31 | 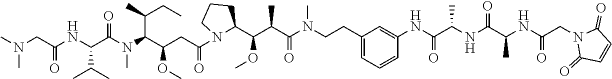 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)CN(C)C)C(C)C] |
| 32 | 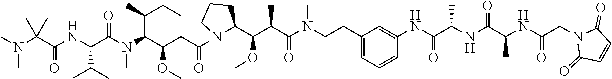 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)C(C)(C)N(C)C)C(C)C] |
| 33 | 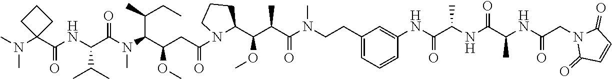 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)C1(N(C)C)CCC1)C(C)C] |
| 34 | 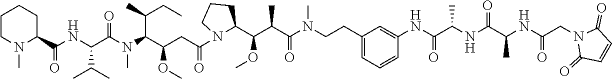 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@@H]1CCCCN1C)C(C)C] |
| 35 | 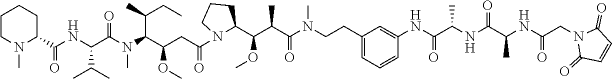 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@H]1CCCCN1C)C(C)C] |
| 36 | 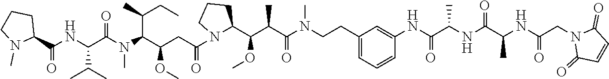 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@@H]1CCCN1C)C(C)C] |
| 37 | 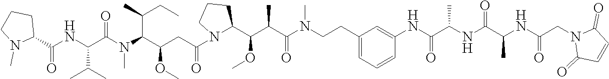 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@H]1CCCN1C)C(C)C] |
| 38 | 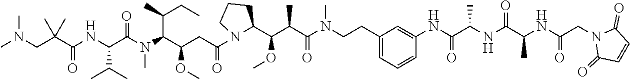 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)C(C)(C)CN(C)C)C(C)C] |
| 39 | 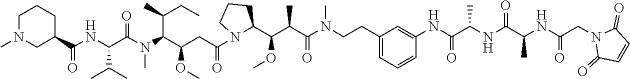 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@@H]1CCCN(C)C1)C(C)C] |
| 40 | 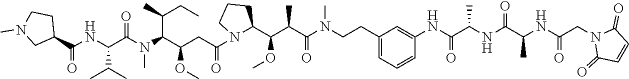 [SMILES: CC[C@H](C)[C@@H]([C@@H](CC(=O)N1CCC[C@H]1[C@H](OC)[C@@H](C)C(=O)N(C)CCc1cccc(NC(=O)[C@H](C)NC(=O)[C@H](C)NC(=O)CN2C(=O)C=CC2=O)c1)OC)N(C)C(=O)[C@@H](NC(=O)[C@@H]1CCN(C)C1)C(C)C] |

Methods for determining binding affinity of a compound to tubulin are known in the art. (See, e.g., Muller et al. 2006 Anal. Chem. 78, 4390-4397; Hamel et al. 1995 Molecular Pharmacology 47: 965-976; Hamel et al. 1990 J. Biological Chemistry 265:28, 17141-17149.)

In some embodiments, auristatin analogs disclosed herein bind tubulin with an affinity ranging from 10-fold lower (weaker) than the binding affinity of monomethyl auristatin E (MMAE) to tubulin to 5-fold, 10-fold, 20-fold, 30-fold, 50-fold or 100-fold higher (stronger) than the binding affinity of MMAE to tubulin.

### Immunoconjugates

A typical ADC is comprised of an antigen binding moiety (Ab), e.g., a monoclonal antibody), a linker (L) and cytotoxic agent or payload (D), as represented below:

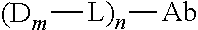

wherein each m and n is an integer. The payload D (e.g., a auristatin analog disclosed herein) can be conjugated to different parts of the Ab and is commonly attached via cysteine or lysine residues. Generally, more than one payload D molecules can be attached to each Ab. When a branched linker is employed, more than one payload D moieties can be attached to each linker L. In some embodiments, n ranges from 1 to 16, 1 to 12, 1 to 10, 1 to 8, 1 to 6, 1 to 5, 1 to 4, 1 to 3, or 1 to 2. In some embodiments, n ranges from 2 to 10, 2 to 8, 2 to 7, 2 to 6, 2 to 5, 2 to 4 or 2 to 3. In other embodiments, n is 1, 2, 3, 4, 5 or 6. In some embodiments, n is 2, 3 or 4. In some embodiments, L is an unbranched linker and m is 1. In some embodiments, L is a branched linker and m can range from 2 to 10, 2 to 8, 2 to 6, or 2 to 4. In some embodiments, m is 2, 3 or 4.

The drug to antibody ratio (DAR) or drug loading may be characterized by conventional means such as UV, mass spectroscopy, ELISA assay, HIC, HPLC or electrophoresis. In exemplary embodiments, DAR ranges from 1 to 16, 2 to 8, 1 to 12, 1 to 10, 1 to 8, 1 to 6, 1 to 5, 1 to 4, 1 to 3, 1 to 2, or about 1.

The DAR of an immunoconjugate may be controlled by various methods, including limiting the molar excess of payload-linker intermediate or linker reagent relative to antigen binding moieties; limiting the conjugation reaction time or temperature; varying reductive conditions for cysteine thiol modification; and modifying the number and positions of cysteine residues and positions of linker-payload attachments. (See, e.g., WO 2006/034488 A2.)

In one aspect, the invention generally relates to an immunoconjugate formed by conjugation of a compound disclosed herein, via a linker, with an antigen binding moiety.

In another aspect, the invention generally relates to an immunoconjugate having the structural formula (VI):

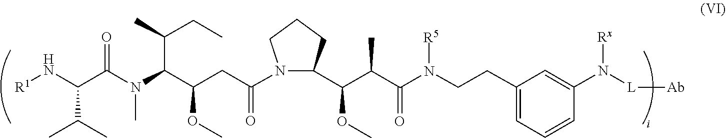

or a pharmaceutically acceptable salt thereof,
wherein
Ab represents an antigen binding moiety;
R¹ is

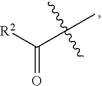

wherein R² is a unsubstituted or substituted C₁-C₆ alkyl, heteroalkyl, cycloalkyl or cycloheteroalkyl;
each of R^{x} and R^{y} is independently selected from R and L-R^{z}, provided that when one of R^{x} and R^{y} is NR^{z}, the other is R;
R⁵ is CR′₃, wherein each R′ is independently H or F;
L is a linker; and
R is H or a C₁-C₃ alkyl; and
i is an integer in the range of 1 to about 20.

In certain embodiments of the immunoconjugate of formula (VI), R⁵ is CH₃ and R^{x} is H, and the immunoconjugate has the structural formula (VI¹):

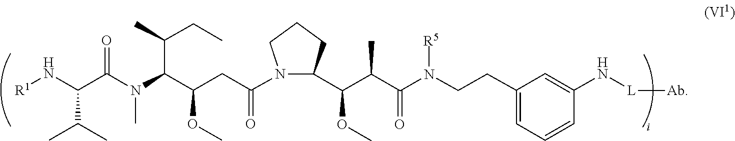

In certain embodiments of formulae (VI)-(VI¹), i is an integer in the range of 1 to 20. In certain embodiments, i is an integer in the range of 1 to 16. In certain embodiments, i is an integer in the range of 1 to 12. In certain embodiments, i is an integer in the range of 1 to 10. In certain embodiments, i is an integer in the range of 1 to 8. In certain embodiments, i is an integer in the range of 1 to 6. In certain embodiments, i is an integer in the range of 1 to 5. In certain embodiments, i is an integer in the range of 1 to about 4. In certain embodiments, i is an integer in the range of 1 to 3. In certain embodiments, i is an integer in the range of 1 to 2. In certain embodiments, i is 1.

In another aspect, the invention generally relates to an immunoconjugate having the structural formula (VII):

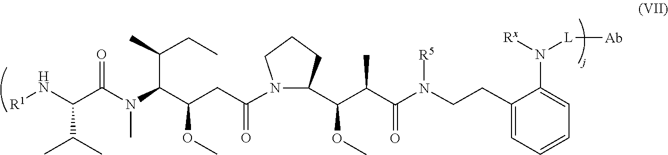

or a pharmaceutically acceptable salt thereof,
wherein
Ab represents an antigen binding moiety;
R¹ is

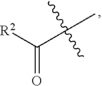

wherein R² is a unsubstituted or substituted C₁-C₆ alkyl, heteroalkyl, cycloalkyl or cycloheteroalkyl;
each of R^{x} and R^{y} is independently selected from R and L-R^{z}, provided that when one of
R^{x} and R^{y} is NR^{z}, the other is R;
R⁵ is CR′₃, wherein each R′ is independently H or F;
L is a linker; and
R is H or a C₁-C₃ alkyl; and
j is an integer in the range of 1 to about 20.

In certain embodiments of the immunoconjugate of formula (VII), R⁵ is CH₃ and R^{x} is H, having the structural formula (VII¹):

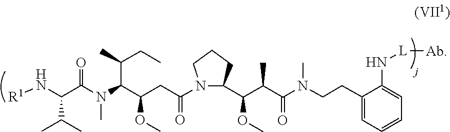

In certain embodiments of formulae (VII)-(VII¹), j is an integer in the range of 1 to 20. In certain embodiments, j is an integer in the range of 1 to 16. In certain embodiments, j is an integer in the range of 1 to 12. In certain embodiments, j is an integer in the range of 1 to 10. In certain embodiments, j is an integer in the range of 1 to 8. In certain embodiments, j is an integer in the range of 1 to 6. In certain embodiments, j is an integer in the range of 1 to 5. In certain embodiments, j is an integer in the range of 1 to about 4. In certain embodiments, j is an integer in the range of 1 to 3. In certain embodiments, j is an integer in the range of 1 to 2. In certain embodiments, j is 1.

In yet another aspect, the invention generally relates to an immunoconjugate having the structural formula (VIII):

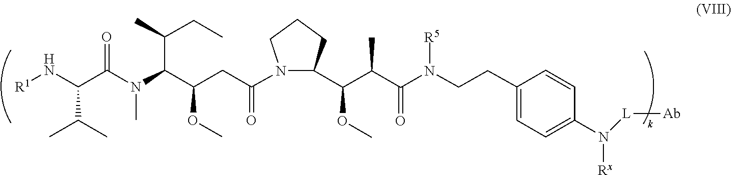

or a pharmaceutically acceptable salt thereof,
wherein
Ab represents an antigen binding moiety;
R¹ is

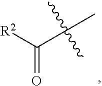

wherein R² is a unsubstituted or substituted C₁-C₆ alkyl, heteroalkyl, cycloalkyl or cycloheteroalkyl;
each of R^{x} and R^{y} is independently selected from R and L-R^{z}, provided that when one of R^{x} and R^{y} is NR^{z}, the other is R;
R⁵ is CR′₃, wherein each R′ is independently H or F;
L is a linker; and
R is H or a C₁-C₃ alkyl; and
k is an integer in the range of 1 to about 20.

In certain embodiments of the immunoconjugate of formula (VIII), R⁵ is CF₃ and R^{x} is H, having the structural formula (VIII¹):

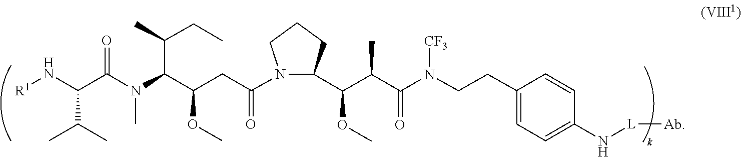

In certain embodiments of formulae (VIII)-(VIII¹), k is an integer in the range of 1 to 20. In certain embodiments, k is an integer in the range of 1 to 16. In certain embodiments, k is an integer in the range of 1 to 12. In certain embodiments, k is an integer in the range of 1 to 10. In certain embodiments, k is an integer in the range of 1 to 8. In certain embodiments, k is an integer in the range of 1 to 6. In certain embodiments, k is an integer in the range of 1 to 5. In certain embodiments, k is an integer in the range of 1 to about 4. In certain embodiments, k is an integer in the range of 1 to 3. In certain embodiments, k is 1 or 2. In certain embodiments, k is 1.

All substitution groups, e.g., R¹, R², R³, R⁴, R⁵, R^{x}, R^{y}, R, R′, L, found in formulae (VI)-(VIII¹) can be selected as discussed in the section titled “Auristatin Analogs and Cytotoxins” in connection with formulae (I)-(V⁶) and is herein incorporated in its entirety, including each and all combinations of R¹, R², R³, R⁴, R⁵, R^{x}, R^{y}, R, R′, L and R^{z} and the resulting compounds. The invention thus includes immunoconjugates corresponding to Ab-linked formulae (I)-(V⁶).

In addition to immunoconjugates wherein the antigen-binding moiety is an antibody or an antibody fragment, the invention additionally includes immunoconjugates wherein the antigen-binding moiety is a peptide and wherein the antigen-binding moiety is a small molecule ligand. (See, e.g., Zhuang et al. 2019 Eur. J Med. Chem. 163, 883-895; Patel et al. 2021 New J. Chem. 45, 5291-5321.)

The invention also includes methods for synthesizing immunoconjugates, including intermediates or precursors thereof. The invention additionally includes a composition comprising an immunoconjugate, an intermediate or a precursor thereof.

### Antigen Binding Moieties

To date, numerous unique antigens have been identified and may be potentially used in antibody-based therapy as a target. Several factors are generally considered when selecting an antigen. First, the target antigen should have high expression in the tumor and no or low expression in the healthy cell. An example is the HER2 receptor, which is almost 100-fold higher expressed in the tumor cell compared to the healthy cell. Second, the target antigen should be displayed on the surface of the tumor cell to be available to the circulated monoclonal antibody. In addition, the target antigen should possess internalization properties as it will facilitate the ADC to transport into the cell, which will in turn enhance the efficacy of cytotoxic agent. Though some studies have demonstrated that non-internalized ADC product directed against components of the tumor microenvironment can efficiently detach their drug in the extracellular space and arbitrate a potent therapeutic activity in some cases and that ADCs often induce a strong “bystander effect.” (Strohl WR 2018 Protein & Cell. 9(1):86-120; Damelin et al. 2015 Pharma. Res. 32(11):3494-507; Diamantis et al. 2016 British J. Cancer 114(4):362-7; Tipton et al. 2015 Blood 125(12):1901-9; Donaghy et al. 2016 mAbs. 8(4):659-71; Casi et al. 2015 Molecular Pharmaceutics 12(6):1880-4.)

An antigen-binding moiety can be any moiety that selectively binds to a cell-surface marker found on a targeted cell type. In general, the antibody should preferably possess target specificity and deliver the cytotoxic drug to the tumor cell and possess target binding affinity, i.e., a high binding affinity to the tumor cell-surface antigens. Additionally, the antibody should preferably possess good retention, low immunogenicity, low cross-reactivity, and appropriate linkage binding properties. (Peters et al. 2015 Bioscience Reports 35(4); Hughes B 2010 Nature Reviews Drug Discovery 9(9):665-7.)

In certain embodiments, Ab is an antibody.

In certain embodiments, Ab is a monoclonal antibody.

In certain embodiments, Ab is a chimeric antibody.

In certain embodiments, Ab is a humanized antibody.

In certain embodiments, Ab is a bispecific antibody.

In certain embodiments, Ab is an antibody fragment.

In certain embodiments, Ab is a Fab fragment.

In certain embodiments, Ab is a peptide.

In certain embodiments, Ab is a small molecule ligand.

In some aspects, Ab is an antibody or antibody fragment (e.g. antigen binding fragment of an antibody) that specifically binds to an antigen predominantly or preferentially found on the surface of cancer cells, e.g., a tumor-associated antigen.

In some aspects, Ab is an antibody or antibody fragment (e.g., antigen binding fragment) that specifically binds to a cell surface receptor protein or other cell surface molecules, a cell survival regulatory factor, a cell proliferation regulatory factor, a molecules associated with, known or suspected to contribute functionally to, tissue development or differentiation, a lymphokine, a cytokine, a molecule involved in cell cycle regulation, a molecule involved in vasculogenesis or a molecule associated with, known or suspected to contribute functionally to, angiogenesis.

Thus, antigen-binding moieties useful in immunoconjugates of the invention include, but not limited to, antibodies against cell surface receptors and tumor-associated or tumor-specific antigens, which are well known in the art and can be prepared for use in generating antibodies using methods and information known in the art.

In attempts to discover effective cellular targets for cancer diagnosis and therapy, researchers have sought to identify transmembrane or otherwise tumor-associated or tumor-specific polypeptides that are specifically expressed on the surface of one or more particular type(s) of cancer cell as compared to on one or more normal non-cancerous cell(s). Tumor-associated polypeptides are more abundantly expressed on the surface of the cancer cells as compared to on the surface of the non-cancerous cells, whereas tumor-specific polypeptides are specifically expressed on the surface of one or more particular type(s) of cancer cell but not on non-cancerous cell(s). The identification of such cell surface antigen polypeptides has given rise to the ability to specifically target cancer cells for destruction via antibody-based therapies. (See, e.g., Liu et al. 2017 Eur. J. Cancer Care (Engl). 2017 Sep.; 26(5), doi: 10.1111/ecc.12446; WO 2016/192527 A1.)

A tumor-associated antigen may be a cluster differentiation factor (e.g., a CD protein). In some aspects of the invention, the antigen binding moiety of the invention specifically binds to one antigen. In some aspects of the invention, the antigen binding moiety of the invention specifically binds to two or more antigens described herein, for example, the antigen binding moiety of the invention is a bispecific or multispecific antibody or antigen binding fragment thereof.

Non-limiting examples of antibodies or antigen binding fragments include anti-estrogen receptor antibody, anti-progesterone receptor antibody, anti-p53 aniibody, anti-HER-2 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB3-2 antibody, anti-P-glycoprotein antibody, anti-CEA antibody, anti-retinoblastona protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD1-antibody, anti-CD11 c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD331 antibody, anti-CD33 antibody, anti-CD34 antibody, anti-CD35 antibody, anti-CD38 antibody, anti-CD39 antibody, anti-CD41 antibody, anti-LCA/CD45 antibody, anti-CD45RO antibody, anti-CD45RA antibody, anti-CD71 antibody, anti-D95/Fas antibody, anti-CD99 antibody, anti-CD100 antibody, anti-S-100 antibody, anti-CD106 antibody, anti-ubiquitin antibody, anti-c-myc antibody, anti-cytokeratin antibody, ani-lambda light chains antibody, anti-melanosomes antibody, ani-prostate specific antigen antibody, anti-tau antigen antibody, anti-fibrin antibody, anti-keratins antibody, and anti-Tn-antigen antibody.

Antibodies and antibody fragments useful for the immunoconjugates of the invention include modified or engineered antibodies, such as an antibody modified to introduce a cysteine residue, or other reactive amino acid, including Pel, pyrrolysine, peptide tags, and non-natural amino acids, in place of at least one amino acid of the native sequence, thus providing a reactive site on the antibody or antigen binding fragment for conjugation to a cytotoxic agent.

The location of the drug moiety may be designed, controlled and known. For example, cysteine amino acids may be engineered at reactive sites in an antibody and which do not form intrachain or intermolecular disulfide linkages. (Junutula, et al. 2008 Nature Biotech. 26(8):925-932; Dornan et al. 2009 Blood 114(13):2721-2729; U.S. Pat. No. 7,521,541 B2; U.S. Pat. No. 7,723,485 B2; WO 2009/052249 A2.) The engineered cysteine thiols may react with linker reagents or the drug-linker reagents of the present invention which have thiol-reactive, electrophilic groups such as maleimide or alpha-halo amides to form ADC with cysteine engineered antibodies and the drug moieties.

Additionally, the antibodies or antibody fragments can be modified to incorporate Pel or pyrrolysine or unnatural amino acids as sites for conjugation to a drug. Peptide tags for enzymatic conjugation methods can be introduced into an antibody. (Junutula et al. 2008 Nat. Biotechnol 26:925-932; Ou et al. 2011 PNAS 108 (26), 10437-10442; Axup et al. 2012 Proc. Natl. Acad. Sci. USA, 109, 16101-16106; Liu et a. 2010 Annu. Rev. Biochem. 79, 413-444 Kim et al. 2013 Curr. Opin. Chem. Biol. 17, 412-A419; Strop et al. 2013 Chem. Biol. 20(2):161-7: Rabuka 2010 Curr. Opin. Chem. Biol. 14(6):790-6; Rabuka et al. 2012 Nat. Protoc 7(6): 1052-67; WO 2015/095301 A2: WO 2013/184514 A2.)

Antibodies and antibody fragments can be readily produced by any methods known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.. (See, e g., Carvalho et al. 2016 “Production Processes for Monoclonal Antibodies”, DOI: 10.5772/64263 (https://www.intechopen.con/chapters/S1512); Monoclonal Antibody Production, Committee on Methods of Producing Monoclonal Antibodies, Institute for Laboratory Animal Research. National Research Council, NATIONAL ACADEMY PRESS Washington, D C 1999; Jakobovits 1998 Adv. Drug Del. Rev. 31:33-42; Marks et al. 1991 J. Mol. Biol. 222:581; Cole et al. 1985 Monoclonal Antibodies And Cancer Therapy 77-96; Teng et al. 1983 Proc. Natl. Acad Sci. USA. 80:7308-7312; Kozbor et al., 19831Immunology Today 4:72-79; Olsson et al. 1982 Meth. Enzymol. 92:3-16; U.S. Pat. No. 6,657,103 B2.)

### Linkers and Linking Technologies

The cytotoxic agents disclosed herein are suitable for use as payloads in immunoconjugates. The auristatin analogs of the invention can be attached to a linker or directly to an antigen binding moiety. Linkers in ADCs are typically designed to achieve high stability in the circulation and, in the case of cleavable linkers, specific release of payload in the target tissue.

Suitable linkers and linking techniques for use in building an immunoconjugate are well known in the art and can be used in making the immunoconjugate conjugates of the invention. In general, a linker may be attached to the antigen binding moiety at any suitable available position on the antigen binding moiety, for examples, attached to an available amino nitrogen atom (e.g., a primary or secondary amine) or a hydroxylic oxygen atom, or to an available sulfhydryl, such as on a cysteine. The attachment of a linker to the cytotoxic auristatin analog disclosed herein can be at the N-terminus or at the C-terminus of the cytotoxic agent.

Various linkers and linking strategies are known and can be employed in making immunoconjugates of the invention. (See, e.g., Kang et al. 2021 “Recent developments in chemical conjugation strategies targeting native amino acids in proteins and their applications in antibody-drug conjugates” Chemical Science Royal Soc. of Chem., DOI: 10.1039/d1sc02973h; Su et al. 2021 “Antibody-drug conjugates: Recent advances in linker chemistry” Acta Pharmaceutica Sinica B, https://doi.org/10.1016/j.apsb.2021.03.042; Drago et al. 2021 Nature Reviews 18, 327-344; Mckertish et al. 2021 Biomedicines 9, 872; Bargh et al. 2019 “Cleavable linkers in antibody-drug conjugates” Chem. Soc. Rev. 48, 4361, DOI: 10.1039/c8cs00676h; Lash 2011 “Antibody-Drug Conjugates: the Next Generation of Moving Parts” Start-Up, Dec. 2011, 1-6; WO 2021/055865 A1; WO 2016/192527 A1; WO 2015/095301 A2; WO 2011/097627 A1, WO 2004/010957 A1, U.S. Pub. No. 20060074008 A2, U.S. Pub. No. 20050238649 A2, and U.S. Pub. No. 20060024317 A2.)

A linker may be classified as either cleavable or non-cleavable. In the case of ADCs with noncleavable linkers, the release is typically via internalization of the ADC followed by degradation of the antibody in the lysosome, resulting in the release of the payload still attached via the linker to an antibody amino acid residue. Examples of noncleavable linker include maleimidoca-proyl (MC) and 4-(N-maleimidomethyl) cyclohexane-1-carboxylate (MCC) linkers. Examples of cleavable linkers include Val-Cit, N-Succinimidyl-4-(2-pyridyldithio) butanoate (SPDB), N-succinimidyl-4-(2-pyridyldithio) pentanoate (SPP) and hydrazide.

For the immunoconjugates of comprising a cleavable linker, the linker is substantially stable in vivo until the immunoconjugate binds to or enters a cell, at which point either intracellular enzymes or intracellular chemical conditions (pH, reduction capacity) cleave the linker to free the cytotoxic peptide.

Cleavable linkers may further be classified based on the cleavage mechanism into chemically cleavable linkers (such as acid-cleavable linkers, reducible disulfide linkers and exogeneous stimuli triggered linkers) and enzyme cleavable linkers (such as dipeptide Val-Cit-containing linkers, glycosidase-cleavable linkers, phosphatase-cleavable linkers). Acid cleavable linkers (a.k.a. pH-sensitive linkers) are designed to exploit the acidity of the endosomes (pH 5.5-6.2) and lysosomes (pH 4.5-5.0), while maintaining stability in circulation at pH 7.4. An example of an acid-cleavable linkers is an acid-sensitive N-acyl hydrazine linkage that, upon acid catalysis, hydrolyses to a ketone and a hydrazide-payload. Acid cleavable linkers containing other functional groups have also been reported, such as a carbonate linker. Glycosidase-cleavable linkers include β-Glucuronidase-cleavable linkers, β-Galactosidase-cleavable linkers, phosphatase-cleavable linkers. (See, e.g., Bargh et al. 2019 “Cleavable linkers in antibody-drug conjugates” Chem. Soc. Rev. 48, 4361, DOI: 10.1039/c8cs00676h; Ducry, et al. 2010 Bioconjugate Chem., vol. 21, 5-13; Jeffrey et al. 2006 Bioconjugate Chem. 17, 831-840; Burke et al. 2009 Bioconjugate Chem. 20, 1242-1250; Kolodych et al. 2017 J. Med. Chem. 142, 376-382; Kern et al. 2016 Bioconjugate Chem. 27, 2081-2088; Stenton et al. 2018 Chem. Sci. 9, 4185-4189; Pillow et al. 2017 Mol. Cancer Ther. 16, 871-878; Dubowchik et al. 1998 Bioorg. Med. Chem. Lett. 8, 3341-3346; Dubowchik et al. 1998 Bioorg. Med. Chem. Lett. 8, 3347-3352; WO 2021/055865 A1; WO 2016/192527 A1; WO 2015/095301 A2; US 2021/0138077 A1; WO 2013/173393 A1; WO 2011/097627 A1.)

### Linker-Antibody and Linker-Payload Attachments

Various attachment strategies have been developed over the years including site-specific conjugation technologies, antibody engineering and chemical modifications.

Major attachment approaches include maleimide attachment (e.g., N-alkyl maleimide, N-phenyl maleimide), bis(vinylsulfonyl)piperazine attachment, N-methyl-N-phenylvinylsulfonamide attachment, and Pt(II)-based attachment. (See, e.g., Su et al. 2021 “Antibody-drug conjugates: Recent advances in linker chemistry” Acta Pharmaceutica Sinica B, https://doi.org/10.1016/j.apsb.2021.03.042; Mckertish et al. 2021 Biomedicines 9, 872; Patterson et al. 2015 Bioconjug. Chem. 26:2243e8; Lyu et al. 2018 ACS Chem. Biol. 13:958e64; Zhou 2017 Biomedicines 5:64; Christie et al. 2017 Antibodies (Basel) 6:20; Sun et al. 2019 Org. Biomol. Chem. 17: 2005e12; Huang et al. 2018 Org. Lett. 20: 6526e9; Sijbrandi et al. 2017 Cancer Res. 77: 257e67; Merkul et al. 2020 Angew Chem. Int. Ed. Engl. 60:3008e15; Merkul et al. 2019 Expert Opin. Drug Deliv. 16:783e93; WO 2015/095301 A2; US 2021/0138077 A1; WO 2013/173393 A1; WO 2016/192527 A1; WO 2021/055865 A1.)

Various linker-payload attachment strategies have been reported, such as carbamate attachment and carbonate attachment. (See, e.g., Wahby et al. 2020 Clin. Cancer Res. Available from: https://doi.10.1158/1078-0432.CCR-20-3119; Perini et al. 2013 Biol. Ther. 3:15e23; Burke et al. 2016 Mol. Cancer Ther. 15:938e45; WO 2015/095301 A2; US 2021/0138077 A1; WO 2013/173393 A1; WO 2016/192527 A1; WO 2021/055865 A1.)

Non-limiting examples of attachment strategies and reactive groups are provided in Table 3. (See, e.g., WO 2015/095301 A2; U.S. Pat. No. 9,988,420 B2.)

TABLE 3
Exemplary Reactive Groups and Moieties

| Reactive Group 1 | Reactive Group 2 | Chemical Moiety |
|---|---|---|
| a thiol | a thiol | —S—S— |
| a thiol | a maleimide | 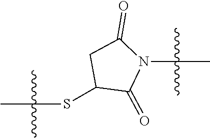 [SMILES: *SC1CC(=O)N(*)C1=O] |
| a thiol | a haloacetamide | 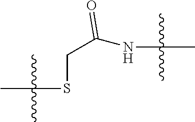 [SMILES: *NC(=O)CS*] |
| an azide | an alkyne | 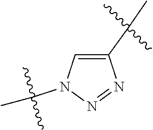 [SMILES: *c1cn(*)nn1] |
| | | or |
| | | 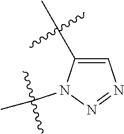 [SMILES: *c1cnnn1*] |
| an azide | a triaryl phosphine | 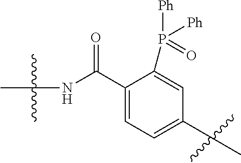 [SMILES: *NC(=O)c1ccc(*)cc1P(=O)(c1ccccc1)c1ccccc1] |
| an azide | a cyclooctene | 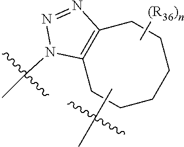 |
| | | 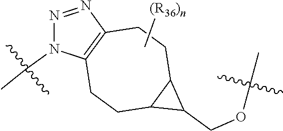 |
| | | or |
| | | 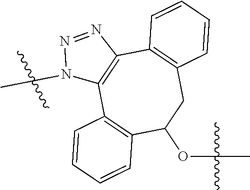 [SMILES: *OC1Cc2ccccc2-c2nnn(*)c2-c2ccccc21] |
| an azide | an oxanobornadiene | 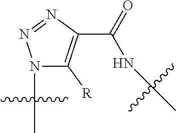 |
| a triaryl phosphine | an azide | 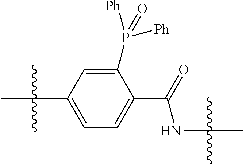 [SMILES: *NC(=O)c1ccc(*)cc1P(=O)(c1ccccc1)c1ccccc1] |
| an oxanobornadiene | an azide | 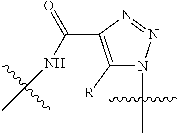 |
| an alkyne | an azide | 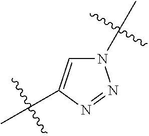 [SMILES: *c1cn(*)nn1] |
| | | 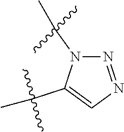 [SMILES: *c1cnnn1*] |
| a cyclooctene | azide | 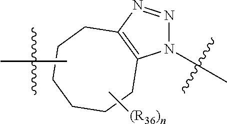 |
| | | or |
| | | 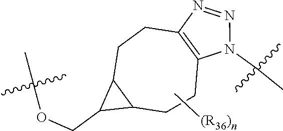 |
| | | or |
| | | 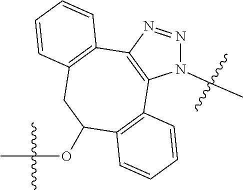 [SMILES: *OC1Cc2ccccc2-c2nnn(*)c2-c2ccccc21] |
| a cyclooctene | a diaryl tetrazine | 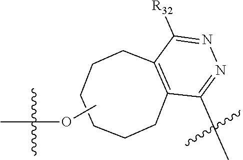 |
| | | or |
| | | 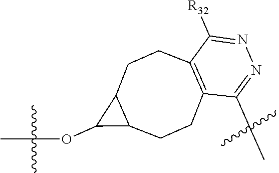 |
| a diaryl tetrazine | a cyclooctene | 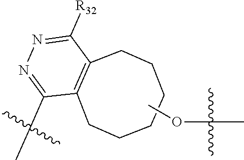 |
| | | or |
| | | 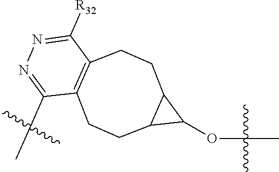 |
| a monoaryl tetrazine | a norbornene | 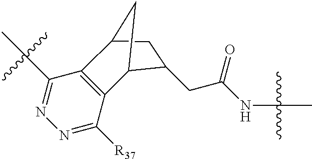 |
| a norbornene | a monoaryl tetrazine | 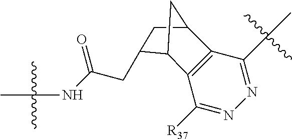 |
| an aldehyde | a hydroxylamine | 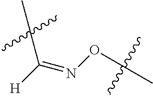 [SMILES: */C=N\O*] |
| an aldehyde | a hydrazine | 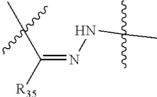 |
| an aldehyde | NH₂—NH—C(═O)— | 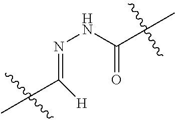 [SMILES: */C=N/NC(*)=O] |
| a ketone | a hydroxylamine | 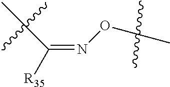 |
| a ketone | a hydrazine | 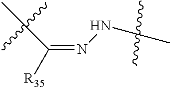 |
| a ketone | NH₂—NH—C(═O)— | 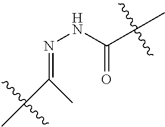 [SMILES: *C(=O)N/N=C(/*)C] |
| a hydroxylamine | an aldehyde | 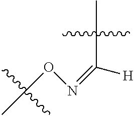 [SMILES: */C=N\O*] |
| a hydroxylamine | a ketone | 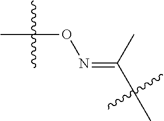 [SMILES: *O/N=C(/*)C] |
| a hydrazine | an aldehyde | 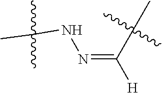 [SMILES: */C=N\N*] |
| a hydrazine | a ketone | 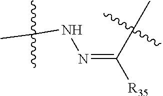 |
| NH₂—NH—C(═O)— | an aldehyde | 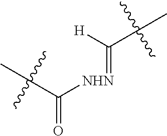 |
| NH₂—NH—C(═O)— | a ketone | 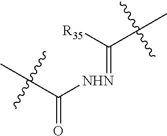 |
| a haloacetamide | a thiol | 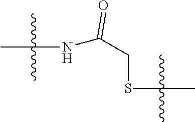 [SMILES: *NC(=O)CS*] |
| a maleimide | a thiol | 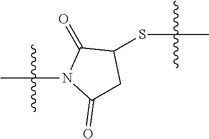 [SMILES: *SC1CC(=O)N(*)C1=O] |
| a vinyl sulfone | a thiol | 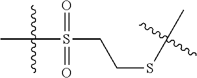 [SMILES: *SCCS(*)(=O)=O] |
| a thiol | a vinyl sulfone | 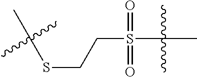 [SMILES: *SCCS(*)(=O)=O] |
| an aziridine | a thiol | 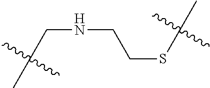 [SMILES: *CNCCS*] |
| | | or |
| | | 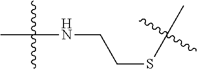 [SMILES: *NCCS*] |
| a thiol | an aziridine | 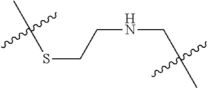 [SMILES: *CNCCS*] |
| | | or |
| | | 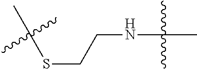 [SMILES: *NCCS*] |
| 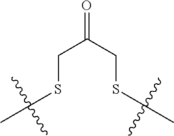 [SMILES: *SCC(=O)CS*] | hydroxylamine | 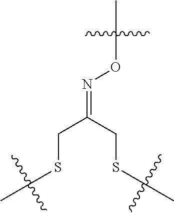 [SMILES: *ON=C(CS*)CS*] |
| 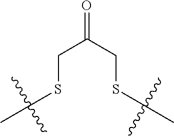 [SMILES: *SCC(=O)CS*] | hydroxylamine | 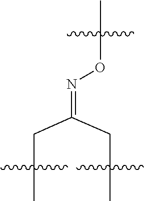 [SMILES: *CC(C*)=NO*] |
| 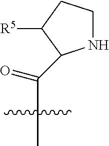 | 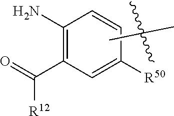 | 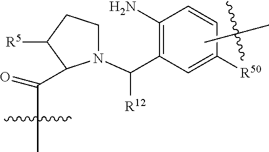 |

### Pharmaceutical Compositions and Methods of Use

### Pharmaceutical Compositions

In another aspect, the invention generally relates to a composition comprising a compound disclosed herein, such as according to any one of formulae (I)-(V⁶) and in Table 1 and Table 2, or a pharmaceutically acceptable salt thereof, and optionally a pharmaceutically acceptable excipient, carrier or diluent.

In yet another aspect, the invention generally relates to a pharmaceutical composition comprising an immunoconjugate disclosed herein, such as according to any one of formulae (VI)-(VIII¹), or a pharmaceutically acceptable salt thereof, and a pharmaceutically acceptable excipient, carrier or diluent.

The invention thus provides a pharmaceutical preparation comprising a therapeutically effective amount of a compound or immunoconjugate according to the invention.

Examples of excipients that may be useful include, but not limited to, water, saline, dextrose, mannitol, lactose, lecithin, albumin, sodium glutamate, cysteine hydrochloride, starches, celluloses and gums. In a preferred embodiment, the pharmaceutical composition of the invention is formulated in a pharmaceutical form for administration as a solid (for example tablets, capsules, lozenges, granules, suppositories, crystalline or amorphous sterile solids that can be reconstituted to provide liquid forms, etc.), liquid (for example solutions, suspensions, emulsions, elixirs, lotions, unguents, etc.) or semi-solid (gels, ointments, creams and similar). The pharmaceutical compositions of the invention can be administered by any route, including, without limitation, oral, intravenous, intramuscular, intraarterial, intramedullary, intratecal, intraventricular, transdermic, subcutaneous, intraperitoneal, intranasal, enteric, topical, sublingual or rectal route. A revision of the different forms of administration of active principles, the excipients to be used and their manufacturing procedures can be found in Remington's Pharmaceutical Sciences (A. R. Gennaro, Ed.), 20^{th} edition, Williams & Wilkins PA, USA (2000) Examples of pharmaceutically acceptable vehicles are known in the state of the technique and include saline solutions buffered with phosphate, water, emulsions, such as oil/water emulsions, different types of humidifying agents, sterile solutions, etc. The compositions comprising said vehicles can be formulated by conventional procedures known in the state of the technique. Preservatives, stabilizers, dyes and even flavoring agents, antioxidants and/or suspending agents can be provided in the pharmaceutical composition. For example, sodium benzoate, ascorbic acid and esters of p-hydroxybenzoic acid can be added as preservatives.

The invention also contemplates a kit comprising at least an immunoconjugate disclosed herein and a syringe and/or vial or ampoule in which the immunoconjugate and/or pharmaceutical composition is disposed.

### Methods of Use

In yet another aspect, the invention generally relates to a method for treating or reducing a disease or condition, comprising administering to a subject in need thereof a therapeutically effective amount of an immunoconjugate disclosed herein.

In certain embodiments, the disease or condition is cancer.

In certain embodiments, the method further comprises administering one or more of chemotherapy and radiotherapy on the subject.

In yet another aspect, the invention generally relates to use of an immunoconjugate disclosed herein for the manufacture of a medicament.

In certain embodiments, an immunoconjugate disclosed herein is used for treating a disease or condition, wherein the disease or condition is cancer.

In yet another aspect, the invention generally relates to use of an immunoconjugate disclosed herein for use in treating cancer.

Exemplary cancers include: carcinomas, sarcomas, leukemias, and lymphomas. An exhaustive list of cancer types and cancers by body location can be found at National Cancer Institute's website, e.g., https://www.cancer.gov/types and https://www.cancer.gov/types/by-body-location, each of which is incorporated herein by reference in its entirety.

In certain embodiments, the disease or disorder is one or more cancer selected from gastric cancer, myeloid cancer, colon cancer, nasopharyngeal cancer, esophageal cancer, and prostate cancer, glioma, neuroblastoma, breast cancer, lung cancer, ovarian cancer, colorectal cancer, thyroid cancer, leukemia (e.g., myelogenous leukemia, lymphocytic leukemia, acute myelogenous leukemia, chronic myeloid leukemia, acute lymphoblastic leukemia, T-lineage acute lymphoblastic leukemia or T-ALL chronic lymphocytic leukemia, myelodysplastic syndrome, hairy cell leukemia), lymphoma (Hodgkin's lymphoma, non-Hodgkin's lymphoma), multiple myeloma, bladder cancer, renal cancer, gastric (e.g., gastrointestinal stromal tumors), liver cancer, melanoma and pancreatic cancer, and sarcoma.

Immunoconjugates may generally be administered by the systemic route, in particular by the intravenous route, by the intramuscular, intradermal, intraperitoneal or subcutaneous route, or by the oral route. Immunoconjugates are typically administered intravenously into the blood stream of a subject in order to avoid gastric acids or proteolytic enzymes degradation of the antibody. In some embodiments, the composition comprising the immunoconjugates disclosed herein will be administered several times, in a sequential manner.

### Combination Therapies

In yet another aspect, the invention generally relates to a combination comprising a therapeutically effective amount of an immunoconjugate disclosed herein, and one or more therapeutically active co-agent(s) and/or adjuvant(s).

Co-agents include, but are not limited to, chemotherapeutic agents, growth factor inhibitors, biological response modifiers, anti-hormonal therapy, selective estrogen receptor modulators (SERMs), angiogenesis inhibitors and anti-androgens.

Adjuvants include, but are not limited to, those known in the art. (See, e.g., Temizoz et al. 2016 Int. Immunol. 28(7): 329-338.)

As used herein, the term “chemotherapeutic agent” refers to a chemical compound useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (1994 Angew Chem. Intl. Ed. Engl. 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esonibicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP—16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylomithine (DMFO); retinoids such as retinoic acid; and pharmaceutically acceptable salts, acids and derivatives of any of the above.

In certain embodiments, the therapeutic methods disclosed herein can enable the use of reduced dosages of chemotherapy (or other therapies) and/or less frequent administration, an advantage for all patients and particularly for those that do not tolerate the toxicity of the chemotherapeutic agent well.

Additionally, growth factor inhibitors, biological response modifiers, anti-hormonal therapy, selective estrogen receptor modulators (SERMs), angiogenesis inhibitors, and anti-androgens may be used. For example, anti-hormones, for example anti-estrogens, e.g., Nolvadex (tamoxifen) or, anti-androgens such as Casodex (4′-cyano-3-(4-fluorophenylsulphonyl)-2-hydroxy-2-methyl-3-′-(trifluoromethyl)propionanilide) may be used.

Additional examples of the second, third or further agent(s) or therapies may include, but are not limited to, immunotherapies (e.g. PD-1 inhibitors (pembrolizumab, nivolumab, cemiplimab), PD-L1 inhibitors (atezolizumab, avelumab, durvalumab), CTLA4 antagonists, cell signal transduction inhibitors (e.g., imatinib, gefitinib, bortezomib, erlotinib, sorafenib, sunitinib, dasatinib, vorinostat, lapatinib, temsirolimus, nilotinib, everolimus, pazopanib, trastuzumab, bevacizumab, cetuximab, ranibizumab, pegaptanib, panitumumab and the like), mitosis inhibitors (e.g., paclitaxel, vincristine, vinblastine and the like), alkylating agents (e.g., cisplatin, cyclophosphamide, chromabucil, carmustine and the like), anti-metabolites (e.g., methotrexate, 5-FU and the like), intercalating anticancer agents, (e.g., actinomycin, anthracycline, bleomycin, mitomycin-C and the like), topoisomerase inhibitors (e.g., irinotecan, topotecan, teniposide and the like), immunotherapic agents (e.g., interleukin, interferon and the like) and antihormonal agents (e.g., tamoxifen, raloxifene and the like).

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Thus, isotope derivative compounds having one or more hydrogen atoms (e.g., 1, 2, 4, 5, 6, 7, 8, 9, 10, etc.) replaced with deuterium atoms are contemplated in the presented invention. In certain embodiments, isotope derivative compounds of the invention have one hydrogen atom replaced with a deuterium atom.

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

The following examples are meant to be illustrative of the practice of the invention and not limiting in any way.

### EXAMPLES

### Synthesis

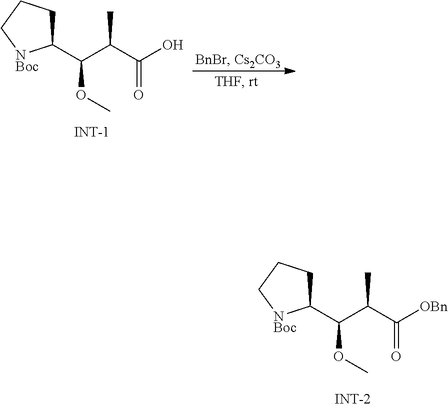

### (S)-tert-butyl 2-((1R,2R)-3-(benzyloxy)-1-methoxy-2-methyl-3-oxopropyl)pyrrolidine-1-carboxylate INT-2

To a solution of (2R,3R)-3-((S)-1-(tert-butoxycarbonyl)pyrrolidin-2-yl)-3-methoxy-2-methylpropanoic acid INT-1 (19 g, 66.12 mmol; Leyan) in 190 mL dry THF was added Cs₂CO₃ (28 g, 85.96 mmol), followed by BnBr (7.27 mL, 78.02 mmol) added. The resulting mixture was stirred at room temperature for 16 h. LCMS showed completion. The mixture was filtrated directly, and the filter cake was washed with THF (30 mL*3). The filtrate was collected and concentrated to afford the crude INT-2 (˜33 g, >100% yield, containing BnBr) as a yellow liquid, which was used directly without further purification.

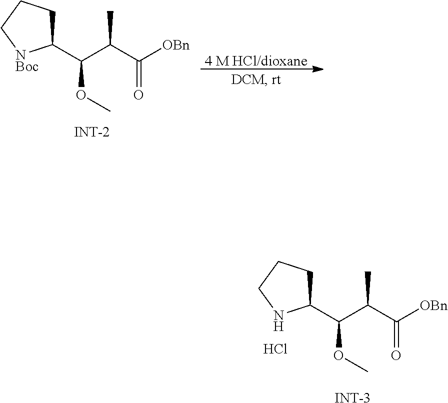

### (2R,3R)-benzyl 3-methoxy-2-methyl-3-((S)-pyrrolidin-2-yl)propanoate hydrochloride INT-3

The above INT-2 (66.12 mmol) was dissolved in 160 mL DCM, followed by 4 M HCl/dioxane (80 mL, 320 mmol) added. The resulting mixture was stirred at room temperature for 3 h. TLC showed completion. The reaction was concentrated to dry directly, and then re-dissolved in DCM (30 mL), followed by MTBE (300 mL) added. This mixture was stirred at 0° C. for 0.5 h, during which time white solid precipated. The white solid was collected by filtration and washed with MTBE/DCM=10:1 (20 mL*3) to give INT-3 (19.5 g, 94% yield): LCMS (ESI): m/z 278.2 [M+H]⁺; ¹H NMR (400 MHz, CDCl₃) δ 10.27 (s, 1H), 9.08 (s, 1H), 7.44-7.26 (m, 5H), 5.13 (q, J=12.3 Hz, 2H), 4.11-4.01 (m, 1H), 3.75-3.63 (m, 1H), 3.58 (s, 3H), 3.37-3.21 (m, 2H), 2.88-2.78 (m, 1H), 1.99-1.82 (m, 4H), 1.28 (d, J=7.0 Hz, 3H).

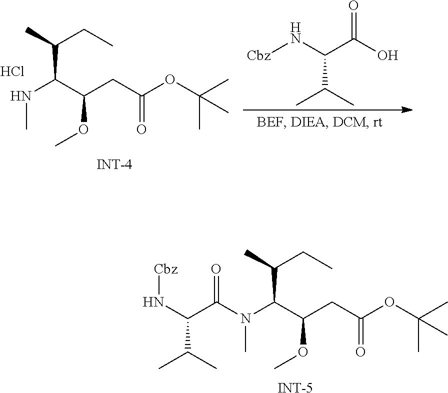

### (3R,4S,5S)-tert-butyl 4-((S)-2-(((benzyloxy)carbonyl)amino)-N,3-dimethylbutanamido)-3-meth oxy-5-methylheptanoate INT-5

To a solution of (3R,4S,5S)-tert-butyl 3-methoxy-5-methyl-4-(methylamino)heptanoate hydrochloride INT-4 (60 g, 185.9 mmol; Leyan) in 1 L dry DCM was added DIEA (141.3 mL, 743.6 mmol). The mixture was stirred at room temperature for 10 min, then cooled down to 0° C. Cbz-Val-OH (61.2 g, 223.1 mmol; Leyan) and BEP (74.98 g, 250.9 mmol) were added. The resulting mixture was allowed to warm up to room temperature naturally and stirred for 16 h. LCMS showed completion. The reaction was washed with H₂O (1 L*2) and brine, dried over Na₂SO₄, filtrated and concentrated to dry. The residue was purified by Flash Chromatography (petroleum ether: EtOAc=4:1-2:1, v/v) to afford INT-5 (79.5 g, 86% yield) as a light yellow oil. LCMS (ESI): m/z 493.0 [M+H]⁺.

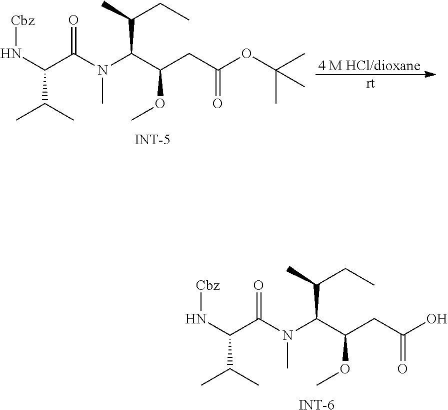

### (3R,4S,5S)-4-((S)-2-(((benzyloxy)carbonyl)amino)-N,3-dimethylbutanamido)-3-methoxy-5-met hylheptanoic acid INT-6

To a solution of INT-5 (79.5 g, 161.5 mmol) in 320 mL DCM was added 4 M HCl/dioxane (480 mL, 1.9 mmol) drop-wise in 15 min below 20° C. The reaction was then stirred at room temperature for 16 h. LCMS showed completion. The mixture was concentrated to dry, and the residue was purified by reverse phase column (H₂O/CH₃CN) to afford INT-6 (55.4 g, 78.6% yield) as a white solid. LCMS (ESI): m/z 437.1 [M+H]⁺.

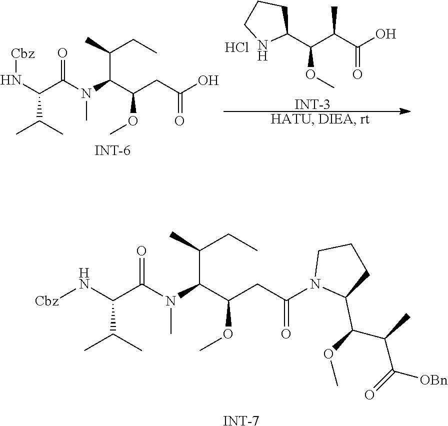

### (2R,3R)-benzyl 3-((S)-1-((3R,4S,5S)-4-((S)-2-(((benzyloxy)carbonyl)amino)-N,3-dimethylbuta namido)-3-methoxy-5-methylheptanoyl)pyrrolidin-2-yl)-3-methoxy-2-methylpropanoate INT-7

To a solution of INT-6 (9.7 g, 22.22 mmol) and INT-3 (7.32 g, 23.33 mmol) in 200 mL DMF was added HATU (16.9 g, 44.44 mmol) at room temperature. The reaction was cooled down to 0° C., and DIEA (16.5 mL, 0.1 mmol) was added. The resulting mixture was allowed to warm up to room temperature naturally and stirred for 3 h. LCMS showed completion. The reaction was diluted with DCM (500 mL), washed with H₂O (1 L*2), dried over Na₂SO₄, filtrated and concentrated to dry. The residue was purified by reverse phase column (H₂O/CH₃CN) to afford INT-7 (12.5 g, 81% yield) as yellow oil. LCMS (ESI): m/z 696.3 [M+H]⁺.

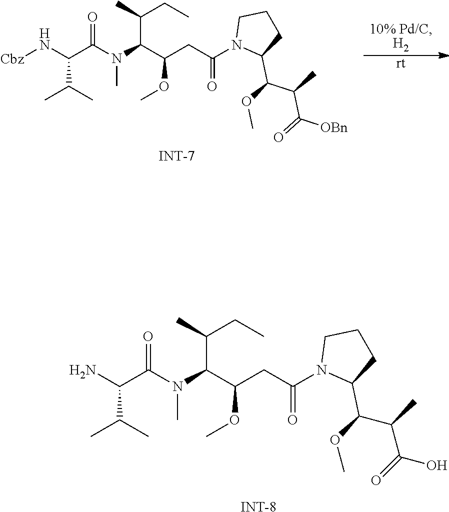 [SMILES: CC[C@H](C)[C@@H]([C@@H](CC(=O)N1CCC[C@H]1[C@H](OC)[C@@H](C)C(=O)OCc1ccccc1)OC)N(C)C(=O)[C@@H](NC(=O)OCc1ccccc1)C(C)C ; "10% Pd/C,H2" ; "rt" ; "INT-7" ; CC[C@H](C)[C@@H]([C@@H](CC(=O)N1CCC[C@H]1[C@H](OC)[C@@H](C)C(=O)O)OC)N(C)C(=O)[C@@H](N)C(C)C ; "INT-8"]

### (2R,3R)-3-((S)-1-((3R,4S,5S)-4-((S)-2-amino-N,3-dimethylbutanamido)-3-methoxy-5-methylhe ptanoyl)pyrrolidin-2-yl)-3-methoxy-2-methylpropanoic acid INT-8

To a solution of INT-7 (12.5 g, 17.96 mmol) in 125 mL MeOH was added 10% Pd/C (3.75 g). The reaction was then stirred under a H₂ atmosphere (1 atm) at room temperature for 4 h. LCMS showed completion. The mixture was filtrated, and the filter cake was washed with DCM/MeOH=1:1 (v/v) (50 mL*3). The combined filtrate was concentrated to afford the INT-8 (8 g, 94% yield) as a white solid. LCMS (ESI): m/z 472.1 [M+H]⁺; HPLC (NH₂ column): 99.7% @210 nm, Rₜ=6.59 min.

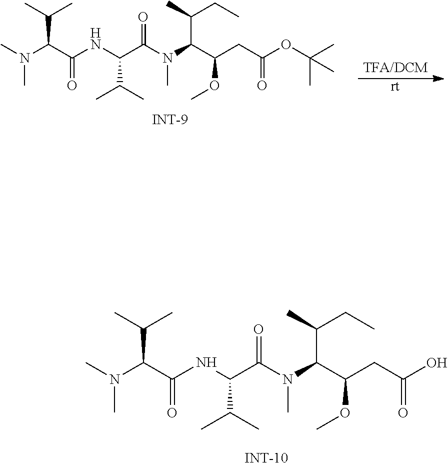

### (3R,4S,5S)-4-((S)-2-((S)-2-(dimethylamino)-3-methylbutanamido)-N,3-dimethylbutanamido)-3-methoxy-5-methylheptanoic acid INT-10

To a solution of INT-9 (8 g, 16.47 mmol) in 100 mL DCM was added TFA (34.5 mL, 461.2 mmol). The mixture was stirred at room temperature for 6 h. HPLC showed completion. The reaction was concentrated directly to afford the crude INT-10 (9.5 g, 100% yield) as a light yellow oil, which was used directly in next step without further purification. LCMS (ESI): m/z 430.2[M+H]⁺.

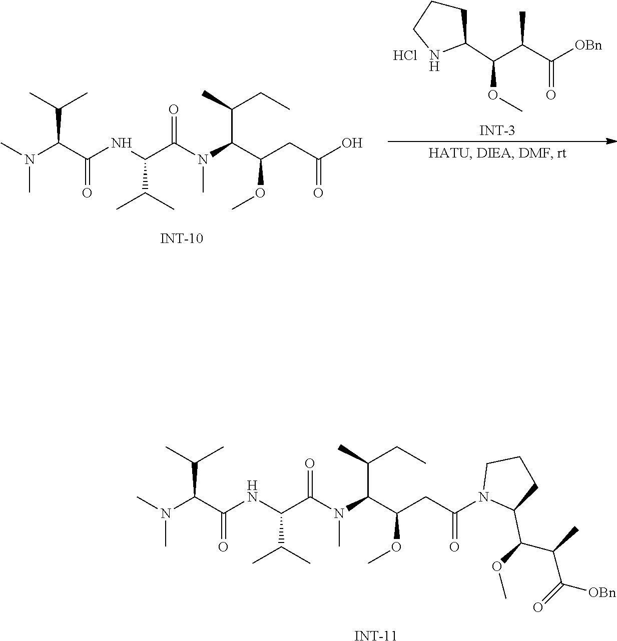

### (2R,3R)-benzyl 3-((S)-1-((3R,4S,5S)-4-((S)-2-((S)-2-(dimethylamino)-3-methylbutanamido)-N, 3-dimethylbutanamido)-3-methoxy-5-methylheptanoyl)pyrrolidin-2-yl)-3-methoxy-2-methylpro panoate INT-11

To a solution of the crude INT-10 (9.5 g, 16.47 mmol; see above) in 50 mL DMF was added HATU (12.39 g, 32.6 mmol) and DIEA(12.1 mL, 73.33 mmol). The reaction was stirred at room temperature for 0.5 h, then INT-3 (6.9 g, 22 mmol) was added. The resulting mixture was stirred at room temperature for 20 h. LCMS showed completion. The reaction was concentrated, and the residue was purified by reverse phase column (H₂O/CH₃CN) to afford INT-11 (6.57 g, 58.5% yield for 2 steps) as a white solid. LCMS (ESI): m/z 689.5 [M+H]⁺.

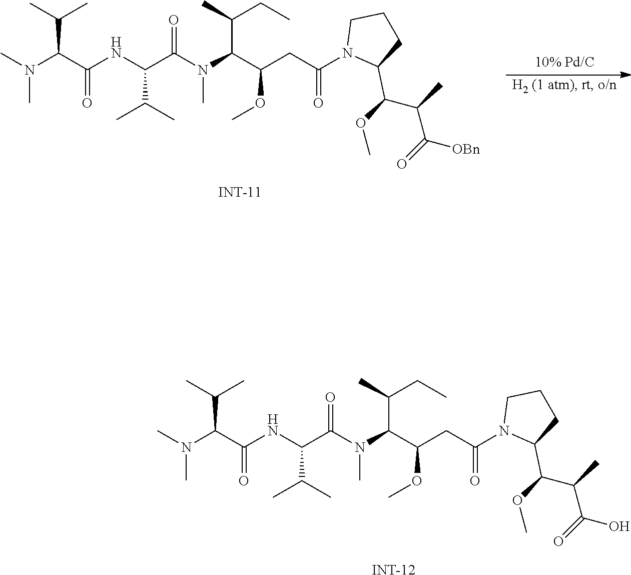 [SMILES: CC[C@H](C)[C@@H]([C@@H](CC(=O)N1CCC[C@H]1[C@H](OC)[C@@H](C)C(=O)OCc1ccccc1)OC)N(C)C(=O)[C@@H](NC(=O)[C@H](C(C)C)N(C)C)C(C)C ; "10% Pd/C" ; "H2 (1 atm), rt, o/n" ; "INT-11" ; CC[C@H](C)[C@@H]([C@@H](CC(=O)N1CCC[C@H]1[C@H](OC)[C@@H](C)C(=O)O)OC)N(C)C(=O)[C@@H](NC(=O)[C@H](C(C)C)N(C)C)C(C)C ; "INT-12"]

### (2R,3R)-3-((S)-1-((3R,4S,5S)-4-((S)-2-((S)-2-(dimethylamino)-3-methylbutanamido)-N,3-dimet hylbutanamido)-3-methoxy-5-methylheptanoyl)pyrrolidin-2-yl)-3-methoxy-2-methylpropanoic acid INT-12

To a solution of INT-11 (6.4 g, 9.29 mmol) in 315 mL MeOH/DCM (v/v=20:1) was added 10% Pd/C (1.6 g). The reaction was then stirred under a H₂ atmosphere (1 atm) at room temperature for 16 h. LCMS showed completion. The mixture was filtrated, and the filtrate was concentrated to afford INT-12 (5.61 g, 100% yield) as a white solid. LCMS (ESI): m/z 599.3 [M+H]⁺; HPLC: 97.8% @210 nm, Rₜ=8.61 min; ¹H NMR (400 MHz, DMSO) δ 12.29 (s, 1H), 9.51 (s, 1H), 8.91 (s, 1H), 4.66 (bs, 1H), 4.61-4.50 (m, 1H), 4.03-3.95 (m, 2H), 3.92-3.76 (m, 1H), 3.74-3.61 (m, 1H), 3.56-3.48 (m, 1H), 3.29 (s, 3H), 3.22-3.14 (m, 4H), [3.04 (s, 0.6H), 3.01 (s, 2.4H)], 2.82-2.68 (m, 6H), 2.48-2.43 (m, 1H), 2.39-2.26 (m, 3H), 2.08-1.83 (m, 4H), 1.83-1.68 (m, 3H), 1.35-1.27 (m, 1H), [1.18 (d, J=6.9 Hz, 0.6H), 1.11 (d, J=6.8 Hz, 2.4H)], 0.97-0.84 (m, 15H), 0.77 (t, J=7.1 Hz, 3H).

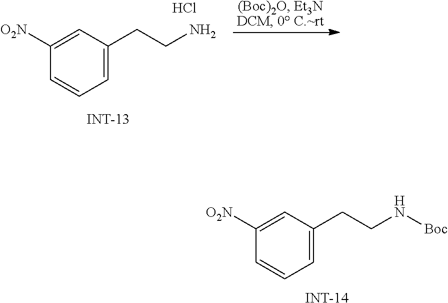

### Tert-butyl 3-nitrophenethylcarbamate INT-14

To a solution of 2-(3-nitrophenyl)ethanamine hydrochloride INT-13 (5.5 g, 27.14 mmol) in 110 mL dry DCM was added Et₃N (11.32 mL/8.24 g, 81.43 mmol) at room temperature under a N₂ atmosphere. The mixture was cooled down to 0° C. by an ice bath, then Boc₂O (2.37 g, 10.86 mmol) was added. The reaction was allowed to worm up to room temperature naturally and stirred for 16 h. TLC showed completion (petroleum ether:EtOAc=1:1, R_{f}=0.75). The mixture was quenched by adding 120 mL H₂O, and then extracted with DCM (70 mL*3). The combined organic layers were washed with H₂O and brine, dried over Na₂SO₄, filtrated and concentrated to dry. The residue was purified by Flash Chromatography (petroleum ether: EtOAc=10:1˜5:1, v/v) to afford INT-14 (7.23 g, 100% yield) as a light yellow oil. ¹H NMR (400 MHz, CDCl₃) δ 8.11-8.04 (m, 2H), 7.55-7.51 (m, 1H), 7.50-7.44 (m, 1H), 4.60 (bs, 1H), 3.41 (q, J=6.7 Hz, 2H), 2.92 (t, J=7.0 Hz, 2H), 1.42 (s, 9H).

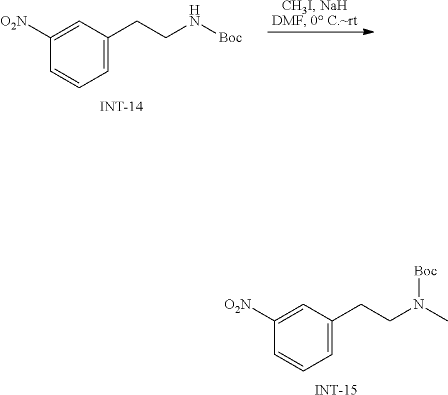

### Tert-butyl methyl(3-nitrophenethyl)carbamate INT-15

To a solution of tert-butyl 3-nitrophenethylcarbamate INT-14 (7.23 g, 27.13 mmol) in 80 mL dry DMF was added 60% NaH (1.63 g, 40.7 mmol) portion wise for 3 times at 0° C. under a N₂ atmosphere in 20 min. The mixture was stirred at 0° C. for 10 min, then CH₃I (2.87 mL, 46.12 mmol) was added. The reaction was allowed to worm up to room temperature naturally and stirred for 3 h. TLC showed completion (petroleum ether: EtOAc=5:1, R_{f}=0.6). The mixture was quenched by adding 100 mL H₂O slowly at 0° C., then extracted with EtOAc (60 mL*3). The combined organic layers were washed with H₂O and brine, dried over Na₂SO₄, filtrated and concentrated to dry. The residue was purified by Flash Chromatography (petroleum ether: EtOAc=50:1˜40:1˜30:1, v/v) to afford INT-15 (6.56 g, 86% yield) as a yellow oil. ¹H NMR (400 MHz, CDCl₃) δ 8.12-8.03 (m, 2H), 7.60-7.41 (m, 2H), 3.48 (t, J=7.2 Hz, 2H), 2.99-2.88 (m, 2H), 2.84 (s, 3H), 1.37 (s, 9H).

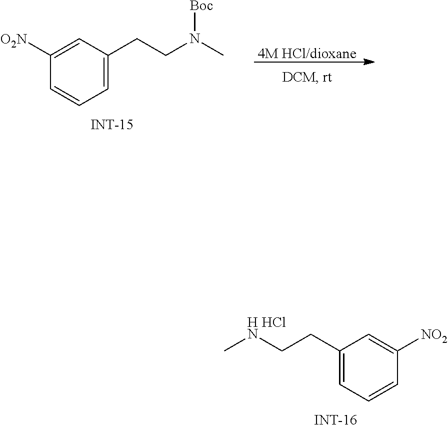

### N-methyl-2-(3-nitrophenyl)ethanamine hydrochloride INT-16

To a solution of tert-butyl methyl(3-nitrophenethyl)carbamate INT-15 (6.56 g, 23.40 mmol) in 60 mL DCM was added 4 M HCl/dioxane (30 mL, 120 mmol). The reaction was stirred at room temperature for 2 h, during which time much white solid precipated. TLC showed completion. The mixture was concentrated to dry. The residue was slurried with MTBE (40 mL) 3 times to afford INT-16 (4.92 g, 97% yield) as a light yellow solid: LCMS (ESI): m/z 181.1 [M+H]⁺; HPLC: 99.2% @210 nm, Rₜ=11.90 min; ¹H NMR (400 MHz, DMSO-d₆) δ 9.22 (bs, 2H), 8.17 (t, J=1.8 Hz, 1H), 8.15-8.09 (m, 1H), 7.77 (d, J=7.7 Hz, 1H), 7.64 (t, J=7.9 Hz, 1H), 3.25-3.08 (m, 4H), 2.54 (s, 3H).

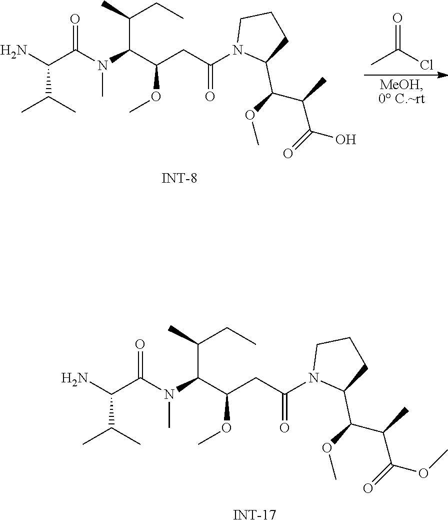

### (2R,3R)-methyl 3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate INT-17

Acetyl chloride (1.16 mL, 16.22 mmol) was added drop wise to dry MeOH (17 mL) at 0° C. under a N₂ atmosphere. The solution was stirred at 0° C. for 1 h, followed by INT-8 (1.5 g, 3.18 mmol) added in one portion. The reaction was allowed to warm up to room temperature naturally and stirred for 16 h. LCMS showed completion. The mixture was concentrated to dryness under the reduced pressure at 35° C. The residue was slurried by MTBE (15 mL*2) to afford INT-17 (1.55 g, 100% yield) as a yellow foam solid: LCMS (ESI): m/z 486.1 [M+H]⁺; HPLC (NH₂ column): 96.1% @210 nm, Rₜ=11.30 min; ¹H NMR (400 MHz, CDCl₃) δ 8.67-8.07 (m, 2H), 4.90-4.51 (m, 1H), 4.50-4.30 (m, 1H), 4.22-4.09 (m, 1H), 4.02-3.88 (m, 1H), 3.87-3.73 (m, 1H), 3.71 (s, 3H), 3.60-3.37 (m, 6H), 3.32 (s, 3H), 3.21-3.04 (m, 2H), 3.03-2.89 (m, 1H), 2.62-2.54 (m, 1H), 2.53-2.42 (m, 2H), 2.36-2.22 (m, 1H), 2.13-2.01 (m, 2H), 1.96-1.81 (m, 2H), 1.60-1.47 (m, 1H), 1.36-1.21 (m, 7H), 1.15-1.00 (m, 7H), 0.95-0.82 (m, 3H).

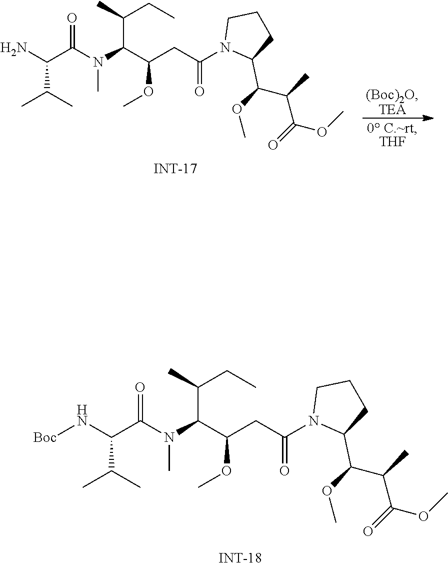

### (2R,3R)-methyl 3-((S)-1-((3R,4S,5S)-4-((S)-2-((tert-butoxycarbonyl)amino)-N,3-dimethylbutan amido)-3-methoxy-5-methylheptanoyl)pyrrolidin-2-yl)-3-methoxy-2-methylpropanoate INT-18

To a solution of INT-17 (3.96 g, 7.58 mmol) in 40 mL dry THE was added Et₃N (3.15 mL/2.30 g, 22.73 mmol) at room temperature under a N₂ atmosphere. The mixture was cooled down to 0° C. by an ice bath, then Boc₂0 (1.82 g, 8.33 mmol) was added. The reaction was allowed to worm up to room temperature naturally and stirred for 16 h. LCMS showed completion. The solvent of THE was removed by concentration, then 100 mL EtOAc added. The resulting mixture was washed with H₂O (30 mL*2) and brine (30 mL), dried over Na₂SO₄, filtrated and concentrated. The residue was purified by Flash Chromatography (petroleum ether: EtOAc=5:1˜3:1˜1:1, v/v) to afford INT-18 (4.55 g, 76% yield) as a colorless oil. LCMS (ESI): m/z 586.3 [M+H]⁺.

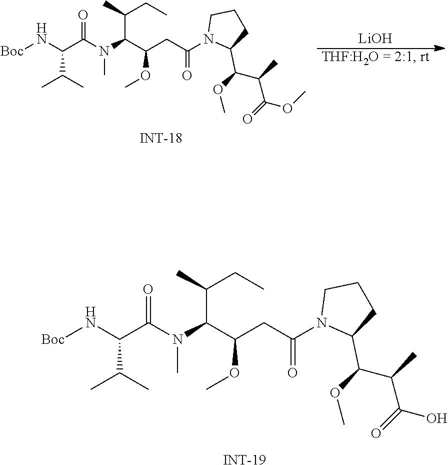

### (2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoic acid INT-19

To a solution of INT-18 (1.61 g, 2.75 mmol) in 20 mL THF was added 10 mL LiOH (330 mg, 13.78 mmol) aqueous. The mixture was stirred at room temperature for 16 h. HPLC showed completion (<5% de-Boc byproduct detected). 4 M HCl/dioxane (˜4 mL) was added slowly to adjust the PH to ˜2. The resulting mixture was diluted with H₂O (15 mL), then extracted with DCM (30 mL*3). The combined organic layers were washed with H₂O (15 mL) and brine (15 mL), dried over Na₂SO₄, filtrated and concentrated. The residue was purified by reverse phase column (H₂O:CH₃CN) to afford INT-19 (1.06 g, 67% yield) as a colorless oil. LCMS (ESI): m/z 572.3 [M+H]⁺.

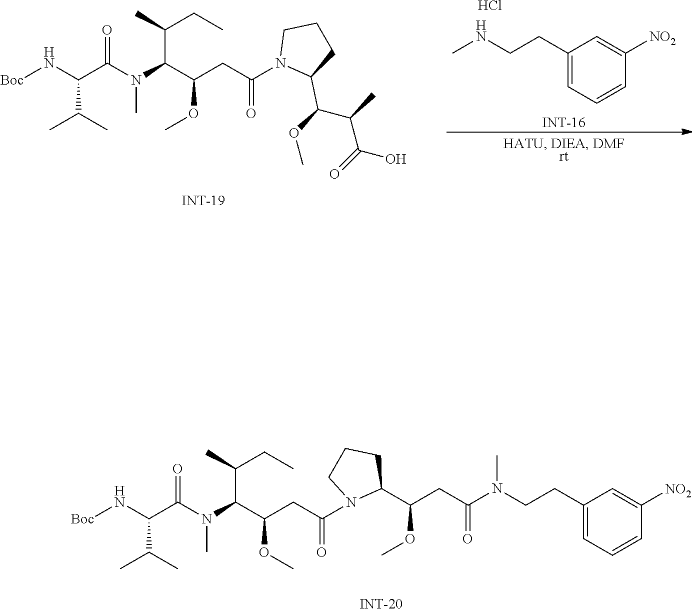

### tert-butyl ((S)-2-(((2S,3R)-5-(ethyl((2S,3R)-3-methoxy-5-((3-nitrophenethyl)amino)-5-oxopent an-2-yl)amino)-3-methoxy-5-oxopentan-2-yl)(methyl)amino)-4-methylpent-1-en-3-yl)carbamate INT-20

Tube A: To a solution of INT-16 (524 mg, 2.42 mmol) in DMF (3 mL) was added DIEA (0.8 mL, 4.7 mmol). The mixture was stirred at room temperature for 0.5 h to form solution A.
Tube B: To another solution of INT-19 (1.06 g, 1.86 mmol) in 10 mL DMF was added HATU (1.42 g, 3.72 mmol) at room temperature. The mixture was stirred at room temperature for 0.5 h, solution A was added, followed by DIEA (0.6 mL, 3.75 mmol) added. The resulting mixture was stirred at room temperature for 2 h. LCMS showed completion. The reaction was purified by reverse phase column (H₂O/CH₃CN) directly to afford INT-20 (1.16 g, 85% yield) as yellow oil. LCMS (ESI): m/z 734.1 [M+H]⁺.

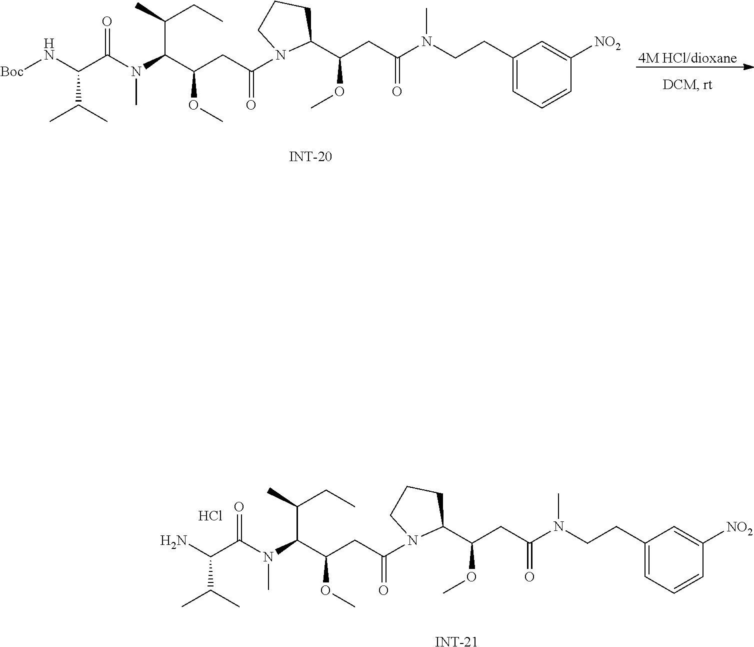

### (3R,4S)-4-((S)-2-amino-N,3-dimethylbutanamido)-N-ethyl-3-methoxy-N-((2S,3R)-3-methoxy-5-((3-nitrophenethyl)amino)-5-oxopentan-2-yl)pentanamide hydrochloride INT-21

To a solution of INT-20 (1.16 g, 1.58 mmol) in 12 mL DCM was added 4 M HCl/dioxane (6 mL, 24 mmol). The reaction was stirred at room temperature for 2 h. TLC showed completion. The mixture was concentrated to dry. The residue was slurried with MTBE (10 mL*3) then freeze dried to afford INT-21 (1.1 g, 100% yield) as a yellow solid. LCMS (ESI): m/z 634.2 [M+H]⁺.

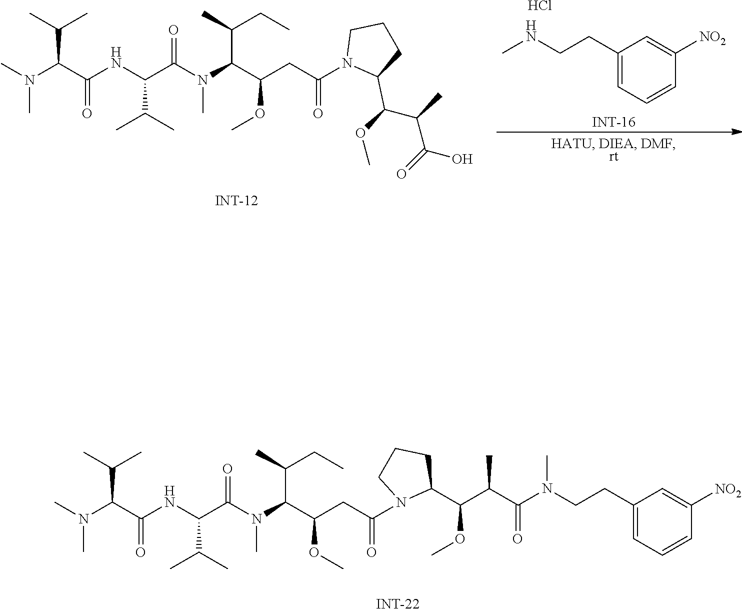

### (S)-2-((S)-2-(dimethylamino)-3-methylbutanamido)-N-((3R,4S,5S)-3-methoxy-1-((S)-2-((1R,2R)-1-methoxy-2-methyl-3-(methyl(3-nitrophenethyl)amino)-3-oxopropyl)pyrrolidin-1-yl)-5-methyl-1-oxoheptan-4-yl)-N,3-dimethylbutanamide INT-22

To a solution of INT-16 (178 mg, 0.818 mmol) in 7 mL DMF was added DIEA (227 mg, 1.753 mmol) dropwise, followed by the addition of HATU (334 mg, 0.877 mmol), INT-12 (350 mg, 0.584 mmol) and DMF (7 mL). The mixture was stirred at room temperature for 2 h, HPLC showed completion. The reaction mixture was added EtOAc (100 mL) then washed with brine (50 mL*3). The organic layer was dried over Na₂SO₄, filtrated and concentrated. The residue was purified by silica gel column (DCM:MeOH=100:1 to 10:1, v/v) then Prep-TLC (DCM: MeOH=12:1, v/v; R_{f}=0.6) to afford INT-22 (277 mg, 62% yield) as a yellow oil.

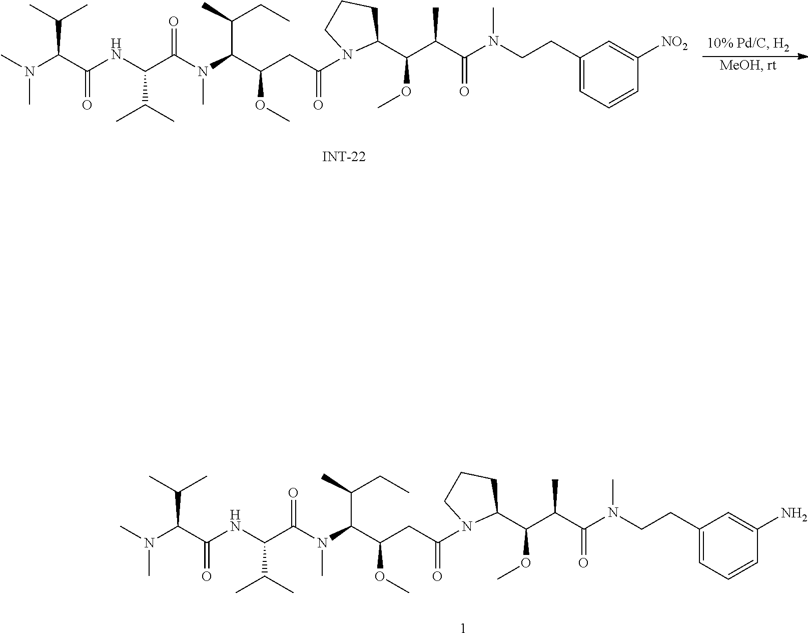

### (S)-N-((3R,4S,5S)-1-((S)-2-((1R,2R)-3-((3-aminophenethyl)(methyl)amino)-1-methoxy-2-methyl-3-oxopropyl)pyrrolidin-1-yl)-3-methoxy-5- methyl-1-oxoheptan-4-yl)-2-((S)-2-(dimethylamino)-3-methylbutanamido)-N,3-dimethylbutanamide 1

To a solution of INT-22 (277 mg, 0.364 mmol) in 25 mL MeOH was added 10% Pd/C (50 mg). The reaction was then stirred under a H₂ atmosphere (1 atm) at room temperature for 16 h. TLC (DCM: MeOH=12:1, v/v, R_{f}=0.6) showed completion. The mixture was filtrated, and the filtrate was concentrated. The residue was purified by prep-TLC (DCM: MeOH=12:1, v/v, R_{f}=0.6) to afford 1 (122 mg, 46% yield) as an off-white solid. LCMS (ESI): m/z 731.0 [M+H]+; HPLC: 98.6% @210 nm, Rₜ=8.10 min; ¹H NMR (400 MHz, CDCl₃) δ 7.15-7.03 (m, 1H), 7.03-6.94 (m, 1H), 6.61-6.53 (m, 1H), 6.53-6.41 (m, 2H), 4.97-4.89 (m, 1H), 4.89-4.66 (m, 1H), 4.27-4.14 (m, 1H), 4.01-3.88 (m, 1H), 3.86-3.68 (m, 2H), 3.60-3.53 (m, 1H), 3.45-3.37 (m, 4H), 3.37-3.29 (m, 4H), 3.27-3.22 (m, 1H), 3.17-3.14 (m, 1H), 3.04-2.98 (m, 1H), 2.94-2.85 (m, 3H), 2.80-2.68 (m, 3H), 2.62-2.45 (m, 3H), 2.38-2.27 (m, 6H), 2.12-2.05 (m, 2H), 2.04-1.97 (m, 2H), 1.95-1.90 (m, 1H), 1.86-1.81 (m, 1H), 1.80-1.74 (m, 1H), 1.73-1.66 (m, 1H), 1.55-1.44 (m, 1H), 1.20-1.12 (m, 3H), 1.06-0.96 (m, 9H), 0.96-0.90 (m, 6H), 0.85-0.78 (m, 3H).

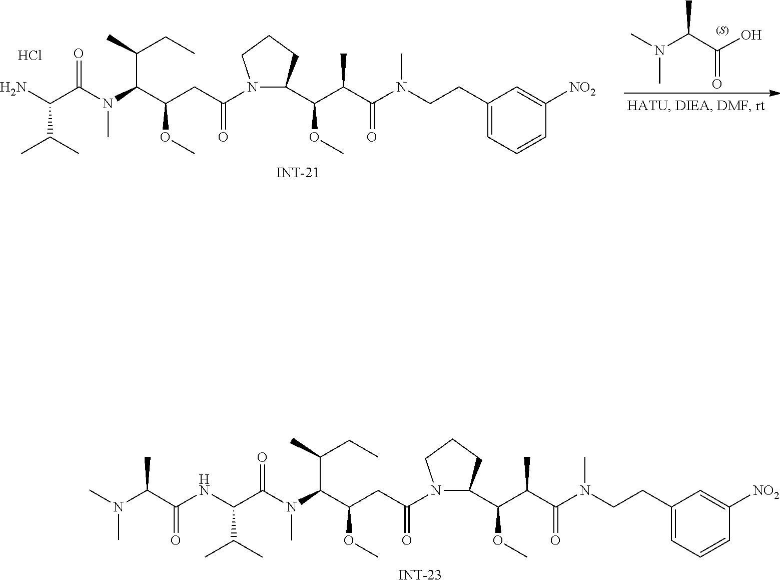

### (S)-2-((S)-2-(dimethylamino)propanamido)-N-((3R,4S,5S)-3-methoxy-1-((S)-2-((1R,2R)-1-methoxy-2-methyl-3-(methyl(3-nitrophenethyl)amino)-3-oxopropyl)pyrrolidin-1-yl)-5-methyl-1-oxoheptan-4-yl)-N,3-dimethylbutanamide INT-23

To a solution of INT-21 (200 mg, 0.32 mmol) and (S)-2-(dimethylamino)propanoic acid (44 mg, 0.38 mmol) in 5 mL DMF was added HATU (1.42 g, 3.72 mmol), followed by DIEA (124 mg, 0.96 mmol) added. The mixture was stirred at room temperature for 1 h, LCMS showed completion (LCMS (ESI): m/z 733.1 [M+H]⁺). The reaction was quenched by 20 mL H₂O, extracted with DCM (15 mL*3). The combined organic layers were washed with H₂O and brine, dried over Na₂SO₄, filtrated and concentrated. The residue was purified by Prep-TLC (DCM: MeOH=12:1, v/v; R_{f}=0.7) to afford INT-23 (210 mg, 91% yield) as a light yellow oil (87% purity @210 nm on HPLC), which was used directly without re-purification.

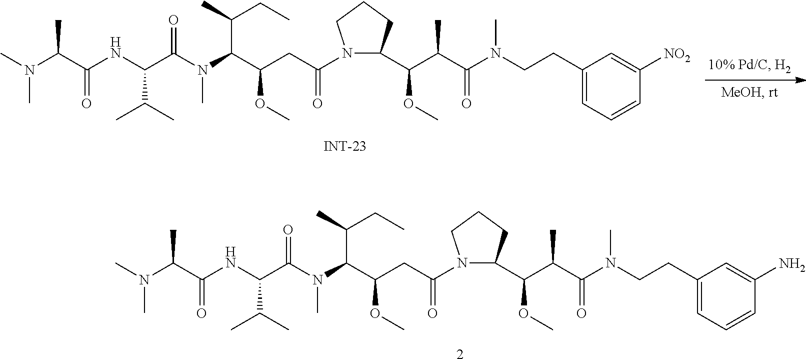

### Step 2: (S)-N-((3R,4S,5S)-1-((S)-2-((1R,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)-2-((S)-2-(dimethylamino)propanamido)-N,3-dimethylbutanamide 2

To a solution of INT-23 (210 mg, 0.287 mmol) in 5 mL MeOH was added 10% Pd/C (42 mg). The reaction was then stirred under a H₂ atmosphere (1 atm) at room temperature for 3 h. LCMS showed completion. The mixture was filtrated, and the filtrate was concentrated. The residue was purified by reverse phase column (H₂O:CH₃CN) to afford 2 (180 mg, 94% yield) as an off-white solid. LCMS (ESI): m/z 703.4 [M+H]⁺; HPLC: 95.1% @210 nm, Rₜ=9.32 min; ¹H NMR (400 MHz, DMSO) δ 7.73 (t, J=10.0 Hz, 1H), 6.95-6.85 (m, 1H), 6.45-6.31 (m, 3H), 5.01-4.84 (m, 2H), 4.79-4.51 (m, 2H), 4.05-3.93 (m, 1H), 3.93-3.76 (m, 1H), 3.75-3.67 (m, 1H), 3.67-3.55 (m, 1H), 3.54-3.41 (m, 2H), 3.40-3.35 (m, 1H), 3.30-3.24 (m, 2H), 3.24-3.05 (m, 5H), 2.94 (s, 1H), 2.91-2.88 (m, 2H), 2.88-2.74 (m, 2H), 2.68-2.54 (m, 3H), 2.49-2.38 (m, 1H), 2.26-2.14 (m, 6H), 2.07-1.78 (m, 4H), 1.76-1.55 (m, 2H), 1.33-1.21 (m, 2H), 1.12-1.05 (m, 4H), 1.04-0.95 (m, 2H), 0.94-0.81 (m, 9H), 0.80-0.73 (m, 3H).

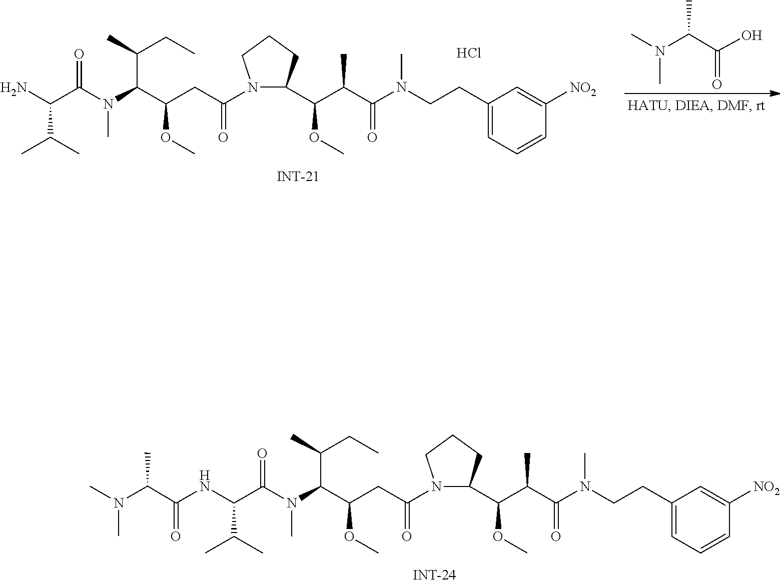

### (S)-2-((R)-2-(dimethylamino)propanamido)-N-((3R,4S,5S)-3-methoxy-1-((S)-2-((1R,2R)-1-methoxy-2-methyl-3-(methyl(3-nitrophenethyl)amino)-3-oxopropyl)pyrrolidin-1-yl)-5-methyl-1-oxoheptan-4-yl)-N,3-dimethylbutanamide INT-24

To a solution of INT-21 (165 mg, 0.246 mmol) and N, N-Dimethyl-L-Alanine (38 mg, 0.32 mmol) in 3 mL DMF was added HATU (187 mg, 0.492 mmol), followed by the addition of DIEA (0.16 mL, 0.985 mmol). The mixture was stirred at room temperature under a N₂ atmosphere for 2 h, LCMS showed completion. The reaction mixture was purified by reverse phase column (H₂O:CH₃CN) directly to afford INT-24 (130 mg, 72% yield) as a colorless oil. LCMS (ESI): m/z 733.1 [M+H]⁺.

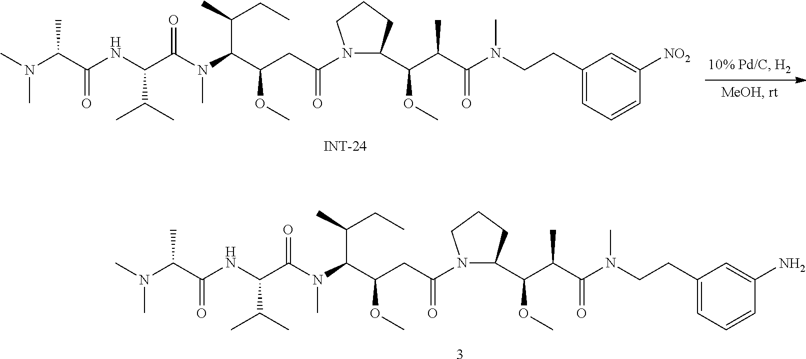

### (S)-N-((3R,4S,5S)-1-((S)-2-((IR,2R)-3-((3-aminophenethyl)(methyl)amino)-1-methoxy-2-meth yl-3-oxopropyl)pyrrolidin-1-yl)-3-methoxy-5-methyl-1-oxoheptan-4-yl)-2-((R)-2-(dimethylamino)propanamido)-N,3-dimethylbutanamide 3

To a solution of INT-24 (130 mg, 0.177 mmol) in 3 mL MeOH was added 10% Pd/C (40 mg). The reaction then was stirred under a H₂ atmosphere (1 atm) at room temperature for 3 h. LCMS showed completion. The mixture was filtrated, and the filtrate was concentrated. The residue was freeze-dried to afford 3 (115 mg, 92% yield) as an off-white solid. LCMS (ESI): m/z 703.2 [M+H]⁺; HPLC: 99.4% @210 nm, Rₜ=7.94 min; ¹H NMR (400 MHz, DMSO) δ 7.85-7.74 (m, 1H), 6.96-6.86 (m, 1H), 6.53 (bs, 1H), 6.47-6.31 (m, 3H), 4.93 (dd, J₁=29.9 Hz, J₂=10.8 Hz, 2H), 4.79-4.62 (m, 1H), 4.60-4.49 (m, 1H), 4.07-3.95 (m, 1H), 3.93-3.67 (m, 2H), 3.66-3.43 (m, 3H), 3.40-3.34 (m, 1H), 3.30-3.24 (m, 2H), 3.21-3.05 (m, 5H), 2.97-2.94 (m, 1H), 2.93-2.87 (m, 2H), 2.86-2.73 (m, 2H), 2.68-2.54 (m, 3H), 2.49-2.37 (m, 1H), 2.18-2.12 (m, 6H), 2.01-1.78 (m, 4H), 1.72-1.54 (m, 2H), 1.35-1.26 (m, 1H), 1.10-0.96 (m, 6H), 0.94-0.82 (m, 10H), 0.81-0.73 (m, 3H).

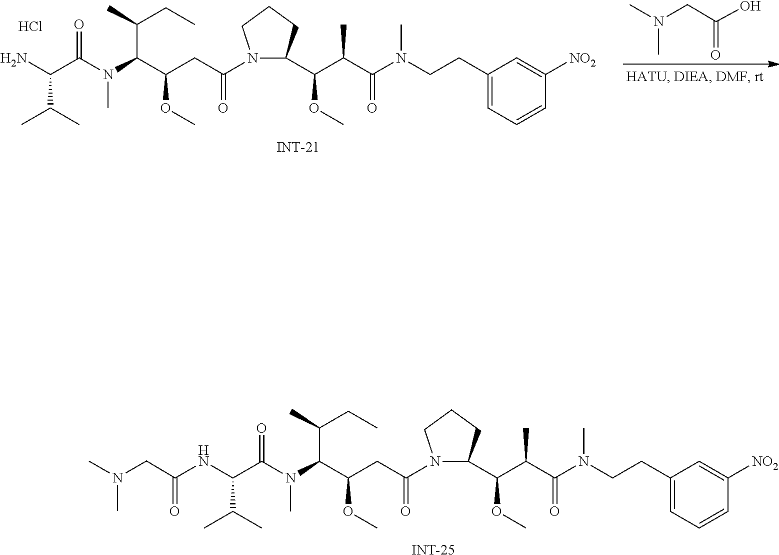

### (S)-2-(2-(dimethylamino)acetamido)-N-((3R,4S,5S)-3-methoxy-1-((S)-2-((1R,2R)-1-methoxy-2-methyl-3-(methyl(3-nitrophenethyl)amino)-3-oxopropyl)pyrrolidin-1-yl)-5-methyl-1-oxoheptan-4-yl)-N,3-dimethylbutanamide INT-21

To a solution of INT-21 (210 mg, 0.332 mmol) and 2-(dimethylamino)acetic acid (41 mg, 0.398 mmol) in 5 mL DMF was added HATU (189 mg, 0.497 mmol), followed by DIEA (128 mg, 0.992 mmol) added. The mixture was stirred at room temperature for 1 h, LCMS showed completion (LCMS (ESI): m/z 719.1 [M+H]⁺). The reaction was diluted with 20 mL H₂O, then extracted with DCM (15 mL*3). The combined organic layers were washed with H₂O (10 mL) and brine (10 mL*3), dried over Na₂SO₄, filtrated and concentrated. The residue was purified by Prep-TLC (DCM:MeOH=13:1, v/v; R_{f}=0.7) to afford INT-25 (190 mg, 79.8% yield) as a colorless oil.

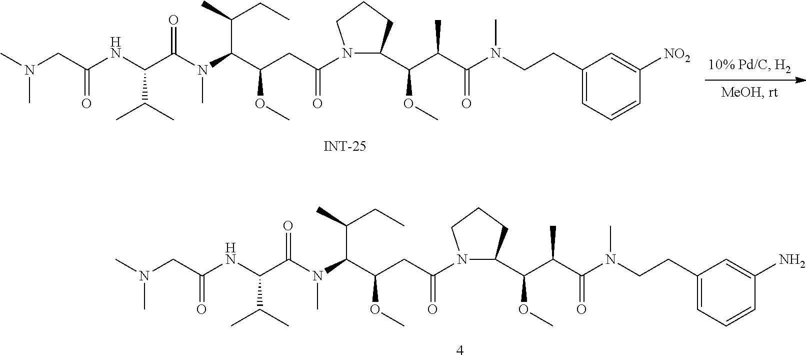

### (S)-N-((3R,4S,5S)-1-((S)-2-((1R,2R)-3-((3-aminophenethyl)(methyl)amino)-1-methoxy-2-meth yl-3-oxopropyl)pyrrolidin-1-yl)-3-methoxy-5-methyl-1-oxoheptan-4-yl)-2-(2-(dimethylamino)acetamido)-N,3-dimethylbutanamide 4

To a solution of INT-25 (190 mg, 0.264 mmol) in 5 mL MeOH was added 10% Pd/C (38 mg). The reaction was then stirred under a H₂ atmosphere (1 atm) at room temperature for 3 h. LCMS showed completion. The mixture was filtrated, and the filtrate was concentrated. The residue was purified by reverse phase column (H₂O:CH₃CN) to afford 4 (103 mg, 56.6% yield) as a white solid. LCMS (ESI): m/z 689.2 [M+H]⁺; HPLC: 99.2% @210 nm, Rₜ=11.16 min; ¹H NMR (400 MHz, DMSO) δ 7.69-7.60 (m, 1H), 6.96-6.86 (m, 1H), 6.45-6.31 (m, 3H), 4.93 (dd, J₁=31.4 Hz, J₂=12.4 Hz, 2H), 4.82-4.55 (m, 2H), 4.06-3.94 (m, 1H), 3.93-3.63 (m, 2H), 3.60-3.42 (m, 2H), 3.39-3.35 (m, 1H), 3.30-3.24 (m, 2H), 3.22-3.15 (m, 3H), 3.15-3.04 (m, 2H), 3.01-2.92 (m, 2H), 2.91-2.85 (m, 2H), 2.85-2.77 (m, 2H), 2.67-2.54 (m, 3H), 2.48-2.38 (m, 1H), 2.25-2.15 (m, 6H), 2.03-1.77 (m, 4H), 1.74-1.56 (m, 2H), 1.33-1.21 (m, 2H), 1.09-0.99 (m, 2H), 0.97-0.70 (m, 14H).

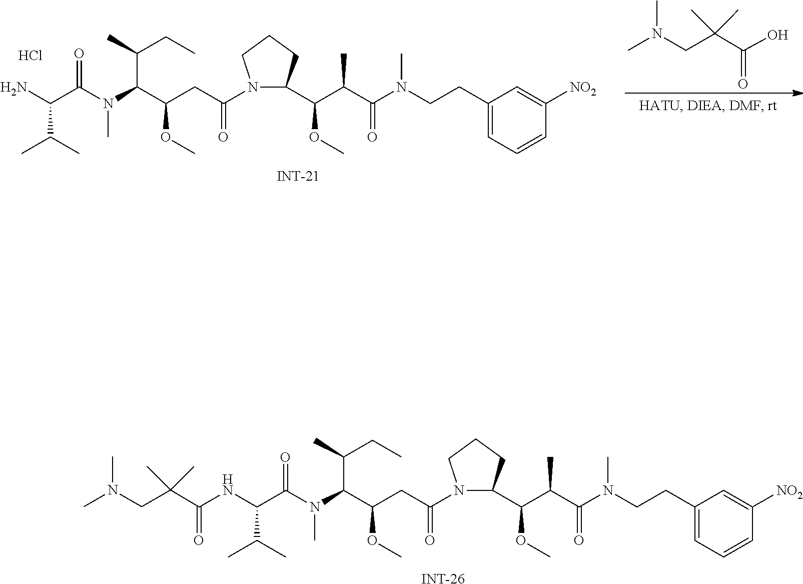

### (S)-2-(3-(dimethylamino)-2,2-dimethylpropanamido)-N-((3R,4S,5S)-3-methoxy-1-((S)-2-((1R,2R)-1-methoxy-2-methyl-3-(methyl(3-nitrophenethyl)amino)-3-oxopropyl)pyrrolidin-1-yl)-5-methyl-1-oxoheptan-4-yl)-N,3-dimethylbutanamide INT-26

To a solution of INT-21 (200 mg, 0.30 mmol) in 4 mL DMF was added DIEA (174 mg, 1.34 mmol). The mixture was stirred at room temperature for 5 min, then 3-(dimethylamino)-2,2-dimethylpropanoic acid (52 mg, 0.36 mmol) and HATU (170 mg, 0.448 mmol) were added. The resulting mixture was stirred at room temperature for 1 h, LCMS showed completion. The reaction was concentrated directly. The residue was purified by reverse phase column (H₂O:CH₃CN) to afford crude INT-26 (223 mg, 98% yield) as a yellow oil, which was used directly without re-purification. LCMS (ESI): m/z 761.0 [M+H]⁺.

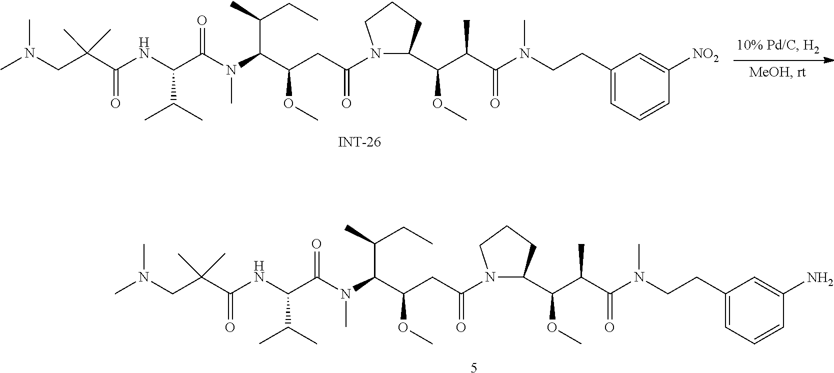

### Step 2: (S)-N-((3R,4S,5S)-1-((S)-2-((1R,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)-2-(3-(dimethylamino)-2,2-dimethylpropanamido)-N,3-dimethylbutanamide 5

To a solution of INT-26 (223 mg, 0.293 mmol) in 4 mL MeOH was added 10% Pd/C (45 mg). The reaction was then stirred under a H₂ atmosphere (1 atm) at room temperature for 2 h. LCMS showed completion. The mixture was filtrated, and the filtrate was concentrated. The residue was purified by reverse phase column (H₂O:CH₃CN) to afford 5 (184 mg, 85.6% yield) as an off-white solid. LCMS (ESI): m/z 731.2 [M+H]⁺; HPLC: 96.7% @210 nm, Rₜ=12.84 min; ¹H NMR (400 MHz, DMSO) δ 7.93 (s, 1H), 6.98-6.86 (m, 1H), 6.48-6.31 (m, 3H), 5.16-4.79 (m, 1H), 4.78-4.59 (m, 1H), 4.58-4.39 (m, 1H), 4.09-3.96 (m, 1H), 3.95-3.57 (m, 2H), 3.56-3.39 (m, 2H), 3.31-3.24 (m, 4H), 3.23-3.12 (m, 5H), 3.10-2.95 (m, 3H), 2.91-2.86 (m, 2H), 2.85-2.75 (m, 2H), 2.72-2.54 (m, 7H), 2.47-2.39 (m, 1H), 2.36-2.20 (m, 1H), 2.14-2.03 (m, 1H), 1.96-1.75 (m, 3H), 1.74-1.56 (m, 2H), 1.31-1.14 (m, 6H), 1.12-0.96 (m, 3H), 0.96-0.82 (m, 10H), 0.82-0.59 (m, 5H).

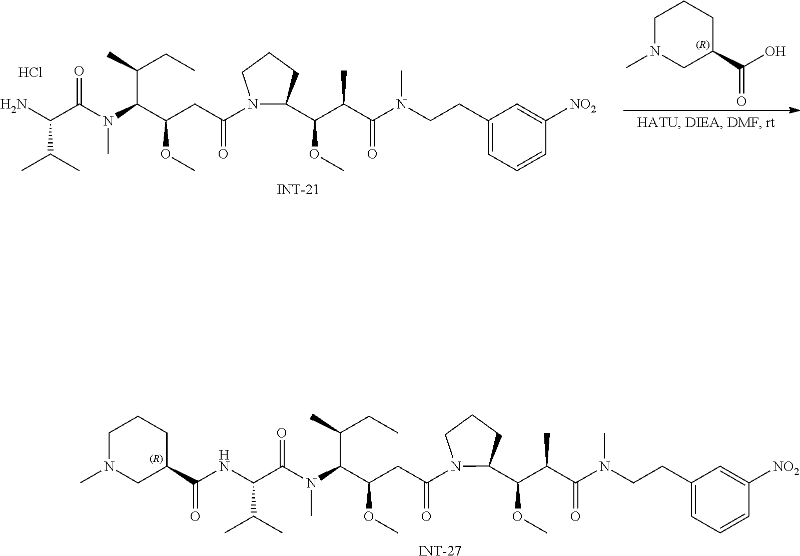

### (R)-N-((S)-1-(((3R,4S,5S)-3-methoxy-1-((S)-2-((1R,2R)-1-methoxy-2-methyl-3-(methyl(3-nitro phenethyl)amino)-3-oxopropyl)pyrrolidin-1-yl)-5-methyl-1-oxoheptan-4-yl)(methyl)amino)-3-methyl-1-oxobutan-2-yl)-1-methylpiperidine-3-carboxamide INT-27

To a solution of INT-21 (200 mg, 0.30 mmol) in 4 mL DMF was added DIEA (174 mg, 1.34 mmol). The mixture was stirred at room temperature for 5 min, then (R)-1-methylpiperidine-3-carboxylic acid (51 mg, 0.36 mmol) and HATU (170 mg, 0.448 mmol) were added. The resulting mixture was stirred at room temperature for 1 h, LCMS showed completion. The reaction was concentrated. The residue was purified by reverse phase column (H₂O:CH₃CN) to afford INT-27 (232 mg, 100% yield) as a light yellow oil. LCMS (ESI): m/z 759.0 [M+H]⁺.

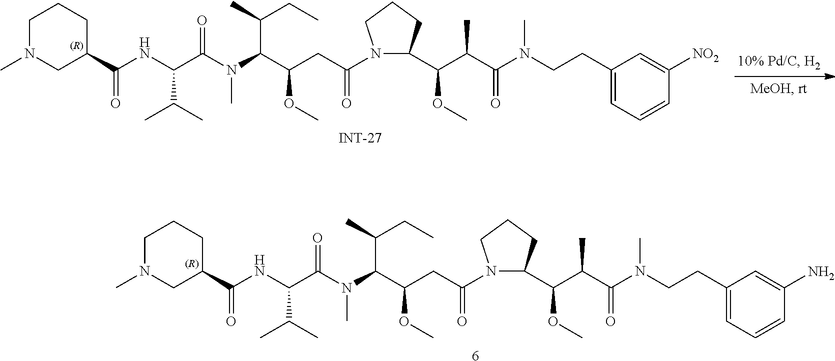

### (R)-N-((S)-1-(((3R,4S,5S)-1-((S)-2-((IR,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-3-carboxamide 6

To a solution of INT-27 (232 mg, 0.293 mmol) in 4 mL MeOH was added 10% Pd/C (46 mg). The reaction was then stirred under a H₂ atmosphere (1 atm) at room temperature for 3 h. LCMS showed completion. The mixture was filtrated, and the filtrate was concentrated. The residue was purified by reverse phase column (H₂O:CH₃CN) to afford 6 (191 mg, 85.7% yield) as an off-white solid. LCMS (ESI): m/z 729.1 [M+H]⁺; HPLC: 97.7% @210 nm, Rₜ=12.00 min; ¹H NMR (400 MHz, DMSO) δ 8.46-8.25 (m, 1H), 7.02-6.82 (m, 1H), 6.44-6.29 (m, 3H), 5.07-4.83 (m, 1H), 4.79-4.59 (m, 1H), 4.56-4.42 (m, 1H), 4.07-3.97 (m, 1H), 3.96-3.69 (m, 2H), 3.65-3.56 (m, 1H), 3.54-3.45 (m, 2H), 3.29-3.26 (m, 2H), 3.23-3.12 (m, 6H), 3.08-2.93 (m, 3H), 2.92-2.87 (m, 2H), 2.85-2.78 (m, 2H), 2.77-2.69 (m, 2H), 2.65-2.57 (m, 5H), 2.47-2.38 (m, 1H), 2.04-1.82 (m, 4H), 1.81-1.69 (m, 3H), 1.67-1.56 (m, 2H), 1.47-1.36 (m, 1H), 1.32-1.25 (m, 1H), 1.12-0.95 (m, 4H), 0.95-0.81 (m, 11H), 0.81-0.73 (m, 3H).

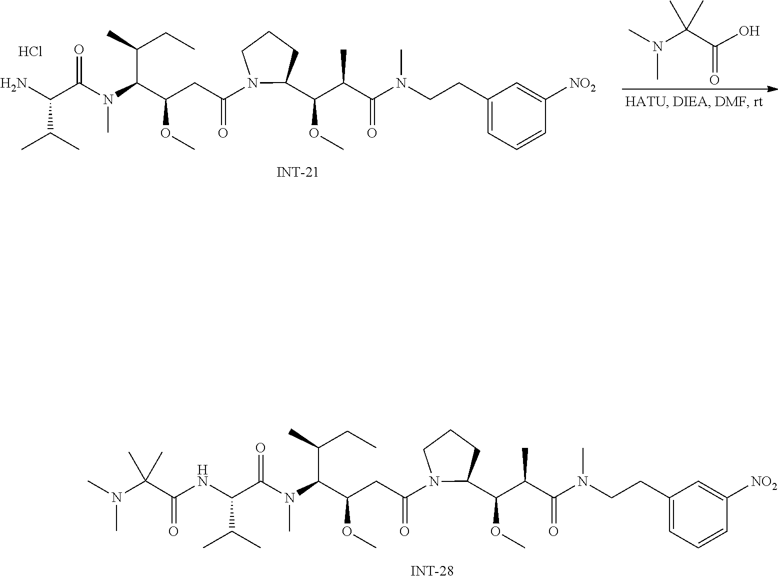

### (S)-2-(2-(dimethylamino)-2-methylpropanamido)-N-((3R,4S,5S)-3-methoxy-1-((S)-2-((1R,2R)-1-methoxy-2-methyl-3-(methyl(3-nitrophenethyl)amino)-3-oxopropyl)pyrrolidin-1-yl)-5-methyl-1-oxoheptan-4-yl)-N,3-dimethylbutanamide INT-28

To a solution of 2-(dimethylamino)-2-methylpropanoic acid (78 mg, 0.597 mmol) and HATU (227 mg, 0.597 mmol) in 3 mL DMF was added DIEA (0.2 mL, 1.2 mmol). The mixture was stirred at room temperature for 30 min under a N₂ atmosphere, and then INT-21 (200 mg, 0.298 mmol) was added. The resulting mixture was stirred at room temperature for 4 h. LCMS showed completion. The reaction mixture was purified by reverse phase column (H₂O:CH₃CN) directly to afford INT-28 (140 mg, 63% yield) as a light yellow solid. LCMS (ESI): m/z 747.0 [M+H]⁺.

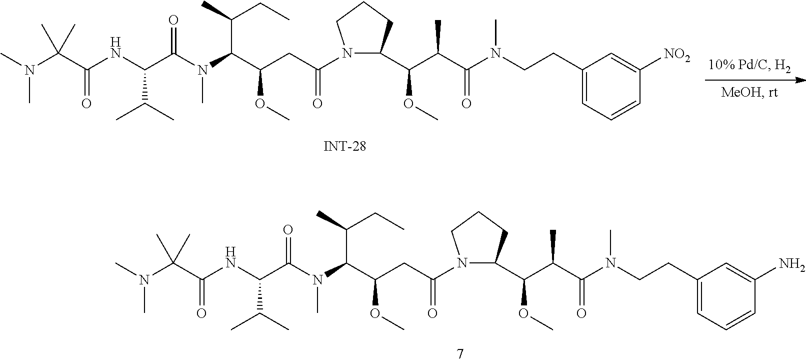

### (S)-N-((3R,4S,5S)-1-((S)-2-((IR,2R)-3-((3-aminophenethyl)(methyl)amino)-1-methoxy-2-meth yl-3-oxopropyl)pyrrolidin-1-yl)-3-methoxy-5-methyl-1-oxoheptan-4-yl)-2-(2-(dimethylamino)-2-methylpropanamido)-N,3-dimethylbutanamide 7

To a solution of INT-28 (140 mg, 0.187 mmol) in 3 mL MeOH was added 10% Pd/C (40 mg). The reaction was then stirred under a H₂ atmosphere (1 atm) at room temperature for 4 h. LCMS showed completion. The mixture was filtrated, and the filtrate was concentrated. The residue was freeze-dried to afford 7 (125 mg, 93% yield) as a yellow solid. LCMS (ESI): m/z 717.1 [M+H]⁺; HPLC: 97.7% @210 nm, Rₜ=12.62 min; ¹H NMR (400 MHz, DMSO) δ 7.71-7.55 (m, 1H), 6.96-6.85 (m, 1H), 6.96-6.85 (m, 1H), 6.48-6.31 (m, 3H), 5.04-4.85 (m, 2H), 4.78-4.49 (m, 2H), 4.04-3.96 (m, 1H), 3.95-3.68 (m, 2H), 3.66-3.55 (m, 1H), 3.54-3.42 (m, 2H), 3.41-3.36 (m, 1H), 3.34-3.25 (m, 2H), 3.24-3.10 (m, 4H), 3.10-2.92 (m, 2H), 2.92-2.84 (m, 2H), 2.81 (d, J=11.2 Hz, 1H), 2.69-2.53 (m, 4H), 2.48-2.38 (m, 1H), 2.31-2.05 (m, 6H), 2.03-1.76 (m, 4H), 1.76-1.55 (m, 2H), 1.32-1.22 (m, 2H), 117-1.02 (m, 5H), 1.01-0.95 (m, 3H), 0.94-0.72 (m, 12H).

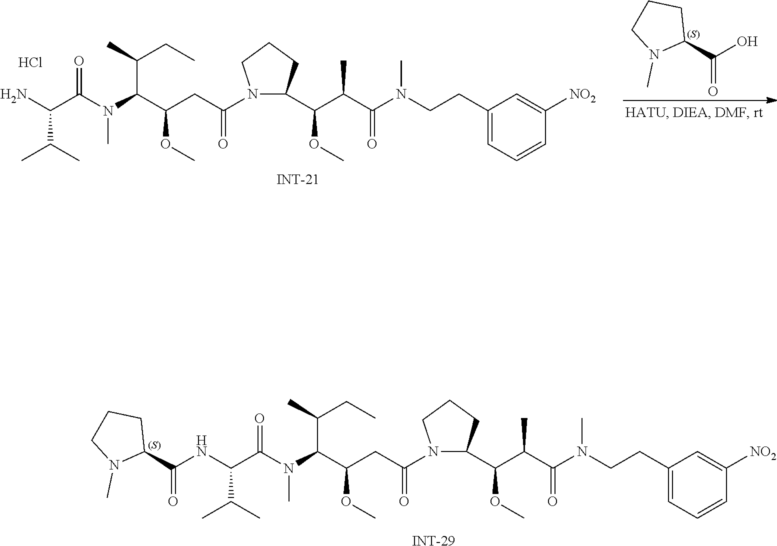

### (S)-N-((S)-1-(((3R,4S,5S)-3-methoxy-1-((S)-2-((1R,2R)-1-methoxy-2-methyl-3-(methyl(3-nitro phenethyl)amino)-3-oxopropyl)pyrrolidin-1-yl)-5-methyl-1-oxoheptan-4-yl)(methyl)amino)-3-methyl-1-oxobutan-2-yl)-1-methylpyrrolidine-2-carboxamide INT-29

To a solution of N-methyl-L-proline monohydrate (50 mg, 0.388 mmol) and HATU (227 mg, 0.597 mmol) in 3 mL DMF was added DIEA (0.22 mL, 1.34 mmol). The mixture was stirred at room temperature for 30 min under a N₂ atmosphere, then INT-21 (200 mg, 0.298 mmol) was added. The resulting mixture was stirred at room temperature for 2 h. LCMS showed completion. The reaction mixture was concentrated and the residue was re-dissolved in 70 mL EtOAc. The organic layer was washed with H₂O (15 mL*2) and brine (10 mL), dried over Na₂SO₄, filtrated and concentrated to afford crude INT-29 (260 mg, >100% yield) as a yellow foam solid, which was used directly without further purification. LCMS (ESI): m/z 745.1 [M+H]⁺.

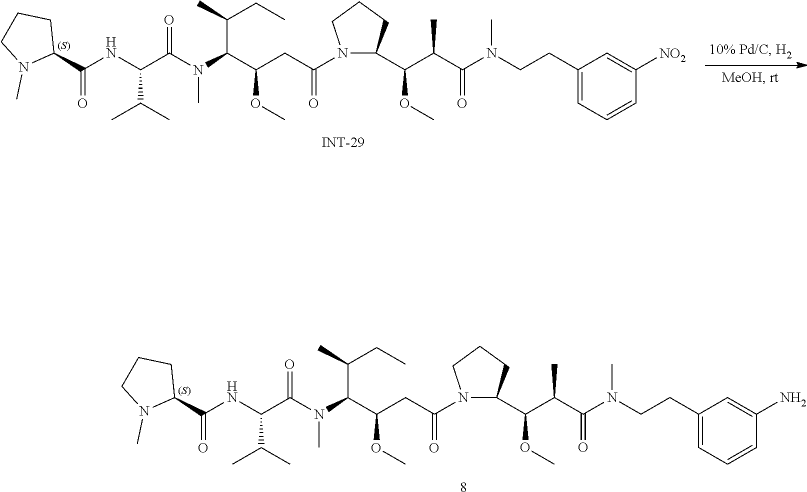

### (S)-N-((S)-1-(((3R,4S,5S)-1-((S)-2-((1R,2R)-3-((3-aminophenethyl)((methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-2-carboxamide 8

To a solution of INT-29 (220 mg, 0.295 mmol; see above) in 5 mL MeOH was added 10% Pd/C (60 mg). The reaction was then stirred under a H₂ atmosphere (1 atm) at room temperature for 5 h. LCMS showed completion. The mixture was filtrated, and the filtrate was concentrated. The residue was purified by reverse phase column (H₂O:CH₃CN) to afford 8 (150 mg, 71% yield) as an off-white solid. LCMS (ESI): m/z 715.1 [M+H]⁺; HPLC: 98.2% @210 nm, Rₜ=11.96 min; ¹H NMR (400 MHz, DMSO) δ 9.06-8.95 (m, 1H), 7.08-6.96 (m, 1H), 6.68-6.49 (m, 3H), 4.80-4.50 (m, 2H), 4.19-3.81 (m, 3H), 3.79-3.71 (m, 1H), 3.69-3.43 (m, 5H), 3.29-3.25 (m, 2H), 3.22-3.09 (m, 6H), 3.01-2.86 (m, 4H), 2.84-2.76 (m, 4H), 2.72-2.59 (m, 3H), 2.47-2.40 (m, 1H), 2.40-2.17 (m, 1H), 2.12-1.78 (m, 6H), 1.78-1.56 (m, 3H), 1.36-1.15 (m, 2H), 1.12-1.01 (m, 2H), 1.01-0.84 (m, 11H), 0.84-0.70 (m, 3H).

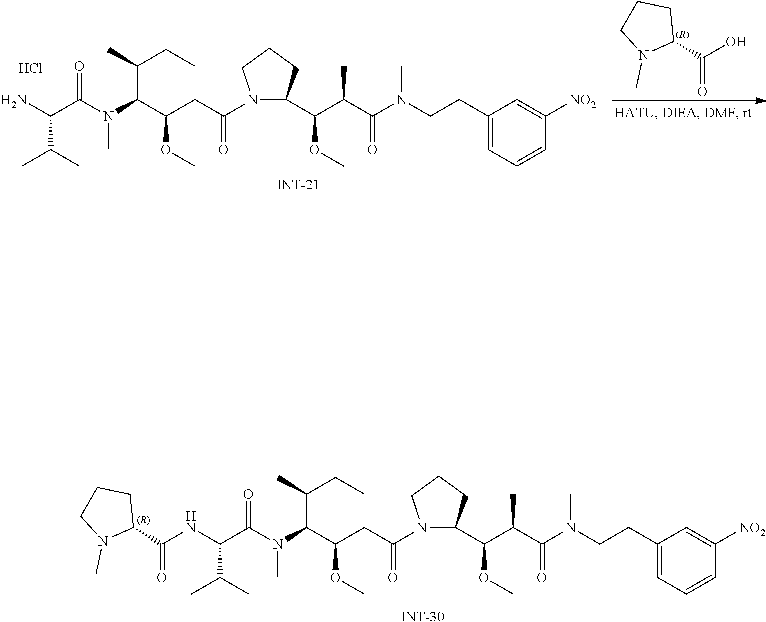

### (R)-N-((S)-1-(((3R,4S,5S)-3-methoxy-1-((S)-2-((1R,2R)-1-methoxy-2-methyl-3-(methyl(3-nitro phenethyl)amino)-3-oxopropyl)pyrrolidin-1-yl)-5-methyl-1-oxoheptan-4-yl)(methyl)amino)-3-methyl-1-oxobutan-2-yl)-1-methylpyrrolidine-2-carboxamide INT-30

To a solution of INT-21 (200 mg, 0.3 mmol) and (R)-1-methylpyrrolidine-2-carboxylic acid (50 mg, 0.387 mmol) in 5 mL DMF was added HATU (182 mg, 0.48 mmol), followed by DIEA (124 mg, 0.96 mmol) added. The mixture was stirred at room temperature for 1 h, LCMS showed completion (LCMS (ESI): m/z 745.0 [M+H]⁺). The reaction was quenched by 0.5 mL H₂O, and concentrated directly. The residue was purified by Prep-TLC (DCM: MeOH=12:1, v/v; Rf=0.7) to afford crude INT-30 (220 mg, 93.6% yield) as a colorless oil.

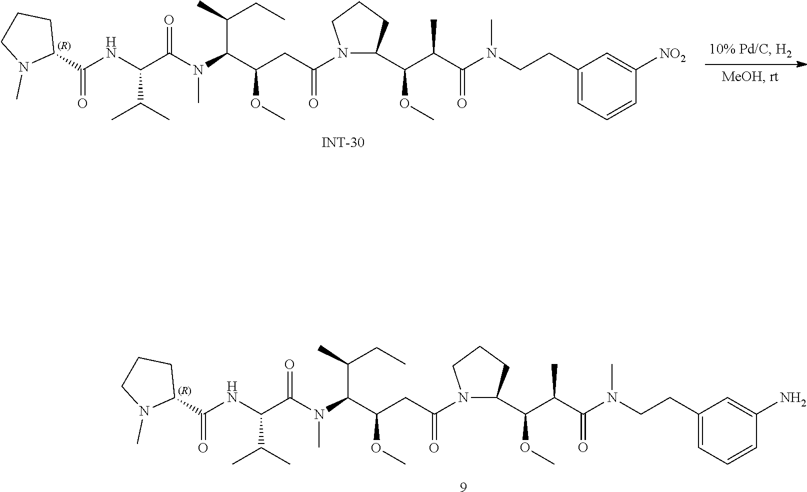

### (R)-N-((S)-1-(((3R,4S,5S)-1-((S)-2-((1R,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-2-carboxamide 9

To a solution of INT-30 (220 mg, 0.296 mmol; see above) in 5 mL MeOH was added 10% Pd/C (50 mg). The reaction was then stirred under a H₂ atmosphere (1 atm) at room temperature for 3 h. LCMS showed completion. The mixture was filtrated, and the filtrate was concentrated. The residue was purified by reverse phase column (H₂O:CH₃CN) to afford 9 (168 mg, 79.6% yield) as a white solid. LCMS (ESI): m/z 715.3 [M+H]⁺; HPLC: 98.2% @210 nm, Rt=11.88 min; ¹H NMR (400 MHz, DMSO) δ 7.71-7.59 (m, 1H), 6.96-6.85 (m, 1H), 6.46-6.30 (m, 3H), 4.99-4.86 (m, 2H), 4.76-4.50 (m, 2H), 4.13-3.87 (m, 2H), 3.78-3.60 (m, 2H), 3.59-3.41 (m, 3H), 3.30-3.23 (m, 2H), 3.22-3.10 (m, 4H), 3.09-2.92 (m, 3H), 2.91-2.83 (m, 2H), 2.83-2.76 (m, 2H), 2.68-2.55 (m, 3H), 2.49-2.37 (m, 1H), 2.34-2.22 (m, 4H), 2.11-1.95 (m, 2H), 1.95-1.77 (m, 3H), 1.75-1.48 (m, 5H), 1.33-1.18 (m, 2H), 1.11-1.01 (m, 2H), 1.00-0.82 (m, 10H), 0.80-0.71 (m, 3H).

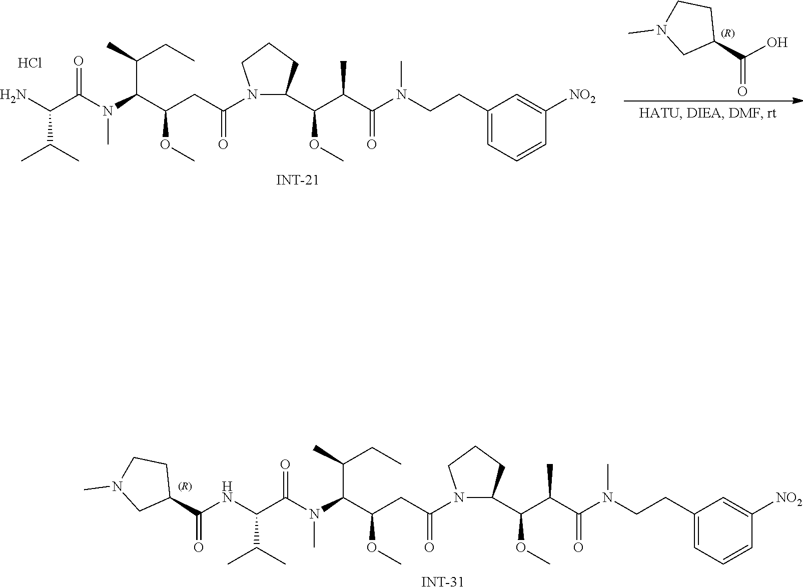

### (R)-N-((S)-1-(((3R,4S,5S)-3-methoxy-1-((S)-2-((1R,2R)-1-methoxy-2-methyl-3-(methyl(3-nitro phenethyl)amino)-3-oxopropyl)pyrrolidin-1-yl)-5-methyl-1-oxoheptan-4-yl)(methyl)amino)-3-methyl-1-oxobutan-2-yl)-1-methylpyrrolidine-3-carboxamide INT-31

To a solution of (R)-1-methylpyrrolidine-3-carboxylic acid (50 mg, 0.39 mmol) and HATU (227 mg, 0.6 mmol) in 3 mL DMF was added DIEA (0.22 mL, 1.34 mmol). The mixture was stirred at room temperature for 30 min under a N₂ atmosphere, INT-21 (200 mg, 0.3 mmol) was then added. The resulting mixture was stirred at room temperature for 2 h. LCMS showed completion. The reaction was concentrated directly, and the residue was re-dissolved in 70 mL EtOAc. The organic layer was washed with H₂O (15 mL*2) and brine (10 mL), dried over Na₂SO₄, filtrated and concentrated to afford crude INT-31 (260 mg, ˜76% purity on LCMS) as a yellow foam solid, which was used directly without further purification. LCMS (ESI): m/z 745.0 [M+H]⁺.

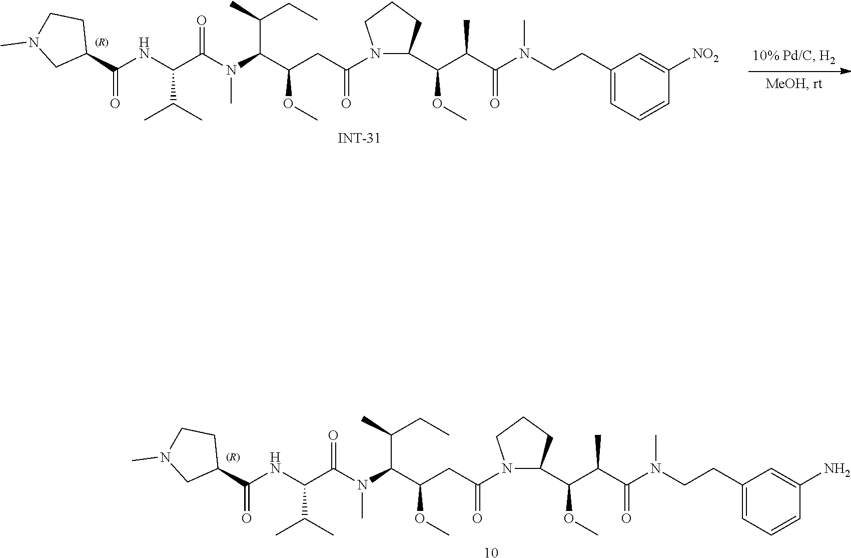

### (R)-N-((S)-1-(((3R,4S,5S)-1-((S)-2-((IR,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-3-carboxamide 10

To a solution of crude INT-31 (150 mg, 0.2 mmol; see above) in 5 mL MeOH was added 10% Pd/C (40 mg). The reaction was then stirred under a H₂ atmosphere (1 atm) at room temperature for 4 h. LCMS showed completion. The mixture was filtrated, and the filtrate was concentrated. The residue was purified by reverse phase column (H₂O:CH₃CN) to afford 10 (130 mg, 90.3% yield) as a yellow solid. LCMS (ESI): m/z 715.1 [M+H]⁺; HPLC: 96.2% @210 nm, Rₜ=7.87 min; ¹H NMR (400 MHz, DMSO) δ 8.33-8.21 (m, 1H), 6.95-6.86 (m, 1H), 6.46-6.31 (m, 3H), 5.08-4.88 (m, 1H), 4.76-4.59 (m, 1H), 4.57-4.42 (m, 1H), 4.10-3.89 (m, 2H), 3.82-3.68 (m, 2H), 3.65-3.53 (m, 2H), 3.51-3.46 (m, 2H), 3.45-3.43 (m, 1H), 3.42-3.40 (m, 1H), 3.20-3.13 (m, 5H), 3.09-2.94 (m, 4H), 2.91-2.86 (m, 2H), 2.84-2.74 (m, 2H), 2.68-2.54 (m, 6H), 2.49-2.19 (m, 2H), 2.13-1.96 (m, 2H), 1.95-1.90 (m, 1H), 1.89-1.75 (m, 3H), 1.74-1.57 (m, 2H), 1.35-1.23 (m, 2H), 1.11-1.01 (m, 2H), 1.00-0.92 (m, 2H), 0.91-0.82 (m, 8H), 0.81-0.74 (m, 3H).

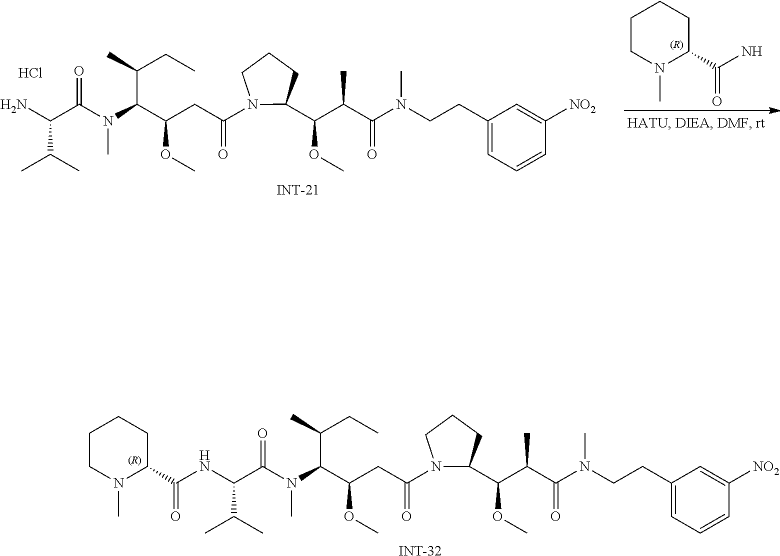

### (R)-N-((S)-1-(((3R,4S,5S)-3-methoxy-1-((S)-2-((1R,2R)-1-methoxy-2-methyl-3-(methyl(3-nitro phenethyl)amino)-3-oxopropyl)pyrrolidin-1-yl)-5-methyl-1-oxoheptan-4-yl)(methyl)amino)-3-methyl-1-oxobutan-2-yl)-1-methylpiperidine-2-carboxamide INT-32

To a solution of INT-21 (200 mg, 0.3 mmol) in 4 mL DMF was added DIEA (174 mg, 1.34 mmol). The mixture was stirred at room temperature for 5 min, and (R)-1-methylpiperidine-2-carboxylic acid (51 mg, 0.36 mmol) was then added, followed by addition of HATU (170 mg, 0.448 mmol). The resulting mixture was stirred at room temperature for 1 h, LCMS showed completion. The reaction was concentrated. The residue was purified by reverse phase column (H₂O:CH₃CN) to afford INT-32 (206 mg, 86% yield) as a light yellow oil, which was used directly without re-purification. LCMS (ESI): m/z 759.0 [M+H]⁺

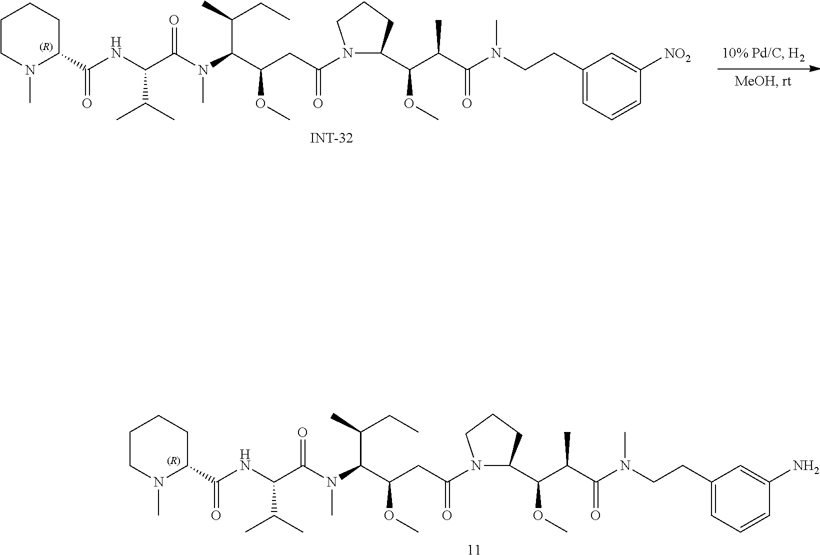

### (R)-N-((S)-1-(((3R,4S,5S)-1-((S)-2-((1R,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-2-carboxamide 11

To a solution of INT-32 (156 mg, 0.206 mmol) in 5 mL MeOH was added 10% Pd/C (30 mg). The reaction was then stirred under a H₂ atmosphere (1 atm) at room temperature for 16 h. LCMS showed completion. The mixture was filtrated, and the filtrate was concentrated. The residue was purified by reverse phase column (H₂O:CH₃CN) to afford 11 (85 mg, 55.7% yield) as an off-white solid. LCMS (ESI): m/z 365.2 [M/2+H]⁺; HPLC: 96.6% @210 nm, Rₜ=11.81 min; ¹H NMR (400 MHz, DMSO) δ 7.41 (t, J=8.8 Hz, 1H), 7.02-6.83 (m, 1H), 6.46-6.30 (m, 3H), 5.14-4.92 (m, 1H), 4.92-4.53 (m, 2H), 4.39-4.27 (m, 1H), 4.09-3.87 (m, 2H), 3.83-3.69 (m, 1H), 3.68-3.57 (m, 1H), 3.55-3.44 (m, 2H), 3.43-3.35 (m, 1H), 3.30-3.25 (m, 2H), 3.24-3.16 (m, 4H), 3.14-3.07 (m, 3H), 3.05-2.95 (m, 4H), 2.93-2.89 (m, 3H), 2.87-2.76 (m, 7H), 2.69-2.53 (m, 4H), 2.49-2.40 (m, 1H), 2.34-2.21 (m, 1H), 2.04-1.88 (m, 2H), 1.87-1.75 (m, 2H), 1.73-1.45 (m, 2H), 1.33-1.17 (m, 2H), 1.16-1.00 (m, 4H), 0.99-0.87 (m, 10H), 0.85-0.77 (m, 3H).

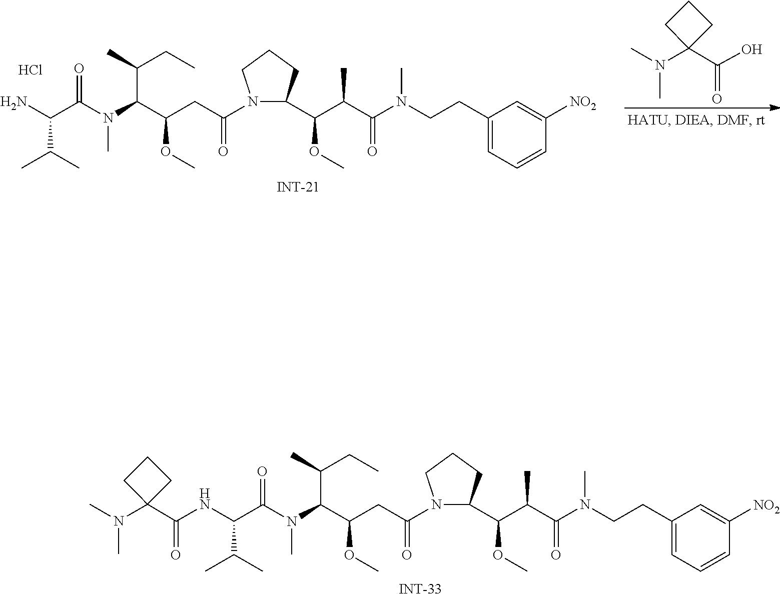

### 1-(dimethylamino)-N-((S)-1-(((3R,4S,5S)-3-methoxy-1-((S)-2-((1R,2R)-1-methoxy-2-methyl-3-(methyl(3-nitrophenethyl)amino)-3-oxopropyl)pyrrolidin-1-yl)-5-methyl-1-oxoheptan-4-yl)(methyl)amino)-3-methyl-1-oxobutan-2-yl)cyclobutanecarboxamide INT-33

To a solution of 1-(dimethylamino)cyclobutanecarboxylic acid (85 mg, 0.597 mmol) in 3 mL DMF was added DIEA (0.2 mL, 1.19 mmol) and HATU (227 mg, 0.597 mmol). The mixture was stirred at room temperature for 0.5 h, and then INT-21 (200 mg, 0.298 mmol) was added. The resulting mixture was stirred at room temperature for 1.5 h. LCMS showed completion. The reaction was purified by Reverse Phase Column directly (H₂O/CH₃CN) to afford INT-33 (178 mg, 78.6% yield) as a white solid. LCMS (ESI): m/z 759.2 [M+H]⁺

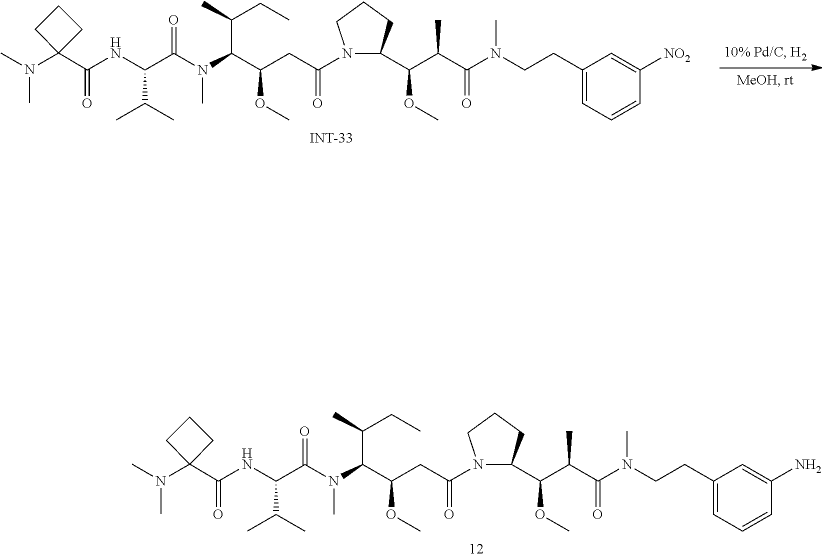

### N-((S)-1-(((3R,4S,5S)-1-((S)-2-((IR,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-(dimethylamino)cyclobutanecarboxamide 12

To a solution of INT-33 (178 mg, 0.235 mmol) in 5 mL MeOH was added 10% Pd/C (36 mg). The reaction was then stirred under a H₂ atmosphere (1 atm) at room temperature for 2 h. LCMS showed completion. The mixture was filtrated, and the filtrate was concentrated. The residue was purified by prep-TLC (DCM: MeOH=14:1, v/v, R_{f}=0.5) then reverse phase column (H₂O:CH₃CN) to afford 12 (97 mg, 56.6% yield) as an off-white solid. LCMS (ESI): m/z 729.5 [M+H]⁺; HPLC: 99.9% @210 nm, Rₜ=8.13 min; ¹H NMR (400 MHz, DMSO) δ 7.43 (m, 1H), 6.91 (m, 1H), 6.46-6.31 (m, 3H), 4.98 (d, J=6.4 Hz, 1H), 4.90 (d, J=13.8 Hz, 1H), 4.77-4.50 (m, 2H), 4.00 (m, 1H), 3.95-3.75 (m, 1H), 3.75-3.66 (m, 1H), 3.66-3.54 (m, 1H), 3.54-3.40 (m, 2H), 3.39-3.33 (m, 1H), 3.30-3.25 (m, 2H), 3.24-3.07 (m, 5H), 2.98 (bs, 1H), 2.89 (d, J=3.1 Hz, 2H), 2.84-2.79 (m, 1H), 2.79-2.65 (m, 1H), 2.65-2.51 (m, 3H), 2.49-2.38 (m, 1H), 2.31-2.14 (m, 2H), 2.14-2.08 (m, 7H), 2.08-1.99 (m, 2H), 1.99-1.70 (m, 4H), 1.68-1.57 (m, 3H), 1.36-1.25 (m, 1H), 1.08 (d, J=6.7 Hz, 1H), 1.04 (d, J=6.7 Hz, 1H), 0.98 (d, J=6.6 Hz, 1H), 0.95-0.82 (m, 9H), 0.81-0.72 (m, 3H).

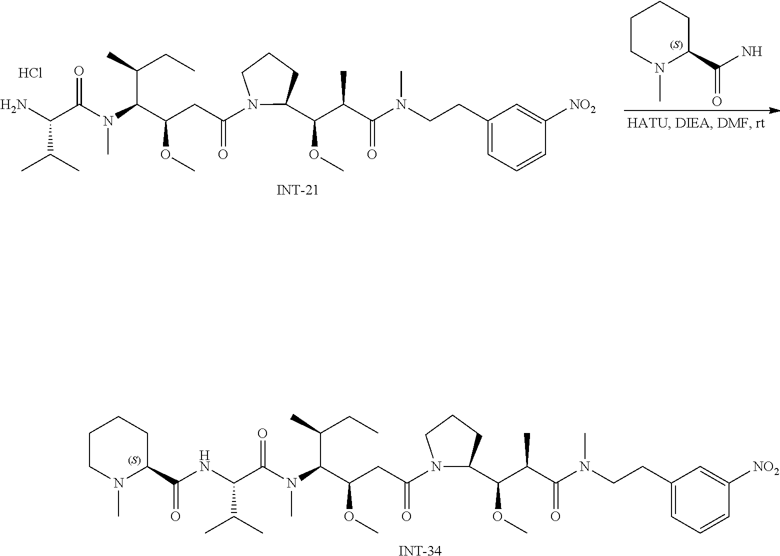

### (S)-N-((S)-1-(((3R,4S,5S)-3-methoxy-1-((S)-2-((1R,2R)-1-methoxy-2-methyl-3-(methyl(3-nitro phenethyl)amino)-3-oxopropyl)pyrrolidin-1-yl)-5-methyl-1-oxoheptan-4-yl)(methyl)amino)-3-methyl-1-oxobutan-2-yl)-1-methylpiperidine-2-carboxamide INT-34

To a solution of (S)-1-methylpiperidine-2-carboxylic acid (54 mg, 0.38 mmol) in 5 mL DMF was added HATU (182 mg, 0.48 mmol), followed by addition of DIEA (124 mg, 0.96 mmol).The reaction mixture was stirred at room temperature for 0.5 h, then INT-21 (200 mg, 0.3 mmol) was added. The resulting mixture was stirred at room temperature for 1 h. LCMS showed completion (LCMS (ESI): m/z 759.2 [M+H]⁺). The reaction was quenched by 50 mL H₂O, extracted with DCM (20 mL*3). The combined organic layers were washed with H₂O and brine, dried over Na₂SO₄, filtrated and concentrated to afford crude product INT-34 (350 mg, >100% yield) as a yellow oil.

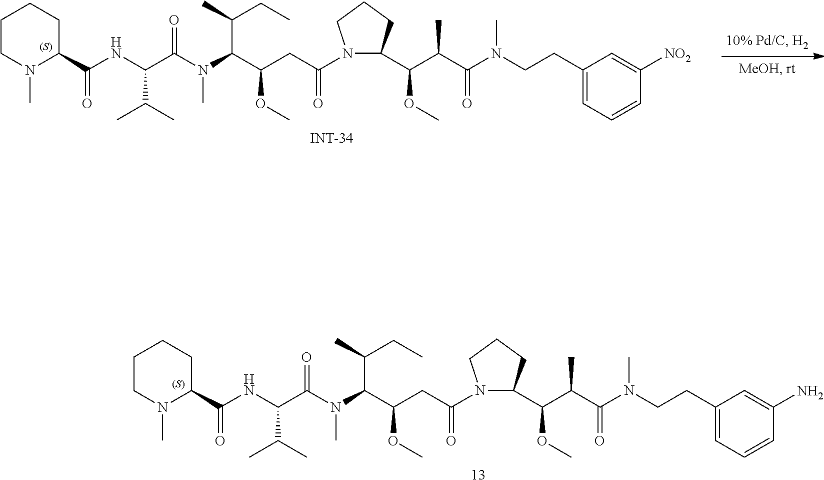

### (S)-N-((S)-1-(((3R,4S,5S)-1-((S)-2-((IR,2R)-3-((3-aminophenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-2-carboxamide 13

To a solution of INT-34 (270 mg, 0.356 mmol) in 10 mL MeOH was added 10% Pd/C (54 mg). The reaction was then stirred under a H₂ atmosphere (1 atm) at room temperature for 2 h. LCMS showed completion. The mixture was filtrated, and the filtrate was concentrated. The residue was purified by reverse phase column (H₂O:CH₃CN) to afford 13 (224.22 mg, 86.6% yield) as an off-white solid. LCMS (ESI): m/z 729.5 [M+H]⁺; HPLC: 96.8% @210 nm, Rt=12.03 min. ¹H NMR (400 MHz, DMSO) δ 7.54-7.43 (m, 1H), 6.95-6.85 (m, 1H), 6.47-6.30 (m, 3H), 4.97 (d, J=5.7 Hz, 1H), 4.90 (d, J=17.0 Hz, 1H), 4.79-4.48 (m, 2H), 4.04-3.95 (m, 1H), 3.95-3.66 (m, 2H), 3.66-3.54 (m, 1H), 3.54-3.41 (m, 2H), 3.40-3.35 (m, 1H), 3.30-3.25 (m, 2H), 3.23-3.05 (m, 5H), 2.94 (bs, 1H), 2.89 (d, J=5.0 Hz, 2H), 2.87-2.70 (m, 3H), 2.69-2.52 (m, 4H), 2.47-2.20 (m, 2H), 2.07-2.01 (m, 3H), 2.01-1.88 (m, 3H), 1.88-1.75 (m, 2H), 1.72-1.55 (m, 4H), 1.53-1.38 (m, 2H), 1.33-1.25 (m, 1H), 1.22-1.12 (m, 1H), 1.08 (d, J=6.7 Hz, 1H), 1.04 (d, J=6.7 Hz, 1H), 1.00-0.92 (m, 2H), 0.91-0.80 (m, 8H), 0.80-0.72 (m, 3H).

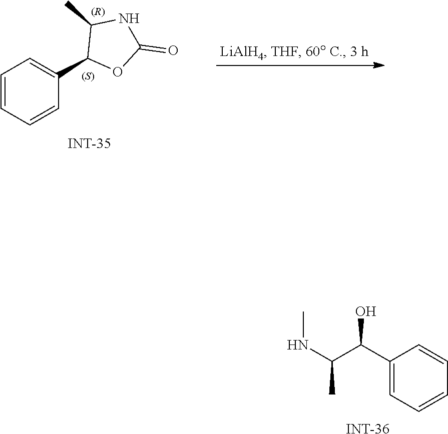

### (1S,2R)-2-(methylamino)-1-phenylpropan-1-ol INT-XX

To a solution of (4R,5S)-4-methyl-5-phenyl-1,3-oxazolidin-2-one INT-35 (100 mg, 0.56 mmol) in THF (4 Ml) stirred under nitrogen at 25° C. was added LiAlH₄ (43 mg, 1.13 mmol). The reaction mixture was stirred at 60° C. for 4h. It was quenched with Na₂SO₄*10 H₂O (1 g), filtrated and concentrated. The residue was purified by Combi-Flash (petroleum ether: EtOAc=3/1) to give INT-36 (100 mg, 90.47%) as a white solid. LCMS (ESI): m/z 166.1 (M+H)⁺.

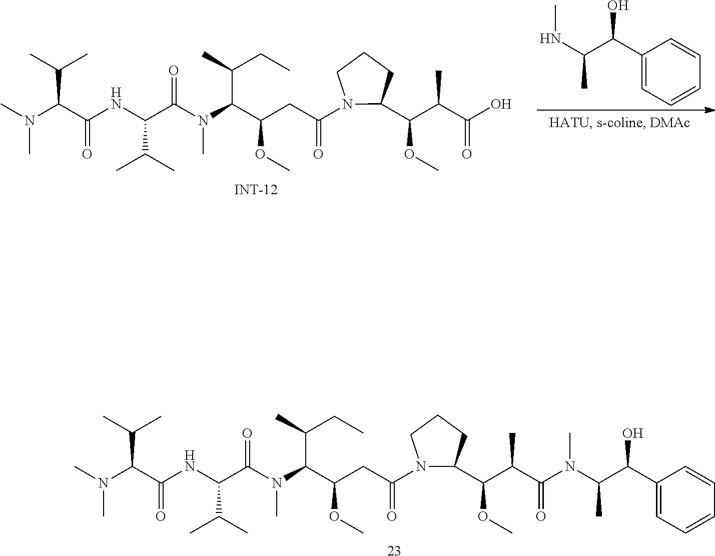

### (S)-2-((S)-2-(dimethylamino)-3-methylbutanamido)-N-((3R,4S,5S)-1-((S)-2-((1R,2R)-3-(((1S,2R)-1-hydroxy-1-phenylpropan-2-yl)(methyl)amino)-1-methoxy-2-methyl-3-oxopropyl)pyrrolidi n-1-yl)-3-methoxy-5-methyl-1-oxoheptan-4-yl)-N,3-dimethylbutanamide 23

To a solution of INT-12 (54 mg, 0.09 mmol), N,N,N′,N′-Tetramethyl-O-(7-azabenzotriazol-1-yl)uranium (41 mg, 0.10 mmol) and 2,4,6-Collidine (22 mg, 0.18 mmol) in DMAc (0.5 mL) stirred under nitrogen at 25° C. was added a solution of INT-36 (15 mg, 0.09 mmol) in DMAc (0.5 mL). The reaction mixture was stirred at 25° C. for 30 mins. Filtrated and directly purified by prep-HPLC (ACN-H₂O(0.1% TFA)) to give 23 (45.1 mg, 63.55%) as a white solid. LCMS (ESI): m/z 746.3 (M+H)+; 1H NMR (400 MHz, DMSO) δ 9.57 (s, 1H), 8.92 (d, J=8.3 Hz, 1H), 7.32-7.28 (m, 2H), 7.24-7.19 (m, 2H), 4.91-4.42 (m, 6H), 4.09 (d, J=4.3 Hz, 1H), 3.99 (s, 1H), 3.76 (s, 1H), 3.71 (s, 1H), 3.65-3.57 (m, 2H), 3.37 (t, J=9.4 Hz, 1H), 3.29-3.21 (m, 6H), 3.20-3.06 (m, 3H), 3.04-2.98 (m, 1H), 2.95-2.89 (m, 1H), 2.81-2.74 (m, 6H), 2.72 (d, J=4.5 Hz, 2H), 2.42 (d, J=4.4 Hz, 1H), 2.34-2.14 (m, 3H), 1.94-1.83 (m, 1H), 1.72-1.60 (m, 1H), 1.55-1.44 (m, 1H), 1.41-1.29 (m, 2H), 1.26-1.09 (m, 3H), 1.08-0.97 (m, 6H), 0.97-0.93 (m, 6H), 0.92-0.85 (m, 6H), 0.78 (dd, J=15.0, 7.4 Hz, 3H).

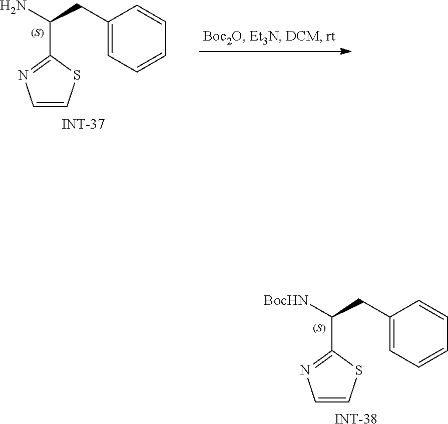

### tert-butyl (S)-(2-phenyl-1-(thiazol-2-yl)ethyl)carbamate INT-38

To a stirred solution of (1S)-2-phenyl-1-(1,3-thiazol-2-yl)ethanamine INT-37 (204 mg, 1.00 mmol) and Et₃N (202 mg, 2.00 mmol) in DCM (5 mL) was added (Boc)₂O (327 mg, 1.50 mmol). The mixture was stirred at 25° C. for 16h. It was diluted with water (10 mL), extracted with DCM (2*10 mL), combined organic phase was dried over Na₂SO₄, filtrated and concentrated in vacuo. The residue was purified by Combi-Flash (petroleum ether: EtOAc=5/1) to give INT-38 (250 mg, 82.2%) as a white solid. LCMS (ESI): m/z 305 (M+H)⁺.

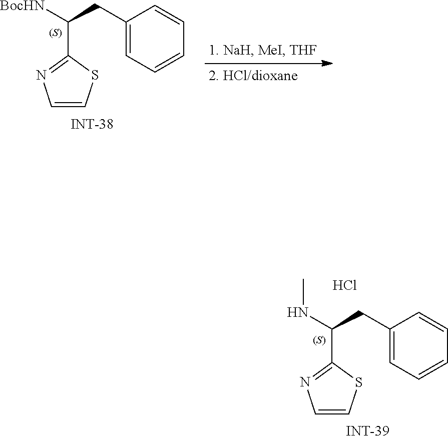

### tert-butyl (S)-methyl(2-phenyl-1-(thiazol-2-yl)ethyl)carbamate INT-39

To a solution of INT-38 (100 mg, 0.328 mmol) in THF (2 mL) was added Sodium hydride (27 mg, 0.657 mmol, 60% in mineral oil) at 0° C. the reaction was stirred at 0° C. for 10 min, then Mel (93 mg, 0.657 mmol) was added at 0° C. It was stirred at 25° C. for 2h, which was quenched with water (10 mL), extracted with EtOAc (10 mL*3), the combined organic phase was dried over sodium sulfate, filtered, concentrated. purified by Combi-Flash (petroleum ether:EtOAc=5:1) to give the methylation product (60 mg). LCMS (ESI): m/z 319.2 (M+H)⁺. The methylation product was dissolved into HCl/dioxane (3 mL, 1N), stirred at 25° C. for 1 h, concentrated to give the INT-39 (48 mg, crude) as a white solid.

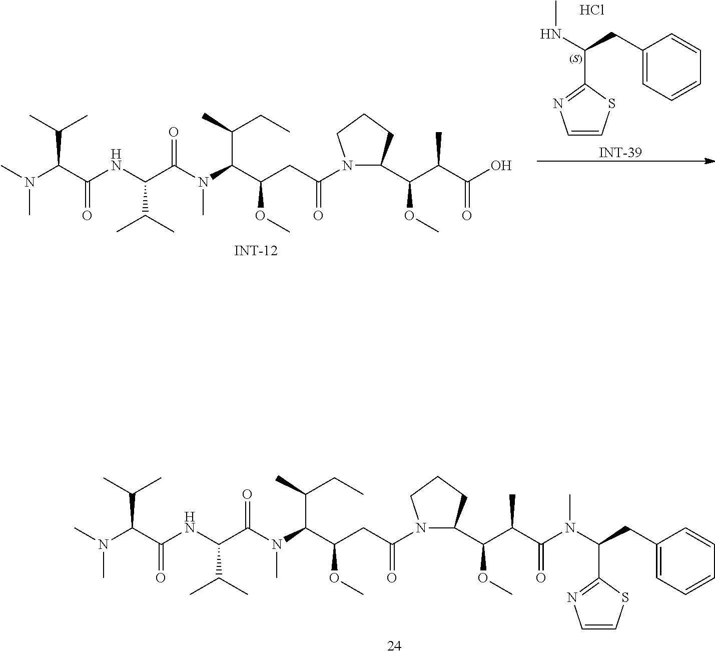

### (S)-2-((S)-2-(dimethylamino)-3-methylbutanamido)-N-((3R,4S,5S)-3-methoxy-1-((S)-2-((1R,2R)-1-methoxy-2-methyl-3-(methyl((S)-2-phenyl-1-(thiazol-2-yl)ethyl)amino)-3-oxopropyl)pyrrolidin-1-yl)-5-methyl-1-oxoheptan-4-yl)-N,3-dimethylbutanamide 24

To a solution of INT-12 (30 mg, 0.05 mmol), INT-39 (11 mg, 0.05 mmol) and HATU (23 mg, 0.06 mmol) in DMAc (1.5 mL) was added DIEA (13 mg, 0.10 mmol), the reaction was stirred 25° C. for 2h. directly purified by prep-HPLC (ACN-H20(0.1% TFA)) to give 24 (21 mg, 52.3%) as a white solid. LCMS (ESI): m/z 799.5 (M+H)⁺; ¹H NMR (400 MHz, DMSO) δ 9.64 (s, 1H), 9.00-8.85 (m, 1H), 7.90 (m, 2H), 7.31-7.19 (m, 4H), 7.18-7.00 (m, 1H), 6.50-6.30 (m, 1H), 4.90-4.50 m, 3H), 3.66-3.40 (m, 4H), 3.37-3.29 (m, 1H), 3.28 (s, 3H), 3.23-3.12 (m, 3H), 3.11-2.91 (m, 3H), 2.84-2.70 (m, 8H), 2.46-2.41 (m, 1H), 2.34-2.23 (m, 1H), 2.19-2.09 (m, 1H), 2.08 (m, 6H), 1.94-1.64 (m, 2H), 1.59-1.16 (m, 3H), 1.12-0.69 (m, 21H).

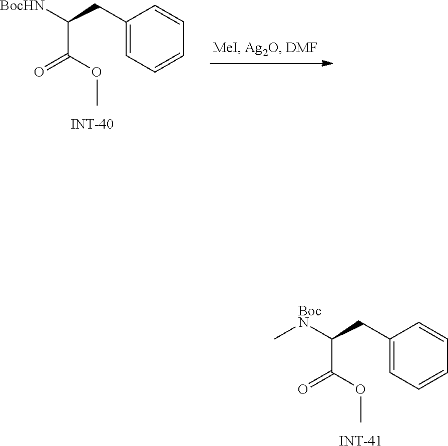

### methyl N-(tert-butoxycarbonyl)-N-methyl-L-phenylalaninate INT-41

To a mixture of (4R,5S)-4-methyl-5-phenyloxazolidin-2-one INT-40 (100 mg, 0.36 mmol) and Ag20 (413 mg, 1.78 mmol) in DMF (4 mL) stirred under nitrogen at 25° C. was added a solution of Mel (101 mg, 0.71 mmol) in DMF (1 mL) dropwise. The reaction mixture was stirred at 25° C. for 12 h. It was diluted with water (20 mL), extracted with EtOAc (10 mL*3). The organic layers were combined, washed with brine (10 mL), dried over Na₂SO₄, filtrated and concentrated. The residue was purified by Combi-Flash (petroleum ether: EtOAc=3/1) to give INT-41 (100 mg, 90.5%) as a white solid. LCMS (ESI): m/z 316 (M+Na)+.

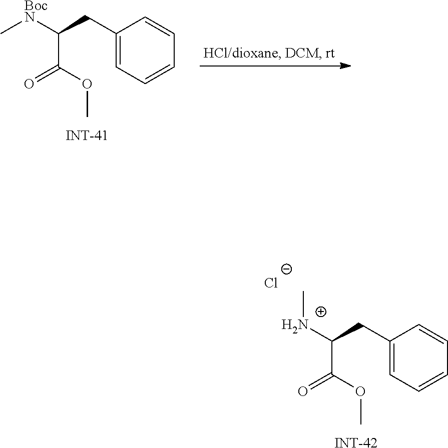

### methyl methyl-L-phenylalaninate hydrochloride INT-42

To a solution of INT-41 (100 mg, 0.34 mmol) in DCM (4 mL) stirred at 25° C. was added 1,4-dioxane/HCl (1 mL, 4N). The reaction mixture was stirred at 25° C. for 2h. concentrated to give INT-42 (60 mg, crude) as a white solid. LCMS (ESI): m/z 194.2 (M+H)+.

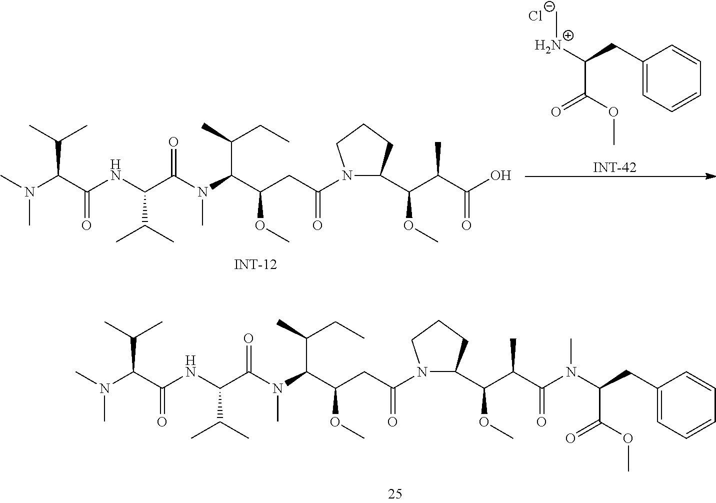

### Methyl N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanoyl)-N-methyl-L-phenylalaninate 25

To a solution of INT-12 (30 mg, 0.05 mmol), DIEA (18 mg, 0.15 mmol) and HATU (29 mg, 0.07 mmol) in DMAc(1.5 mL) stirred under nitrogen at 25° C. was added a solution of INT-42 (17 mg, 0.07 mmol) in DMAc (0.5 mL). The reaction mixture was stirred at 25° C. for 2h. directly purified by prep-HPLC (ACN-H20(0.1% TFA)) to give 25 (13.3 mg, 32.9%) as a white solid. LCMS (ESI): m/z 774.2 (M+H)+; 1H NMR (400 MHz, DMSO) δ 9.59 (s, 1H), 8.93 (t, J=8.1 Hz, 1H), 7.28 (dd, J=15.0, 6.7 Hz, 1H), 7.20 (s, 4H), 5.43 (d, J=7.2 Hz, 1H), 5.08 (ddd, J=38.3, 11.0, 5.0 Hz, 1H), 4.80-4.55 (m, 2H), 4.02 (d, J=41.8 Hz, 1H), 3.79-3.56 (m, 6H), 3.45 (dd, J=17.3, 8.2 Hz, 1H), 3.24 (t, J=10.9 Hz, 5H), 3.17 (d, J=8.8 Hz, 4H), 3.10 (d, J=11.0 Hz, 2H), 3.04-2.95 (m, 2H), 2.85-2.68 (m, 10H), 2.48-2.39 (m, 2H), 2.29 (dt, J=16.0, 8.0 Hz, 1H), 2.18-1.96 (m, 1H), 1.88 (s, 1H), 1.79-1.68 (m, 1H), 1.61 (dd, J=12.1, 6.3 Hz, 1H), 1.47-1.13 (m, 2H), 1.05 (d, J=6.6 Hz, 3H), 1.02-0.92 (m, 9H), 0.92-0.89 (m, 3H), 0.88-0.84 (m, 3H), 0.79 (t, J=7.4 Hz, 3H).

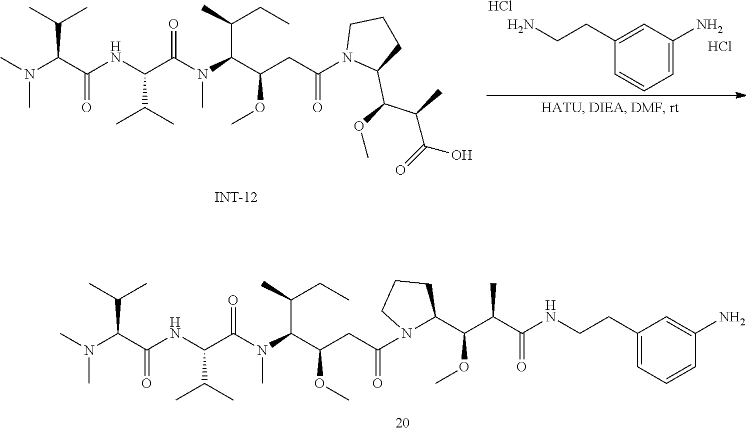

### (S)-N-((3R,4S,5S)-1-((S)-2-((1R,2R)-3-((3-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide 20

To a solution of 3-(2-amino-ethyl)-phenylamine dihydrochloride (1.67 g, 8.01 mmol) in DMF (100 mL) was added DIEA (4.6 mL, 26.72 mmol). The mixture was stirred at room temperature for 0.5 h, then INT-12 (4 g, 6.68 mmol) was added, followed by addition of HATU (3.3 g, 8.68 mmol). The resulting mixture was stirred at room temperature for 2 h. LCMS showed completion. The reaction was quenched by H₂O (150 mL), then extracted with EtOAc (100 mL*3). The combined organic layers were washed with H₂O (50 mL) and brine (50 mL), dried over Na₂SO₄, filtrated and concentrated to dry. The residue was purified by reverse phase column (H₂O/CH₃CN) to afford 20 (3.4 g, 71% yield) as white solid. LCMS (ESI): m/z 717.2 [M+H]⁺; HPLC: 99.48% @210 nm, Rₜ=10.72 min; ¹H NMR (400 MHz, DMSO-d₆) δ 8.09-7.96 (m, 1H), 7.82 (t, J=5.6 Hz, 1H), 6.89 (t, J=8.0 Hz, 1H), 6.42-6.36 (m, 2H), 6.33 (t, J=8.2 Hz, 1H), 4.90 (d, J=14.7 Hz, 2H), 4.79-4.61 (m, 1H), 4.61-4.48 (m, 1H), 4.04-3.94 (m, 1H), 3.88-3.80 (m, 1H), 3.77-3.70 (m, 1H), 3.61-3.48 (m, 1H), 3.46-3.36 (m, 1H), 3.29 (d, 3H), 3.27-3.22 (m, 1H), 3.18 (d, 3H), [3.15 (s, 1.5H); 3.00 (s, 1.5H)], 3.14-3.09 (m, 1H), 2.68-2.53 (m, 3H), 2.46-2.40 (m, 1H), 2.34-2.22 (m, 1H), 2.22-2.13 (m, 7H), 1.97-1.82 (m, 4H), 1.73-1.56 (m, 2H), 1.36-1.25 (m, 1H), 1.10-1.03 (m, 3H), 0.94-0.82 (m, 13H), 0.78-0.67 (m, 6H).

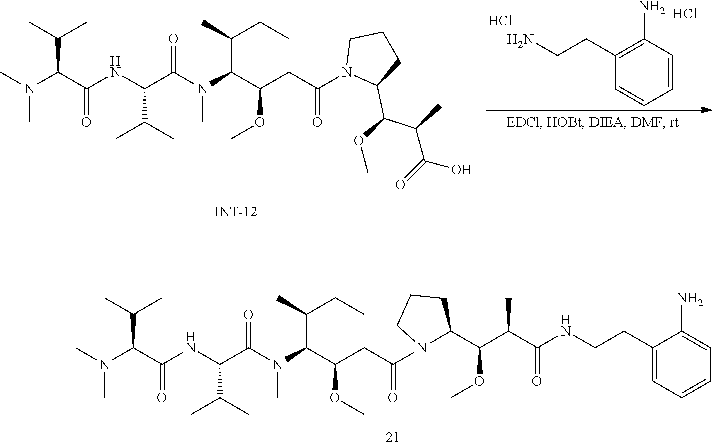

### (S)-N-((3R,4S,5S)-1-((S)-2-((1R,2R)-3-((2-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide 21

To a solution of 2-(2-Amino-ethyl)-phenylamine dihydrochloride (20.9 mg, 100.2 umol) in DMF (2 mL) was added DIEA (53.9 mg, 417.5 umol). The mixture was stirred at room temperature for 0.5 h. Then EDCI (24 mg, 125.2 umol) and HOBt (22.6 mg, 167 umol) were added, followed by INT-12 (50 mg, 83.5 umol)/DMF (0.5 mL) added drop-wise. The resulting mixture was stirred at room temperature for 3 h. LCMS showed completion. The reaction was purified by reverse phase column (CH₃CN/H₂O) directly to afford 21 (50 mg, 83.5% yield) as an off-white solid. LCMS (ESI): m/z 717.0 [M+H]⁺; HPLC: 99.6% @210 nm, Rₜ=11.04 min; ¹H NMR (400 MHz, CDCl₃) δ 7.11-6.94 (m, 3H), 6.94-6.82 (m, 1H), 6.75-6.62 (m, 2H), 4.93-4.69 (m, 2H), 4.39-4.05 (m, 4H), 4.03-3.67 (m, 2H), 3.56-3.44 (m, 2H), 3.43-3.25 (m, 8H), [3.15 (s, 0.8H); 3.03 (s, 2.2H)], 2.79-2.70 (m, 2H), 2.46-2.32 (m, 3H), 2.30-2.18 (m, 6H), 2.12-1.94 (m, 4H), 1.87-1.77 (m, 2H), 1.41-1.30 (m, 1H), 1.28-1.20 (m, 3H), 1.10-0.88 (m, 16H), 0.82 (t, J=6.9 Hz, 3H).

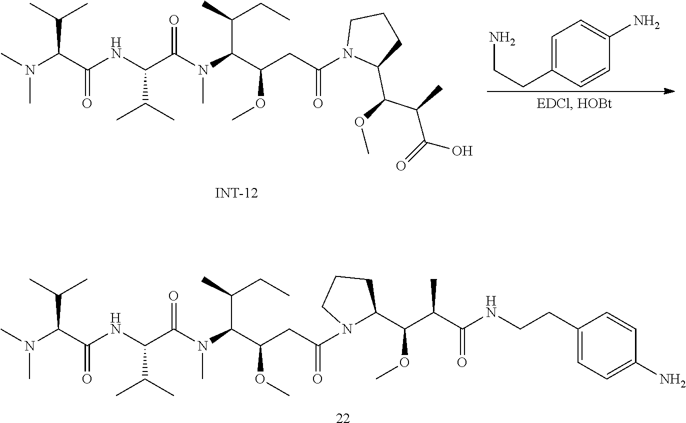

### (S)-N-((3R,4S,5S)-1-((S)-2-((1R,2R)-3-((4-aminophenethyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide 20

To a solution of 4-(2-aminoethyl)aniline (13.7 mg, 100.2 umol) in DMF (2 mL) were added EDCI (24 mg, 0.125 mmol), HOBt (16.9 mg, 0.125 mmol) and DIEA (21.6 mg, 0.167 mmol) subsequently. Then INT-12 (50 mg, 83.5 umol)/DMF (0.5 mL) was added drop wise in 2 min. The reaction was stirred at room temperature for 4 h. LCMS showed completion. The mixture was purified by prep-HPLC (H₂O/CH₃CN) directly to afford 22 (7 mg, 12% yield) as an off-white solid. LCMS (ESI): m/z 717.1 [M+H]⁺; HPLC: 96.4% @210 nm, Rₜ=10.29 min; ¹H NMR (400 MHz, CDCl₃) δ 6.98 (d, J=8.3 Hz, 2H), 6.92 (dd, J=21.8, 8.7 Hz, 2H), 6.64 (d, J=8.5 Hz, 1H), 6.61 (d, J=8.3 Hz, 2H), 6.40 (s, 1H), 4.96-4.83 (m, 1H), 4.83-4.70 (m, 2H), 4.16-4.07 (m, 2H), 3.88-3.84 (m, 1H), 3.83-3.74 (m, 1H), 3.62-3.56 (m, 1H), 3.46-3.43 (m, 2H), 3.37-3.31 (m, 8H), [3.14 (s, 1H); 3.02 (s, 2H)], 2.71 (t, J=7.0 Hz, 2H), 2.46-2.41 (m, 2H), 2.37-2.34 (m, 1H), 2.26-2.23 (m, 6H), 2.08-1.93 (m, 5H), 1.38-1.28 (m, 2H), 1.23-1.20 (m, 3H), 1.01-0.93 (m, 15H), 0.83-0.79 (m, 3H).

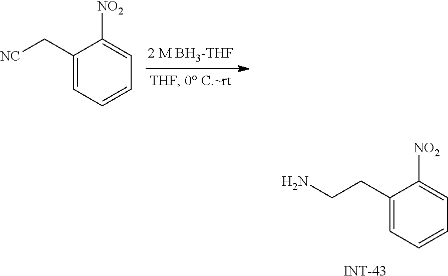 [SMILES: N#CCc1ccccc1[N+](=O)[O-] ; NCCc1ccccc1[N+](=O)[O-] ; "INT-43"]

### 2-(2-Ntrophenyl)ethanamine INT-43

To a solution of 2-(2-nitrophenyl)acetonitrile (3 g, 18.5 mmol) in 50 mL dry THF pre-cooled to 0° C. by an ice bath was added 2 M BH₃-THF (21.3 mL, 42.6 mmol) under a N₂ atmosphere. The mixture was allowed to worm up to room temperature naturally and stirred for 9 h. TLC showed completion (DCM:MeOH=10:1, R_{f}=0.4). The mixture was cooled to 0° C. again, then quenched by adding 30 mL MeOH slowly, and then concentrated to dry to afford crude INT-43 (3.07 g, 100% yield) as a brown solid: LCMS (ESI): m/z 167.1 [M+H], which was used directly without further purification.

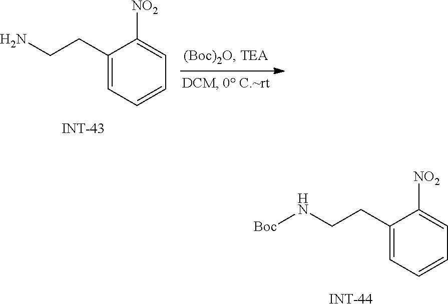 [SMILES: NCCc1ccccc1[N+](=O)[O-] ; "INT-43" ; CC(C)(C)OC(=O)NCCc1ccccc1[N+](=O)[O-] ; "INT-44"]

### Tert-butyl 2-nitrophenethylcarbamate INT-44

To a solution of 2-(2-nitrophenyl)ethanamine INT-43 (3.07 g crude, 18.5 mmol) in 40 mL dry DCM was added Et₃N (7.8 mL, 56.0 mmol) at room temperature under a N₂ atmosphere. The mixture was cooled down to 0° C. by an ice bath, then Boc₂O (4.7 mL, 20.52 mmol)/in 20 mL DCM was added dropwise. The reaction was allowed to worm up to room temperature naturally and stirred for 16 h. TLC showed completion (DCM:MeOH=10:1, R_{f}=0.95). The mixture was quenched by adding 100 mL H₂O, and then extracted with DCM (50 mL*3). The combined organic layers were washed with H₂O and brine, dried over Na₂SO₄, filtrated and concentrated to dry. The residue was purified by Flash Chromatography (petroleum ether: EtOAc=40:1˜30:1˜20:1, v/v) to afford INT-44 (3.85 g, 78% yield for 2 steps) as a light yellow oil. LCMS (ESI): m/z 167.1 [M−t−Bu].

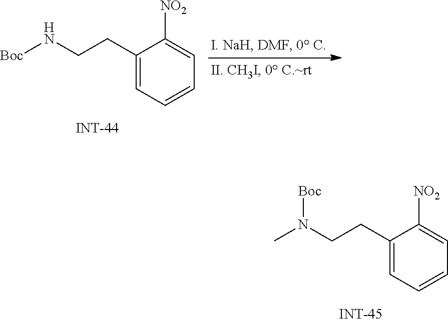 [SMILES: CC(C)(C)OC(=O)NCCc1ccccc1[N+](=O)[O-] ; "INT-44" ; CN(CCc1ccccc1[N+](=O)[O-])C(=O)OC(C)(C)C ; "INT-45"]

### Tert-butyl methyl(2-nitrophenethyl)carbamate INT-45

To a solution of tert-butyl 2-nitrophenethylcarbamate INT-44 (2 g, 7.51 mmol) in 40 mL dry DMF was added 60% NaH (601 mg, 15 mmol) at 0° C. under a N₂ atmosphere. The mixture was stirred at 0° C. for 30 min, then CH₃I (1.1 mL, 17.3 mmol) was added. The reaction was allowed to worm up to room temperature naturally and stirred for overnight. TLC showed completion (petroleum ether: EtOAc=5:1, R_{f}=0.7). The mixture was quenched by adding 80 mL H₂O slowly at 0° C., then extracted with EtOAc (30 mL*3). The combined organic layers were washed with H₂O and brine, dried over Na₂SO₄, filtrated and concentrated to dry. The residue was purified by Flash Chromatography (petroleum ether: EtOAc=40:1˜30:1˜25:1, v/v) to afford INT-45 (1.44 g, 62% yield). LCMS (ESI): m/z 181.1 [M−t−Bu].

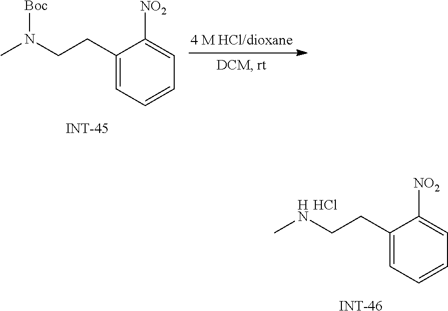 [SMILES: CN(CCc1ccccc1[N+](=O)[O-])C(=O)OC(C)(C)C ; "INT-45" ; CNCCc1ccccc1[N+](=O)[O-] ; Cl ; "INT-46"]

### N-methyl-2-(2-nitrophenyl)ethanamine hydrochloride INT-46

To a solution of tert-butyl methyl(2-nitrophenethyl)carbamate INT-45 (1.34 g, 4.78 mmol) in 15 mL DCM was added 4 M HCl/dioxane (10 mL, 40 mmol). The reaction was stirred at room temperature for 2.5 h. TLC showed completion. The mixture was concentrated to dry. The residue was slurred with MTBE (20 mL) 3 times to afford INT-46 (920 mg, 64% yield) as a light yellow solid: LCMS (ESI): m/z 181.1 [M+H]⁺; ¹H NMR (400 MHz, DMSO-d₆) δ 9.14 (s, 2H), 8.02 (dd, J=8.2, 1.2 Hz, 1H), 7.73 (td, J=7.6, 1.3 Hz, 1H), 7.63-7.52 (m, 2H), 3.25-3.15 (m, 4H), 2.57 (s, 3H).

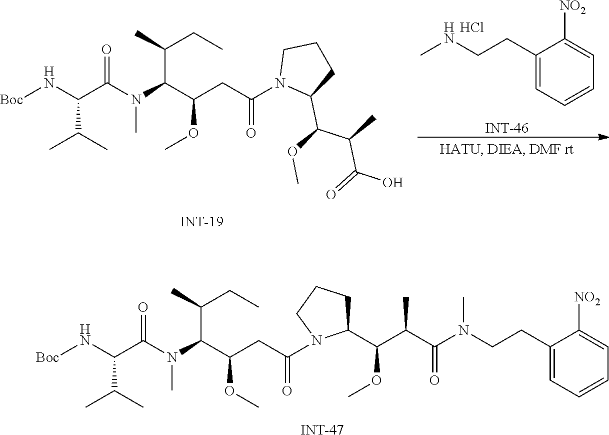

### Tert-butyl ((S)-1-(((3R,4S,5S)-3-methoxy-1-((S)-2-((1R,2R)-1-methoxy-2-methyl-3-(methyl(2-nitrophenethyl)amino)-3-oxopropyl)pyrrolidin-1-yl)-5-methyl-1-oxoheptan-4-yl)(methyl)amino)-3-methyl-1-oxobutan-2-yl)carbamate INT-47

Tube A: To a solution of INT-46 (870 mg, 4.02 mmol) in DMF (30 mL) was added DIEA (2.3 mL, 13.9 mmol). The mixture was stirred at room temperature for 20 min to form solution A.
Tube B: To another solution of INT-19 (1.77 g, 3.09 mmol) in 20 mL DMF were added HATU (2.35 g, 6.18 mmol) and DIEA (2.3 mL, 13.9 mmol) at room temperature. The mixture was stirred at room temperature for 0.5 h, then solution A added. The resulting mixture was stirred at room temperature for 3.5 h. LCMS showed completion. The reaction was purified by reverse phase column (H₂O/CH₃CN) directly to afford INT-47 (1.74 g, 73% yield) as yellow oil. LCMS (ESI): m/z 734.2 [M+H]⁺.

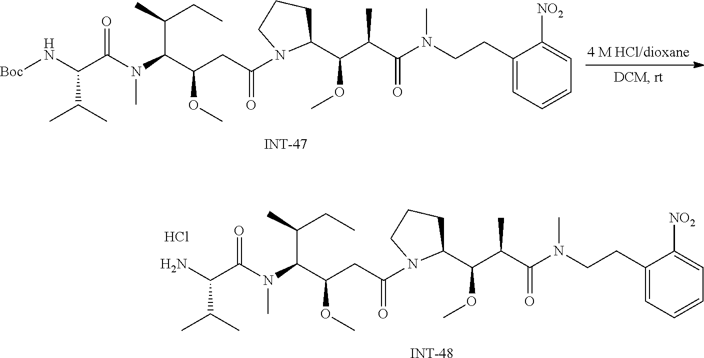

### (S)-2-amino-N-((3R,4S,5S)-3-methoxy-1-((S)-2-((1R,2R)-1-methoxy-2-methyl-3-(methyl(2-nitrophenethyl)amino)-3-oxopropyl)pyrrolidin-1-yl)-5-methyl-1-oxoheptan-4-yl)-N,3-dimethylbutanamide hydrochloride INT-48

To a solution of INT-47 (1.54 g, 2.1 mmol) in 20 mL DCM was added 4 M HCl/dioxane (5 mL, 20 mmol). The reaction was stirred at room temperature for 3 h. TLC showed completion. The mixture was concentrated to dry. The residue was slurred with MTBE (30 mL*3) then freeze dried to afford INT-48 (1.27 g, 90% yield) as a yellow solid. LCMS (ESI): m/z 634.2 [M+H]⁺; HPLC: 98.2% @210 nm, Rₜ=11.74 min.

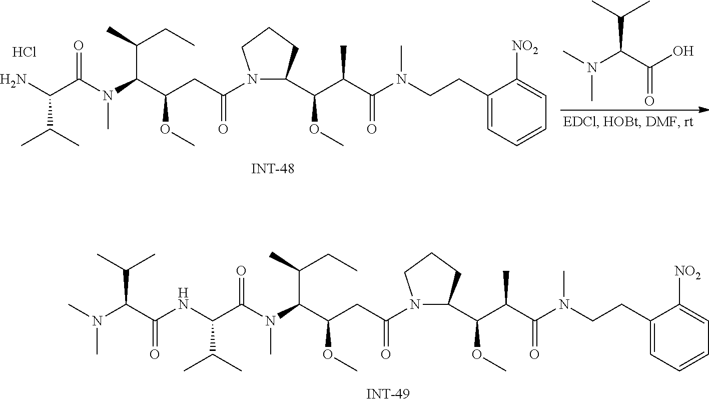

### (S)-2-((S)-2-(dimethylamino)-3-methylbutanamido)-N-((3R,4S,5S)-3-methoxy-1-((S)-2-((1R,2R)-1-methoxy-2-methyl-3-(methyl(2-nitrophenethyl)amino)-3-oxopropyl)pyrrolidin-1-yl)-5-methyl-1-oxoheptan-4-yl)-N,3-dimethylbutanamide INT-49

To a solution of INT-48 (100 mg, 0.149 mmol) and (S)-2-(dimethylamino)-3-methylbutanoic acid (26 mg, 0.179 mmol) in 2 mL DMF was added EDCI (43 mg, 0.224 mmol) and HOBt (40 mg, 0.298 mmol), followed by the addition of DIEA (87 mg,0.671 mmol). The mixture was stirred at room temperature under a N₂ atmosphere for 2 h, LCMS showed completion. The reaction mixture was purified by reverse phase column (H₂O:CH₃CN) directly to afford INT-49 (96 mg, 85% yield) as a colorless oil. LCMS (ESI): m/z 760.9 [M+H]⁺.

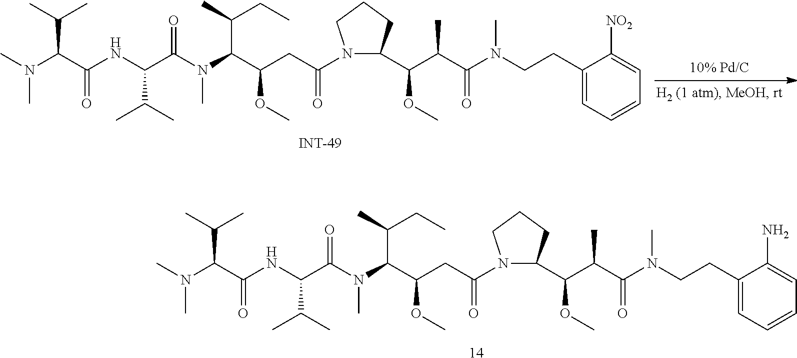

### (S)-N-((3R,4S,5S)-1-((S)-2-((1R,2R)-3-((2-aminophenethyl)(methyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide 14

To a solution of INT-49 (96 mg, 0.126 mmol) in 4 mL MeOH was added 10% Pd/C (20 mg). The reaction then was stirred under a H₂ atmosphere (1 atm) at room temperature for 2.5 h. LCMS showed completion. The mixture was filtrated, and the filtrate was concentrated. The residue was freeze-dried to afford 14 (33 mg, 36% yield) as a white solid. LCMS (ESI): m/z 730.9 [M+H]⁺; HPLC: 95.5% @210 nm, Rₜ=13.51 min; ¹H NMR (400 MHz, DMSO) δ 8.14 (d, J=8.9 Hz, 1H), 8.01 (d, J=8.3 Hz, 1H), 6.96-6.79 (m, 2H), 6.68-6.58 (m, 1H), 6.54-6.41 (m, 1H), 5.14-4.91 (m, 2H), 4.75-4.47 (m, 2H), 4.14-3.73 (m, 3H), 3.70-3.39 (m, 3H), 3.34 (s, 3H), 3.30-3.23 (m, 2H), 3.23-3.19 (m, 1H), 3.19-3.07 (m, 4H), 3.01-2.92 (m, 3H), 2.86-2.81 (m, 1H), 2.76-2.52 (m, 4H), 2.41 (d, J=20.7 Hz, 1H), 2.30-2.14 (m, 6H), 2.03-1.84 (m, 4H), 1.84-1.55 (m, 3H), 1.36-1.25 (m, 1H), 1.10 (dd, J=21.8, 6.7 Hz, 2H), 0.95-0.80 (m, 12H), 0.79-0.69 (m, 6H).

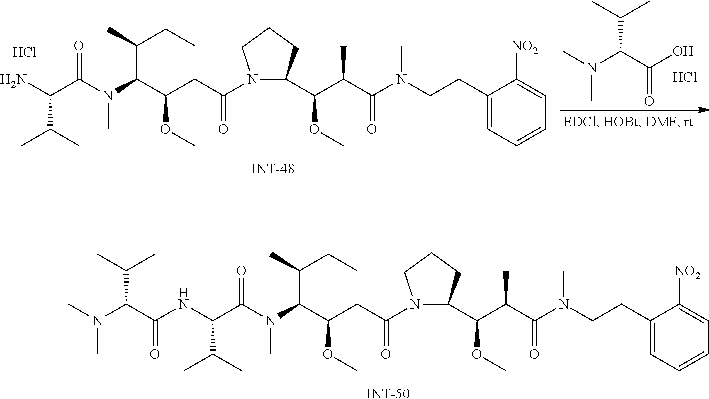

### (S)-2-((R)-2-(dimethylamino)-3-methylbutanamido)-N-((3R,4S,5S)-3-methoxy-1-((S)-2-((1R,2R)-1-methoxy-2-methyl-3-(methyl(2-nitrophenethyl)amino)-3-oxopropyl)pyrrolidin-1-yl)-5-methyl-1-oxoheptan-4-yl)-N,3-dimethylbutanamide INT-50

To a solution of INT-48 (100 mg, 0.149 mmol) and (R)-2-(dimethylamino)-3-methylbutanoic acid (32 mg, 0.179 mmol) in 2 mL DMF was added EDCI (43 mg, 0.224 mmol) and HOBt (40 mg, 0.298 mmol), followed by the addition of DIEA (87 mg,0.671 mmol). The mixture was stirred at room temperature under a N₂ atmosphere for 3 days, LCMS showed completion. The reaction mixture was purified by reverse phase column (H₂O:CH₃CN) directly to afford INT-50 (100 mg, 88% yield) as a colorless oil. LCMS (ESI): m/z 761.1 [M+H]⁺.

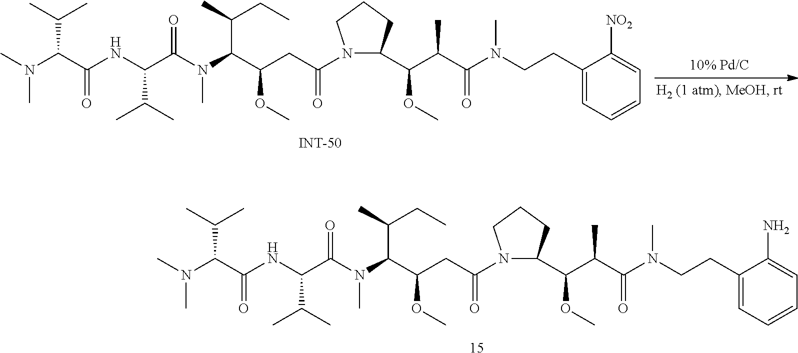

### (S)-N-((3R,4S,5S)-1-((S)-2-((1R,2R)-3-((2-aminophenethyl)(methyl)amino)-1-methoxy-2-methyl-3-oxopropyl)pyrrolidin-1-yl)-3-methoxy-5-methyl-1-oxoheptan-4-yl)-2-((R)-2-(dimethylamino)-3-methylbutanamido)-N,3-dimethylbutanamide 15

To a solution of INT-50 (100 mg, 0.131 mmol) in 3 mL MeOH was added 10% Pd/C (20 mg). The reaction was then stirred under a H₂ atmosphere (1 atm) at room temperature for 2 h. LCMS showed completion. The mixture was filtrated, and the filtrate was concentrated. The residue was freeze-dried to afford 15 (62 mg, 64% yield) as an off-white solid. LCMS (ESI): m/z 731.1 [M+H]⁺; HPLC: 97.4% @210 nm, Rₜ=8.67 min; ¹H NMR (400 MHz, DMSO-d₆) δ 8.14 (d, J=9.0 Hz, 1H), 8.01 (d, J=8.7 Hz, 1H), 6.98-6.79 (m, 2H), 6.66-6.57 (m, 1H), 6.51-6.41 (m, 1H), 5.14-4.91 (m, 2H), 4.78-4.60 (m, 1H), 4.59-4.44 (m, 1H), 4.10-3.81 (m, 2H), 3.81-3.46 (m, 3H), 3.46-3.35 (m, 1H), 3.34-3.32 (m, 3H), 3.31-3.30 (m, 1H), 3.30-3.23 (m, 2H), 3.23-3.10 (m, 5H), 3.03-2.95 (m, 3H), 2.85-2.81 (m, 1H), 2.78-2.53 (m, 4H), 2.41 (d, J=20.1 Hz, 1H), 2.34-2.18 (m, 1H), 2.17-2.11 (m, 5H), 2.00-1.84 (m, 4H), 1.78-1.60 (m, 2H), 1.41-1.29 (m, 1H), 1.10 (dd, J=22.2, 6.7 Hz, 2H), 0.96-0.83 (m, 11H), 0.82-0.73 (m, 7H).

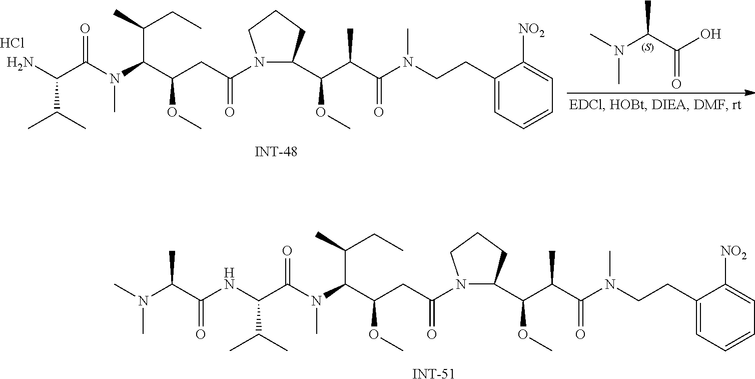

### (S)-2-((S)-2-(dimethylamino)propanamido)-N-((3R,4S,5S)-3-methoxy-1-((S)-2-((1R,2R)-1-methoxy-2-methyl-3-(methyl(2-nitrophenethyl)amino)-3-oxopropyl)pyrrolidin-1-yl)-5-methyl-1-oxoheptan-4-yl)-N,3-dimethylbutanamide INT-51

To a solution of INT-48 (100 mg, 0.149 mmol) and (S)-2-(dimethylamino)propanoic acid (22 mg, 0.188 mmol) in 3 mL DMF was added EDCI (45 mg, 0.235 mmol) and HOBt (32 mg, 0.237 mmol), followed by the addition of DIEA (71 mg,0.55 mmol). The mixture was stirred at room temperature under a N₂ atmosphere for 3 h, LCMS showed completion. The mixture was quenched by adding 15 mL H₂O, then extracted with DCM (20 mL*3). The combined organic layers were washed with H₂O and brine, dried over Na₂SO₄, filtrated and concentrated. The residue was purified by Prep-TLC (DCM:MeOH=10:1, R_{f}=0.7) to afford INT-51 (100 mg, 91% yield) as a light yellow oil. LCMS (ESI): m/z 733.5 [M+H]⁺.

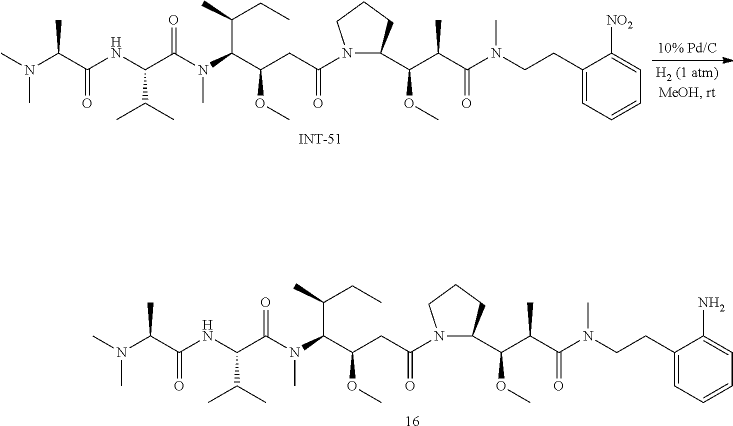

### (S)-N-((3R,4S,5S)-1-((S)-2-((1R,2R)-3-((2-aminophenethyl)(methyl)amino)-1-methoxy-2-methyl-3-oxopropyl)pyrrolidin-1-yl)-3-methoxy-5-methyl-1-oxoheptan-4-yl)-2-((S)-2-(dimethylamino)propanamido)-N,3-dimethylbutanamide 16

To a solution of INT-51 (110 mg, 0.15 mmol) in 5 mL MeOH was added 10% Pd/C (22 mg). The reaction was then stirred under a H₂ atmosphere (1 atm) at room temperature for 1.5 h. LCMS showed completion. The mixture was filtrated, and the filtrate was concentrated. The residue was freeze-dried to afford 16 (90 mg, 86% yield) as an off-white solid. LCMS (ESI): m/z 703.3 [M+H]⁺; HPLC: 96.6% @210 nm, Rₜ=8.52 min;¹H NMR (400 MHz, DMSO-d₆) δ 7.78 (dd, J=25.1, 8.5 Hz, 1H), 6.94-6.76 (m, 2H), 6.69-6.57 (m, 1H), 6.51-6.41 (m, 1H), 5.20-4.87 (m, 2H), 4.76-4.50 (m, 2H), 4.14-3.84 (m, 2H), 3.84-3.72 (m, 1H), 3.71-3.60 (m, 1H), 3.59-3.49 (m, 1H), 3.49-3.37 (m, 2H), 3.31-3.27 (m, 2H), 3.27-3.22 (m, 1H), 3.22-3.19 (m, 1H), 3.19-3.15 (m, 2H), 3.15-3.02 (m, 2H), 3.00-2.89 (m, 4H), 2.89-2.74 (m, 2H), 2.73-2.60 (m, 2H), 2.59-2.52 (m, 1H), 2.48-2.35 (m, 1H), 2.24-2.15 (m, 6H), 2.03-1.86 (m, 3H), 1.83-1.55 (m, 3H), 1.34-1.26 (m, 1H), 1.14-1.02 (m, 5H), 0.95-0.80 (m, 9H), 0.80-0.66 (m, 5H).

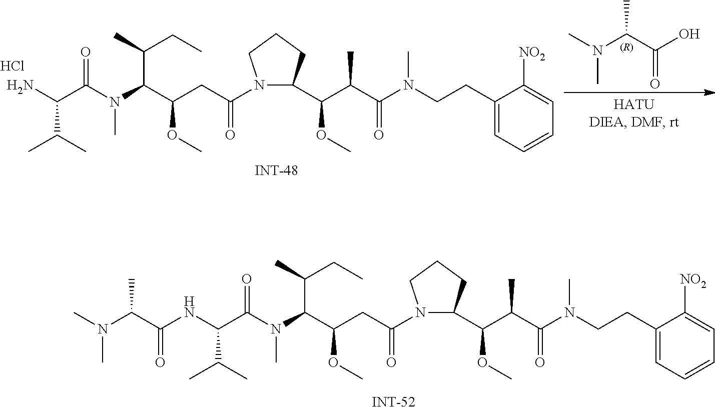

### (S)-2-((R)-2-(dimethylamino)propanamido)-N-((3R,4S,5S)-3-methoxy-1-((S)-2-((1R,2R)-1-methoxy-2-methyl-3-(methyl(2-nitrophenethyl)amino)-3-oxopropyl)pyrrolidin-1-yl)-5-methyl-1-oxoheptan-4-yl)-N,3-dimethylbutanamide INT-52

To a solution of INT-48 (100 mg, 0.149 mmol) and (R)-2-(dimethylamino)propanoic acid (21 mg, 0.174 mmol) in 4 mL DMF was added HATU (78 mg, 0.205 mmol), followed by the addition of DIEA (51 mg, 0.395 mmol). The mixture was stirred at room temperature under a N₂ atmosphere for 4 h, LCMS showed completion. The mixture was concentrated to dry directly to afford the crude. The crude residue was diluted with 15 mL H₂O, then extracted with EtoAc (10 mL*4). The combined organic layers were washed with H₂O and brine, dried over Na₂SO₄, filtrated and concentrated to afford crude INT-52 (180 mg) as yellow oil. LCMS (ESI): m/z 733.1 [M+H]⁺.

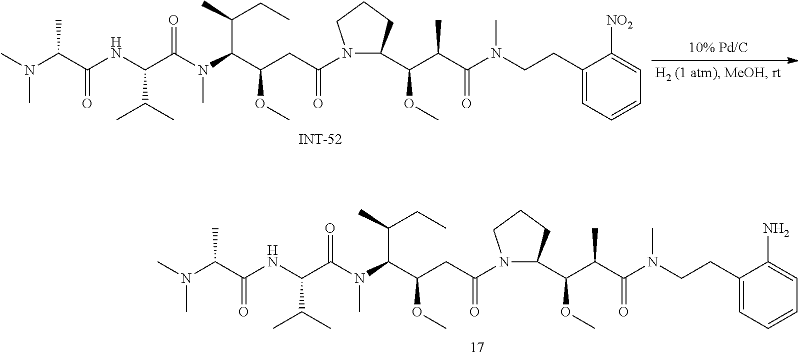

### (S)-N-((3R,4S,5S)-1-((S)-2-((1R,2R)-3-((2-aminophenethyl)(methyl)amino)-1-methoxy-2-methyl-3-oxopropyl)pyrrolidin-1-yl)-3-methoxy-5-methyl-1-oxoheptan-4-yl)-2-((R)-2-(dimethylamino)propanamido)-N,3-dimethylbutanamide 17

To a solution of crude INT-52 (180 mg) in 5 mL MeOH was added 10% Pd/C (20 mg). The reaction was then stirred under a H₂ atmosphere (1 atm) at room temperature for 4 h. LCMS showed completion. The mixture was filtrated, and the filtrate was concentrated. The residue was purified by reverse phase column (H₂O:CH₃CN) then freeze-dried to afford 17 (60 mg, 54% yield for 2 steps) as a white solid. LCMS (ESI): m/z 703.4 [M+H]⁺; HPLC: 99.4% @210 nm, Rₜ=12.52 min; ¹H NMR (400 MHz, DMSO-d₆) δ 9.04-8.90 (m, 1H), 7.00-6.81 (m, 2H), 6.70-6.58 (m, 1H), 6.58-6.43 (m, 1H), 4.79-4.58 (m, 1H), 4.57-4.42 (m, 1H), 4.06-3.97 (m, 1H), 3.96-3.89 (m, 1H), 3.89-3.73 (m, 1H), 3.73-3.56 (m, 1H), 3.56-3.39 (m, 2H), 3.33-3.28 (m, 5H), 3.27-3.10 (m, 6H), 3.09-2.85 (m, 4H), 2.84-2.79 (m, 1H), 2.79-2.52 (m, 9H), 2.41 (d, J=20.2 Hz, 1H), 2.37-2.17 (m, 1H), 2.09-1.85 (m, 3H), 1.85-1.55 (m, 3H), 1.49-1.34 (m, 3H), 1.34-1.25 (m, 1H), 1.09 (dd, J=17.8, 6.7 Hz, 2H), 0.97-0.80 (m, 9H), 0.80-0.62 (m, 5H).

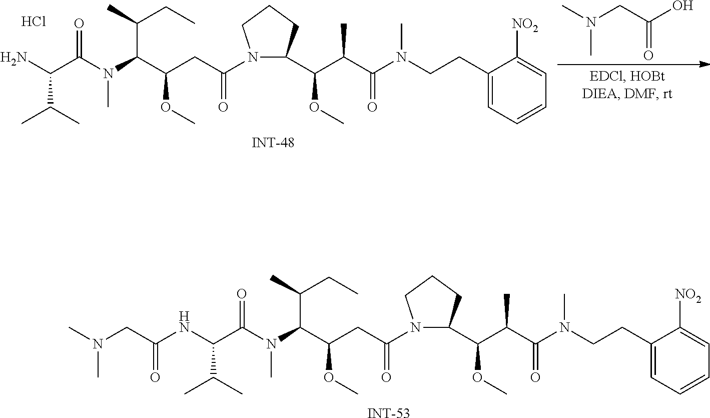

### (S)-2-(2-(dimethylamino)acetamido)-N-((3R,4S,5S)-3-methoxy-1-((S)-2-((1R,2R)-1-methoxy-2-methyl-3-(methyl(2-nitrophenethyl)amino)-3-oxopropyl)pyrrolidin-1-yl)-5-methyl-1-oxoheptan-4-yl)-N,3-dimethylbutanamide INT-53

To a solution of INT-48 (100 mg, 0.149 mmol) and 2-(dimethylamino)acetic acid (20 mg, 0.189 mmol) in 5 mL DMF was added EDCI (45 mg, 0.235 mmol) and HOBt (32 mg, 0.237 mmol), followed by the addition of DIEA (71 mg,0.55 mmol). The mixture was stirred at room temperature under a N₂ atmosphere for 3 h, LCMS showed completion. The mixture was concentrated to dry directly under reduced pressure. The residue was purified by Prep-TLC (DCM:MeOH=10:1, R_{f}=0.7) to afford crude INT-53 (117 mg, 100% yield) as yellow oil. LCMS (ESI): m/z 719.0 [M+H]⁺.

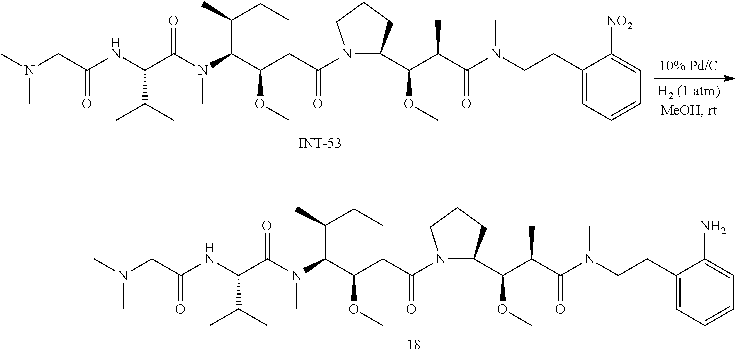

### (S)-N-((3R,4S,5S)-1-((S)-2-((1R,2R)-3-((2-aminophenethyl)(methyl)amino)-1-methoxy-2-methyl-3-oxopropyl)pyrrolidin-1-yl)-3-methoxy-5-methyl-1-oxoheptan-4-yl)-2-(2-(dimethylamino)acetamido)-N,3-dimethylbutanamide 18

To a solution of INT-53 (117 mg, 0.163 mmol) in 5 mL MeOH was added 10% Pd/C (23 mg). The reaction was then stirred under a H₂ atmosphere (1 atm) at room temperature for 2 h. LCMS showed completion. The mixture was filtrated, and the filtrate was concentrated to afford 18 (103 mg, 92% yield) as a white foam solid. LCMS (ESI): m/z 690.2 [M+H]⁺, 345.2 [M+2H]2+; HPLC: 98.6% @210 nm, Rₜ=13.22 min;¹H NMR (400 MHz, DMSO-d₆) δ 7.73-7.61 (m, 1H), 6.95-6.79 (m, 2H), 6.66-6.57 (m, 1H), 6.52-6.41 (m, 1H), 5.15-4.91 (m, 2H), 4.80-4.55 (m, 2H), 4.08-3.83 (m, 2H), 3.82-3.57 (m, 2H), 3.56-3.40 (m, 2H), 3.39-3.33 (m, 2H), 3.31-3.26 (m, 2H), 3.26-3.13 (m, 4H), 3.13-3.06 (m, 1H), 3.06-2.99 (m, 1H), 2.99-2.97 (m, 2H), 2.95-2.92 (m, 3H), 2.91-2.77 (m, 1H), 2.73-2.66 (m, 2H), 2.66-2.52 (m, 1H), 2.47-2.34 (m, 1H), 2.22-2.19 (m, 5H), 2.00-1.84 (m, 3H), 1.84-1.56 (m, 3H), 1.35-1.18 (m, 2H), 1.09 (dd, J=15.8, 6.7 Hz, 2H), 1.01-0.84 (m, 7H), 0.84-0.79 (m, 3H), 0.79-0.68 (m, 4H).

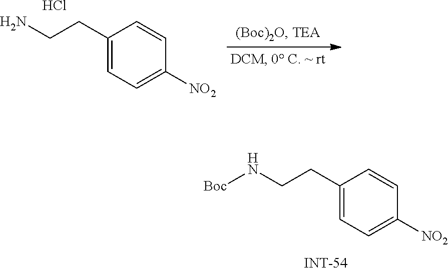 [SMILES: Cl ; NCCc1ccc([N+](=O)[O-])cc1 ; "(Boc)2O, TEA" ; CC(C)(C)OC(=O)NCCc1ccc([N+](=O)[O-])cc1 ; "INT-54"]

### Tert-butyl 4-nitrophenethycarbamate INT-54

To a solution of 2-(4-nitrophenyl)ethanamine hydrochloride (3 g, 14.8 mmol) in 50 mL dry DCM was added Et₃N (4.49 g, 44.41 mmol) at room temperature under a N₂ atmosphere. The mixture was cooled down to 0° C. by an ice bath, then Boc₂O (4.85 g, 22.21 mmol) was added. The reaction was allowed to worm up to room temperature naturally and stirred for 16 h. TLC showed completion (petroleum ether:EtOAc=3:1, R_{f}=0.45). The mixture was quenched by adding 30 mL H₂O, and then extracted with EtOAc (30 mL*3). The combined organic layers were washed with H₂O and brine, dried over Na₂SO₄, filtrated and concentrated to dry. The residue was purified by Flash Chromatography (petroleum ether: EtOAc=5:1, v/v) to afford INT-54 (3.8 g, 96% yield) as a light yellow oil. ¹H NMR (400 MHz, CDCl₃) δ ¹H NMR (400 MHz, CDCl₃) δ 8.19-8.14 (m, 2H), 7.36 (d, J=8.6 Hz, 2H), 4.57 (s, 1H), 3.41 (dd, J=13.2, 6.6 Hz, 2H), 2.92 (t, J=7.0 Hz, 2H), 1.43 (s, 9H).

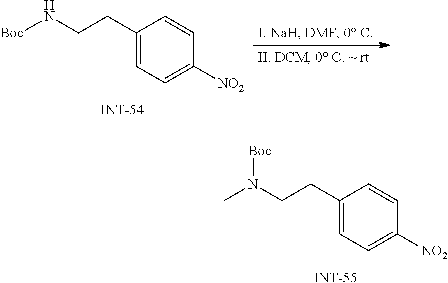 [SMILES: CC(C)(C)OC(=O)NCCc1ccc([N+](=O)[O-])cc1 ; "I. NaH, DMF, 0° C." ; "INT-54" ; CN(CCc1ccc([N+](=O)[O-])cc1)C(=O)OC(C)(C)C ; "INT-55"]

### Tert-butyl methyl(4-nitrophenethyl)carbamate INT-55

A solution of tert-butyl methyl(4-nitrophenethyl)carbamate INT-54 (3.2 g, 12 mmol) in 60 mL dry DMF was cooled down to 0° C. by an ice bath for 20 min under a N₂ atmosphere. To this solution, 60% NaH (0.72 g, 18 mmol) was added portion wise for 3 times at 0° C. in 5 min, followed by CH₃I (2.87 mL, 46.12 mmol) added immediately. The reaction was allowed to worm up to room temperature naturally and stirred for 3 h. TLC showed completion (petroleum ether: EtOAc=5:1, R_{f}=0.75). The mixture was quenched by adding 150 mL H₂O slowly at 0° C., then extracted with EtOAc (60 mL*3). The combined organic layers were washed with H₂O and brine, dried over Na₂SO₄, filtrated and concentrated to afford INT-55 (2.4 g, 71% yield) as yellow oil. LCMS (ESI): m/z 181.1 [M−t−Bu].

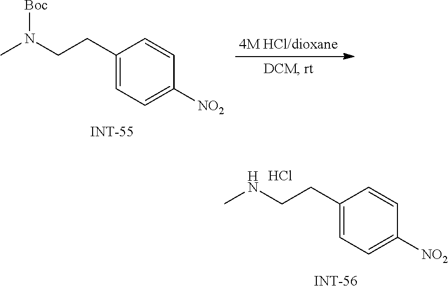 [SMILES: CN(CCc1ccc([N+](=O)[O-])cc1)C(=O)OC(C)(C)C ; "4M HCl/dioxane" ; "INT-55" ; Cl ; CNCCc1ccc([N+](=O)[O-])cc1 ; "INT-56"]

### N-methyl-2-(4-nitrophenyl)ethanamine hydrochloride INT-56

To a solution of tert-butyl methyl(4-nitrophenethyl)carbamate INT-55 (2.4 g, 9 mmol) in 40 mL DCM was added 4 M HCl/dioxane (20 mL, 80 mmol). The reaction was stirred at room temperature for 2 h. TLC showed completion. The mixture was concentrated to dry. The residue was slurred with MTBE (20 mL) 3 times to afford INT-56 (1.69 g, 91% yield) as a light yellow solid: LCMS (ESI): m/z 181.1 [M+H]⁺; ¹H NMR (400 MHz, DMSO-d₆) δ¹H NMR (400 MHz, DMSO) δ 9.28 (s, 2H), 8.27-8.14 (m, 2H), 7.57 (m, 2H), 3.13 (m, 4H), 2.54 (s, 3H).

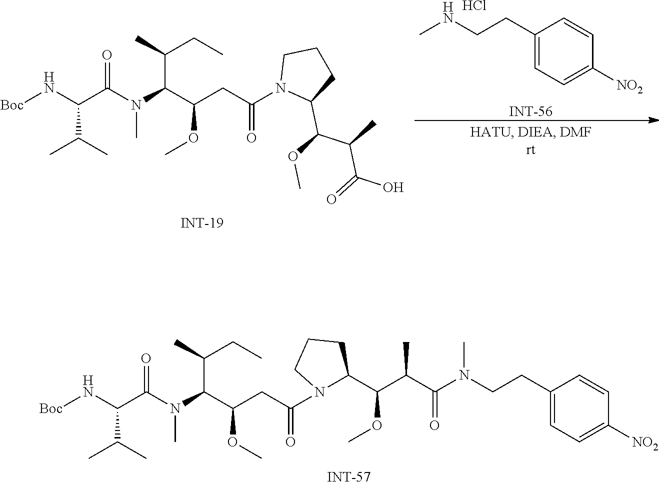

### tert-butyl ((S)-1-(((3R,4S,5S)-3-methoxy-1-((S)-2-((1R,2R)-1-methoxy-2-methyl-3-(methyl(4-nitrophenethyl)amino)-3-oxopropyl)pyrrolidin-1-yl)-5-methyl-1-oxoheptan-4-yl)(methyl)amino)-3-methyl-1-oxobutan-2-yl)carbamate INT-57

Tube A: To a solution of INT-56 (500 mg, 2.3 mmol) in DMF (10 mL) was added DIEA (640 mg, 3.99 mmol). The mixture was stirred at room temperature for 0.5 h to form solution A.
Tube B: To another solution of INT-19 (1.02 g, 1.78 mmol) in 30 mL DMF were added HATU (1.35 g, 3.55 mmol) and DIEA (640 mg, 3.99 mmol) at room temperature. The mixture was stirred at room temperature for 0.5 h, followed by solution A added. The resulting mixture was stirred at room temperature for 4 h. LCMS showed completion. The reaction was purified by reverse phase column (H₂O/CH₃CN) directly to afford INT-57 (1.01 g, 75% yield) as yellow oil. LCMS (ESI): m/z 734.1 [M+H]⁺.

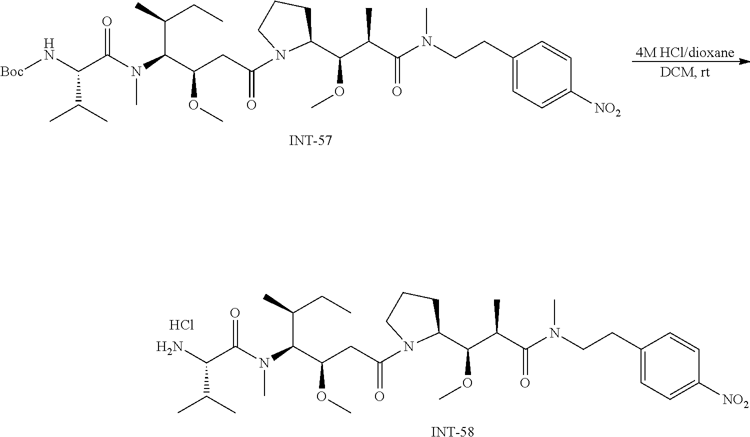

### (S)-2-amino-N-((3R,4S,5S)-3-methoxy-1-((S)-2-((1R,2R)-1-methoxy-2-methyl-3-(methyl(4-nitrophenethyl)amino)-3-oxopropyl)pyrrolidin-1-yl)-5-methyl-1-oxoheptan-4-yl)-N,3-dimethylbutanamide hydrochloride INT-58

To a solution of INT-57 (1.0 g, 1.36 mmol) in 10 mL DCM was added 4 M HCl/dioxane (5 mL, 20 mmol). The reaction was stirred at room temperature for 3 h. TLC showed completion. The mixture was concentrated to dry. The residue was slurred with MTBE (10 mL*3) then freeze dried to afford INT-58 (0.76 g, 86% yield) as a yellow solid. LCMS (ESI): m/z 634.2 [M+H]⁺; HPLC: 97.1% @210 nm, Rₜ=9.36 min.

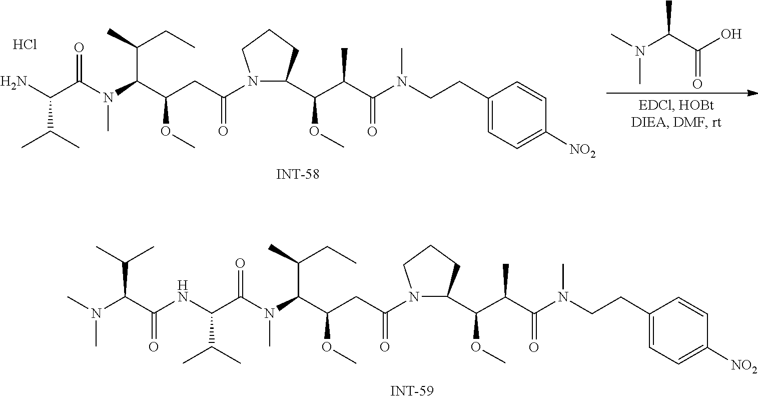

### (S)-2-((S)-2-(dimethylamino)-3-methylbutanamido)-N-((3R,4S,5S)-3-methoxy-1-((S)-2-((1R,2R)-1-methoxy-2-methyl-3-(methyl(4-nitrophenethyl)amino)-3-oxopropyl)pyrrolidin-1-yl)-5-methyl-1-oxoheptan-4-yl)-N,3-dimethylbutanamide INT-59

To a solution of INT-58 (100 mg, 0.149 mmol) and (S)-2-(dimethylamino)-3-methylbutanoic acid (33 mg, 0.224 mmol) in 2 mL DMF was added EDCI (46 mg, 0.235 mmol) and HOBt (32 mg, 0.328 mmol), followed by the addition of DIEA (124 mg,0.969 mmol). The mixture was stirred at room temperature under a N₂ atmosphere for 2 h, LCMS showed completion. The reaction mixture was purified by reverse phase column (H₂O:CH₃CN) directly to afford INT-59 (120 mg, 100% yield) as a colorless oil. LCMS (ESI): m/z 761.1[M+H]⁺.

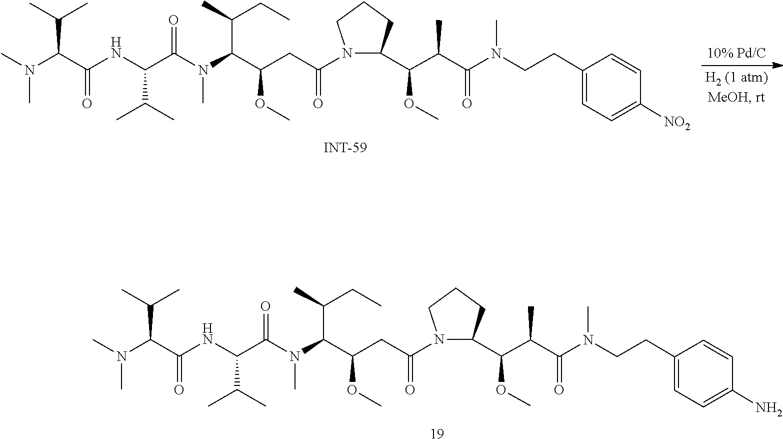

### (S)-N-((3R,4S,5S)-1-((S)-2-((1R,2R)-3-((4-aminophenethyl)(methyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide 19

To a solution of INT-59 (120 mg, 0.149 mmol) in 3 mL MeOH was added 10% Pd/C (24 mg). The reaction was then stirred under a H₂ atmosphere (1 atm) at room temperature for 3 h. LCMS showed completion. The mixture was filtrated, and the filtrate was concentrated.. The residue was purified by Prep-TLC (DCM:MeOH=12:1, R_{f}=0.5)then freeze-dried to afford 19 (45 mg, 41% yield) as an off-white solid. LCMS (ESI): m/z 366.2 [M+2H]²⁺; HPLC: 95.6% @210 nm, Rₜ=12.36 min;¹H NMR (400 MHz, DMSO-d₆) δ 8.01 (d, J=8.7 Hz, 1H), 6.90-6.81 (m, 2H), 6.53-6.42 (m, 2H), 4.90-4.75 (m, 2H), 4.73-4.47 (m, 2H), 4.17-3.79 (m, 2H), 3.79-3.59 (m, 2H), 3.55-3.41 (m, 2H), 3.40-3.33 (m, 2H), 3.29-3.25 (m, 2H), 3.24-3.16 (m, 4H), 3.15-2.93 (m, 3H), 2.91-2.82 (m, 2H), 2.82-2.77 (m, 1H), 2.67-2.58 (m, 3H), 2.57-2.52 (m, 1H), 2.49-2.39 (m, 1H), 2.24-2.17 (m, 6H), 1.99-1.83 (m, 4H), 1.82-1.56 (m, 3H), 1.34-1.25 (m, 1H), 1.11-1.00 (m, 2H), 0.99-0.94 (m, 1H), 0.93-0.84 (m, 13H), 0.79-0.68 (m, 6H).

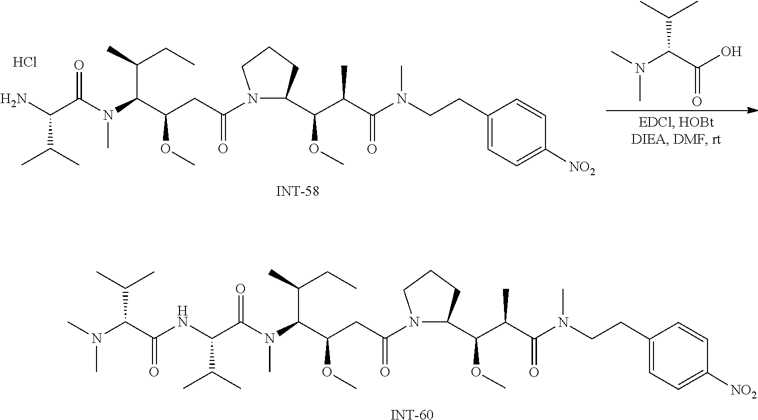

### (S)-2-((R)-2-(dimethylamino)-3-methylbutanamido)-N-((3R,4S,5S)-3-methoxy-1-((S)-2-((1R,2R)-1-methoxy-2-methyl-3-(methyl(4-nitrophenethyl)amino)-3-oxopropyl)pyrrolidin-1-yl)-5-methyl-1-oxoheptan-4-yl)-N,3-dimethylbutanamide INT-60

To a solution of INT-58 (100 mg, 0.149 mmol) and (R)-2-(dimethylamino)-3-methylbutanoic acid (49 mg, 0.268 mmol) in 3 mL DMF was added EDCI (68 mg, 0.355 mmol) and HOBt (64 mg, 0.473 mmol), followed by the addition of DIEA (0.19 mL,1.06 mmol). The mixture was stirred at room temperature under a N₂ atmosphere for 2 days, LCMS showed near completion. The reaction mixture was purified by reverse phase column (H₂O:CH₃CN) directly to afford INT-60 (60 mg, 53% yield) as a colorless oil. LCMS (ESI): m/z 761.1[M+H]⁺.

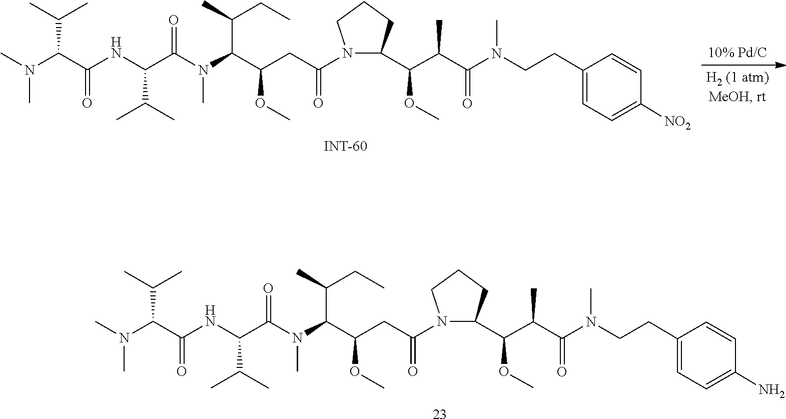

### (S)-N-((3R,4S,5S)-1-((S)-2-((1R,2R)-3-((4-aminophenethyl)(methyl)amino)-1-methoxy-2-methyl-3-oxopropyl)pyrrolidin-1-yl)-3-methoxy-5-methyl-1-oxoheptan-4-yl)-2-((R)-2-(dimethylamino)-3-methylbutanamido)-N,3-dimethylbutanamide 23

To a solution of INT-60 (60 mg, 0.788 mmol) in 2 mL MeOH was added 10% Pd/C (18 mg). The reaction was then stirred under a H₂ atmosphere (1 atm) at room temperature for overnight. LCMS showed completion. The mixture was filtrated, and the filtrate was concentrated. The residue was freeze-dried to afford 23 (45 mg, 78% yield) as a white solid. LCMS (ESI): m/z 731.4 [M+H]⁺; HPLC: 95.9% @210 nm, Rₜ=12.13 min;¹H NMR (400 MHz, DMSO-d₆) δ 8.01 (d, J=8.7 Hz, 1H), 6.91-6.79 (m, 2H), 6.53-6.41 (m, 2H), 4.89-4.74 (m, 2H), 4.73-4.44 (m, 2H), 4.15-3.84 (m, 2H), 3.77-3.58 (m, 2H), 3.57-3.38 (m, 3H), 3.30-3.23 (m, 3H), 3.23-3.16 (m, 4H), 3.16-2.95 (m, 3H), 2.89-2.82 (m, 2H), 2.82-2.77 (m, 1H), 2.69-2.54 (m, 4H), 2.43 (d, J=15.3 Hz, 1H), 2.28-2.12 (m, 6H), 2.01-1.84 (m, 4H), 1.81-1.57 (m, 3H), 1.40-1.29 (m, 1H), 1.05 (dd, J=17.7, 6.6 Hz, 2H), 0.98-0.94 (m, 1H), 0.93-0.83 (m, 13H), 0.82-0.70 (m, 6H).

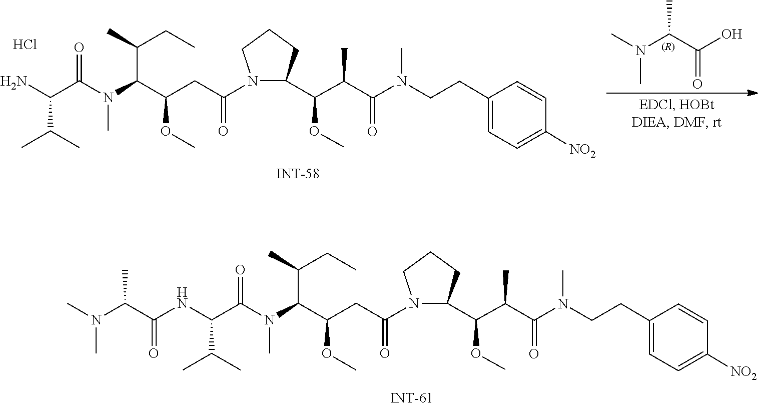

### (S)-2-((R)-2-(dimethylamino)propanamido)-N-((3R,4S,5S)-3-methoxy-1-((S)-2-((1R,2R)-1-methoxy-2-methyl-3-(methyl(4-nitrophenethyl)amino)-3-oxopropyl)pyrrolidin-1-yl)-5-methyl-1-oxoheptan-4-yl)-N,3-dimethylbutanamide INT-61

To a solution of INT-58 (100 mg, 0.149 mmol) and (R)-2-(dimethylamino)propanoic acid (24 mg, 0.194 mmol) in 2 mL DMF was added EDCI (46 mg, 0.239 mmol) and HOBt (43 mg, 0.313 mmol), followed by the addition of DIEA (0.12 mL, 0.744 mmol). The mixture was stirred at room temperature under a N₂ atmosphere for 2 days, LCMS showed 30% STM remained. The reaction was stopped and purified by reverse phase column (H₂O:CH₃CN) directly to afford crude INT-61 (120 mg) as a yellow oil. LCMS (ESI): m/z 733 [M+H]⁺.

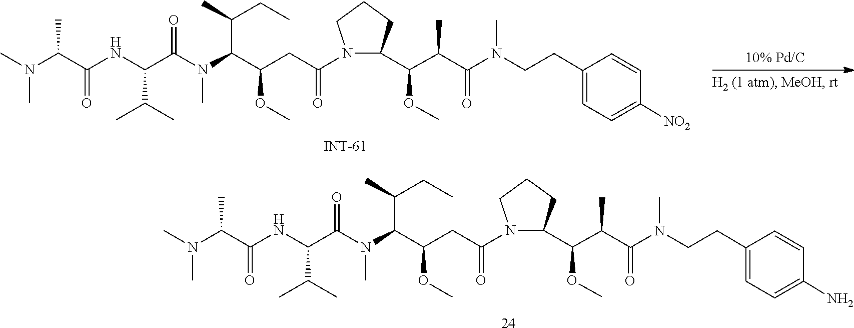

### (S)-N-((3R,4S,5S)-1-((S)-2-((1R,2R)-3-((4-aminophenethyl)(methyl)amino)-1-methoxy-2-methyl-3-oxopropyl)pyrrolidin-1-yl)-3-methoxy-5-methyl-1-oxoheptan-4-yl)-2-((R)-2-(dimethylamino)propanamido)-N,3-dimethylbutanamide 24

To a solution of INT-61 (120 mg crude) in 3 mL MeOH was added 10% Pd/C (24 mg). The reaction was then stirred under a H₂ atmosphere (1 atm) at room temperature for 2 h. LCMS showed completion. The mixture was filtrated, and the filtrate was concentrated. The residue was purified by Prep-TLC (DCM:MeOH=13:1, R_{f}=0.5) to afford 24 (37 mg, 35% yield for 2 steps) as an off-white solid. LCMS (ESI): m/z 703.8 [M+H]⁺; HPLC: 97.7% @210 nm, Rt=11.99 min;1H NMR (400 MHz, DMSO-d₆) δ 7.80 (d, J=9.2 Hz, 1H), 6.90-6.79 (m, 2H), 6.54-6.39 (m, 2H), 4.93-4.78 (m, 2H), 4.74-4.47 (m, 2H), 4.05-3.75 (m, 2H), 3.74-3.56 (m, 2H), 3.55-3.41 (m, 2H), 3.30-3.23 (m, 3H), 3.19-3.14 (m, 3H), 3.13-2.91 (m, 4H), 2.86-2.73 (m, 3H), 2.65-2.53 (m, 3H), 2.48-2.37 (m, 1H), 2.34-2.17 (m, 1H), 2.17-2.10 (m, 6H), 2.02-1.79 (m, 4H), 1.76-1.59 (m, 2H), 1.34-1.26 (m, 1H), 1.08-1.00 (m, 5H), 0.97-0.71 (m, 15H).

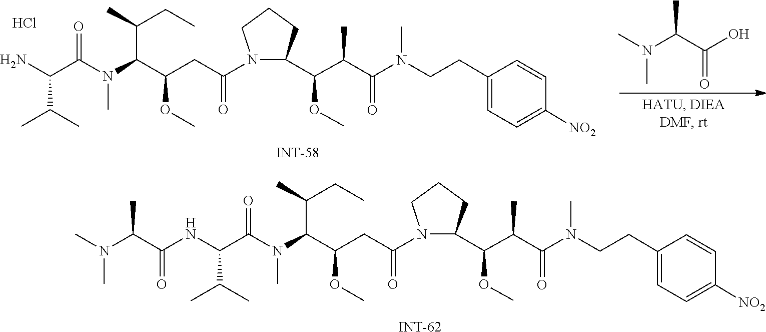

### (S)-2-((S)-2-(dimethylamino)propanamido)-N-((3R,4S,5S)-3-methoxy-1-((S)-2-((1R,2R)-1-methoxy-2-methyl-3-(methyl(4-nitrophenethyl)amino)-3-oxopropyl)pyrrolidin-1-yl)-5-methyl-1-oxoheptan-4-yl)-N,3-dimethylbutanamide INT-62

To a solution of INT-58 (100 mg, 0.149 mmol) and (S)-2-(dimethylamino)propanoic acide (21 mg, 0.174 mmol) in 3 mL DMF was added HATU (78 mg, 0.205 mmol), followed by the addition of DIEA (51 mg, 0.395 mmol). The mixture was stirred at room temperature under a N₂ atmosphere for 3 h, LCMS showed completion. The mixture was concentrated to dry directly to afford the crude. The crude residue was diluted with 10 mL H₂O, then extracted with EtoAc (10 mL*4). The combined organic layers were washed with H₂O and brine, dried over Na₂SO₄, filtrated and concentrated to afford crude INT-62 (185 mg) as yellow oil. LCMS (ESI): m/z 733.3 [M+H]⁺.

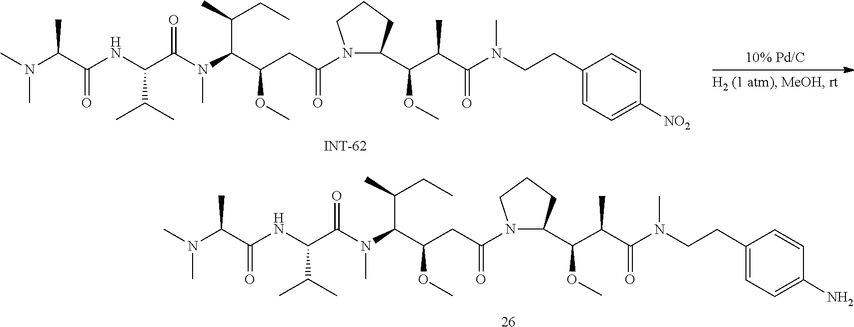

### (S)-N-((3R,4S,5S)-1-((S)-2-((1R,2R)-3-((4-aminophenethyl)(methyl)amino)-1-methoxy-2-methyl-3-oxopropyl)pyrrolidin-1-yl)-3-methoxy-5-methyl-1-oxoheptan-4-yl)-2-((S)-2-(dimethylamino)propanamido)-N,3-dimethylbutanamide 26

To a solution of INT-62 (185 mg crude) in 5 mL MeOH was added 10% Pd/C (30 mg). The reaction was then stirred under a H₂ atmosphere (1 atm) at room temperature for overnight. LCMS showed completion. The mixture was filtrated, and the filtrate was concentrated. The residue was purified by reverse phase column (H₂O:CH₃CN) then freeze-dried to afford 26 (30 mg, 28% yield for 2 steps) as a light pink solid. LCMS (ESI): m/z 725.8 [M+Na]⁺, 352.6 [M+2H]²⁺; HPLC: 92.9% @210 nm, Rₜ=11.53 min

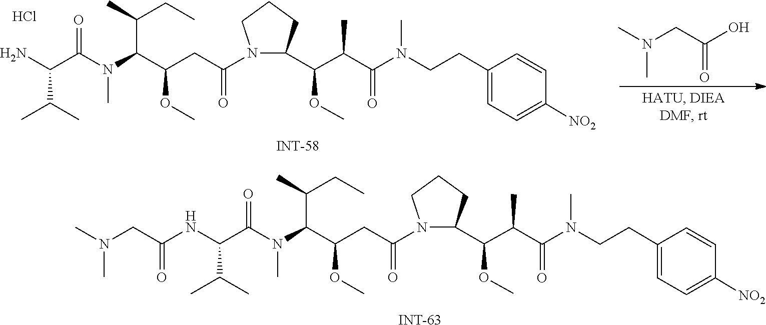

### (S)-2-(2-(dimethylamino)acetamido)-N-((3R,4S,5S)-3-methoxy-1-((S)-2-((1R,2R)-1-methoxy-2-methyl-3-(methyl(4-nitrophenethyl)amino)-3-oxopropyl)pyrrolidin-1-yl)-5-methyl-1-oxoheptan-4-yl)-N,3-dimethylbutanamide INT-63

To a solution of INT-58 (100 mg, 0.149 mmol) and 2-(dimethylamino)acetic acid (18 mg, 0.174 mmol) in 4 mL DMF was added HATU (78 mg, 0.205 mmol), followed by the addition of DIEA (51 mg, 0.395 mmol). The mixture was stirred at room temperature under a N₂ atmosphere for 3 h, LCMS showed completion. The mixture was concentrated to dry directly to afford the crude. The crude residue was diluted with 15 mL H₂O, then extracted with EtoAc (10 mL*4). The combined organic layers were washed with H₂O and brine, dried over Na₂SO₄, filtrated and concentrated to afford crude INT-63 (190 mg) as yellow oil. LCMS (ESI): m/z 719.3 [M+H]⁺.

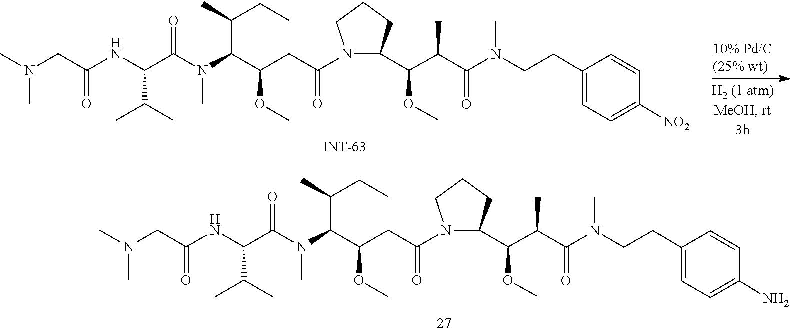

### (S)-N-((3R,4S,5S)-1-((S)-2-((IR,2R)-3-((4-aminophenethyl)(methyl)amino)-1-methoxy-2-methyl-3-oxopropyl)pyrrolidin-1-yl)-3-methoxy-5-methyl-1-oxoheptan-4-yl)-2-(2-(dimethylamino)acetamido)-N,3-dimethylbutanamide 27

To a solution of crude INT-63 (190 mg) in 5 mL MeOH was added 10% Pd/C (30 mg). The reaction was then stirred under a H₂ atmosphere (1 atm) at room temperature for overnight. LCMS showed completion. The mixture was filtrated, and the filtrate was concentrated. The residue was purified by reverse phase column (H₂O:CH₃CN) thenprep-TLC (DCM:MeOH=13:1, R_{f}=0.5) to afford 27 (23 mg, 22% yield for 2 steps) as an off-white solid. LCMS (ESI): m/z 345.3 [M+2H]²⁺; HPLC: 98.0% @210 nm, Rₜ=12.07 min;¹H NMR (400 MHz, DMSO-d₆) δ 7.65 (d, J=6.0 Hz, 1H), 6.93-6.80 (m, 2H), 6.57-6.43 (m, 2H), 4.94-4.78 (m, 2H), 4.77-4.57 (m, 2H), 4.12-3.90 (m, 2H), 3.90-3.69 (m, 2H), 3.66-3.58 (m, 1H), 3.53-3.48 (m, 1H), 3.47-3.43 (m, 1H), 3.31-3.25 (m, 2H), 3.24-3.04 (m, 4H), 3.01-2.91 (m, 2H), 2.88-2.82 (m, 2H), 2.82-2.69 (m, 2H), 2.68-2.53 (m, 3H), 2.48-2.35 (m, 1H), 2.25-2.16 (m, 5H), 2.07-1.91 (m, 2H), 1.90-1.77 (m, 2H), 1.76-1.57 (m, 2H), 1.51-1.34 (m, 1H), 1.33-1.17 (m, 4H), 1.04 (dd, J=15.9, 6.7 Hz, 2H), 0.97-0.73 (m, 12H).

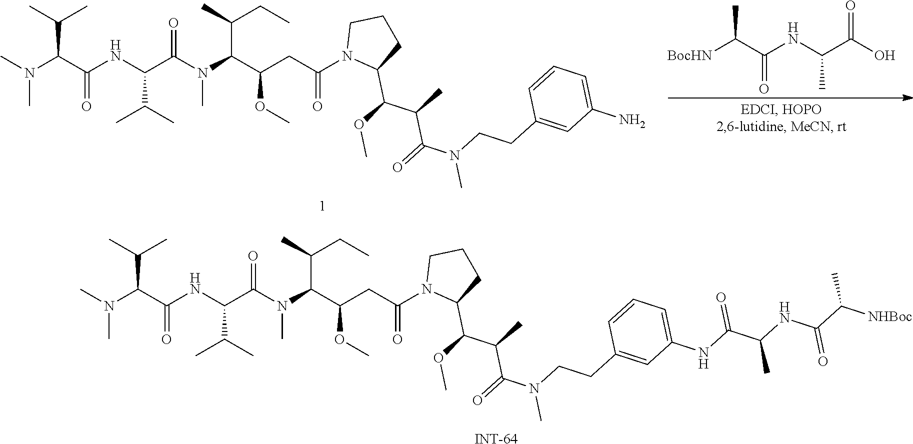

### Tert-butyl ((S)-1-(((S)-1-((3-(2-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate GINT-64

To a solution of 1 (110 mg, 150 umol) and (S)-2-((S)-2-((tert-butoxycarbonyl)amino)propanamido)propanoic acid (47 mg, 181 umol) in 3 mL CH₃CN was added a mixture of EDCI (43 mg, 226 umol) and HOPO (25 mg, 226 mmol), following by the addition of 2,6-lutidine (53 uL, 451 umol). The reaction was stirred at room temperature under a N₂ atmosphere for 16 h, LCMS showed completion. The mixture was concentrated to dry directly, and the residue was purified by Prep-TLC (DCM: MeOH=13:1, R_{f}=0.65) to afford INT-64 (70 mg, 49% yield) as yellow oil. LCMS (ESI): m/z 972.8 [M+H]⁺, 995.7 [M+Na⁺].

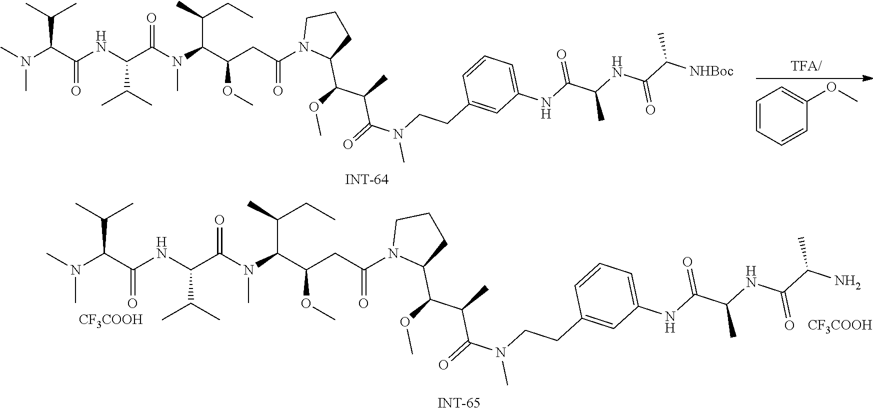

### (S)-N-((3R,4S,5S)-1-((S)-2-((IR,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide bis(2,2,2-trifluoroacetate) INT-65

To a mixture of INT-64 (80 mg, 82 umol) and anisole (45 uL, 411 umol) was added TFA (0.7 mL). The reaction was then stirred at room temperature for 10 min then quenched by adding 350 mL MTBE, during which time, much white solid precipitated. The resulting mixture was filtrated, and the filter cake was collected and dried under reduced pressure to afford INT-65 (70 mg, 76% yield) as an off-white solid. LCMS (ESI): m/z 873.8 [M+H]⁺.

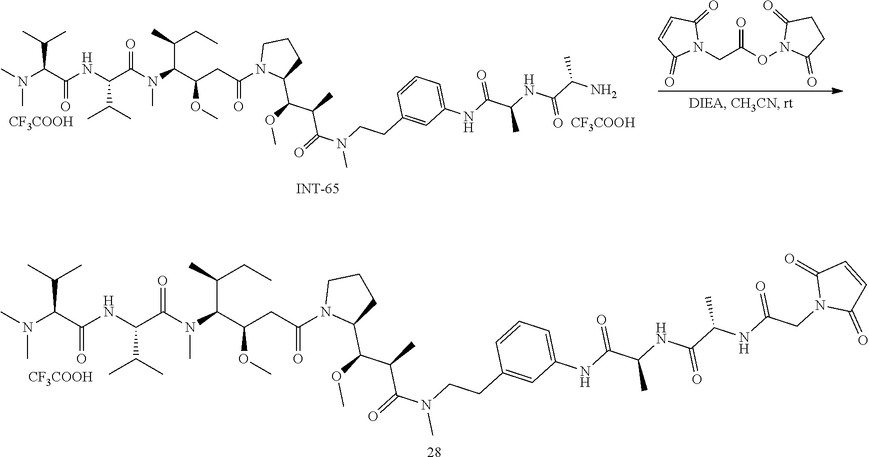

### (S)-2-((S)-2-(dimethylamino)-3-methylbutanamido)-N-((3R,4S,5S)-1-((S)-2-((1R,2R)-3-((3-((S)-2-((S)-2-(2-(2,5-dioxo-2,5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)-N,3-dimethylbutanamide 2,2,2-trifluoroacetate 28

To a solution of INT-65 (70 mg, 64 umol) and 2,5-dioxopyrrolidin-1-yl 2-(2,5-dioxo-2,5-dihydro-1H-pyrrol-1-yl)acetate (26 mg, 104 umol) in 2 mL CH₃CN was added DIEA (27 uL, 160 umol). The reaction was then stirred at room temperature for 45 min. LCMS showed completion. The mixture was quenched by adding TFA (0.02 mL) directly and stirred for 5 min. The resulting mixture was sent to Prep-HPLC (0.1% TFA in H₂O/CH₃CN) instantly then freeze-dried to afford 28 (45 mg, 63% yield) as a white solid. LCMS (ESI): m/z 505.5 [M+2H]²⁺; HPLC: 99.7% @210 nm, Rₜ=8.87 min; ¹H NMR (400 MHz, DMSO) δ 9.84-9.75 (m, 1H), 9.52 (s, 1H), 8.91 (d, J=8.1 Hz, 1H), 8.43 (d, J=7.2 Hz, 1H), 8.19-8.11 (m, 1H), 7.49 (d, J=7.4 Hz, 1H), 7.45-7.35 (m, 1H), 7.23-7.15 (m, 1H), 7.09 (s, 2H), 6.95-6.87 (m, 1H), 4.77-4.64 (m, 1H), 4.63-4.55 (m, 1H), 4.40-4.28 (m, 2H), 4.13-4.04 (m, 2H), 4.03-3.96 (m, 1H), 3.92 (m, 1H), 3.78-3.69 (m, 3H), 3.51-3.49 (m, 1H), 3.48-3.46 (m, 1H), 3.45-3.42 (m, 1H), 3.35-3.24 (m, 4H), 3.22-3.16 (m, 3H), [3.13 (s, 1.5H), 2.99 (s, 1.5H)], 2.90 (d, J=2.2 Hz, 2H), 2.84-2.79 (m, 2H), 2.79-2.70 (m, 6H), 2.70-2.58 (m, 2H), 2.48-2.40 (m, 1H), 2.36-2.18 (m, 2H), 2.06-1.92 (m, 2H), 1.89-1.84 (m, 1H), 1.81-1.56 (m, 3H), 1.30 (dd, J=7.0, 3.2 Hz, 4H), 1.25-1.18 (m, 4H), 1.04 (dd, J=14.9, 6.7 Hz, 2H), 0.98-0.82 (m, 15H), 0.81-0.72 (m, 3H).

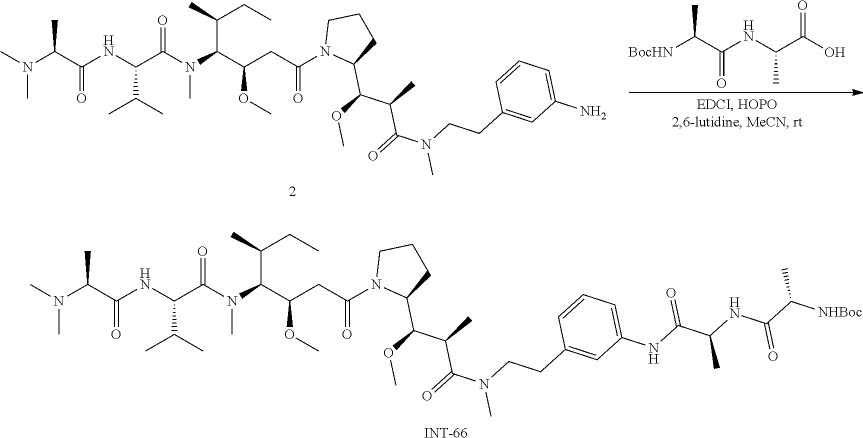

### Tert-butyl ((S)-1-(((S)-1-((3-(2-((2R,3R)-3-((S)-1-((3R,4S,5S)-4-((S)-2-((S)-2-(dimethylamino)propanamido)-N,3-dimethylbutanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-66

To a solution of 2 (110 mg, 156 umol) and (S)-2-((S)-2-((tert-butoxycarbonyl)amino)propanamido)propanoic acid (58 mg, 223 umol) in 5 mL CH₃CN was added a mixture of EDCI (53 mg, 276 umol) and HOPO (31 mg, 279 umol), following by the addition of 2,6-lutidine (60 mg, 560 umol). The reaction was stirred at room temperature under a N₂ atmosphere for 16 h, LCMS showed completion. The mixture was concentrated to dry directly, and the residue was purified by Prep-TLC (DCM: MeOH=10:1, R_{f}=0.75) to afford INT-66 (120 mg, 82% yield) as colorless oil. LCMS (ESI): m/z 473.2 [M+2H]²⁺.

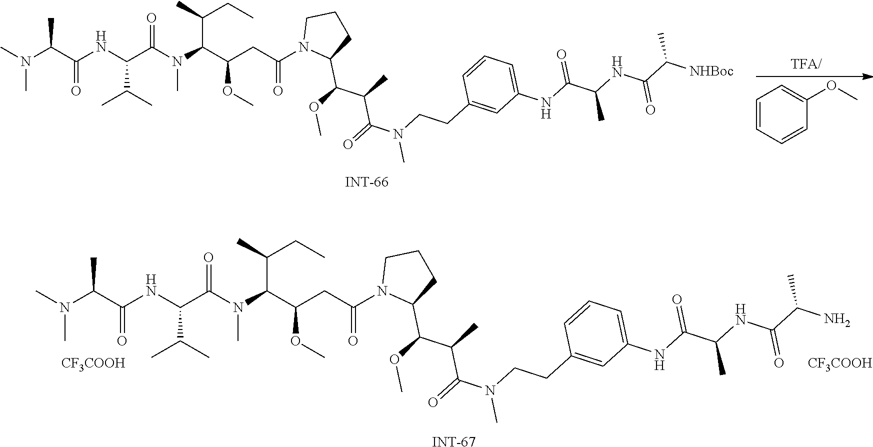

### (S)-N-((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)-2-((S)-2-(dimethylamino)propanamido)-N,3-dimethylbutanamide bis(2,2,2-trifluoroacetate) INT-67

To a mixture of INT-66 (120 mg, 127 umol) and anisole (69 mg, 638 umol) was added TFA (3 mL). The reaction was then stirred at room temperature for 5 min. TLC showed completion (DCM/MeOH=10:1, v/v; R_{f}=0.75 for INT-66). The mixture was diluted with 150 mL MTBE, during which time, much white solid precipated. The resulting mixture was filtrated, and the filter cake was collected and dried under reduced pressure to afford INT-67 (148 mg, 100% yield) as colorless oil. LCMS (ESI): m/z 846.1 [M+H]⁺.

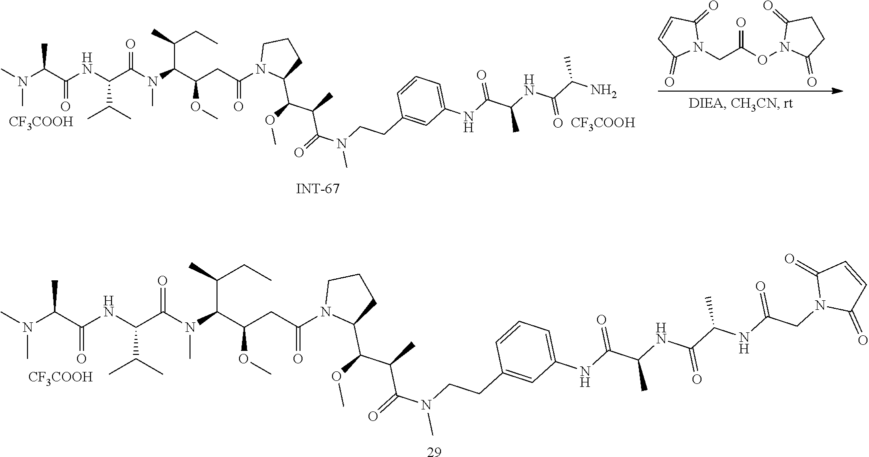

### (S)-2-((S)-2-(dimethylamino)propanamido)-N-((3R,4S,5S)-1-((S)-2-((1R,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)-N,3-dimethylbutanamide 2,2,2-trifluoroacetate 29

To a solution of INT-67 (148 mg, 129 umol) and 2,5-dioxopyrrolidin-1-yl 2-(2,5-dioxo-2,5-dihydro-1H-pyrrol-1-yl)acetate (47 mg, 186 umol) in 5 mL CH₃CN was added DIEA (30 mg, 232 umol). The reaction was then stirred at room temperature for 30 min. LCMS showed completion. The mixture was quenched by adding TFA (0.12 mL) directly. The resulting mixture was sent to Prep-HPLC (0.1% TFA in H₂O/CH₃CN) instantly then freeze-dried to afford 29 (40 mg, 28% yield) as a white solid. LCMS (ESI): m/z 982.9 [M+H]⁺, 491.6 [M+2H]2+; HPLC: 98.5% @210 nm, Rₜ=8.65 min; ¹H NMR (400 MHz, DMSO) δ 9.90-9.70 (m, 2H), 8.97 (d, J=8.4 Hz, 1H), 8.43 (d, J=7.2 Hz, 1H), 8.22-8.10 (m, 1H), 7.54-7.48 (m, 1H), 7.48-7.36 (m, 1H), 7.27-7.17 (m, 1H), 7.09 (s, 2H), 6.98-6.87 (m, 1H), 4.79-4.60 (m, 1H), 4.61-4.49 (m, 1H), 4.43-4.28 (m, 2H), 4.17-4.04 (m, 2H), 4.02-3.89 (m, 2H), 3.80-3.63 (m, 2H), 3.33-3.25 (m, 4H), 3.23-3.13 (m, 4H), [3.11 (s, 1.2H), 2.98 (s, 1.8H)], 2.94-2.87 (m, 2H), 2.86-2.81 (m, 1H), 2.81-2.72 (m, 7H), 2.72-2.60 (m, 2H), 2.45-2.40 (m, 1H), 2.40-2.20 (m, 2H), 2.11-1.93 (m, 2H), 1.93-1.76 (m, 3H), 1.71-1.55 (m, 2H), 1.39-1.28 (m, 6H), 1.22 (d, J=6.3 Hz, 4H), 1.05 (dd, J=13.4, 6.7 Hz, 2H), 1.01-0.82 (m, 11H), 0.81-0.74 (m, 3H).

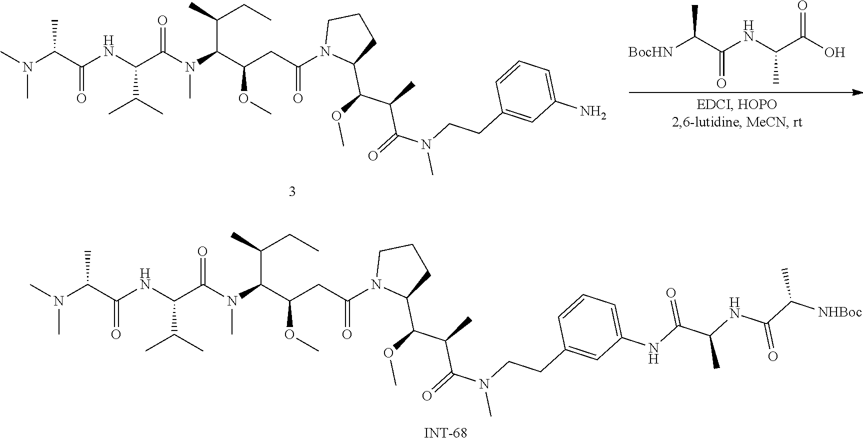

### Tert-butyl ((S)-1-(((S)-1-((3-(2-((2R,3R)-3-((S)-1-((3R,4S,5S)-4-((S)-2-((R)-2-(dimethylamino)propanamido)-N,3-dimethylbutanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-68

To a solution of 3 (90 mg, 128 umol) and (S)-2-((S)-2-((tert-butoxycarbonyl)amino)propanamido)propanoic acid (40 mg, 154 umol) in 3 mL CH₃CN was added a mixture of EDCI (37 mg, 192 umol) and HOPO (22 mg, 192 umol), following by the addition of 2,6-lutidine (45 uL, 384 umol). The reaction was stirred at room temperature under a N₂ atmosphere for 16 h, LCMS showed completion. The mixture was concentrated to dry directly, and the residue was purified by Prep-TLC (DCM: MeOH=13:1, R_{f}=0.65) to afford INT-68 (75 mg, 62% yield) as a yellow solid. LCMS (ESI): m/z 945.1 [M+H]⁺.

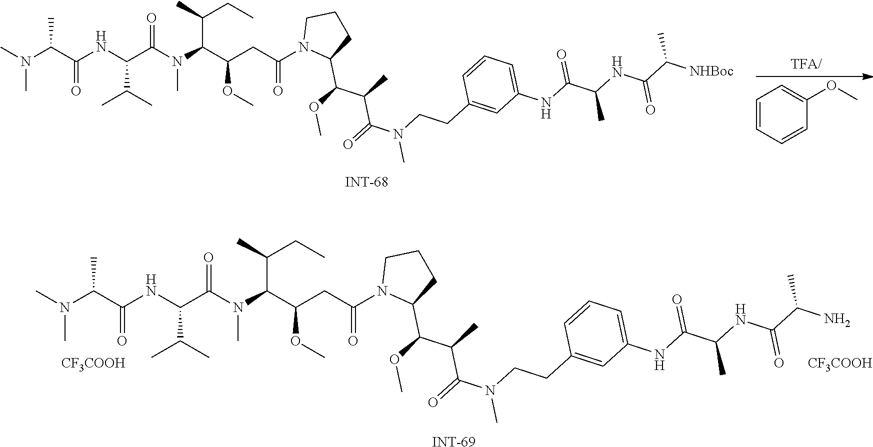

### (S)-N-((3R,4S,5S)-1-((S)-2-((IR,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)-2-((R)-2-(dimethylamino)propanamido)-N,3-dimethylbutanamide bis(2,2,2-trifluoroacetate) INT-69

To a mixture of INT-68 (75 mg, 79 umol) and anisole (43 uL, 400 umol) was added TFA (0.75 mL). The reaction was then stirred at room temperature for 20 min then quenched by adding 40 mL MTBE, during which time, much white solid precipated. The resulting mixture was filtrated, and the filter cake was collected and dried under reduced pressure to afford INT-69 (60 mg, 70% yield) as a yellow solid. LCMS (ESI): m/z 845.1 [M+H]⁺, 423.1 [M+2H]²⁺.

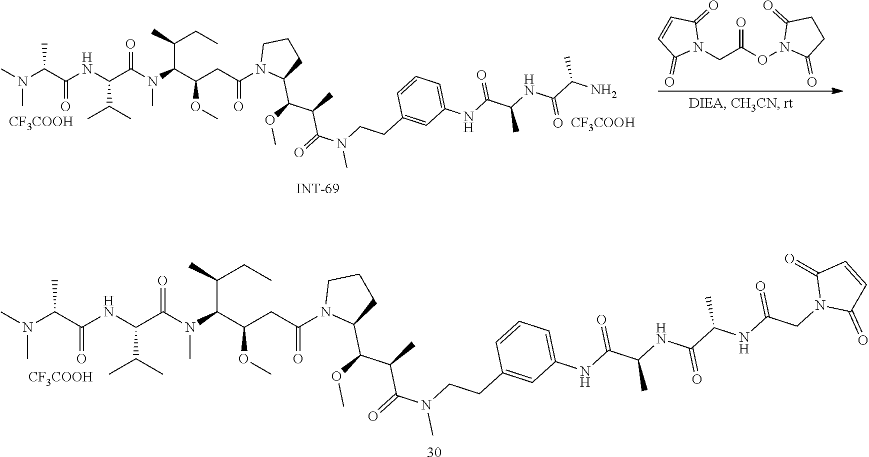

### (S)-2-((R)-2-(dimethylamino)propanamido)-N-((3R,4S,5S)-1-((S)-2-((1R,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)-N,3-dimethylbutanamide 2,2,2-trifluoroacetate 30

To a solution of INT-69 (60 mg, 56 umol) and 2,5-dioxopyrrolidin-1-yl 2-(2,5-dioxo-2,5-dihydro-1H-pyrrol-1-yl)acetate (21 mg, 81 umol) in 2 mL CH₃CN was added DIEA (20 uL, 125 umol). The reaction was then stirred at room temperature for 45 min LCMS showed completion. The mixture was quenched by adding TFA (30 uL) directly. The resulting mixture was sent to Prep-HPLC (0.1% TFA in H₂O/CH₃CN) instantly then freeze-dried to afford 30 (45 mg, 73% yield) as a white solid. LCMS (ESI): m/z 982.6 [M+H]⁺, 492.0 [M+2H]²⁺; HPLC: 98.8% @210 nm, R₁=8.63 min; H NMR (400 MHz, DMSO) δ 9.90-9.71 (m, 2H), 9.01-8.90 (m, 1H), 8.43 (d, J=7.2 Hz, 1H), 8.20-8.11 (m, 1H), 7.53-7.46 (m, 1H), 7.45-7.33 (m, 1H), 7.25-7.14 (m, 1H), 7.09 (s, 2H), 6.97-6.87 (m, 1H), 4.78-4.59 (m, 1H), 4.56-4.44 (m, 1H), 4.41-4.27 (m, 2H), 4.14-4.04 (m, 2H), 4.00-3.89 (m, 2H), 3.80-3.71 (m, 1H), 3.69-3.63 (m, 1H), 3.44-3.41 (m, 1H), 3.34-3.24 (m, 4H), 3.22-3.15 (in, 4H), [3.12 (s, 1.3H), 2.99 (s, 1.7H)], 2.94-2.86 (m, 2H), 2.84-2.74 (m, 5H), 2.73-2.61 (m, 5H), 2.46-2.39 (m, 1H), 2.31-2.18 (m, 1H), 2.08-1.91 (m, 2H), 1.89-1.76 (m, 2H), 1.72-1.55 (m, 2H), 1.46-1.38 (m, 3H), 1.35-1.26 (m, 4H), 1.21 (d, J=7.1 Hz, 4H), 1.04 (dd, J=14.2, 6.7 Hz, 2H), 0.99-0.82 (m, 11H), 0.77 (q, J=7.2 Hz, 3H).

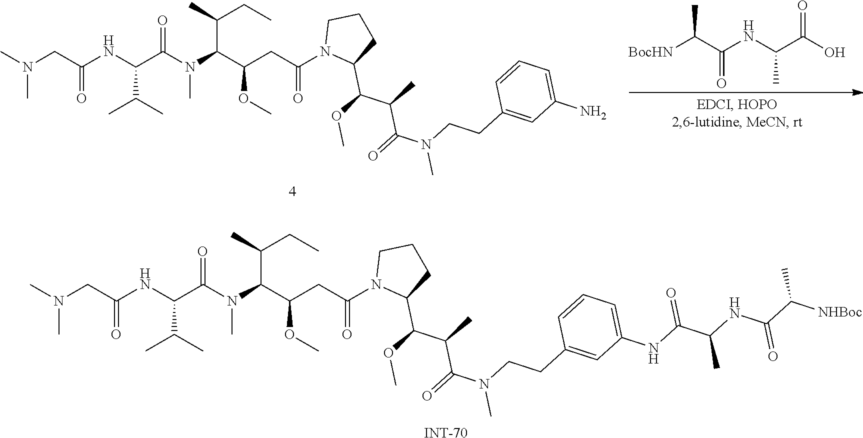

### Tert-butyl ((S)-1-(((S)-1-((3-(2-((2R,3R)-3-((S)-1-((3R,4S,5S)-4-((S)-2-(2-(dimethylamino)acetamido)-N,3-dimethylbutanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-70

To a solution of 4 (80 mg, 116 umol) and (S)-2-((S)-2-((tert-butoxycarbonyl)amino)propanamido)propanoic acid (36 mg, 139 umol) in 5 mL CH₃CN was added a mixture of EDCI (33 mg, 172 umol) and HOPO (20 mg, 180 umol), following by the addition of 2,6-lutidine (37 mg, 345 umol). The reaction was stirred at room temperature under a N₂ atmosphere for 16 h, LCMS showed completion. The mixture was concentrated to dry directly, and the residue was purified by Prep-TLC (DCM: MeOH=10:1, R_{f}=0.75) to afford INT-70 (100 mg, 93% yield) as yellow oil. LCMS (ESI): m/z 931.0 [M+H]⁺.

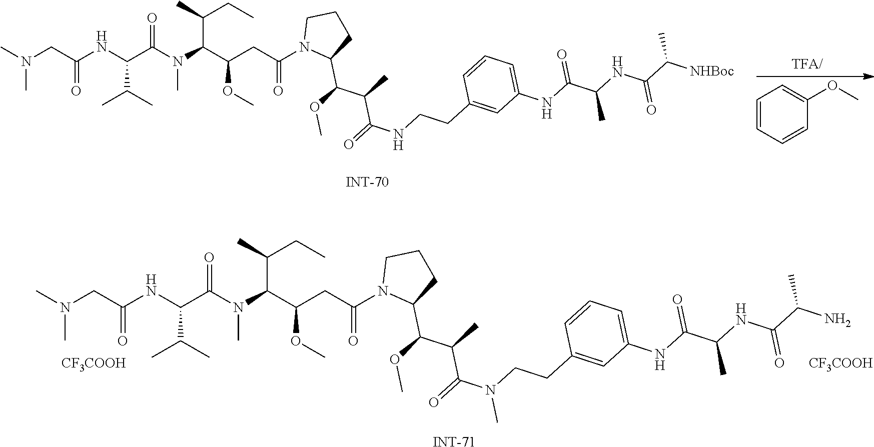

### (S)-N-((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)-2-(2-(dimethylamino)acetamido)-N,3-dimethylbutanamide bis(2,2,2-trifluoroacetate) GEF2101-46-3 (1550-26)

To a mixture of INT-70 (100 mg, 107 umol) and anisole (60 mg, 555 umol) was added TFA (3 mL). The reaction was then stirred at room temperature for 5 min. TLC showed completion (DCM/MeOH=10:1, v/v; R_{f}=0.75 for INT-70). The mixture was diluted with 100 mL MTBE, during which time, much white solid precipated. The resulting mixture was filtrated, and the filter cake was collected and dried under reduced pressure to afford INT-71 (160 mg, >100% yield) as yellow oil, which was used directly without further identification.

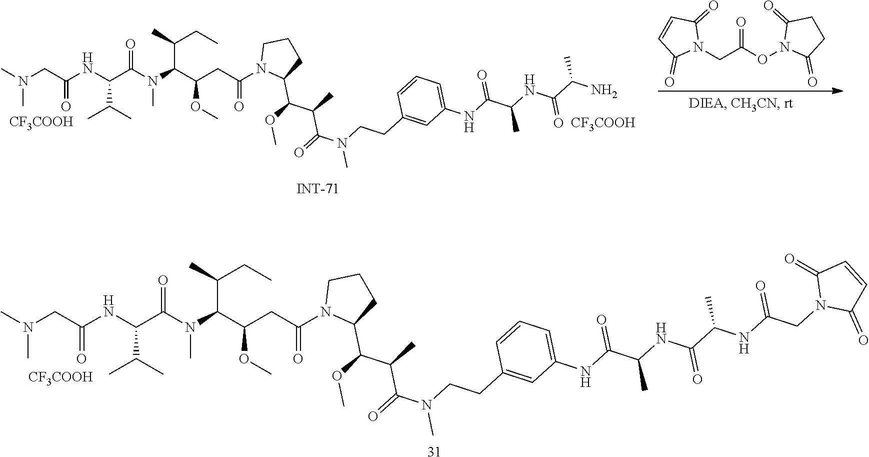

### (S)-2-(2-(dimethylamino)acetamido)-N-((3R,4S,5S)-1-((S)-2-((IR,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)-N,3-dimethylbutanamide 2,2,2-trifluoroacetate 31

To a solution of INT-71 (160 mg crude, 107 umol) and 2,5-dioxopyrrolidin-1-yl 2-(2,5-dioxo-2,5-dihydro-1H-pyrrol-1-yl)acetate (52 mg, 206 umol) in 5 mL CH₃CN was added DIEA (33 mg, 256 umol). The reaction was then stirred at room temperature for 80 min. HPLC showed completion. The mixture was quenched by adding TFA (0.14 mL) directly. The resulting mixture was sent to Prep-HPLC (0.1% TFA in H₂O/CH₃CN) instantly then freeze-dried to afford 31 (65 mg, 56% yield) as a white solid. LCMS (ESI): m/z 484.6 [M+2H]²⁺; HPLC: 99.5% @210 nm, Rₜ=8.61 min; ¹H NMR (400 MHz, DMSO) δ 9.90-9.65 (m, 2H), 8.86 (d, J=8.3 Hz, 1H), 8.43 (d, J=7.2 Hz, 1H), 8.21-8.11 (m, 1H), 7.49 (s, 1H), 7.46-7.34 (m, 1H), 7.24-7.15 (m, 1H), 7.08 (s, 2H), 6.97-6.87 (m, 1H), 4.75-4.52 (m, 2H), 4.41-4.28 (m, 2H), 4.14-4.04 (m, 2H), 4.04-3.95 (m, 2H), 3.94-3.88 (m, 1H), 3.79-3.71 (m, 1H), 3.70-3.62 (m, 1H), 3.61-3.54 (m, 1H), 3.52-3.46 (m, 2H), 3.33-3.23 (m, 4H), 3.21-3.09 (m, 3H), [3.14 (s, 1.5H), 2.97 (s, 1.5H)],2.93-2.87 (m, 2H), 2.83-2.73 (m, 8H), 2.71-2.66 (m, 1H), 2.64-2.57 (m, 1H), 2.49-2.18 (m, 2H), 2.10-1.97 (m, 1H), 1.95-1.76 (m, 3H), 1.75-1.54 (m, 2H), 1.38-1.27 (m, 4H), 1.22 (d, J=6.8 Hz, 3H), 1.04 (dd, J=13.5, 6.7 Hz, 2H), 1.01-0.83 (m, 11H), 0.79 (q, J=7.5 Hz, 3H).

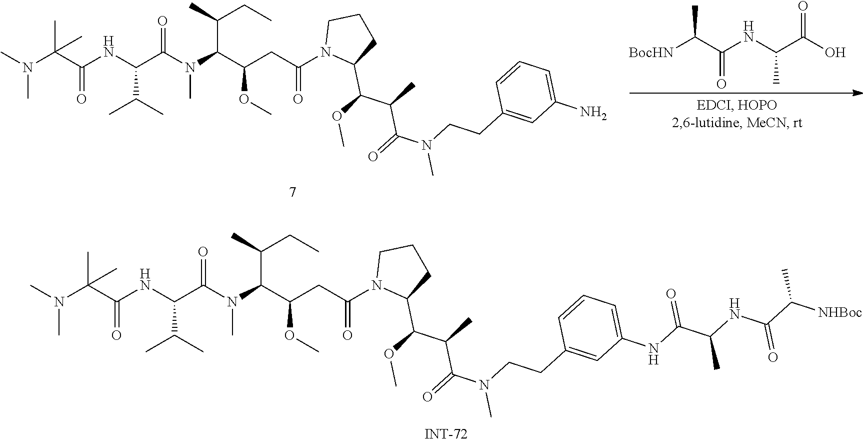

### Tert-butyl ((S)-1-(((S)-1-((3-(2-((2R,3R)-3-((S)-1-((3R,4S,5S)-4-((S)-2-(2-(dimethylamino)-2-methylpropanamido)-N,3-dimethylbutanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-72

To a solution of 7 (110 mg, 153 umol) and (S)-2-((S)-2-((tert-butoxycarbonyl)amino)propanamido)propanoic acid (48 mg, 184 umol) in 3 mL CH₃CN was added a mixture of EDCI (44 mg, 230 umol) and HOPO (26 mg, 230 umol), following by the addition of 2,6-lutidine (54 uL, 460 umol). The reaction was stirred at room temperature under a N₂ atmosphere for 16 h, LCMS showed completion. The mixture was purified by reverse phase column (H₂O:CH₃CN) directly to afford INT-72 (130 mg, 88% yield) as light yellow oil. LCMS (ESI): m/z 959.2 [M+H]⁺.

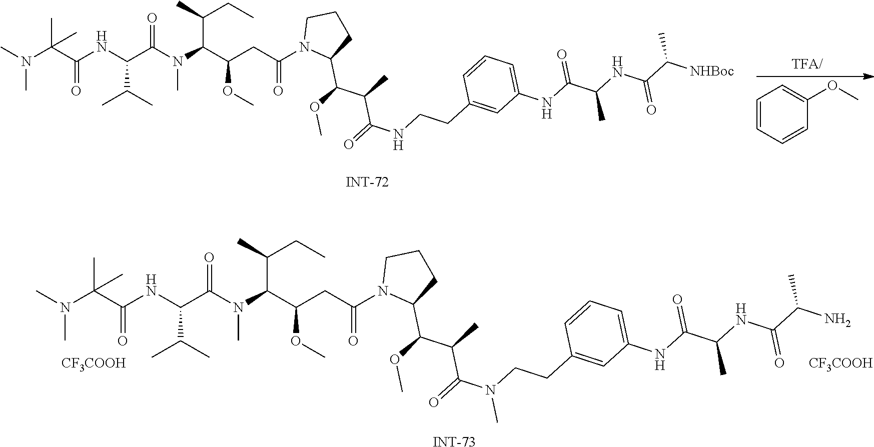

### (S)-N(3R,4S,5S)-1-((S-2-((1R,2R)-3-((3-((S-2-((S-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)-2-(2-(dimethylamino)-2-methylpropanamido)-N,3-dimethylbutanamide bis(2,2,2-trifluoroacetate) INT-73

To a mixture of INT-72 (130 mg, 136 umol) and anisole (75 uL, 691 umol) was added TFA (1.2 mL). The reaction was then stirred at room temperature for 10 min then quenched by adding 60 mL MTBE, during which time, much white solid precipated. The resulting mixture was filtrated, and the filter cake was collected and dried under reduced pressure to afford INT-73 (115 mg, 77% yield) as a light yellow solid. LCMS (ESI): m/z 859.3 [M+H]⁺.

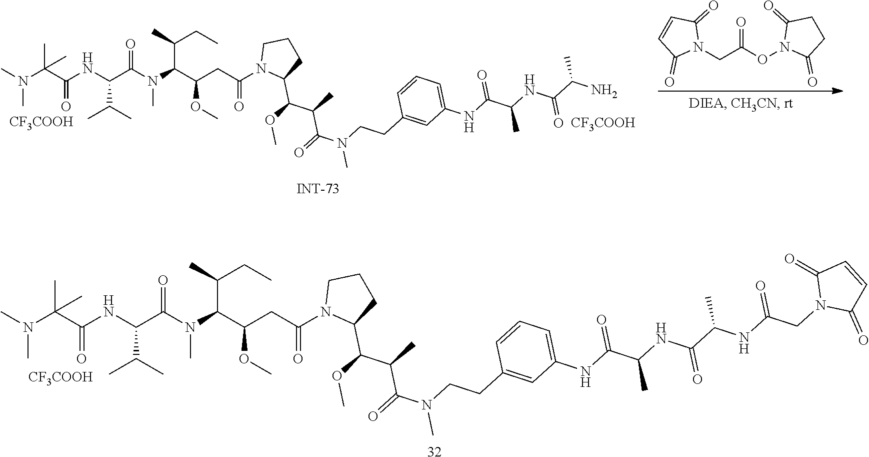

### (S)-2-(2-(dimethylamino)-2-methylpropanamido)-N-((3R,4S,5S)-1-((S)-2-((1R,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)-N,3-dimethylbutanamide 2,2,2-trifluoroacetate 32

To a solution of INT-73 (110 mg, 101 umol) and 2,5-dioxopyrrolidin-1-yl 2-(2,5-dioxo-2,5-dihydro-1H-pyrrol-1-yl)acetate (37 mg, 147 umol) in 2.5 mL CH₃CN was added DIEA (38 uL, 226 umol). The reaction was then stirred at room temperature for 50 min then quenched by adding TFA (70 uL) directly. The resulting mixture was sent to Prep-HPLC (0.1% TFA in H₂O/CH₃CN) instantly then freeze-dried to afford 32 (55 mg, 49% yield) as a white solid. LCMS (ESI): m/z 997.2 [M+H]⁺, 498.8 [M+2H]²⁺; HPLC: 96.3% @210 nm, Rₜ=8.69 min; ¹H NMR (400 MHz, DMSO) δ 9.91-9.75 (m, 1H), 9.68 (s, 1H), 8.56-8.35 (m, 2H), 8.27-8.08 (m, 1H), 7.50 (s, 1H), 7.48-7.33 (m, 1H), 7.29-7.14 (m, 1H), 7.10 (s, 2H), 6.99-6.85 (m, 1H), 4.82-4.58 (m, 1H), 4.57-4.44 (m, 1H), 4.44-4.23 (m, 2H), 4.17-4.04 (m, 2H), 4.02-3.88 (m, 1H), 3.79-3.65 (m, 1H), 3.62-3.36 (m, 3H), 3.33-3.24 (m, 3H), 3.22-3.16 (m, 3H), 3.16-2.96 (m, 3H), 2.96-2.86 (m, 2H), 2.86-2.79 (m, 1H), 2.79-2.72 (m, 1H), 2.72-2.59 (m, 7H), 2.43 (d, J=14.5 Hz, 1H), 2.35-2.20 (m, 1H), 2.20-2.04 (m, 1H), 2.03-1.77 (m, 3H), 1.77-1.58 (m, 2H), 1.54-1.43 (m, 5H), 1.35-1.27 (m, 3H), 1.27-1.14 (m, 4H), 1.05 (dd, J=14.4, 6.7 Hz, 2H), 0.98-0.85 (m, 9H), 0.81-0.68 (m, 3H).

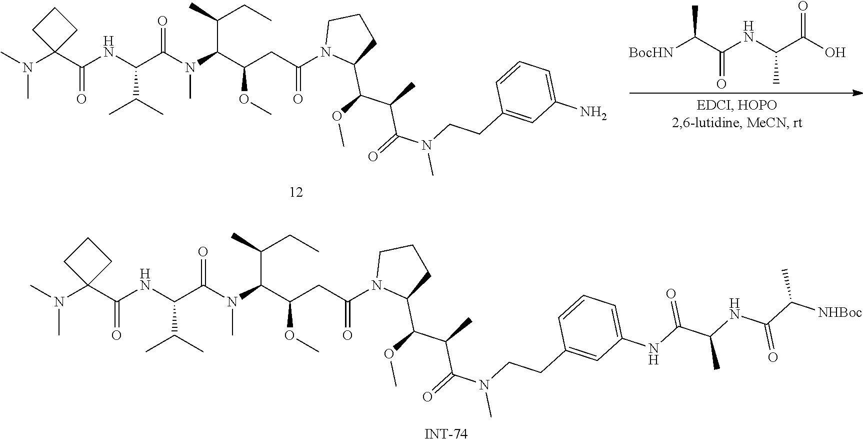

### Tert-butyl ((S)-1-(((S)-1-((3-(2-((2R,3R)-3-((S)-1-((3R,4S,5S)-4-((S)-2-(1-(dimethylamino)cyclobutanecarboxamido)-N,3-dimethylbutanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-74

To a solution of 12 (75 mg, 103 umol) and (S)-2-((S)-2-((tert-butoxycarbonyl)amino)propanamido)propanoic acid 1 (31 mg, 118 umol) in 2 mL CH₃CN was added a mixture of EDCI (30 mg, 154 umol) and HOPO (17 mg, 154 mmol), following by the addition of 2,6-lutidine (33 mg, 309 umol). The reaction was stirred at room temperature under a N₂ atmosphere for 16 h, LCMS showed completion. The mixture was purified by reverse phase column (H₂O:CH₃CN) directly to afford INT-74 (74 mg, 74% yield) as a colorless oil. LCMS (ESI): m/z 971.6 [M+H]⁺.

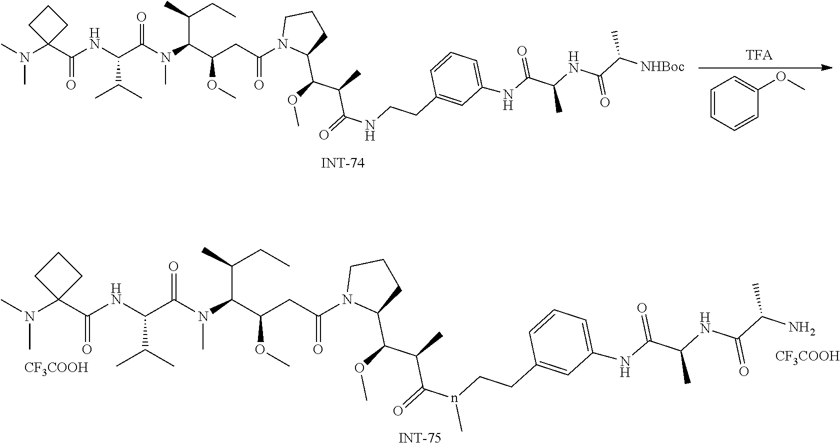

### N-((S)-1-(((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-(dimethylamino)cyclobutanecarboxamide bis(2,2,2-trifluoroacetate) INT-75

To a mixture of INT-74 (72 mg, 75 umol) and anisole (41 mg, 376 umol) was added TFA (0.7 mL). The reaction was then stirred at room temperature for 30 min. TLC showed completion (DCM/MeOH=13:1, v/v; R_{f}=˜0.6 for INT-74). The mixture was diluted with 35 mL MTBE, during which time, much white solid precipated. The resulting mixture was filtrated, and the filter cake was collected and dried under reduced pressure to afford INT-75 (56 mg, 68% yield) as a light yellow solid. LCMS (ESI): m/z 436.4 [M+2H]²⁺.

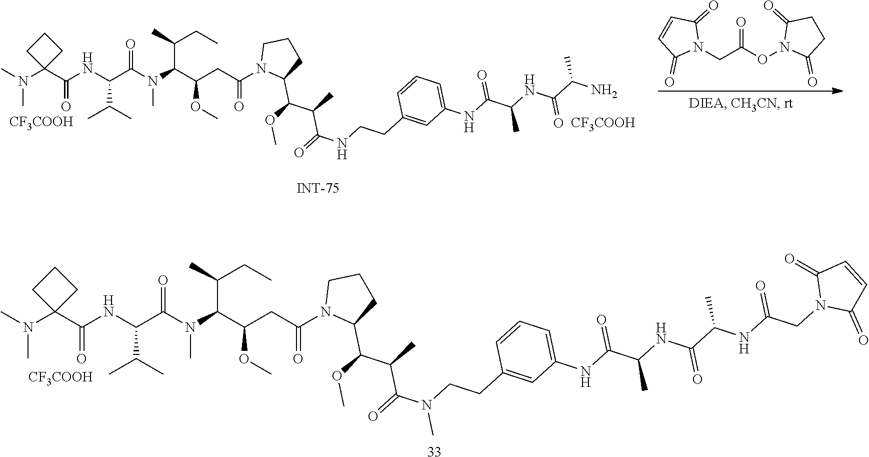

### 1-(dimethylamino)-N-((S)-1-(((3R,4S,5S)-1-((S)-2-((1R,2R)-3-((3-((S)-2-((S)-2-(2-(2,5-dioxo-2,5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)cyclobutanecarboxamide 2,2,2-trifluoroacetate 33

To a solution of INT-75 (56 mg, 51 umol) and 2,5-dioxopyrrolidin-1-yl 2-(2,5-dioxo-2,5-dihydro-1H-pyrrol-1-yl)acetate (19 mg, 76 umol) in 2 mL CH₃CN was added DIEA (17 uL, 102 umol). The reaction was then stirred at room temperature for 50 min. TLC showed completion (DCM/MeOH=7:1, v/v; R_{f}=˜0.15 for INT-75). The mixture was quenched by adding TFA (50 uL) directly and stirred for 5 min. The resulting mixture was sent to Prep-HPLC (0.1% TFA in H₂O/CH₃CN) instantly then freeze-dried to afford 33 (26 mg, 45% yield) as an off-white solid. LCMS (ESI): m/z 505.0 [M+2H]²⁺; HPLC: 99.9% @210 nm, Rₜ=8.92 min; ¹H NMR (400 MHz, DMSO-d₆) δ 10.46 (s, 1H), 9.88-9.74 (m, 1H), 8.64 (s, 1H), 8.42 (d, J=7.2 Hz, 1H), 8.20-8.08 (m, 1H), 7.49 (d, J=7.4 Hz, 1H), 7.47-7.34 (m, 1H), 7.26-7.13 (m, 1H), 7.08 (s, 2H), 6.96-6.87 (m, 1H), 4.81-4.64 (m, 1H), 4.53-4.43 (m, 1H), 4.40-4.29 (m, 2H), 4.11-4.07 (m, 2H), 3.96-3.93 (m, 2H), 3.91-3.86 (m, 2H)], 3.77-3.71 (m, 1H), 3.68-3.61 (m, 1H), 3.58-3.40 (m, 3H), 3.35-3.21 (m, 4H), 3.20-3.05 (m, 5H), [3.03 (s, 1H), 2.91 (s, 2H)], 2.84-2.80 (m, 1H), 2.79-2.56 (m, 10H), 2.48-2.40 (m, 2H), 2.38-2.04 (m, 2H), 1.99-1.77 (m, 4H), 1.75-1.54 (m, 3H), 1.38-1.26 (m, 4H), 1.25-1.20 (m, 3H), 1.05 (dd, J=16.6, 6.7 Hz, 2H), 1.00-0.84 (m, 10H), 0.77 (q, J=7.5 Hz, 3H).

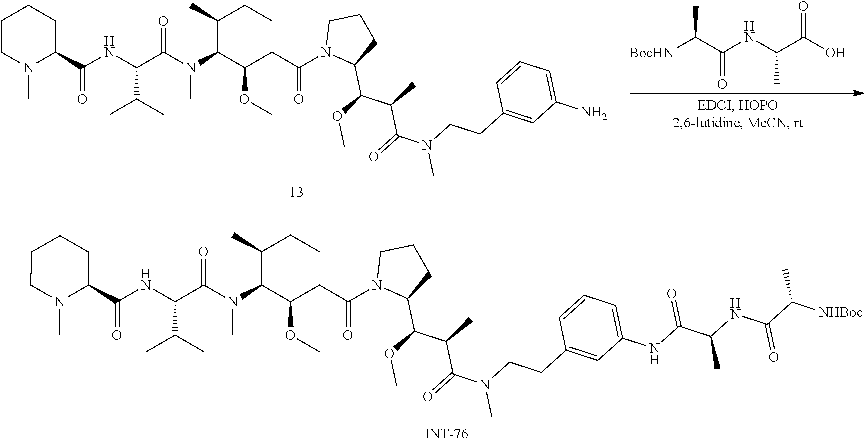

### Tert-butyl ((S)-1-(((S)-1-((3-(2-((2R,3R)-3-((S)-1-((3R,4S,5S)-4-((S)-N,3-dimethyl-2-((S)-1-methylpiperidine-2-carboxamido)butanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-76

To a solution of INT-76 (150 mg, 206 umol) and (S)-2-((S)-2-((tert-butoxycarbonyl)amino)propanamido)propanoic acid (64 mg, 247 umol) in 3 mL CH₃CN was added a mixture of EDCI (59 mg, 309 umol) and HOPO (34 mg, 309 umol), following by the addition of 2,6-lutidine (72 uL, 617 umol). The reaction was stirred at room temperature under a N₂ atmosphere for 16 h, LCMS showed completion. The mixture was purified by reverse phase column (H₂O:CH₃CN) directly to afford INT-76 (120 mg, 60% yield) as a yellow solid. LCMS (ESI): m/z 972.0 [M+H]⁺.

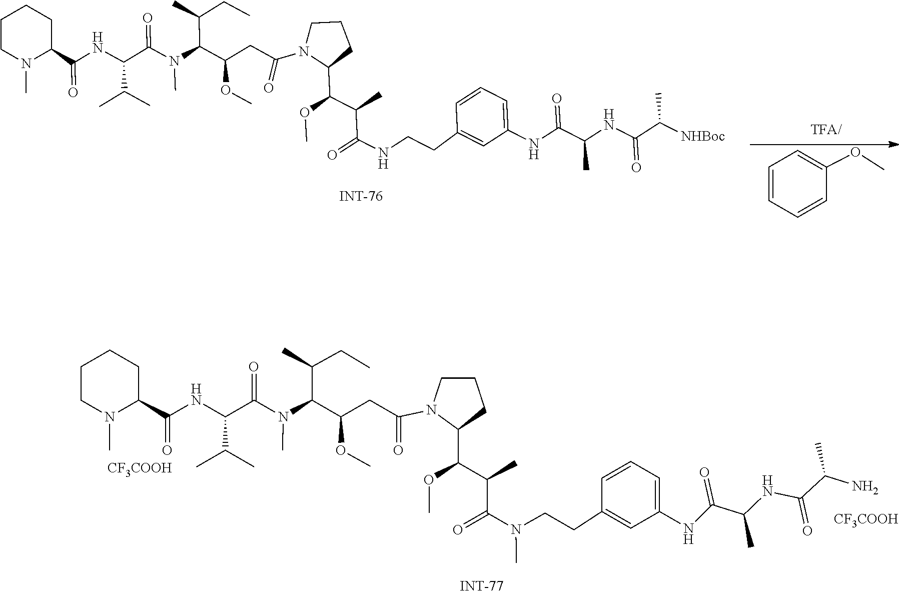

### (S)-N-((S)-1-(((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-2-carboxamide bis(2,2,2-trifluoroacetate) INT-77

To a mixture of INT-76 (90 mg, 93 umol) and anisole (50 uL, 463 umol) was added TFA (0.9 mL). The reaction was then stirred at room temperature for 10 min then quenched by adding 45 mL MTBE, during which time, much white solid precipated. The resulting mixture was filtrated, and the filter cake was collected and dried under reduced pressure to afford INT-77 (85 mg, 82% yield) as an off-white solid. LCMS (ESI): m/z 436.2 [M+2H]²⁺.

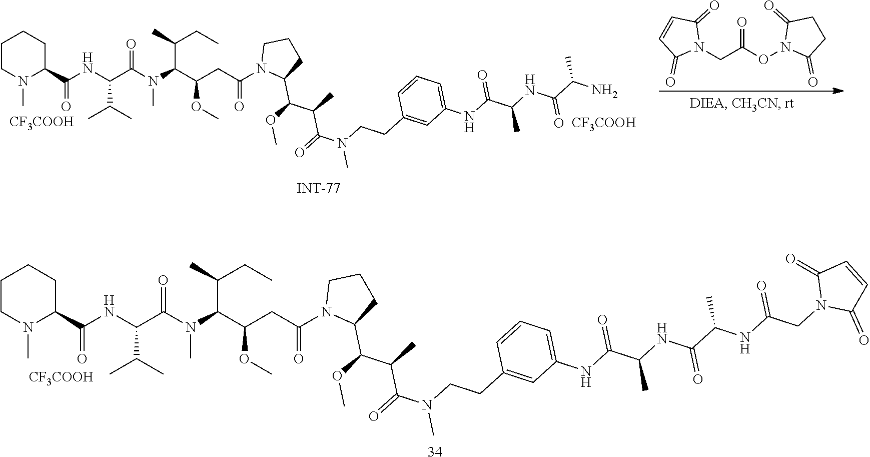

### (S)-N-((S)-1-(((3R,4S,5S)-1-((S)-2-((IR,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-2-carboxamide 2,2,2-trifluoroacetate 34

To a solution of INT-77 (80 mg, 73 umol) and 2,5-dioxopyrrolidin-1-yl 2-(2,5-dioxo-2,5-dihydro-1H-pyrrol-1-yl)acetate (27 mg, 106 umol) in 3 mL CH₃CN was added DIEA (21 mg, 162 umol). The reaction was then stirred at room temperature for 35 min then quenched by adding TFA (60 uL). LCMS showed completion. The resulting mixture was sent to Prep-HPLC (0.1% TFA in H₂O/CH₃CN) instantly then freeze-dried to afford 34 (16 mg, 20% yield) as a white solid. LCMS (ESI): m/z 505.3 [M+2H]²⁺; HPLC: 97.4% @210 nm, Rₜ=14.41 min; ¹H NMR (400 MHz, DMSO) δ 9.91-9.78 (m, 1H), 9.70 (s, 1H), 9.00 (d, J=8.4 Hz, 1H), 8.45 (d, J=7.1 Hz, 1H), 7.49 (s, 1H), 7.46-7.34 (m, 1H), 7.26-7.16 (m, 1H), 7.09 (s, 2H), 6.98-6.86 (m, 1H), 4.78-4.61 (m, 1H), 4.61-4.50 (m, 1H), 4.40-4.27 (m, 2H), 4.13-4.04 (m, 2H), 4.04-3.84 (m, 2H), 3.81-3.69 (m, 2H), 3.69-3.62 (m, 1H), 3.44-3.35 (m, 2H), 3.34-3.23 (m, 4H), 3.23-3.15 (m, 3H), 3.14-2.94 (m, 4H), 2.93-2.87 (m, 2H), 2.84-2.79 (m, 1H), 2.78-2.58 (m, 6H), 2.49-2.18 (m, 2H), 2.08-1.85 (m, 4H), 1.84-1.74 (m, 3H), 1.73-1.53 (m, 3H), 1.51-1.37 (m, 2H), 1.35-1.25 (m, 4H), 1.24-1.17 (m, 4H), 1.04 (dd, J=13.6, 6.6 Hz, 2H), 0.98-0.81 (m, 11H), 0.76 (q, J=7.0 Hz, 3H).

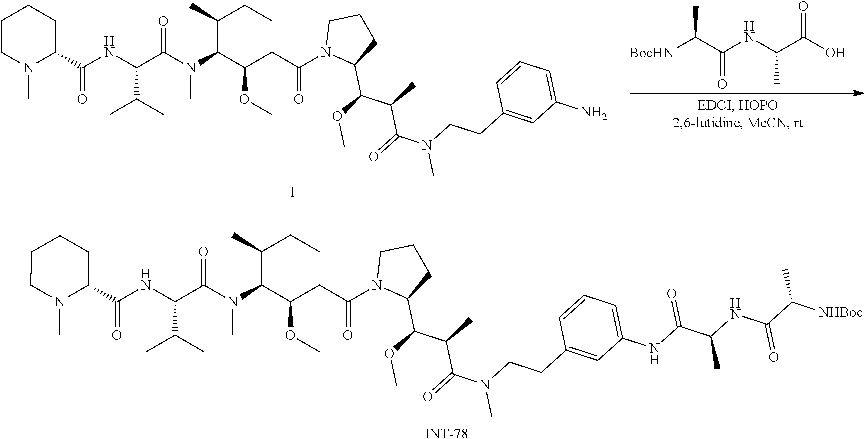

### Tert-butyl ((S)-1-(((S)-1-((3-(2-((2R,3R)-3-((S)-1-((3R,4S,5S)-4-((S)-N,3-dimethyl-2-((R)-1-methylpiperidine-2-carboxamido)butanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-78

To a solution of 11 (100 mg, 137 umol) and (S)-2-((S)-2-((tert-butoxycarbonyl)amino)propanamido)propanoic acid (43 mg, 165 umol) in 5 mL CH₃CN was added a mixture of EDCI (40 mg, 209 umol) and HOPO (23 mg, 207 umol), following by the addition of 2,6-lutidine (44 mg, 410 umol). The reaction was stirred at room temperature under a N₂ atmosphere for 2.5 h, LCMS showed completion. The mixture was purified by reverse phase column (H₂O:CH₃CN) directly to afford INT-78 (120 mg, 90% yield) as yellow oil. LCMS (ESI): m/z 971.2 [M+H]⁺.

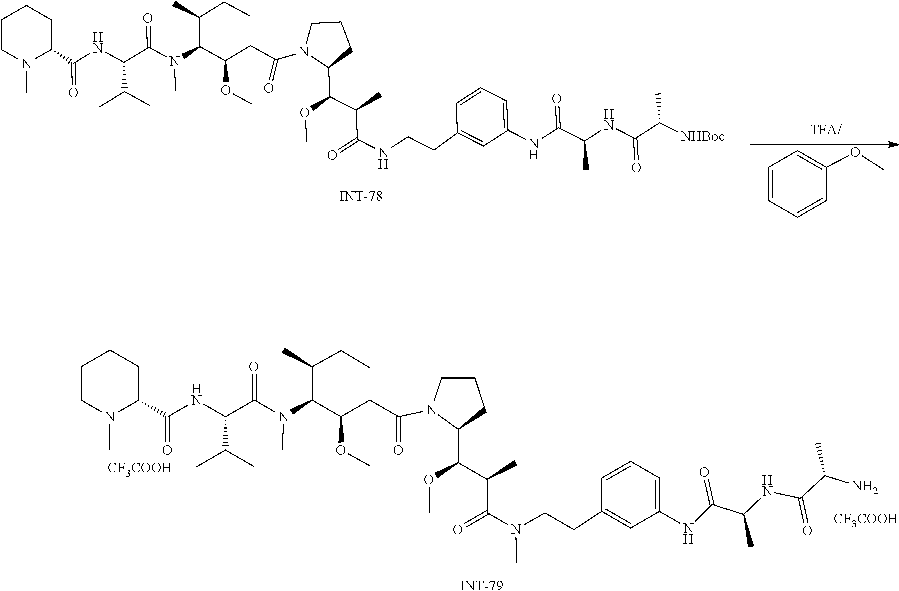

### (R)-N-((S)-1-(((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-2-carboxamide bis(2,2,2-trifluoroacetate) INT-79

To a mixture of INT-78 (120 mg, 124 umol) and anisole (67 mg, 620 umol) was added TFA (3 mL). The reaction was then stirred at room temperature for 5 min. TLC showed completion (DCM/MeOH=8:1, v/v; R_{f}=˜0.55 for INT-78). The mixture was diluted with 200 mL MTBE, during which time, much white solid precipated. The resulting mixture was filtrated, and the filter cake was collected and dried under reduced pressure to afford INT-79 (110 mg, 80% yield) as an off-white solid. LCMS (ESI): m/z 436.2 [M+2H]²⁺.

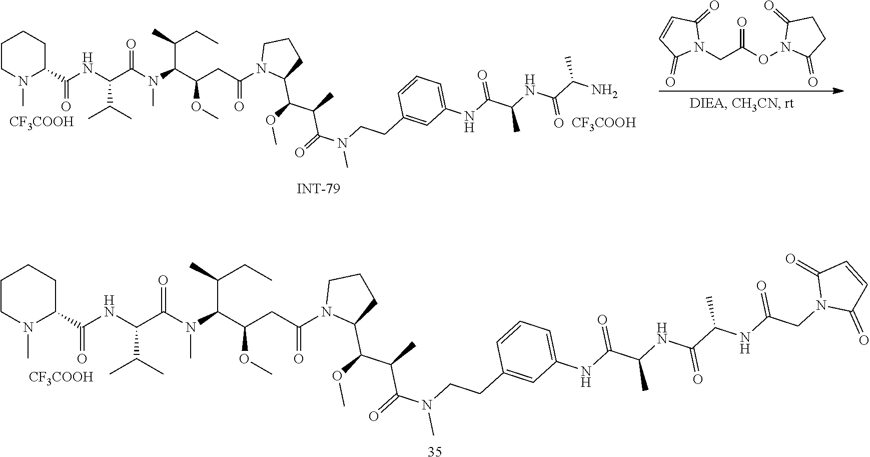

### (R)-N-((S)-1-(((3R,4S,5S)-1-((S)-2-((1R,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-2-carboxamide 2,2,2-trifluoroacetate 35

To a solution of INT-79 (110 mg, 100 umol) and 2,5-dioxopyrrolidin-1-yl 2-(2,5-dioxo-2,5-dihydro-1H-pyrrol-1-yl)acetate (43 mg, 170 umol) in 6 mL CH₃CN was added DIEA (29 mg, 225 umol). The reaction was then stirred at room temperature for 30 min. LCMS showed completion. The mixture was quenched by adding TFA (0.2 mL). The resulting mixture was purified by Prep-HPLC twice (0.1% TFA in H₂O/CH₃CN) then freeze-dried to afford 35 (26 mg, 23% yield) as a white solid. LCMS (ESI): m/z 1030.3 [M+Na⁺], 504.8 [M+2H]²⁺; HPLC: 97.5% @210 nm, Rₜ=19.05 min; ¹H NMR (400 MHz, DMSO) δ 9.85-9.77 (m, 1H), 9.68 (s, 1H), 8.95 (d, J=7.8 Hz, 1H), 8.43 (d, J=7.3 Hz, 1H), 8.20-8.11 (m, 1H), 7.49 (s, 1H), 7.40 (dd, J=24.0, 15.8 Hz, 1H), 7.20 (dd, J=19.6, 11.4 Hz, 1H), 7.09 (s, 2H), 6.96-6.87 (m, 1H), 4.79-4.58 (m, 1H), 4.56-4.45 (m, 1H), 4.43-4.28 (m, 2H), 4.15-4.04 (m, 2H), 4.04-3.84 (m, 2H), 3.81-3.72 (m, 2H), 3.51 (s, 1H), 3.46 (s, 1H), 3.43 (d, J=7.2 Hz, 1H), 3.36-3.23 (m, 5H), 3.20-3.18 (m, 2H), 3.17-2.98 (m, 5H), 2.93-2.87 (m, 2H), 2.83-2.79 (m, 1H), 2.77-2.72 (m, 1H), 2.71-2.66 (m, 1H), 2.64-2.59 (m, 3H), 2.45-2.36 (m, 1H), 2.32-2.18 (m, 1H), 2.07-1.96 (m, 2H), 1.94-1.75 (m, 5H), 1.71-1.54 (m, 4H), 1.45-1.36 (m, 1H), 1.34-1.26 (m, 4H), 1.25-1.19 (m, 4H), 1.04 (dd, J=13.8, 6.7 Hz, 2H), 0.99-0.82 (m, 11H), 0.80-0.74 (m, 3H).

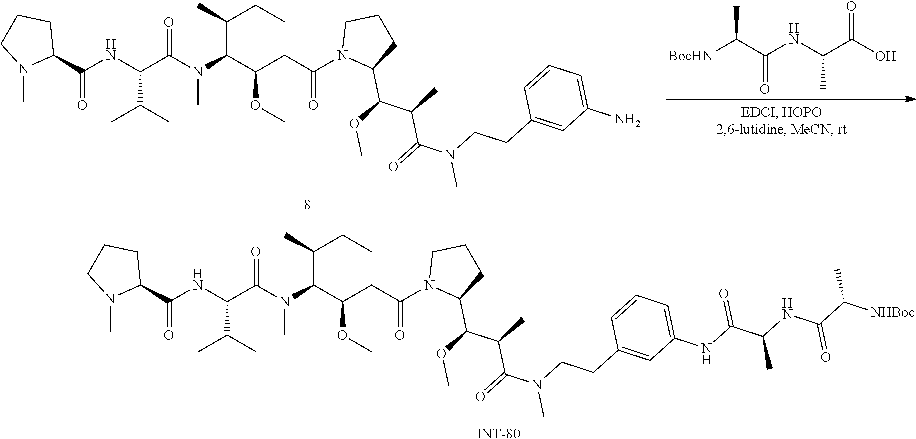

### Tert-butyl ((S)-1-(((S)-1-((3-(2-((2R,3R)-3-((S)-1-((3R,4S,5S)-4-((S)-N,3-dimethyl-2-((S)-1-methylpyrrolidine-2-carboxamido)butanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-80

To a solution of 8 (110 mg, 154 umol) and (S)-2-((S)-2-((tert-butoxycarbonyl)amino)propanamido)propanoic acid (48 mg, 185 umol) in 3 mL CH₃CN was added a mixture of EDCI (25 mg, 231 umol) and HOPO (44 mg, 231 umol), following by the addition of 2,6-lutidine (55 uL, 462 umol). The reaction was stirred at room temperature under a N₂ atmosphere for 16 h, LCMS showed completion. The mixture was purified by reverse phase column (H₂O:CH₃CN) directly to afford INT-80 (96 mg, 85% yield) as a light yellow solid. LCMS (ESI): m/z 957.3 [M+H]⁺.

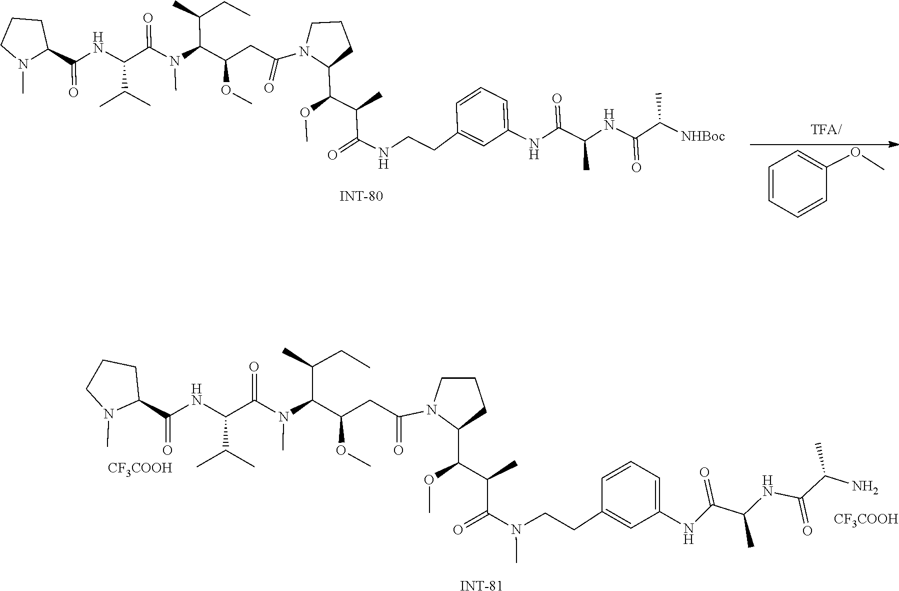

### (S)-N-((S)-1-(((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-2-carboxamide bis(2,2,2-trifluoroacetate) INT-81

To a mixture of INT-80 (110 mg, 115 umol) and anisole (65 uL, 598 umol) was added TFA (1 mL). The reaction was then stirred at room temperature for 10 min then quenched by adding 50 mL MTBE, during which time, much white solid precipated. The resulting mixture was filtrated, and the filter cake was collected and dried under reduced pressure to afford INT-81 (110 mg, 87% yield) as an off-white solid. LCMS (ESI): m/z 857.2 [M+H]⁺.

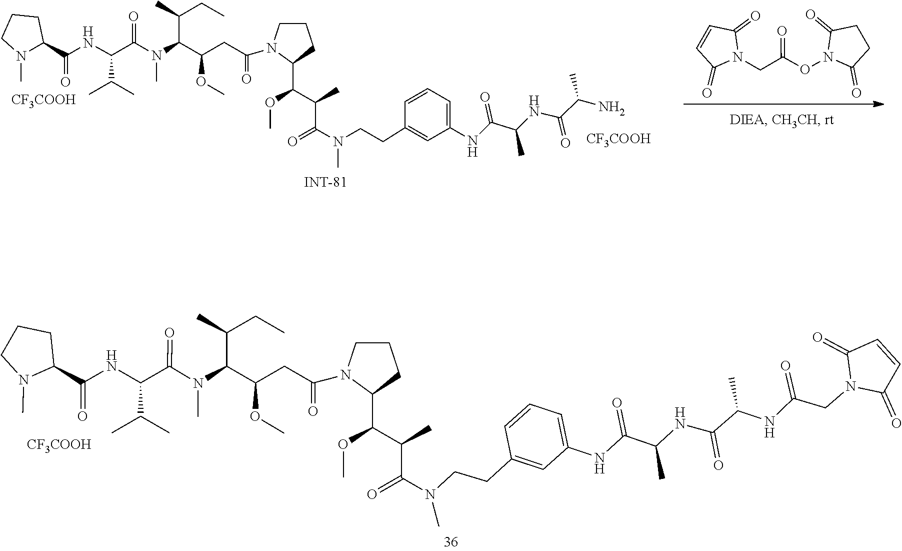

### (S)-N-((S)-1-(((3R,4S,5S)-1-((S)-2-((1R,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-2-carboxamide 2,2,2-trifluoroacetate 36

To a solution of INT-81 (110 mg, 101 umol) and 2,5-dioxopyrrolidin-1-yl 2-(2,5-dioxo-2,5-dihydro-1H-pyrrol-1-yl)acetate (36 mg, 142 umol) in 2 mL CH₃CN was added DIEA (38 uL, 227 umol). The reaction was then stirred at room temperature for 45 min then quenched by adding TFA (70 uL). LCMS showed completion. The resulting mixture was sent to Prep-HPLC (0.1% TFA in H₂O/CH₃CN) instantly then freeze-dried to afford 36 (60 mg, 53% yield) as an off-white solid. LCMS (ESI): m/z 994.1 [M+H]⁺, 497.8 [M+2H]²⁺; HPLC: 98.8% @210 nm, Rₜ=8.62 min; ¹H NMR (400 MHz, DMSO) δ 9.86-9.75 (m, 1H), 9.62 (s, 1H), 9.00 (d, J=8.6 Hz, 1H), 8.43 (d, J=7.2 Hz, 1H), 8.22-8.12 (m, 1H), 7.49 (s, 1H), 7.46-7.34 (m, 1H), 7.26-7.16 (m, 1H), 7.09 (s, 2H), 6.97-6.87 (m, 1H), 4.81-4.65 (m, 2H), 4.65-4.47 (m, 2H), 4.41-4.28 (m, 2H), 4.15-4.01 (m, 3H), 3.99-3.88 (m, 1H), 3.78-3.66 (m, 1H), 3.65-3.51 (m, 2H), 3.51-3.34 (m, 2H), 3.33-3.25 (m, 3H), 3.24-3.16 (m, 3H), 3.16-2.94 (m, 4H), 2.94-2.85 (m, 2H), 2.84-2.72 (m, 5H), 2.71-2.54 (m, 2H), 2.47-2.37 (m, 2H), 2.34-2.19 (m, 1H), 2.10-1.97 (m, 2H), 1.96-1.75 (m, 4H), 1.75-1.56 (m, 3H), 1.33-1.26 (m, 3H), 1.21 (d, J=7.0 Hz, 4H), 1.04 (dd, J=12.9, 6.7 Hz, 2H), 0.99-0.69 (m, 14H).

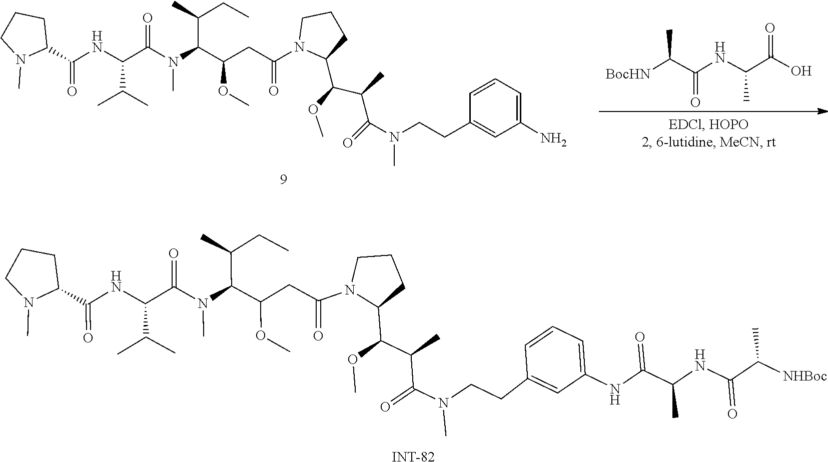

### Tert-butyl ((S)-1-(((S)-1-((3-(2-((2R,3R)-3-((S)-1-((3R,4S,5S)-4-((S)-N,3-dimethyl-2-((R)-1-methylpyrrolidine-2-carboxamido)butanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-82

To a solution of 9 (90 mg, 126 umol) and (S)-2-((S)-2-((tert-butoxycarbonyl)amino)propanamido)propanoic acid (38 mg, 145 umol) in 3 mL CH₃CN was added a mixture of EDCI (36 mg, 189 mmol) and HOPO (21 mg, 189 umol), following by the addition of 2,6-lutidine (40 mg, 378 umol). The reaction was stirred at room temperature under a N₂ atmosphere for 16 h, LCMS showed completion. The mixture was purified by reverse phase column (H₂O:CH₃CN) directly to afford INT-82 (110 mg, 91% yield) as a off-white solid. LCMS (ESI): m/z 957.7 [M+H]⁺.

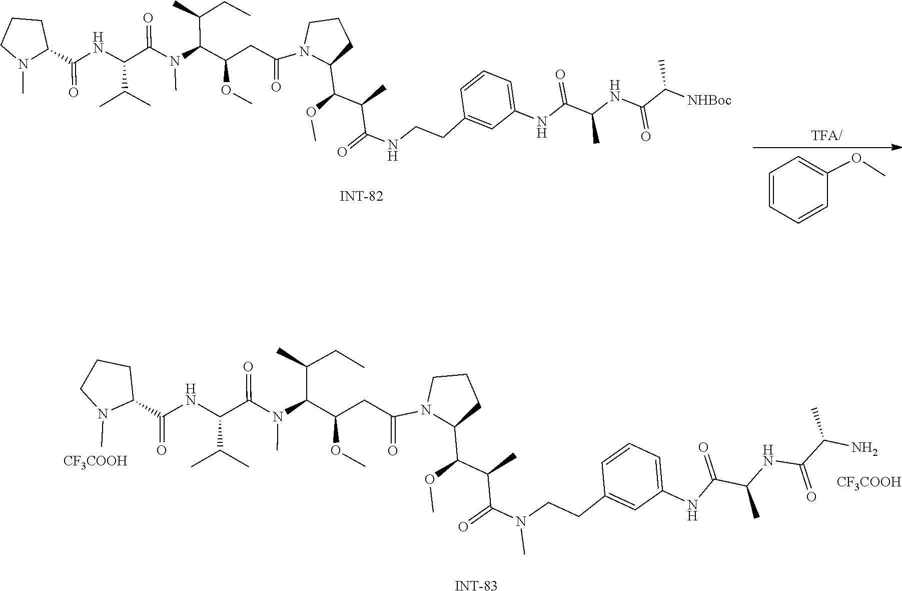

### (R)-N-((S)-1-(((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-2-carboxamide bis(2,2,2-trifluoroacetate) INT-83

To a mixture of INT-82 (110 mg, 117 umol) and anisole (63 mg, 583 umol) was added TFA (1.1 mL). The reaction was then stirred at room temperature for 30 min. TLC showed completion (DCM/MeOH=13:1, v/v; R_{f}=0.4 for INT-82). The mixture was diluted with 50 mL MTBE, during which time, much white solid precipated. The resulting mixture was filtrated, and the filter cake was collected and dried under reduced pressure to afford INT-83 (127 mg, 100% yield) as an off-white solid, which was used directly to next step.

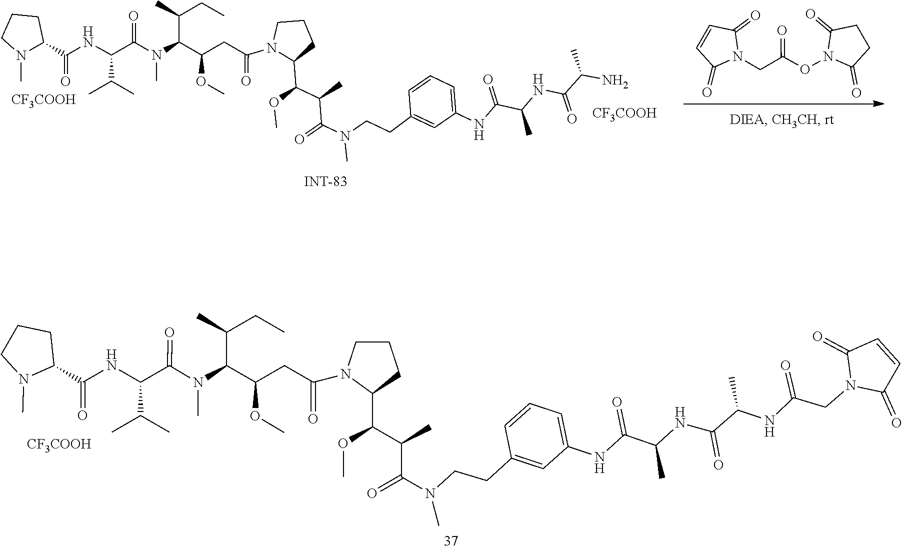

### (R)-N-((S)-1-(((3R,4S,5S)-1-((S)-2-((IR,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-2-carboxamide 2,2,2-trifluoroacetate 37

To a solution of INT-83 (127 mg, 117 umol) and 2,5-dioxopyrrolidin-1-yl 2-(2,5-dioxo-2,5-dihydro-1H-pyrrol-1-yl)acetate (44 mg, 176 umol) in 2 mL CH₃CN was added DIEA (39 uL, 234 umol). The reaction was then stirred at room temperature for 30 min. TLC&LCMS showed completion (DCM/MeOH=6:1, v/v; R_{f}=˜0.15 for INT-83). The mixture was quenched by adding TFA (0.1 mL) directly and stirred for 5 min. The resulting mixture was sent to Prep-HPLC (0.1% TFA in H₂O/CH₃CN) instantly then freeze-dried to afford 37 (29 mg, 22% yield) as an off-white solid. LCMS (ESI): m/z 1016.0 [M+Na⁺]; HPLC: 99.2% @210 nm, Rₜ=8.70 min; ¹H NMR (400 MHz, DMSO-d₆) δ 9.87-9.76 (m, 1H), 9.69 (s, 1H), 9.02 (d, J=8.1 Hz, 1H), 8.43 (d, J=7.1 Hz, 1H), 8.21-8.11 (m, 1H), 7.49 (s, 1H), 7.47-7.34 (m, 1H), 7.25-7.15 (m, 1H), 7.09 (s, 2H), 6.96-6.86 (m, 1H), 4.74-4.64 (m, 1H), 4.58-4.53 (m, 1H), 4.38-4.36 (m, 1H), 4.33-4.30 (m, 1H), 4.11-4.07 (m, 2H), 4.04-4.00 (m, 1H), 3.97-3.88 (m, 1H), 3.78-3.61 (m, 2H), 3.60-3.51 (m, 2H), 3.51-3.39 (m, 2H), 3.36-3.24 (m, 4H), 3.23-3.16 (m, 4H), [3.12 (s, 1.3H), 2.99 (s, 1.7H)], 2.93-2.87 (m, 2H), 2.83-2.80 (m, 1H), 2.79-2.60 (m, 6H), 2.49-2.19 (m, 2H), 2.13-2.00 (m, 2H), 1.99-1.71 (m, 6H), 1.70-1.49 (m, 2H), 1.32-1.28 (m, 3H), 1.24-1.20 (m, 4H), 1.04 (dd, J=13.6, 6.6 Hz, 2H), 1.00-0.85 (m, 10H), 0.84-0.74 (m, 4H).

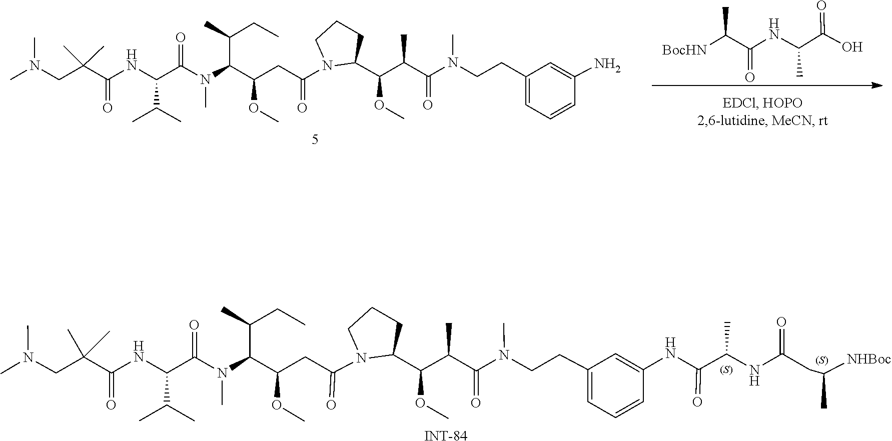

### Tert-butyl ((S)-1-(((S)-1-((3-(2-((2R,3R)-3-((S)-1-((3R,4S,5S)-4-((S)-2-(3-(dimethylamino)-2,2-dimethylpropanamido)-N,3-dimethylbutanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-84

To a solution of 5 (60 mg, 82 umol) and (S)-2-((S)-2-((tert-butoxycarbonyl)amino)propanamido)propanoic acid (26 mg, 98.5 umol) in 3 mL CH₃CN was added a mixture of EDCI (24 mg, 123 umol) and HOPO (14 mg, 123 umol), following by the addition of 2,6-lutidine (29 uL, 246 umol). The reaction was stirred at room temperature under a N₂ atmosphere for 16 h, LCMS showed completion. The mixture was purified by reverse phase column (H₂O:CH₃CN) directly to afford INT-84 (50 mg, 63% yield) as a white solid. LCMS (ESI): m/z 974.2 [M+H]⁺.

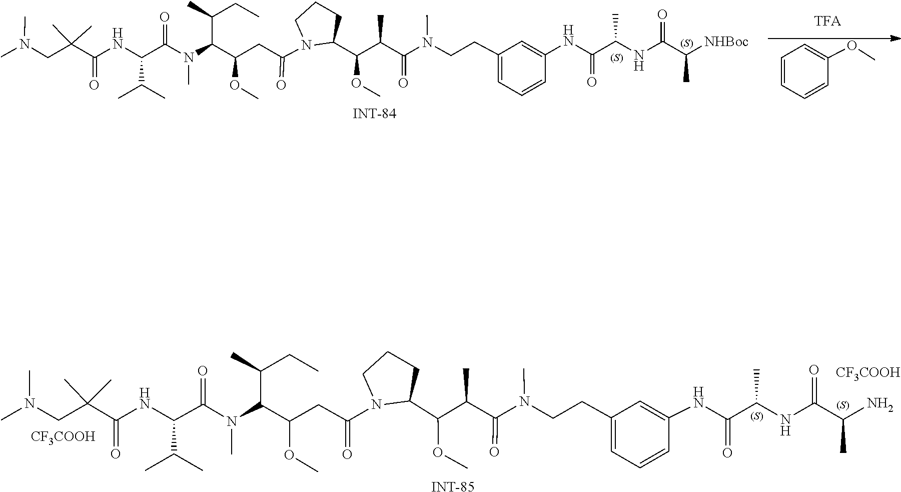

### (S)-N-((3R,4S,5S)-1-((S)-2-((1R,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)-2-(3-(dimethylamino)-2,2-dimethylpropanamido)-N,3-dimethylbutanamide 2,2,2-trifluoroacetate INT-85

To a mixture of INT-84 (50 mg, 51 umol) and anisole (28 mg, 257 umol) was added TFA (0.5 mL). The reaction was then stirred at room temperature for 10 min then quenched by adding 25 mL MTBE, during which time, much white solid precipated. The resulting mixture was filtrated, and the filter cake was collected and dried under reduced pressure to afford INT-85 (50 mg, 88% yield) as a light yellow solid. LCMS (ESI): m/z 873.6 [M+H]⁺, 437.5 [M+2H]²⁺.

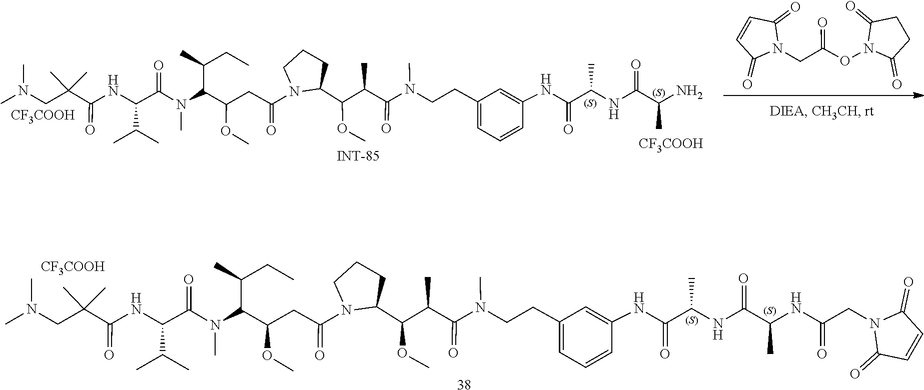

### (S)-2-(3-(dimethylamino)-2,2-dimethylpropanamido)-N-((3R,4S,5S)-1-((S)-2-((1R,2R)-3-((3-((S)-2-((S)-2-(2-(2,5-dioxo-2,5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)-N,3-dimethylbutanamide 2,2,2-trifluoroacetate 38

To a solution of INT-85 (50 mg, 45 umol) and 2,5-dioxopyrrolidin-1-yl 2-(2,5-dioxo-2,5-dihydro-1H-pyrrol-1-yl)acetate (17 mg, 66 umol) in 2 mL CH₃CN was added DIEA (17 uL, 101 umol). The reaction was then stirred at room temperature for 45 min then quenched by adding TFA (0.04 mL) directly. LCMS showed completion. The resulting mixture was sent to Prep-HPLC (0.1% TFA in H₂O/CH₃CN) instantly then freeze-dried to afford 38 (23 mg, 45% yield) as a white solid. LCMS (ESI): m/z 506.0 [M+2H]²⁺; HPLC: 99.9% @210 nm, Rₜ=8.77 min; ¹H NMR (400 MHz, DMSO) δ 9.90-9.73 (m, 1H), 8.91 (s, 1H), 8.43 (d, J=7.1 Hz, 1H), 8.24-8.10 (m, 1H), 7.99-7.84 (m, 1H), 7.49 (s, 1H), 7.47-7.34 (m, 1H), 7.25-7.16 (m, 1H), 7.09 (s, 2H), 6.97-6.87 (m, 1H), 4.76-4.58 (m, 1H), 4.55-4.44 (m, 1H), 4.40-4.31 (m, 2H), 4.11-4.08 (m, 2H), 3.97-3.94 (m, 1H), 3.75-3.74 (m, 1H), 3.60-3.54 (m, 1H), 3.50-3.42 (m, 2H), 3.36-3.23 (m, 6H), 3.22-3.17 (m, 3H), 3.15-2.95 (m, 4H), 2.92-2.88 (m, 2H), 2.82-2.72 (m, 8H), 2.69-2.64 (m, 1H), 2.49-2.19 (m, 2H), 2.14-2.04 (m, 1H), 2.02-1.82 (m, 3H), 1.80-1.72 (m, 1H), 1.71-1.56 (m, 2H), 1.31-1.26 (m, 6H), 1.24-1.19 (m, 7H), 1.04 (dd, J=13.2, 6.7 Hz, 2H), 0.98-0.83 (m, 11H), 0.77 (q, J=7.2 Hz, 3H).

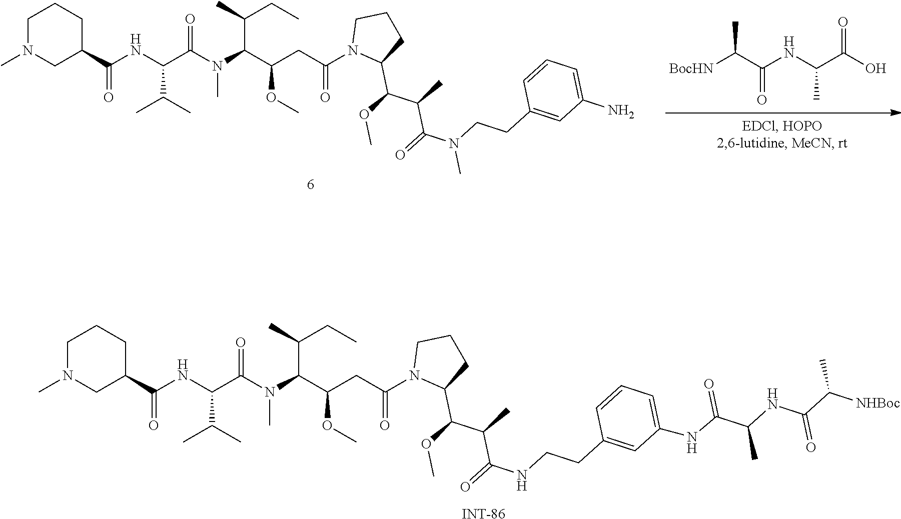

### Tert-butyl ((S)-1-(((S)-1-((3-(2-((2R,3R)-3-((S)-1-((3R,4S,5S)-4-((S)-N,3-dimethyl-2-((R)-1-methylpiperidine-3-carboxamido)butanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-86

To a solution of 6 (50 mg, 69 umol) and (S)-2-((S)-2-((tert-butoxycarbonyl)amino)propanamido)propanoic acid (22 mg, 82 umol) in 2 mL CH₃CN was added a mixture of EDCI (20 mg, 103 umol) and HOPO (12 mg, 103 umol), following by the addition of 2,6-lutidine (24 uL, 206 umol). The reaction was stirred at room temperature under a N₂ atmosphere for 16 h, LCMS showed completion. The mixture was purified by reverse phase column (H₂O:CH₃CN) directly to afford INT-86 (35 mg, 53% yield) as a white solid. LCMS (ESI): m/z 971.2 [M+H]⁺.

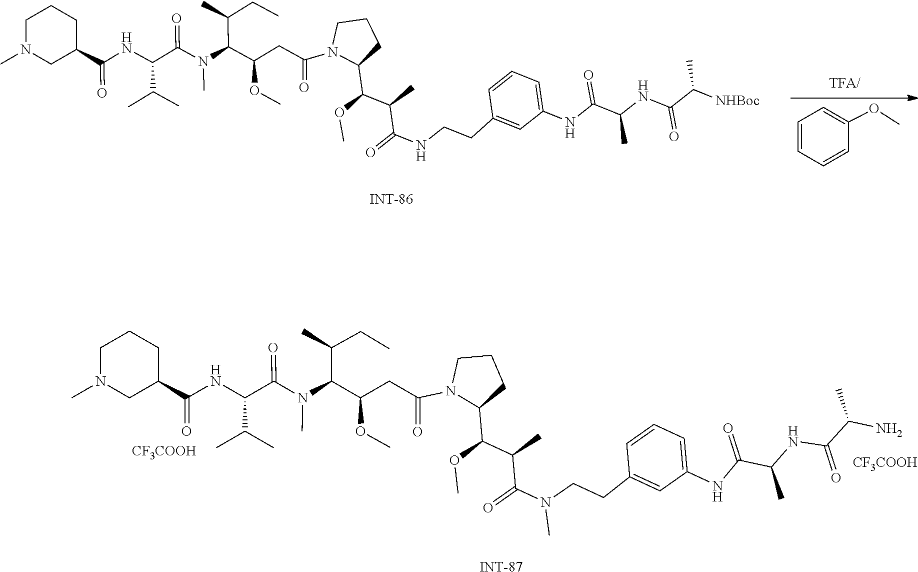

### (R)-N-((S)-1-(((3R,4S,5S)-1-((S)-2-((1R,2R)-3-((3-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-3-carboxamide bis(2,2,2-trifluoroacetate) INT-87

To a mixture of INT-86 (35 mg, 36 umol) and anisole (20 uL, 180 umol) was added TFA (0.35 mL). The reaction was then stirred at room temperature for 10 min then quenched by adding 18 mL MTBE, during which time, much white solid precipitated. The resulting mixture was filtrated, and the filter cake was collected and dried under reduced pressure to afford INT-87 (30 mg, 75% yield) as a light yellow solid, which was used directly without further identification.

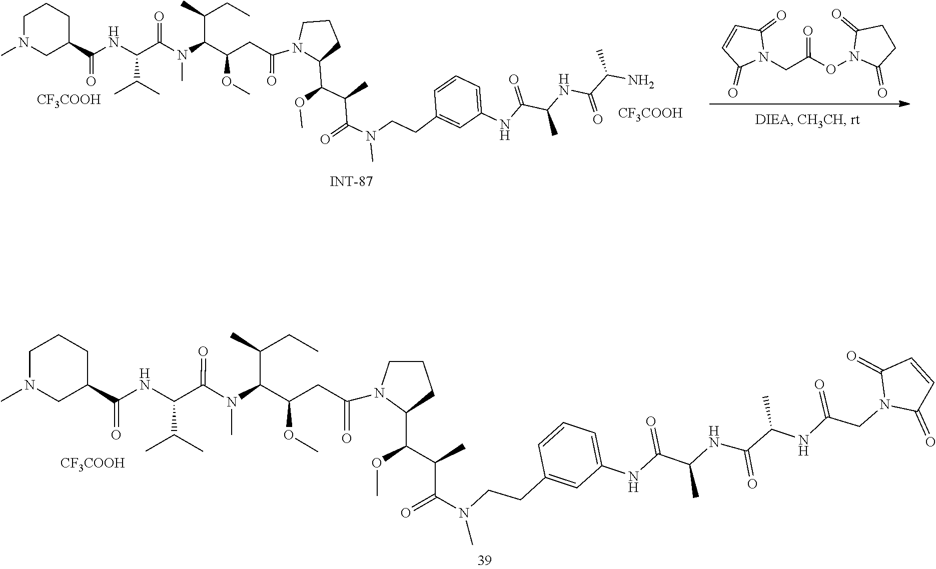

### (R)-N-((S)-1-(((3R,4S,5S)-1-((S)-2-((1R,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpiperidine-3-carboxamide 2,2,2-trifluoroacetate 39

To a solution of INT-87 (35 mg, 36 umol) and 2,5-dioxopyrrolidin-1-yl 2-(2,5-dioxo-2,5-dihydro-1H-pyrrol-1-yl)acetate (12 mg, 46 umol) in 6 mL CH₃CN was added DIEA (12 uL, 71 umol). The reaction was then stirred at room temperature for 30 min. LCMS showed completion. The mixture was quenched by adding TFA (20 uL). The resulting mixture was sent to Prep-HPLC (0.1% TFA in H₂O/CH₃CN) instantly then freeze-dried to afford 39 (5 mg, 14% yield) as a white solid. LCMS (ESI): m/z 505.0 [M+2H]²⁺; HPLC: 99.8% @210 nm, Rₜ=8.62 min.

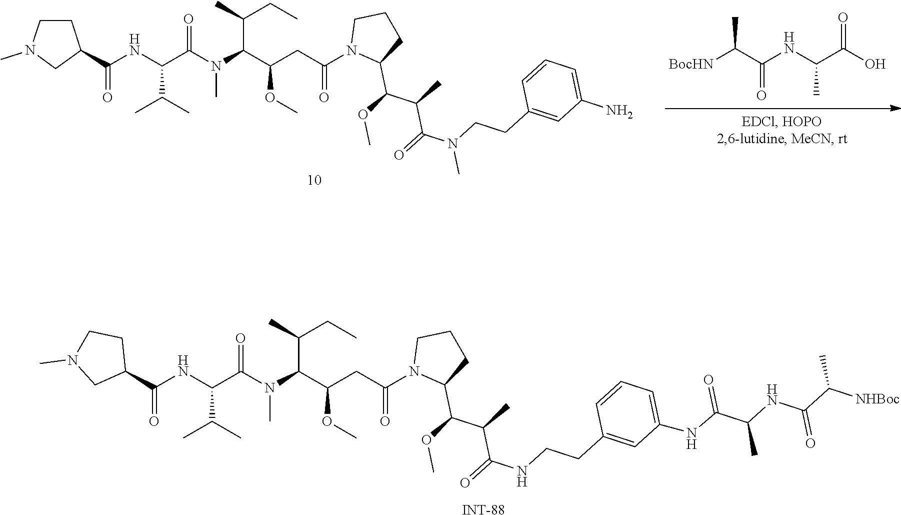

### Step 1: Tert-butyl ((S)-1-(((S)-1-((3-(2-((2R,3R)-3-((S)-1-((3R,4S,5S)-4-((S)-N,3-dimethyl-2-((R)-1-methylpyrrolidine-3-carboxamido)butanamido)-3-methoxy-5-methylheptanoyl)pyrrolidin-2-yl)-3-methoxy-N,2-dimethylpropanamido)ethyl)phenyl)amino)-1-oxopropan-2-yl)amino)-1-oxopropan-2-yl)carbamate INT-88

To a solution of 10 (110 mg, 154 umol) and (S)-2-((S)-2-((tert-butoxycarbonyl)amino)propanamido)propanoic acid (48 mg, 185 umol) in 3 mL CH₃CN was added a mixture of EDCI (44 mg, 231 mmol) and HOPO (26 mg, 231 umol), following by the addition of 2,6-lutidine (54 uL, 462 umol). The reaction was stirred at room temperature under a N₂ atmosphere for 16 h, LCMS showed completion. The mixture was purified by reverse phase column (H₂O:CH₃CN) directly to afford INT-88 (105 mg, 71% yield) as a yellow foam solid. LCMS (ESI): m/z 957.1 [M+H]⁺.

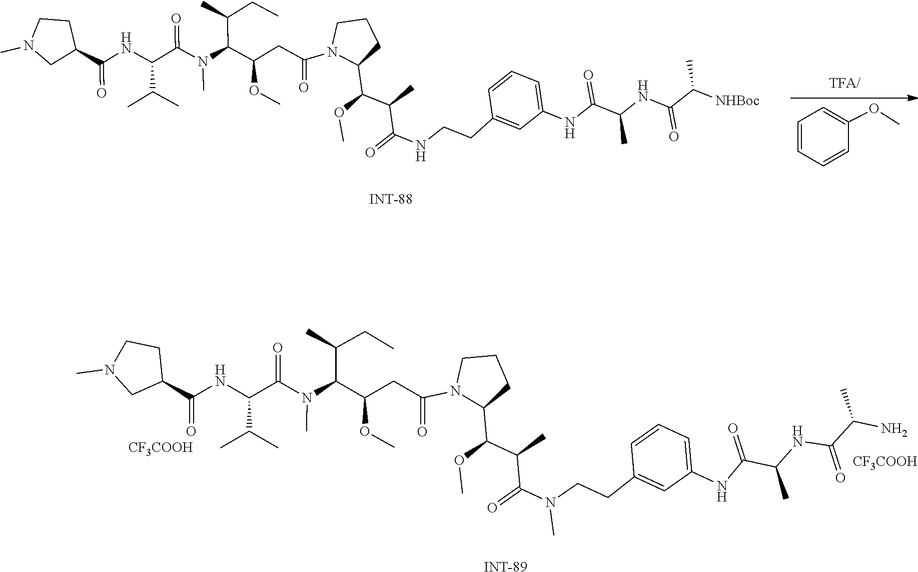

### (R)-N-((S)-1-(((3R,4S,5S)-1-((S)-2-((IR,2R)-3-((3-((S)-2-((S)-2-aminopropanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-3-carboxamide bis(2,2,2-trifluoroacetate) INT-89

To a mixture of INT-88 (100 mg, 104 umol) and anisole (57 uL, 522 umol) was added TFA (1 mL). The reaction was then stirred at room temperature for 10 min then quenched by adding 50 mL MTBE, during which time, much white solid precipitated. The resulting mixture was filtrated, and the filter cake was collected and dried under reduced pressure to afford INT-89 (90 mg, 78% yield) as a light yellow solid. LCMS (ESI): m/z 880.3 [M+Na⁺], 429.1 [M+2H]²⁺.

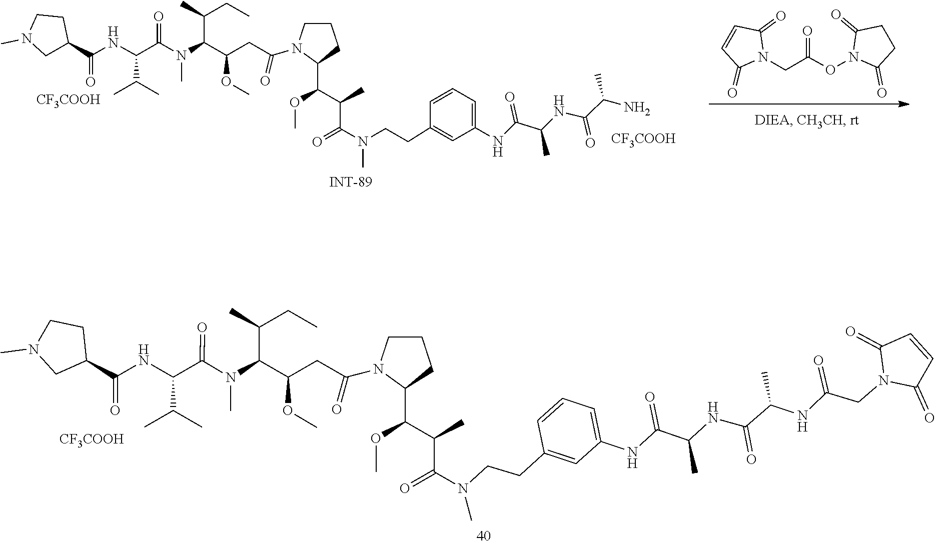

### (R)-N-((S)-1-(((3R,4S,5S)-1-((S)-2-((IR,2R)-3-((3-((S)-2-((S)-2-(2-(2,5-dioxo-2, 5-dihydro-1H-pyrrol-1-yl)acetamido)propanamido)propanamido)phenethyl)(methyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-1-methylpyrrolidine-3-carboxamide 2,2,2-trifluoroacetate 40

To a solution of INT-89 (90 mg, 83 umol) and 2,5-dioxopyrrolidin-1-yl 2-(2,5-dioxo-2,5-dihydro-1H-pyrrol-1-yl)acetate (30 mg, 116 umol) in 2 mL CH₃CN was added DIEA (30 uL, 185 umol). The reaction was then stirred at room temperature for 50 min. LCMS showed completion. The mixture was quenched by adding TFA (60 uL). The resulting mixture was sent to Prep-HPLC (0.1% TFA in H₂O/CH₃CN) instantly then freeze-dried to afford 40 (65 mg, 71% yield) as a white solid. LCMS (ESI): m/z 995.2 [M+H]⁺, 497.7 [M+2H]2+; HPLC: 98.6% @210 nm, Rₜ=8.57 min; ¹H NMR (400 MHz, DMSO) δ 9.92-9.67 (m, 2H), 8.54-8.39 (m, 2H), 8.21-8.10 (m, 1H), 7.49 (s, 1H), 7.46-7.34 (m, 1H), 7.25-7.16 (m, 1H), 7.09 (s, 2H), 6.97-6.87 (m, 1H), 4.75-4.59 (m, 2H), 4.55-4.44 (m, 2H), 4.38-4.31 (m, 2H), 4.13-4.05 (m, 2H), 4.03-3.85 (m, 2H), 3.79-3.57 (m, 3H), 3.55-3.38 (m, 3H), 3.29 (dd, J=13.5, 5.7 Hz, 4H), 3.21-3.16 (m, 3H), 3.13-2.95 (m, 4H), 2.91-2.86 (m, 2H), 2.85-2.78 (m, 3H), 2.77-2.73 (m, 1H), 2.72-2.58 (m, 2H), 2.46-2.32 (m, 2H), 2.30-2.04 (m, 2H), 2.00-1.73 (m, 5H), 1.67-1.42 (m, 2H), 1.33-1.26 (m, 3H), 1.21 (d, J=6.8 Hz, 4H), 1.04 (dd, J=12.4, 6.7 Hz, 2H), 0.98-0.70 (m, 14H).

### Biological Activity

### Assay Protocol

HCC1954 breast ductal carcinoma and T47D cells (ATCC, Manassas, VA, USA) were seeded into 384-well white-walled culture plates and allowed to adhere for 2-4 hours. Cells were then treated at least in duplicate by addition of 5-fold serially diluted test articles prepared at 2× final concentration and incubated at 37° C. for 120 hours. Cell viability following treatment was determined by Cell Titer Glo 2.0 Assay (Promega, Madison, WI, USA) and normalized to non-treated controls. Dose-response relationships were analyzed using GraphPad Prism (La Jolla, CA, USA), and IC50 values were derived from non-linear regression analyses using a 4-parameter logistic equation.

TABLE 4
In vitro Potency of Exemplary Compounds in HCC1954

| Compound | IC₅₀ (nM) |
|---|---|
| 1 | +++ |
| 2 | +++ |
| 3 | +++ |
| 4 | +++ |
| 5 | ++ |
| 6 | + |
| 7 | +++ |
| 8 | +++ |
| 9 | +++ |
| 10 | + |
| 11 | +++ |
| 12 | +++ |
| 13 | +++ |
| 14 | +++ |
| 15 | +++ |
| 16 | +++ |
| 17 | +++ |
| 18 | ++ |
| 19 | +++ |
| 23 | +++ |
| 24 | +++ |
| 26 | ++ |
| 27 | +++ |

+++: <5 nM;
++: 5-10 nM;
+: >10 nM

TABLE 5
Comparison of N-methylated analogs 1,2-/
1,3-/1,4-NH₂ in HCC1954 and T47D assays

IC₅₀ (fold difference)

| Compound | HCC1954 | T47D |
|---|---|---|
| 1 (1,3-NH₂) | 1 | 1 |
| 14 (1,2-NH₂) | 0.8 | 1.3 |
| 19 (1,4-NH₂) | 2.1 | 3.8 |
| 2 (1,3-NH₂) | 1 | 1 |
| 16 (1,2-NH₂) | 1.8 | 3.4 |
| 26 (1,4-NH₂) | 4.2 | 12.7 |

Compared to the 1,4-NH₂ configuration of the P5 moiety in 19 and 26, the 1,2-NH₂ (14 and 16) and 1,3-NH₂ (1 and 2) configurations were surprisingly more potent.

TABLE 6A
NCH₃-amide potency comparison to its NH-amide analogs
in HCC1954 and T47D assays

| | IC₅₀ (fold difference) | |
|---|---|---|
| Compound | HCC1954 | T47D |
| 20 (NH) | 1 | 1 |
| 1 (NMe) | 1.5 | 1.6 |
| 21 (NH) | 1 | 1 |
| 14 (NMe) | 2.0 | 1.8 |
| 22 (NH) | 1 | 1 |
| 19 (NMe) | 2.3 | 6.9 |

20 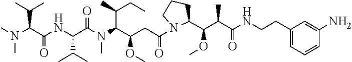 [SMILES: CC[C@H](C)[C@@H]([C@@H](CC(=O)N1CCC[C@H]1[C@H](OC)[C@@H](C)C(=O)NCCc1cccc(N)c1)OC)N(C)C(=O)[C@@H](NC(=O)[C@H](C(C)C)N(C)C)C(C)C]
21 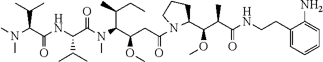 [SMILES: CC[C@H](C)[C@@H]([C@@H](CC(=O)N1CCC[C@H]1[C@H](OC)[C@@H](C)C(=O)NCCc1ccccc1N)OC)N(C)C(=O)[C@@H](NC(=O)[C@H](C(C)C)N(C)C)C(C)C]
22

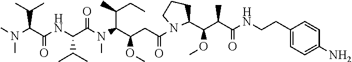 [SMILES: CC[C@H](C)[C@@H]([C@@H](CC(=O)N1CCC[C@H]1[C@H](OC)[C@@H](C)C(=O)NCCc1ccc(N)cc1)OC)N(C)C(=O)[C@@H](NC(=O)[C@H](C(C)C)N(C)C)C(C)C]

These results showed that methyl substitution on the N between P4 and P5 retained potency. This was unexpected and in sharp contrast to other well-known auristatin derivatives when methylation occurs. For examples, N-methylation of the amide between P4 and P5 moieties of Auristatin E, Auristatin PHE and Dolastatin 10 molecules led to 23- to 240-fold decrease in potency. This highlights the unique and unexpected properties of the ethylene functionality of the P5 moiety in compound 1.

TABLE 6B
HCC1954 5-Day Assay

| | IC50[N—Me amide]/ |
|---|---|
| Compound No. | IC50[NH-amide] |
| Auristatin E | 23 |
| 23 | |
| Auristatin PHE | 139 |
| 25 | |
| Dolastatin 10 | 240 |

Exemplary compounds of the invention were conjugated to certain antibodies (e.g., Trastuzumab) and tested for potency. These conjugates showed a favored DAR of about 8 and aggregation (SEC) of about 1%.

These conjugates showed exceptional IC₅₀ in HCC1954 assay.

TABLE 7
Exemplary ADC (Trastuzumab) IC₅₀ in HCC1954 assay

| Compound No. | IC₅₀ |
|---|---|
| 28 | +++ |
| 29 | +++ |
| 30 | +++ |
| 31 | +++ |
| 32 | +++ |
| 33 | + |
| 37 | +++ |
| 38 | + |

+++: <5 nM;
++: 5-10 nM;
+: >10 nM

Applicant's disclosure is described herein in preferred embodiments with reference to the FIGURES, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, manuscripts, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### Equivalents

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula (I): 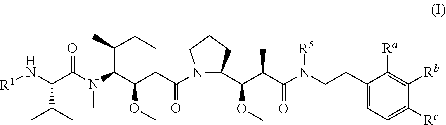
or a pharmaceutically acceptable salt thereof,
wherein
R¹ is 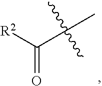
wherein R² is a unsubstituted or substituted C₁-C₆ alkyl, heteroalkyl, cycloalkyl or cycloheteroalkyl;
each of R^{a}, R^{b} and R^{c} is selected from H and NR^{x}R^{y}, provided that only one of R^{a}, R^{b} and R^{c} is NR^{x}R^{y} and each of the others is H;
each of R^{x} and R^{y} is independently selected from R, R^{r} and L-R^{z}, provided that when one of R^{x} and R^{y} is L-R^{z} or R^{r}, the other is R;
R⁵ is CR′₃, wherein each R′ is independently H or F;
L is a linker;
R^{r} is (C═O)—O—(CH₂)ₚ—R^{v} or (C═O)—(CH₂)_{q}—R^{v};
R^{v} is R, OR, NHR, NR₂, an aryl group or an amino acid;
p is 0, 1, 2, 3, 4, 5 or 6;
q is 0, 1, 2, 3, 4, 5 or 6;
R^{z} comprises a functional or reactive group; and
R is H or a C₁-C₃ alkyl.

2. The compound of claim 1, wherein R⁵ is CH₃.

3. The compound of claim 1, wherein R⁵ is CF₃.

4-7. (canceled)

8. The compound of claim 1, wherein R⁵ is CH₃, R^{a} is H, R^{c} is H and R^{b} is NR^{x}R^{y}, having the structural formula (III):

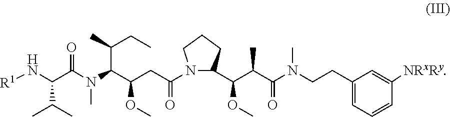

9-19. (canceled)

20. The compound of claim 1, wherein R⁵ is CH₃, R^{a} is H, R^{b} is H, and R^{c} is NR^{x}R^{y}, having the structural formula (V):

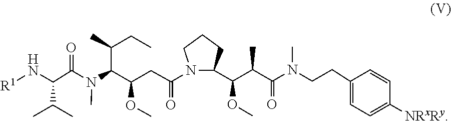

21. The compound of claim 20, wherein R^{x} is H and R^{y} is H, having the structural formula (V¹):

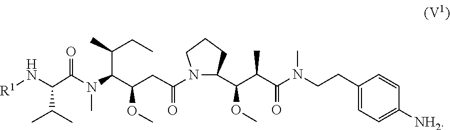

22. The compound of claim 20, wherein R^{x} is H or CH₃ and R^{y} is (C═O)—O—(CH₂)ₚ—R^{v}, wherein R^{v} is R, OR, NHR, NR₂, an aryl group or an amino acid, and p is 0, 1, 2 or 3.

23. The compound of claim 20, wherein R^{x} is H or CH₃ and R^{y} is (C═O)—(CH₂)_{q}—R^{v}, wherein R^{v} is R, OR, NHR, NR₂, an aryl group or an amino acid, and q is 0, 1, 2 or 3.

24. The compound of claim 20, wherein R^{y} is L-R^{z}, having the structural formula (V²):

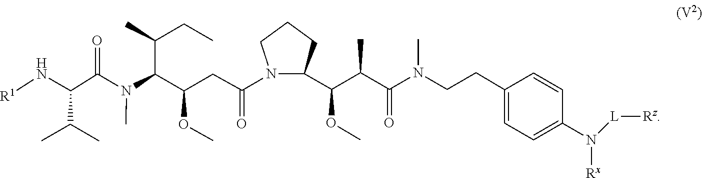

25. The compound of claim 24, wherein R^{x} is H, having the structural formula (V³):

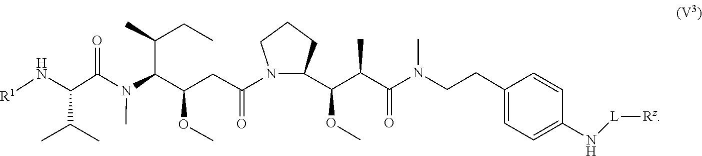

26. The compound of claim 1, wherein R⁵ is CF₃, R^{a} is H, R^{b} is H, and R^{c} is NR^{x}R^{y}, having the structural formula (V⁴):

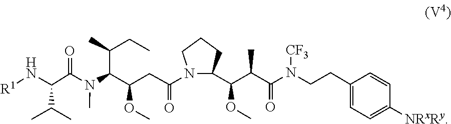

27-30. (canceled)

31. The compound of claim 1, wherein R¹ is 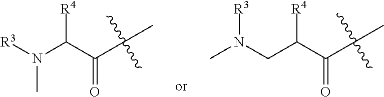
wherein each of R³ and R⁴ is independently H or an unsubstituted or substituted C₁-C₅ alkyl, or together with the N and C atoms they are boned to form a 5- to 7-membered heterocycloalkyl comprising one or more of O, N and S, optionally substituted with one or more of halogen atoms or C₁-C₃ alkyl.

32. (canceled)

33. The compound of claim 31, wherein R⁴ is isopropyl or methyl.

34. (canceled)

35. The compound of claim 31, wherein R³ and R⁴, together with the N and C atoms they are boned to form a 5-membered heterocycloalkyl comprising N, optionally substituted with one or more of halogen atoms or C₁-C₃ alkyl.

36. The compound of claim 31, wherein R³ and R⁴, together with the N and C atoms they are boned to form a 6-membered heterocycloalkyl comprising N, optionally substituted with one or more of halogen atoms or C₁-C₃ alkyl.

37. The compound of claim 1, wherein R¹ is selected from:

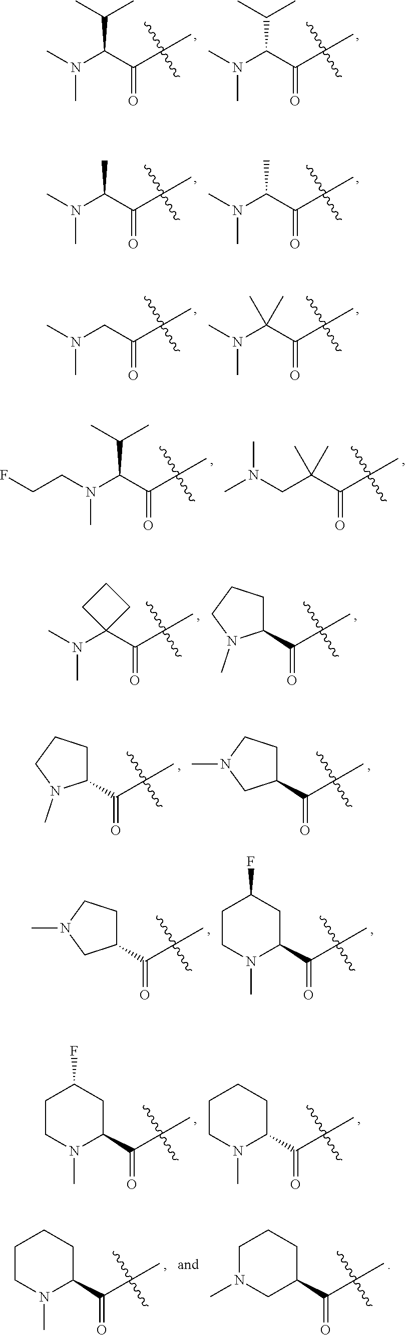

38. The compound of claim 1, wherein L is a noncleavable linker.

39. The compound of claim 1, wherein L is a cleavable linker.

40-44. (canceled)

45. The compound of claim 1, wherein R^{z} comprises a functional or reactive group selected from:
—N₃, —NR^{u}C(═O)CH═CH₂, —SH, —SSR^{t}, —S(═O)₂(CH═CH₂), —(CH₂)₂S(=O)₂(CH═CH₂), —NR^{u}S(=O₂)(CH═CH₂), —NR^{u}C(═O)CH₂R^{w}, —NR^{u}C(═O)CH₂Br, —NR^{u}C(═O)CH₂I, —NHC(═O)CH₂Br, NHC(═O)CH₂I, —ONH₂, —C(═O)NHNH₂, —CO₂H, —NH₂, —NCO, —NCS, 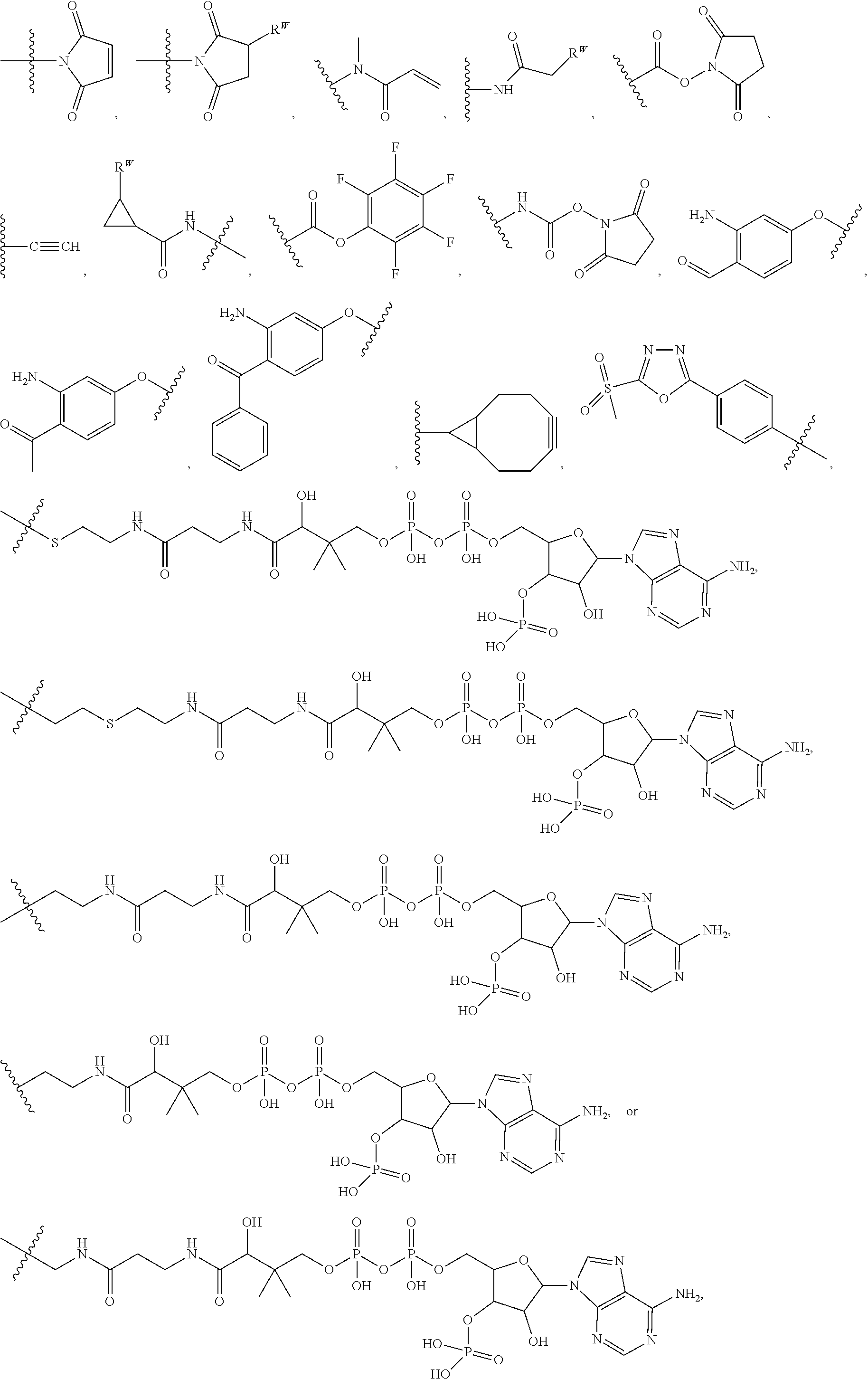
wherein
R^{u} is H or a C₁-C₆ alkyl group,
R^{t} is 2-pyridyl or 4-pyridyl, and
R^{w} is

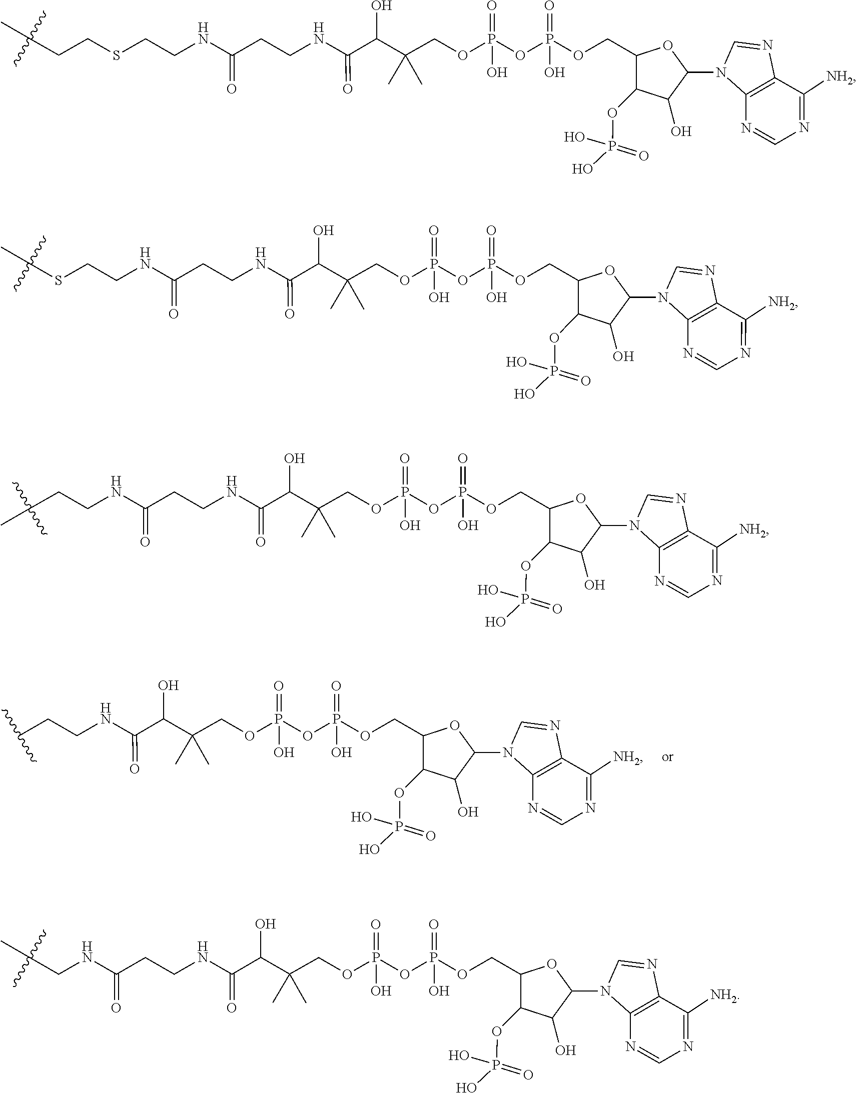

46. A compound selected from:

| Compound | |
|---|---|
| No. | Structure |
| 1 | 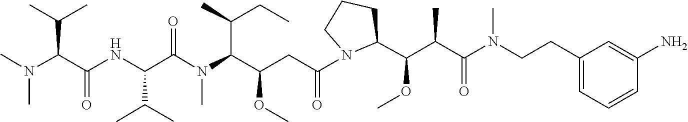 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@H](C(C)C)N(C)C)C(C)C] |
| 2 | 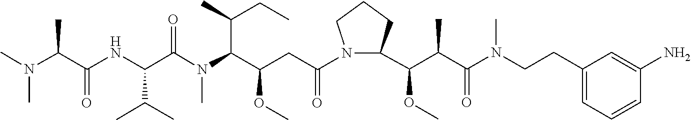 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@H](C)N(C)C)C(C)C] |
| 3 | 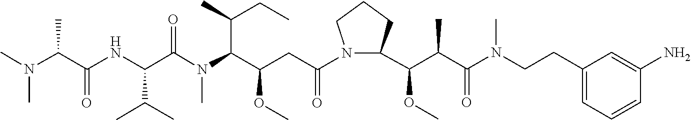 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H](C)N(C)C)C(C)C] |
| 4 | 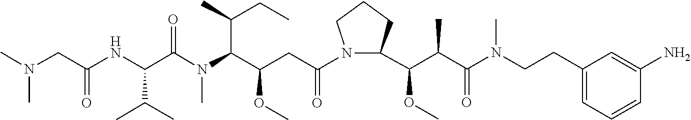 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)CN(C)C)C(C)C] |
| 5 | 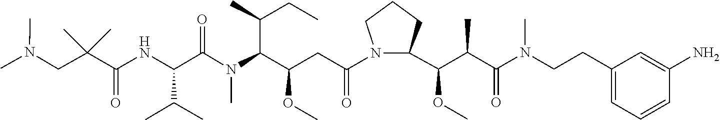 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)C(C)(C)CN(C)C)C(C)C] |
| 6 | 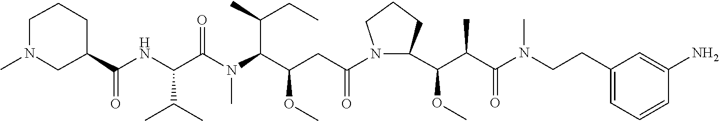 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCCN(C)C1)C(C)C] |
| 7 | 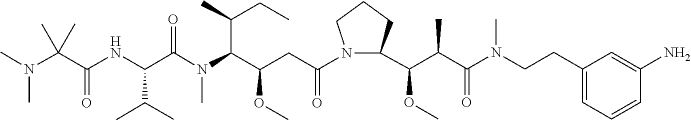 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)C(C)(C)N(C)C)C(C)C] |
| 8 | 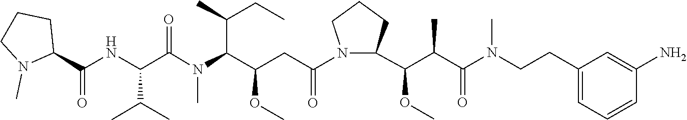 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCCN1C)C(C)C] |
| 9 | 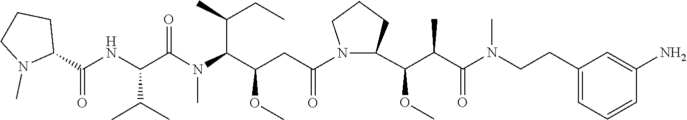 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@H]1CCCN1C)C(C)C] |
| 10 | 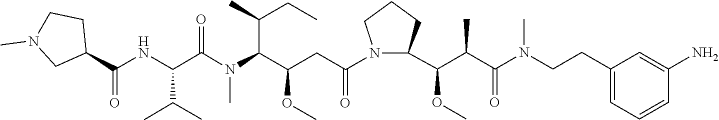 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCN(C)C1)C(C)C] |
| 11 | 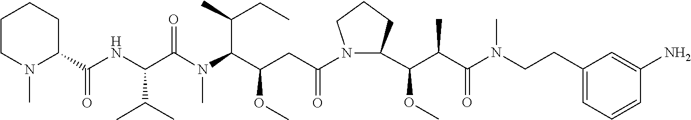 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@H]1CCCCN1C)C(C)C] |
| 12 | 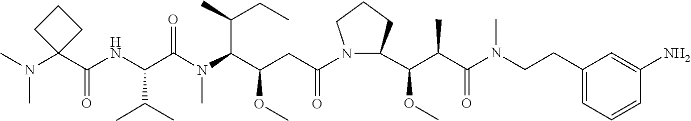 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)C1(N(C)C)CCC1)C(C)C] |
| 13 | 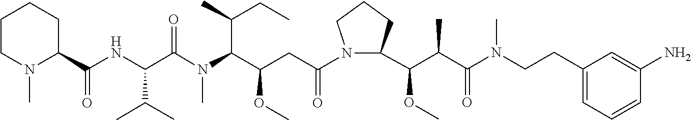 [SMILES: CC[C@H](C)[C@@H]([C@@H](CC(=O)N1CCC[C@H]1[C@H](OC)[C@@H](C)C(=O)N(C)CCc1cccc(N)c1)OC)N(C)C(=O)[C@@H](NC(=O)[C@@H]1CCCCN1C)C(C)C] |
| 14 | 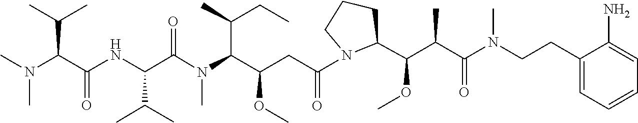 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@H](C(C)C)N(C)C)C(C)C] |
| 15 | 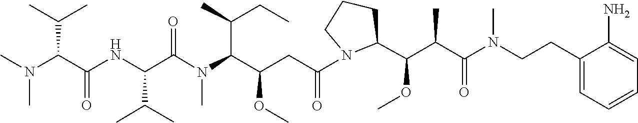 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@@H](C(C)C)N(C)C)C(C)C] |
| 16 | 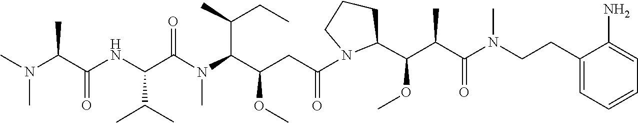 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@H](C)N(C)C)C(C)C] |
| 17 | 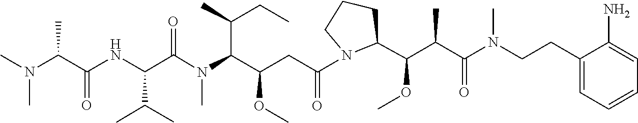 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)[C@@H](C)N(C)C)C(C)C] |
| 18 | 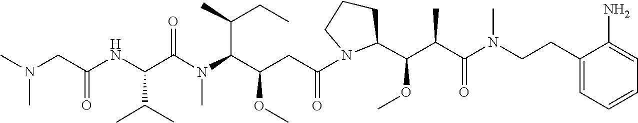 [SMILES: CC[C@H](C)[C@@H]([C@@H](CC(=O)N1CCC[C@H]1[C@H](OC)[C@@H](C)C(=O)N(C)CCc1ccccc1N)OC)N(C)C(=O)[C@@H](NC(=O)CN(C)C)C(C)C] |
| 19 | 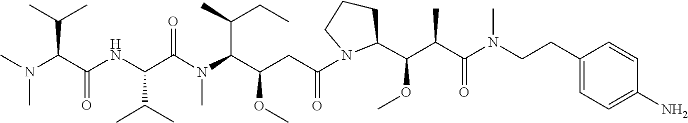 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@H](C(C)C)N(C)C)C(C)C] |
| 23 | 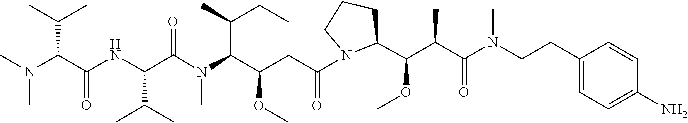 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@@H](C(C)C)N(C)C)C(C)C] |
| 24 | 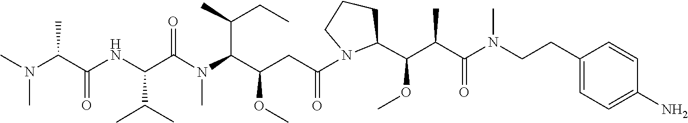 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@@H](C)N(C)C)C(C)C] |
| 26 | 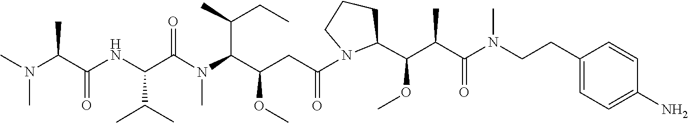 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)[C@H](C)N(C)C)C(C)C] |
| 29 | 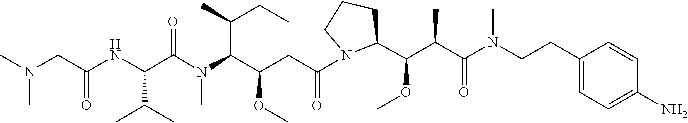 [SMILES: CC[C@H](C)[C@@H]([C@@H](CC(=O)N1CCC[C@H]1[C@H](OC)[C@@H](C)C(=O)N(C)CCc1ccc(N)cc1)OC)N(C)C(=O)[C@@H](NC(=O)CN(C)C)C(C)C] |

47. A drug-linker conjugate formed by conjugation of a compound of claim 1 with a linker.

48. An immunoconjugate formed by conjugation of a compound of claim 1, via a linker, with an antigen binding moiety.

49-54. (canceled)

55. An immunoconjugate having the structural formula (VIII): 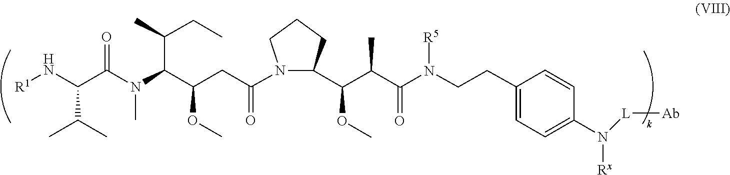
or a pharmaceutically acceptable salt thereof,
wherein
Ab represents an antigen binding moiety;
R¹ is 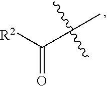
wherein R² is a unsubstituted or substituted C₁-C₆ alkyl, heteroalkyl, cycloalkyl or cycloheteroalkyl;
each of R^{x} and R^{y} is independently selected from R and L-R^{z}, provided that when one of R^{x} and R^{y} is NR^{z}, the other is R;
R⁵ is CR′₃, wherein each R′ is independently H or F;
L is a linker; and
R is H or a C₁-C₃ alkyl; and
k is an integer in the range of 1 to about 20.

56-80. (canceled)

81. A pharmaceutical composition comprising an immunoconjugate of claim 55, or a pharmaceutically acceptable salt thereof, and a pharmaceutically acceptable excipient, carrier or diluent.

82. (canceled)

83. A method for treating cancer, comprising administering to a subject in need thereof a therapeutically effective amount of an immunoconjugate of claim 55.

84-89. (canceled)
